(19) United States
(12) Patent Application Publication
(10) Pub. No.: US 2025/0066385 A1
(43) Pub. Date: Feb. 27, 2025

(54) ORGANIC COMPOUND, ORGANIC ELECTROLUMINESCENT DEVICE, AND ELECTRONIC APPARATUS

(71) Applicant: Shaanxi Lighte Optoelectronics Material Co., Ltd., Xi'an (CN)

(72) Inventors: Tiantian MA, Xi'an (CN); Lei YANG, Xi'an (CN)

(21) Appl. No.: 18/720,295

(22) PCT Filed: Jun. 28, 2023
(86) PCT No.: PCT/CN2023/103475

§ 371 Date: Jun. 14, 2024

(30) Foreign Application Priority Data
Aug. 22, 2022 (CN) 202211008191.7

(51) Int. Cl. C07D 498/04; C07B 59/00; C07D 413/04; C07D 413/10; C07D 413/14; C07F 7/08; C09K 11/06; H10K 85/40; H10K 85/60

(52) U.S. Cl. CPC C07D 498/04; C07B 59/004; C07D 413/04; C07D 413/10; C07D 413/14; C07F 7/0812; C09K 11/06; H10K 85/40; H10K 85/622; H10K 85/654; H10K 85/656; H10K 85/6572; H10K 85/6574; H10K 85/6576; C07B 2200/05

(57) ABSTRACT

The disclosure relates to an organic compound, an organic electroluminescent device, and an electronic apparatus. The organic compound of the disclosure has a structure shown in a formula 1, and when the organic compound is applied to an organic electroluminescent device, the performance of the device can be significantly improved.

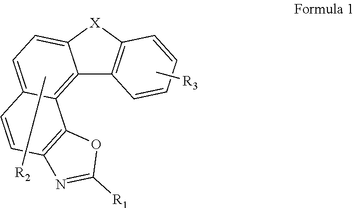

### CROSS-REFERENCE TO RELATED APPLICATIONS

This application is a National Stage Entry of International Application No. PCT/CN2023/103475, filed Jun. 28, 2023, and claims priority to Chinese patent application No. 202211008191.7, filed Aug. 22, 2022, the disclosures of which are incorporated herein by reference in their entireties.

### TECHNICAL FIELD

The disclosure relates to the technical field of organic compounds, in particular to an organic compound, an organic electroluminescent device including the same, and an electronic apparatus.

### BACKGROUND OF THE INVENTION

With the development of electronic technology and the progress of material science, the application range of electronic components for realizing electroluminescence or photoelectric conversion is wider and wider. An organic electroluminescent device (OLED) typically includes a cathode and an anode which are oppositely disposed, and a functional layer disposed between the cathode and the anode. The functional layer is composed of a plurality of organic or inorganic film layers, and generally includes an organic light-emitting layer, a hole transport layer, an electron transport layer, and the like. When a voltage is applied to the cathode and the anode, an electric field is generated between the two electrodes, electrons on a cathode side move towards the organic light-emitting layer and holes on an anode side also move towards the organic light-emitting layer under the action of the electric field. The electrons and the holes are combined in the organic light-emitting layer to form excitons. The excitons are in an excited state and release energy outwards, and then the organic light-emitting layer emits light outwards. According to the statistical theorem of electron spins, singlet excitons and triplet excitons are generated in a ratio of 1:3. The limit of internal quantum efficiency of a fluorescence-type organic electroluminescent device using luminescence using singlet excitons is 25%. On the other hand, it is known that the internal quantum efficiency can be increased to 100% in the case where intersystemcrossing from singlet excitons is performed efficiently in a phosphorescence-type organic electroluminescent device using luminescence using triplet excitons.

Nonetheless, at present, there are still many problems for phosphorescent materials in terms of organic electroluminescent materials. For example: short service life and low efficiency. Thus, it is necessary to develop new materials, improving the performance of electronic components.

The information disclosed by the background part is only used for enhancing the understanding of the background of the disclosure, so the information can include information which does not constitute the prior art known to those of ordinary skill in the art.

### SUMMARY OF THE INVENTION

In order to solve the above problems, an object of the disclosure is to provide an organic compound, an organic electroluminescent device including the organic compound, and an electronic apparatus. The organic compound can improve the performance of the organic electroluminescent device and the electronic apparatus, such as reducing the driving voltage of the device, and improving the efficiency and service life of the device.

In a first aspect of the disclosure, provided is an organic compound, having a structure as shown in a formula 1:

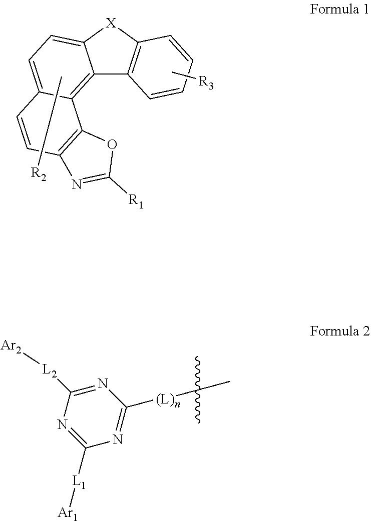

wherein, X is selected from C(R₄R₅), O, S or N(R₆);
R₁ and R₆ are the same or different, and are respectively and independently selected from substituted or unsubstituted aryl with 6 to 30 carbon atoms, substituted or unsubstituted heteroaryl with 3 to 30 carbon atoms, and a group represented by a formula 2;
R₂ and R₃ are the same or different, and are respectively and independently selected from hydrogen, deuterium, cyano, a halogen group, substituted or unsubstituted aryl with 6 to 30 carbon atoms, substituted or unsubstituted heteroaryl with 3 to 30 carbon atoms, and a group represented by the formula 2;
one and only one of R₁, R₆, R₂ and R₃ is the group represented by the formula 2;
R₄ and R₅ are the same or different, and are respectively and independently selected from alkyl with 1 to 10 carbon atoms, substituted or unsubstituted aryl with 6 to 30 carbon atoms, and substituted or unsubstituted heteroaryl with 3 to 30 carbon atoms;
each L, L₁ and L₂ are the same or different, and are respectively and independently selected from a single bond, substituted or unsubstituted arylene with 6 to 30 carbon atoms, and substituted or unsubstituted heteroarylene with 3 to 30 carbon atoms;
n is the number of groups L linked in sequence, and selected from 1, 2 or 3, and when n is greater than 1, any two L are the same or different;
Ar₁ and Ar₂ are the same or different, and are respectively and independently selected from substituted or unsubstituted aryl with 6 to 30 carbon atoms and substituted or unsubstituted heteroaryl with 3 to 30 carbon atoms; and
substituent(s) in R₁, R₂, R₃, R₄, R₅, R₆, L, L₁, L₂, Ar₁ and Ar₂ are the same or different, and are respectively and independently selected from deuterium, a halogen group, cyano, alkyl with 1 to 10 carbon atoms, cycloalkyl with 3 to 20 carbon atoms, heteroaryl with 3 to 20 carbon atoms, aryl with 6 to 20 carbon atoms, deuteroaryl with 6 to 20 carbon atoms, haloaryl with 6 to 20 carbon atoms, trialkylsilyl with 3 to 12 carbon atoms, haloalkyl with 1 to 10 carbon atoms, and deuteroalkyl with 1 to 10 carbon atoms.

In a second aspect of the disclosure, provided is an organic electroluminescent device, including an anode and a cathode which are oppositely disposed, and a functional layer disposed between the anode and the cathode, where the functional layer includes the above organic compound.

In a third aspect of the disclosure, provided is an electronic apparatus, including the organic electroluminescent device according to the second aspect.

A core group of the organic compound of the disclosure is formed by fusing a dibenzo five-membered ring and benzoxazole in a specific manner, the core group having a planar ring-like structure. By bonding this core group to triazine, the compound can have a strong carrier transport capability and a high energy transfer capability while maintaining a high value of a first triplet energy level. When the organic compound of the disclosure is used as a host material for an organic light-emitting layer in a red organic electroluminescent device, the performance of the device can be significantly improved.

Other features and advantages of the disclosure will be described in detail in the subsequent specific embodiments.

### BRIEF DESCRIPTION OF DRAWINGS

The accompanying drawings are used to provide a further understanding of the disclosure and constitute a part of the description, and are used to explain the disclosure together with the following specific embodiments, but do not constitute limitations on the disclosure.

FIG. 1 is a structural schematic diagram of an organic electroluminescent device according to the disclosure.

FIG. 2 is a structural schematic diagram of an electronic apparatus according to the disclosure.

### REFERENCE SIGNS

100, anode; 200, cathode; 300, functional layer; 310, hole injection layer;
320, first hole transport layer; 330, second hole transport layer; 340, organic light-emitting layer;
350, electron transport layer; 360, electron injection layer; 400, electronic apparatus.

### DETAILED DESCRIPTION OF THE INVENTION

In view of the above problems existing in the prior art, an object of the disclosure is to provide an organic compound, an organic electroluminescent device including the organic compound, and an electronic apparatus. The organic compound can improve the performance of the organic electroluminescent device and the electronic apparatus, such as reducing the driving voltage of the device, and improving the efficiency and service life of the device.

In a first aspect of the disclosure, provided is an organic compound, having a structure as shown in a formula 1:

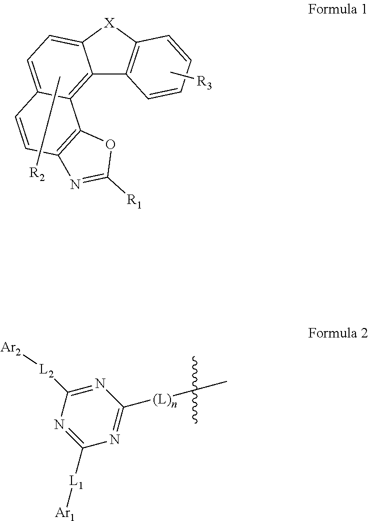

wherein, X is selected from C(R₄R₅), O, S or N(R₆);
R₁ and R₆ are the same or different, and are respectively and independently selected from substituted or unsubstituted aryl with 6 to 30 carbon atoms, substituted or unsubstituted heteroaryl with 3 to 30 carbon atoms, and a group represented by a formula 2;
R₂ and R₃ are the same or different, and are respectively and independently selected from hydrogen, deuterium, cyano, a halogen group, substituted or unsubstituted aryl with 6 to 30 carbon atoms, substituted or unsubstituted heteroaryl with 3 to 30 carbon atoms, and a group represented by the formula 2;
one and only one of R₁, R₆, R₂ and R₃ is the group represented by the formula 2;
R₄ and R₅ are the same or different, and are respectively and independently selected from alkyl with 1 to 10 carbon atoms, substituted or unsubstituted aryl with 6 to 30 carbon atoms, and substituted or unsubstituted heteroaryl with 3 to 30 carbon atoms;
each L, L₁ and L₂ are the same or different, and are respectively and independently selected from a single bond, substituted or unsubstituted arylene with 6 to 30 carbon atoms, and substituted or unsubstituted heteroarylene with 3 to 30 carbon atoms;
n is the number of groups L linked in sequence, and selected from 1, 2 or 3, and when n is greater than 1, any two L are the same or different;
Ar₁ and Ar₂ are the same or different, and are respectively and independently selected from substituted or unsubstituted aryl with 6 to 30 carbon atoms, and substituted or unsubstituted heteroaryl with 3 to 30 carbon atoms; and
substituent(s) in R₁, R₂, R₃, R₄, R₅, R₆, L, L₁, L₂, Ar₁ and Ar₂ are the same or different, and are respectively and independently selected from deuterium, a halogen group, cyano, alkyl with 1 to 10 carbon atoms, cycloalkyl with 3 to 20 carbon atoms, heteroaryl with 3 to 20 carbon atoms, aryl with 6 to 20 carbon atoms, deuteroaryl with 6 to 20 carbon atoms, haloaryl with 6 to 20 carbon atoms, trialkylsilyl with 3 to 12 carbon atoms, haloalkyl with 1 to 10 carbon atoms, and deuteroalkyl with 1 to 10 carbon atoms.

In the disclosure, the adopted description modes “each . . . is independently”, “ . . . is respectively and independently“and” . . . is each independently” can be interchanged, and should be understood in a broad sense, which means that in different groups, specific options expressed between the same symbols do not influence each other, or in a same group, specific options expressed between the same symbols do not influence each other. For example, the meaning of

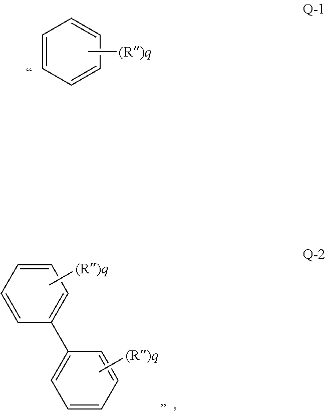

wherein, each q is independently 0, 1, 2 or 3, and each R″ is independently selected from hydrogen, deuterium, fluorine and chlorine” is as follows: a formula Q-1 represents that q substituents R″ exist on a benzene ring, each R″ can be the same or different, and options of each R″ do not influence each other; and a formula Q-2 represents that each benzene ring of biphenyl has q substituents R″, the number q of the substituents R″ on the two benzene rings can be the same or different, each R″ can be the same or different, and options of each R″ do not influence each other.

In the disclosure, the term such as “substituted or unsubstituted” means that a functional group described behind the term may have or not have a substituent (in the following, the substituent is collectively referred to as Rc in order to facilitate description). For example, the “substituted or unsubstituted aryl” refers to aryl having the substituent Rc or unsubstituted aryl. The above substituent, i.e., Rc, may be, for example, deuterium, cyano, a halogen group, alkyl, haloalkyl, deuteroalkyl, trialkylsilyl, aryl, deuteroaryl, heteroaryl, cycloalkyl, or the like. The number of the substituents may be one or more.

In the disclosure, “a plurality of” refers to two or more, e.g., two, three, four, five, six, etc.

In the disclosure, the number of carbon atoms in a substituted or unsubstituted functional group refers to the number of all carbon atoms. For example, if L₁ is substituted arylene with 12 carbon atoms, the number of all carbon atoms of the arylene and substituents on the arylene is 12.

In the disclosure, aryl refers to an optional functional group or substituent derived from an aromatic carbocyclic ring. The aryl may be monocyclic aryl (e.g., phenyl) or polycyclic aryl, in other words, the aryl can be monocyclic aryl, fused aryl, two or more monocyclic aryl conjugatedly connected through carbon-carbon bonds, monocyclic aryl and fused aryl which are conjugatedly connected through a carbon-carbon bond, or two or more fused aryl conjugatedly connected through carbon-carbon bonds. That is, unless otherwise noted, two or more aromatic groups conjugatedly connected through carbon-carbon bonds can also be regarded as the aryl of the disclosure. The fused aryl may include, for example, bicyclic fused aryl (e.g., naphthyl), tricyclic fused aryl (e.g., phenanthryl, fluorenyl, and anthryl), and the like. The aryl does not contain heteroatoms such as B, N, O, S, P, Se and Si. Examples of the aryl can include, but are not limited to, phenyl, naphthyl, fluorenyl, anthryl, phenanthryl, biphenyl, terphenyl, triphenylene, perylenyl, benzo[9,10]phenanthryl, pyrenyl, benzofluoranthenyl, chrysenyl, spirobifluorenyl, and the like. In the disclosure, the arylene involved refers to a divalent group formed by further loss of one hydrogen atom from aryl.

In the disclosure, terphenyl includes

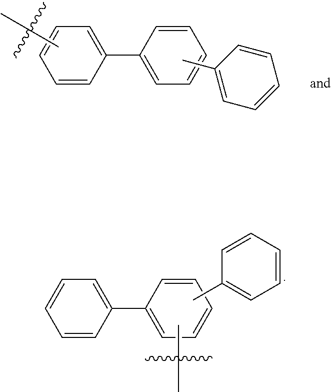

In the disclosure, the number of carbon atoms of the substituted aryl refers to the total number of carbon atoms of the aryl and substituents on the aryl, for example, substituted aryl with 18 carbon atoms means that the total number of carbon atoms of the aryl and substituents is 18.

In the disclosure, the number of carbon atoms of the substituted or unsubstituted aryl may be 6, 10, 12, 13, 14, 15, 16, 17, 18, 20, 25 or 30. In some embodiments, the substituted or unsubstituted aryl is substituted or unsubstituted aryl with 6 to 30 carbon atoms, in other embodiments, the substituted or unsubstituted aryl is substituted or unsubstituted aryl with 6 to 25 carbon atoms, in other embodiments, the substituted or unsubstituted aryl is substituted or unsubstituted aryl with 6 to 20 carbon atoms, in other embodiments, the substituted or unsubstituted aryl is substituted or unsubstituted aryl with 6 to 18 carbon atoms, and in other embodiments, the substituted or unsubstituted aryl is substituted or unsubstituted aryl with 6 to 12 carbon atoms.

In the disclosure, fluorenyl may be substituted by one or more substituents, where any two adjacent substituents may be bonded to each other to form a substituted or unsubstituted spirocyclic structure. In the case where the above fluorenyl is substituted, the substituted fluorenyl may be

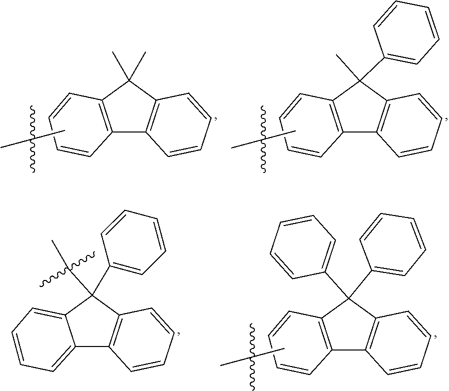

or the like, but is not limited to this.

In the disclosure, specific examples of aryl as a substituent in R₁, R₂, R₃, R₄, R₅, R₆, L, L₁, L₂, Ar₁ and Ar₂ include, but is not limited to, phenyl, naphthyl, and the like.

In the disclosure, heteroaryl refers to a monovalent aromatic ring containing 1, 2, 3, 4, 5 or 6 heteroatoms in the ring or its derivative, and the heteroatom may be one or more of B, O, N, P, Si, Se and S. The heteroaryl may be monocyclic heteroaryl or polycyclic heteroaryl, in other words, the heteroaryl may be a single aromatic ring system or a plurality of aromatic ring systems conjugatedly connected through carbon-carbon bonds, where any one aromatic ring system is one aromatic monocyclic ring or one aromatic fused ring. For example, the heteroaryl may include thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, phenothiazinyl, silafluorenyl, dibenzofuranyl, as well as N-phenylcarbazolyl, N-pyridylcarbazolyl, N-methylcarbazolyl and the like, but is not limited to this.

In the disclosure, the number of carbon atoms of the substituted or unsubstituted heteroaryl may be selected from 3, 4, 5, 6, 7, 8, 9, 10, 11, 12, 13, 14, 15, 16, 17, 18, 20, 21, 22, 23, 24, 25, 26, 27, 28, 29 or 30. In some embodiments, the substituted or unsubstituted heteroaryl is substituted or unsubstituted heteroaryl having a total number of carbon atoms being 5 to 20, and in other embodiments, the substituted or unsubstituted heteroaryl is substituted or unsubstituted heteroaryl having a total number of carbon atoms being 12 to 18.

In the disclosure, the substituted heteroaryl may be that one or two or more hydrogen atoms in the heteroaryl are substituted by groups such as a deuterium atom, a halogen group, —CN, aryl, heteroaryl, trialkylsilyl, alkyl, cycloalkyl, haloalkyl, and the like. It should be understood that the number of carbon atoms of the substituted heteroaryl refers to the total number of carbon atoms of heteroaryl and substituents on the heteroaryl.

In the disclosure, the alkyl with 1 to 10 carbon atoms may include linear alkyl with 1 to 10 carbon atoms and branched alkyl with 3 to 10 carbon atoms. The number of carbon atoms of the alkyl may be, for example, 1, 2, 3, 4, 5, 6, 7, 8, 9, or 10, and specific examples of the alkyl include, but are not limited to, methyl, ethyl, n-propyl, isopropyl, n-butyl, isobutyl, tert-butyl, n-pentyl, isopentyl, neopentyl, n-hexyl, and the like.

In the disclosure, the halogen group may be, for example, fluorine, chlorine, bromine or iodine.

In the disclosure, specific examples of trialkylsilyl include, but are not limited to, trimethylsilyl, and the like.

In the disclosure, specific examples of haloalkyl include, but are not limited to, trifluoromethyl.

In the disclosure, specific examples of deuteroalkyl include, but are not limited to, trideuteromethyl.

In the disclosure, the number of carbon atoms of cycloalkyl with 3 to 20 carbon atoms may be, for example, 3, 4, 5, 6, 7, 8, 10, 12, 16, or the like. Specific examples of the cycloalkyl include, but are not limited to, cyclopentyl, cyclohexyl, and adamantyl.

In the disclosure, an unpositioned connecting bond refers to a single bond

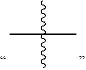

extending from a ring system, which means that one end of the connecting bond can be connected with any position in the ring system through which the bond penetrates, and the other end of the connecting bond is connected with the remaining part of a compound molecule. For example, as shown in the following formula (f), naphthyl represented by the formula (f) is connected to other positions of a molecule through two unpositioned connecting bonds penetrating a dicyclic ring, and its meaning includes any one possible connecting mode represented by formulae (f-1) to (f-10).

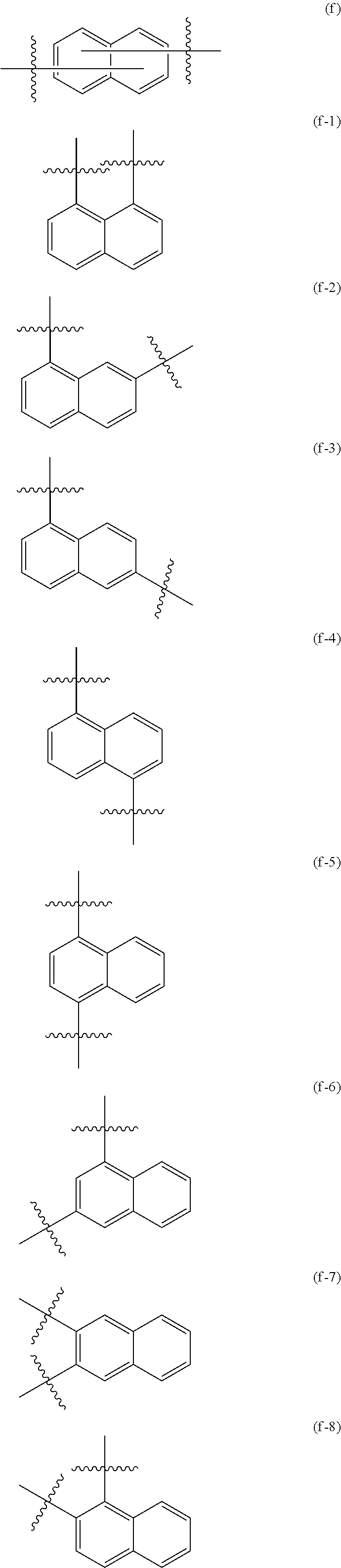

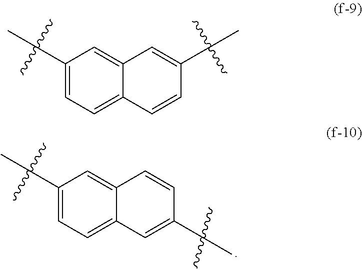 [SMILES: *c1ccc2ccc(*)cc2c1 ; *c1ccc2cc(*)ccc2c1]

For another example, as shown in the following formula (X′), dibenzofuranyl represented by the formula (X′) is connected with other positions of a molecule through one unpositioned connecting bond extending from the middle of a benzene ring on one side, and its meaning includes any one possible connecting mode represented by formulae (X′-1) to (X′-4).

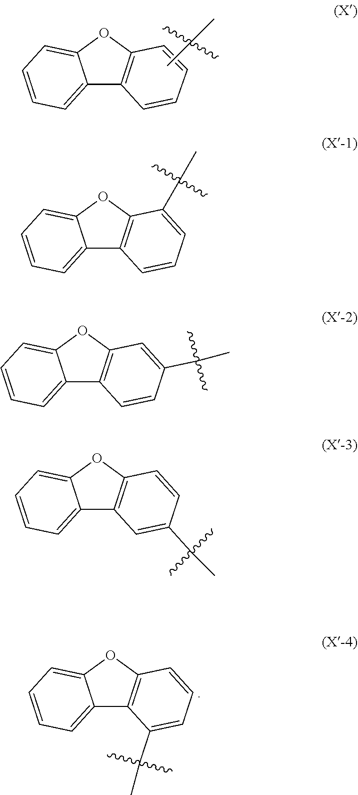

In some embodiments of the disclosure, the substituent(s) in R₁, R₂, R₃, R₄, R₅, R₆, L, L₁, L₂, Ar₁ and Ar₂ are the same or different, and are respectively and independently selected from deuterium, a halogen group, cyano, alkyl with 1 to 10 carbon atoms, cycloalkyl with 3 to 20 carbon atoms, heteroaryl with 3 to 20 carbon atoms, deuteroaryl with 6 to 20 carbon atoms, haloaryl with 6 to 20 carbon atoms, trialkylsilyl with 3 to 12 carbon atoms, haloalkyl with 1 to 10 carbon atoms, and deuteroalkyl with 1 to 10 carbon atoms.

In some embodiments of the disclosure, the organic compound has a structure represented by a formula 1-1, a formula 1-2, a formula 1-3, a formula 1-4, a formula 1-5, a formula 1-6, a formula 1-7, a formula 1-8, a formula 1-9, a formula 1-10, a formula 1-11, a formula 1-12, or a formula 1-13:

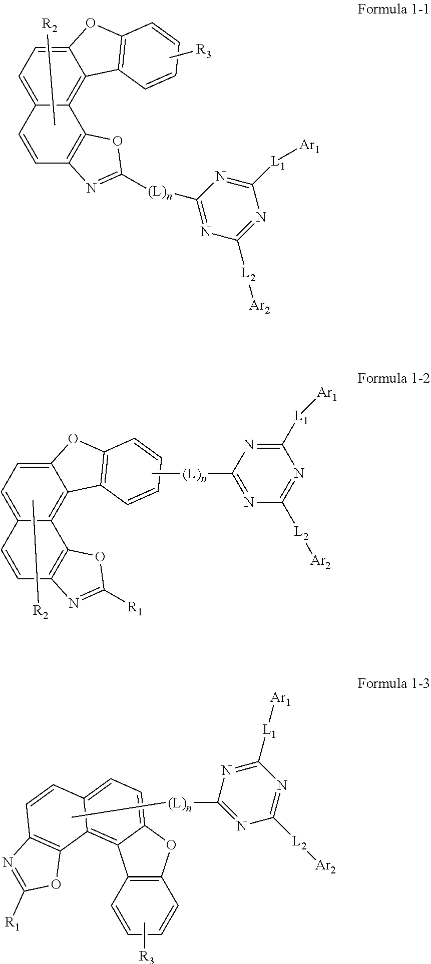

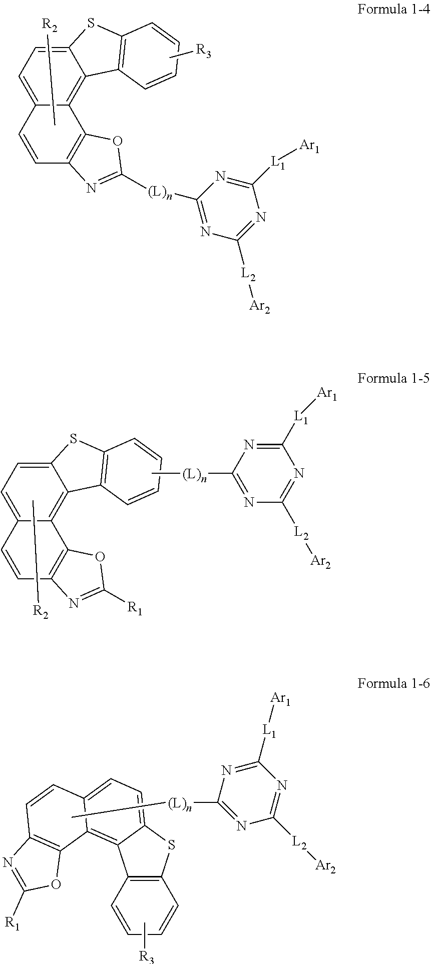

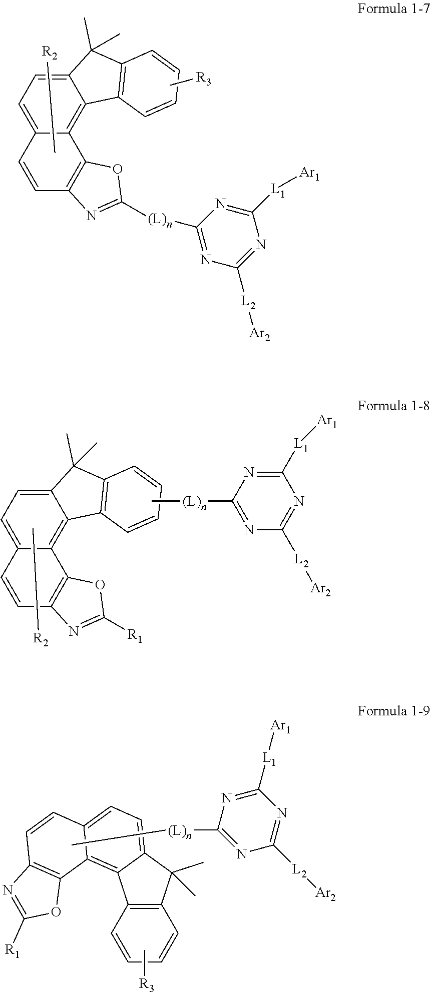

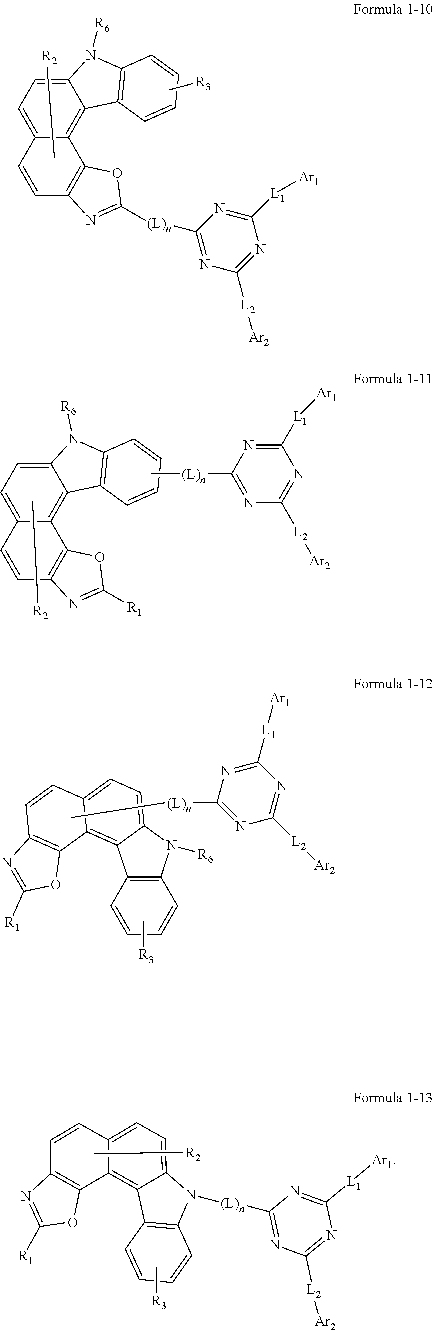

In some embodiments of the disclosure, n is selected from 1 or 2.

In some embodiments of the disclosure, each L is the same or different, and is independently selected from a single bond, and substituted or unsubstituted arylene with 6 to 12 carbon atoms.

Optionally, substituent(s) in L are the same or different, and are respectively and independently selected from deuterium, a halogen group, cyano, alkyl with 1 to 5 carbon atoms and phenyl.

In other embodiments of the disclosure, each L is the same or different, and is independently selected from a single bond, phenylene, naphthylene and biphenylene.

In some embodiments of the disclosure, each L is the same or different, and is independently selected from a single bond or the group consisting of:

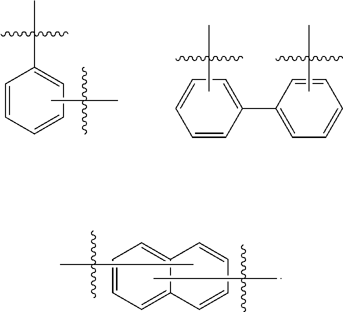

In particular, each L is the same or different, and is independently selected from a single bond or the group consisting of:

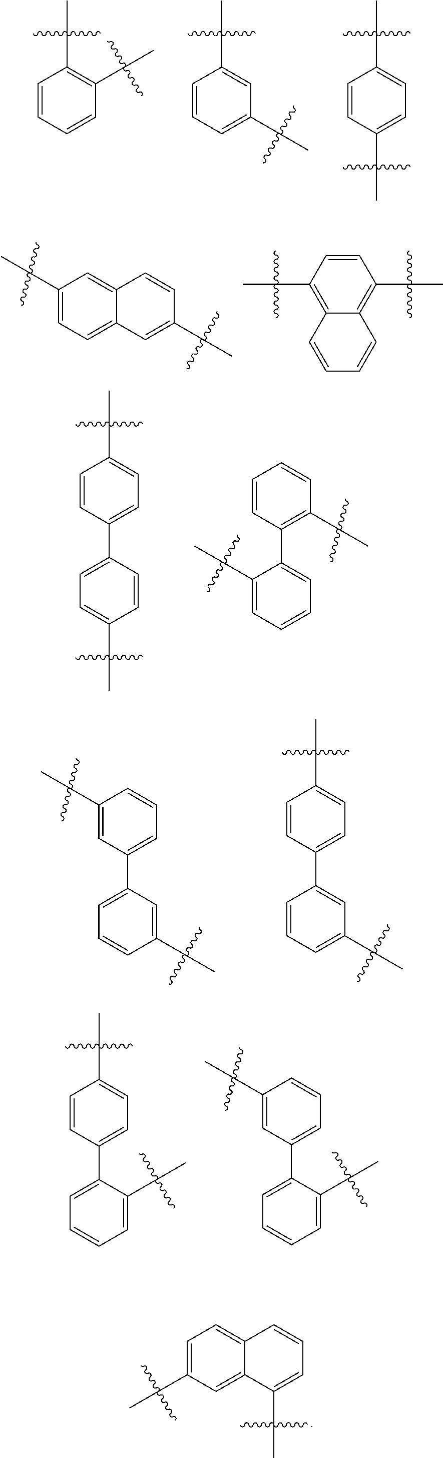

In some embodiments of the disclosure,

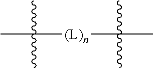

in the formula 2 is selected from a single bond or the group consisting of:

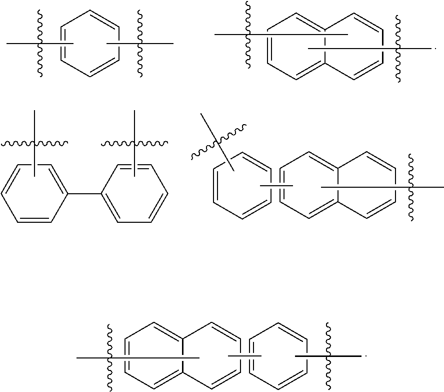

Optionally,

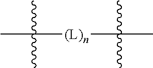

in the formula 2 is selected from a single bond or the group consisting of:

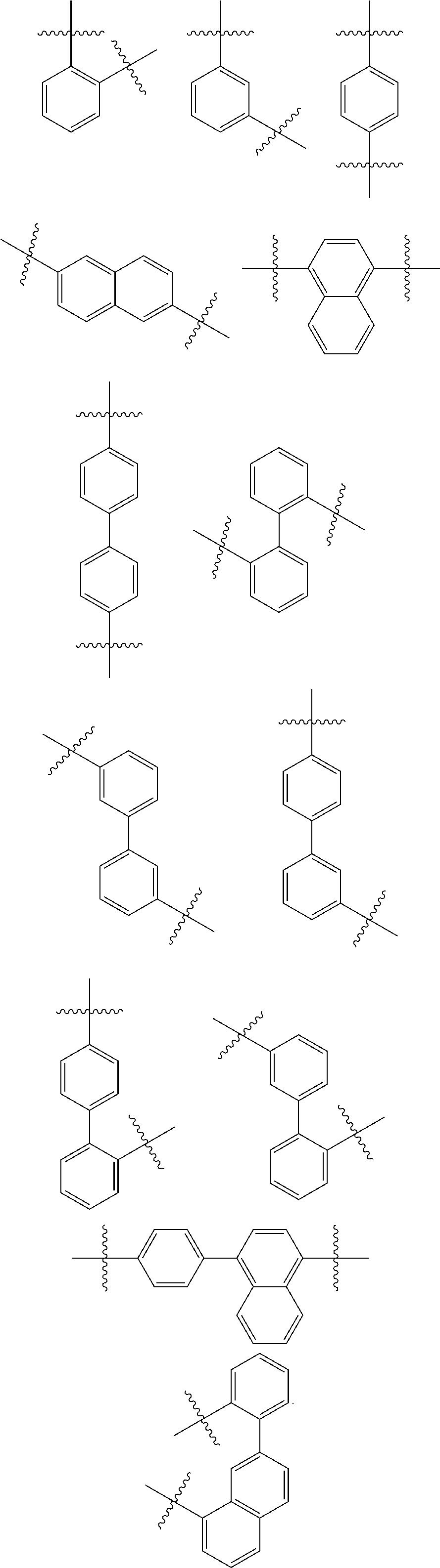

In some embodiments of the disclosure, L₁ and L₂ are the same or different, and are respectively and independently selected from a single bond, substituted or unsubstituted arylene with 6 to 12 carbon atoms, and substituted or unsubstituted heteroarylene with 12 to 18 carbon atoms.

Optionally, substituent(s) in L₁ and L₂ are the same or different, and are respectively and independently selected from deuterium, a halogen group, cyano, alkyl with 1 to 5 carbon atoms and phenyl.

In other embodiments of the disclosure, L₁ and L₂ are the same or different, and are respectively and independently selected from a single bond, substituted or unsubstituted phenylene, substituted or unsubstituted naphthylene, substituted or unsubstituted biphenylene, substituted or unsubstituted carbazolylene, substituted or unsubstituted dibenzofurylene, and substituted or unsubstituted dibenzothenylene.

Optionally, substituent(s) in L₁ and L₂ are the same or different, and are respectively and independently selected from deuterium, fluorine, cyano, methyl, ethyl, n-propyl, isopropyl, tert-butyl and phenyl.

In some embodiments of the disclosure, L₁ and L₂ are the same or different, and are respectively and independently selected from a single bond, and a substituted or unsubstituted group V, and the unsubstituted group V is selected from the group consisting of:

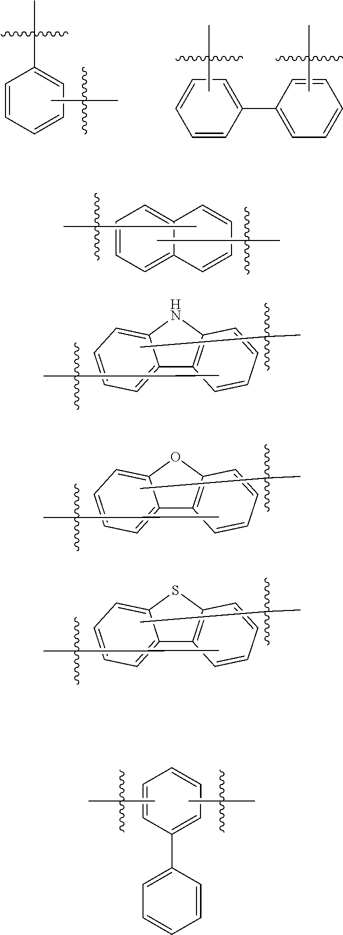

wherein,

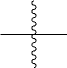

represents a chemical bond; and the substituted group V contains one or more substituents selected from deuterium, fluorine, cyano, methyl, ethyl, n-propyl, isopropyl, tert-butyl and phenyl; and when the substituted group V contains a plurality of substituents, the substituents are the same or different.

In some embodiments of the disclosure, L₁ and L₂ are the same or different, and are respectively and independently selected from a single bond and the group consisting of:

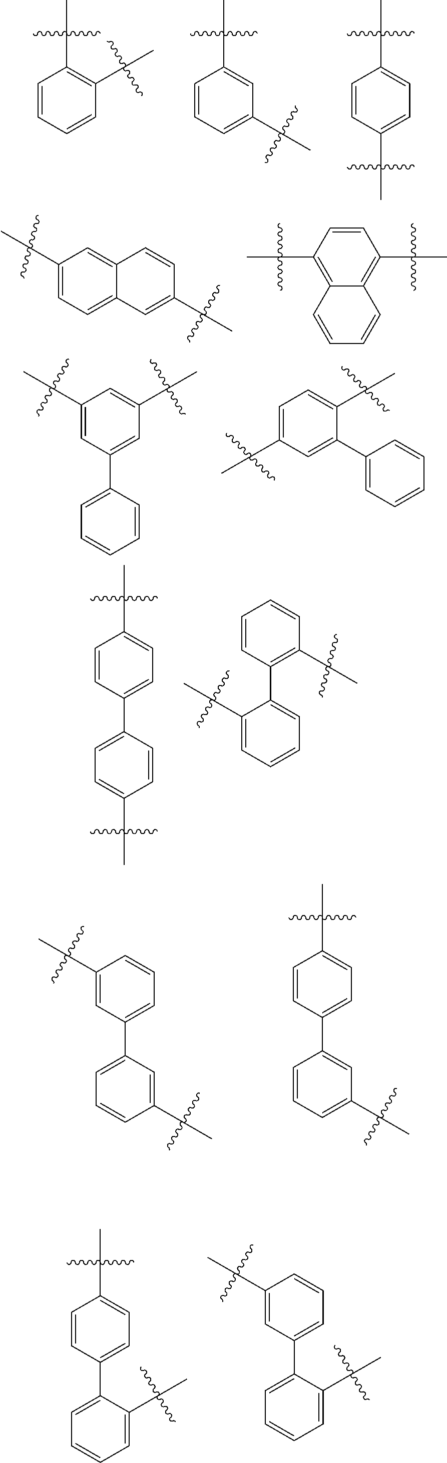

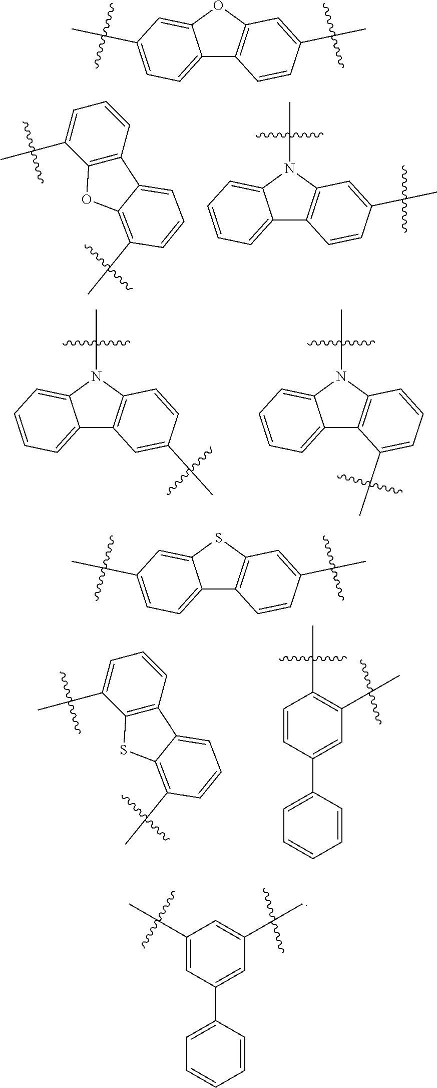

In some embodiments of the disclosure, Ar₁ and Ar₂ are the same or different, and are respectively and independently selected from substituted or unsubstituted aryl with 6 to 20 carbon atoms and substituted or unsubstituted heteroaryl with 5 to 20 carbon atoms.

Optionally, substituent(s) in Ar₁ and Ar₂ are the same or different, and are respectively and independently selected from deuterium, a halogen group, cyano, alkyl with 1 to 5 carbon atoms, haloalkyl with 1 to 5 carbon atoms, cycloalkyl with 1 to 10 carbon atoms, trimethylsilyl and phenyl.

In other embodiments of the disclosure, Ar₁ and Ar₂ are the same or different, and are respectively and independently selected from substituted or unsubstituted terphenyl, substituted or unsubstituted phenyl, substituted or unsubstituted naphthyl, substituted or unsubstituted biphenyl, substituted or unsubstituted phenanthryl, substituted or unsubstituted fluorenyl, substituted or unsubstituted triphenylene, substituted or unsubstituted pyridyl, substituted or unsubstituted carbazolyl, substituted or unsubstituted dibenzofuranyl, and substituted or unsubstituted dibenzothienyl.

Optionally, substituent(s) in Ar₁ and Ar₂ are the same or different, and are respectively and independently selected from deuterium, fluorine, cyano, methyl, ethyl, n-propyl, isopropyl, tert-butyl, cyclohexyl, trifluoromethyl, trimethylsilyl and phenyl.

In some embodiments of the disclosure, Ar₁ and Ar₂ are the same or different, and are respectively and independently selected from a substituted or unsubstituted group W, wherein, the unsubstituted group W is selected from the group consisting of:

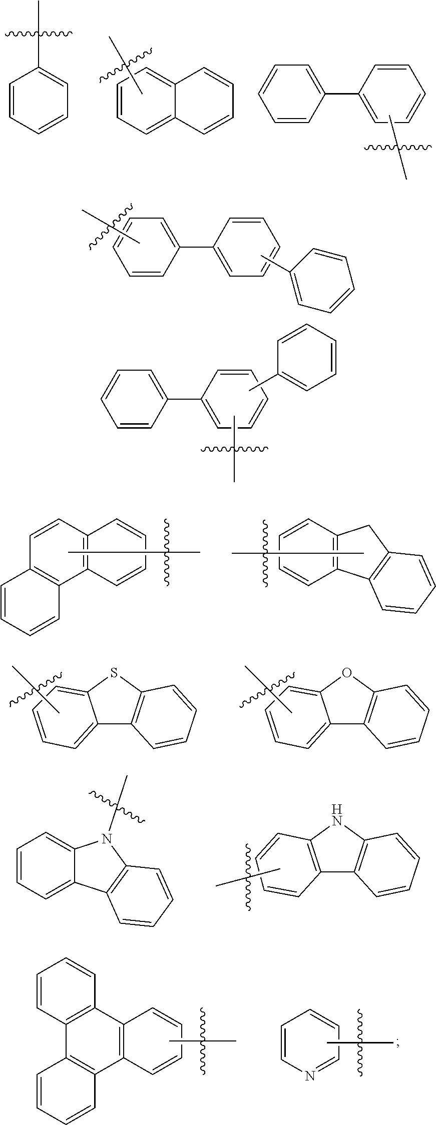

wherein,

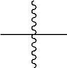

represents a chemical bond; and the substituted group W has one or two or more substituents each independently selected from deuterium, fluorine, cyano, cyclohexyl, methyl, ethyl, n-propyl, isopropyl, tert-butyl, phenyl, trimethylsilyl and trifluoromethyl, and when the number of the substituents on the group W is greater than 1, the substituents are the same or different.

Optionally, Ar₁ and Ar₂ are the same or different, and are respectively and independently selected from the group consisting of:

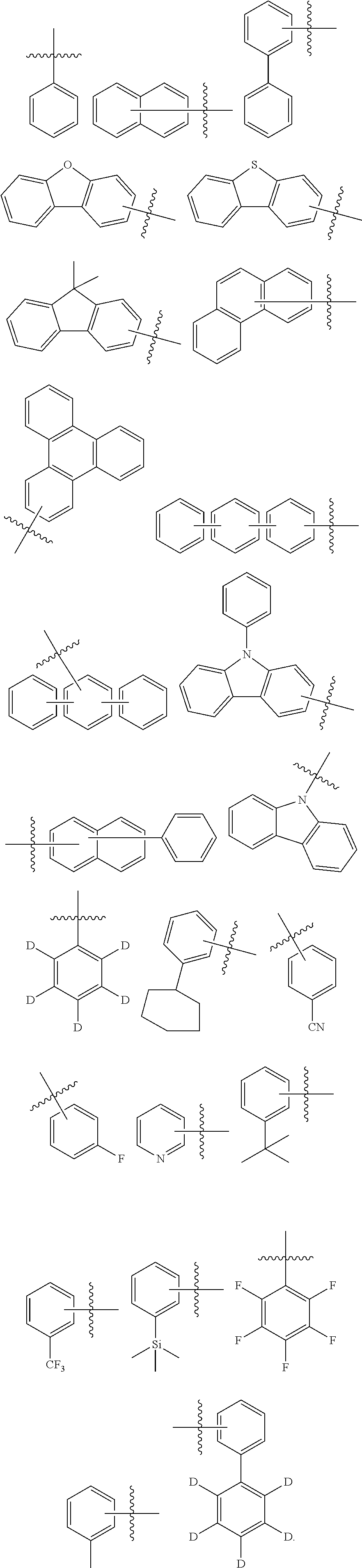

In particular, Ar₁ and Ar₂ are the same or different, and are respectively and independently selected from the group consisting of:

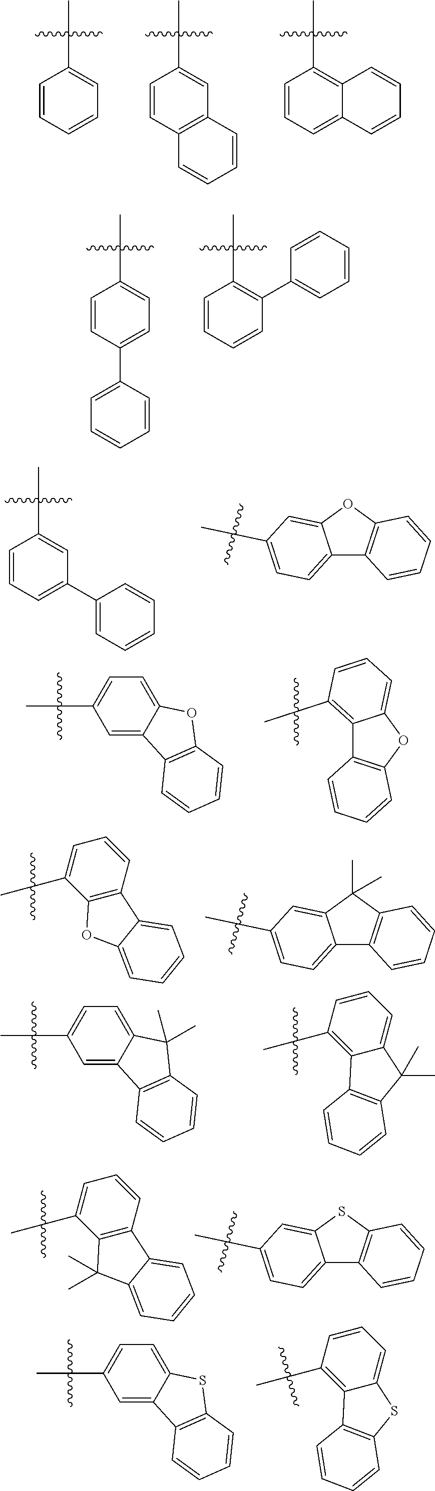

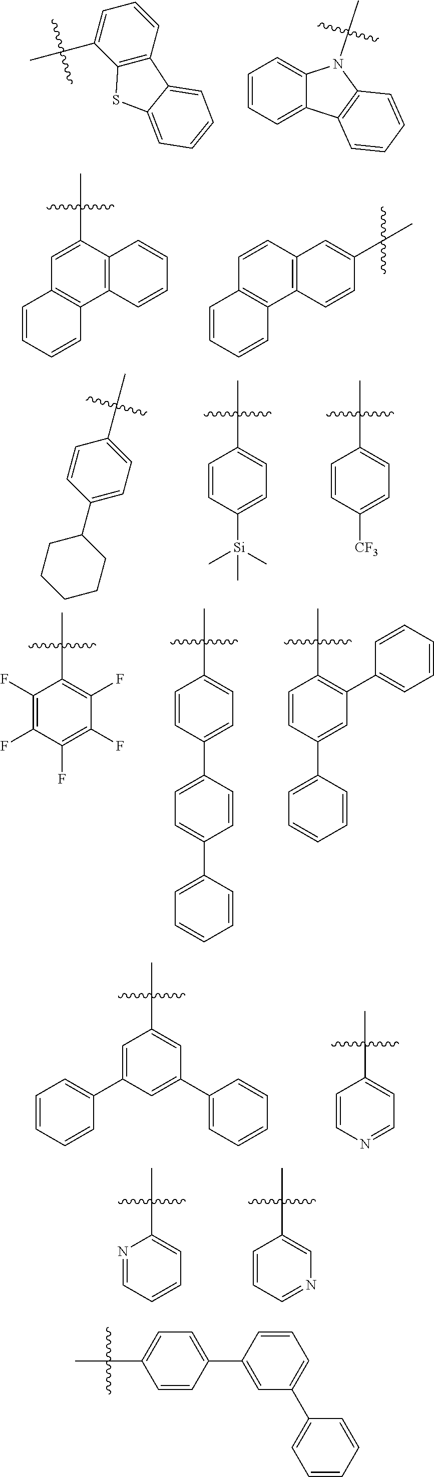

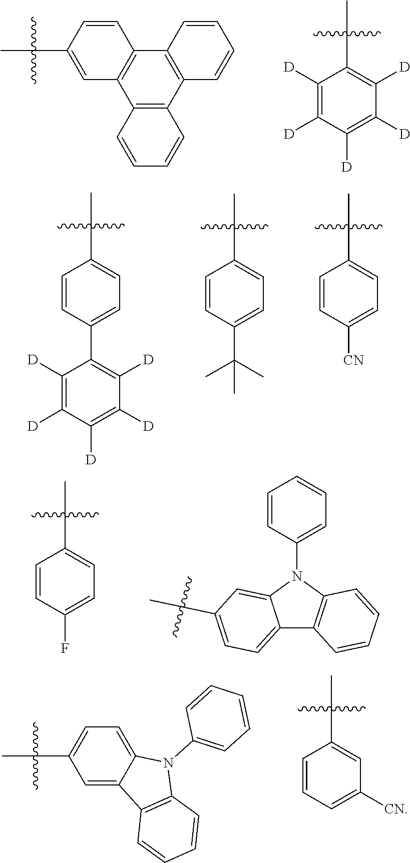

In some embodiments of the disclosure,

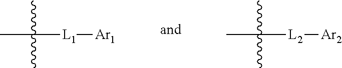

are the same or different, and are each independently selected from the group consisting of:

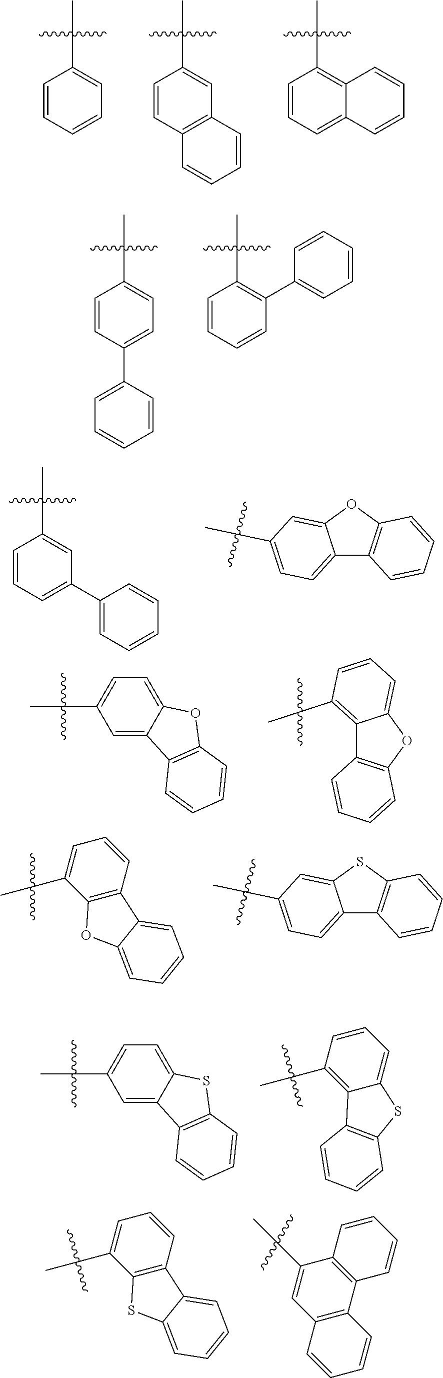

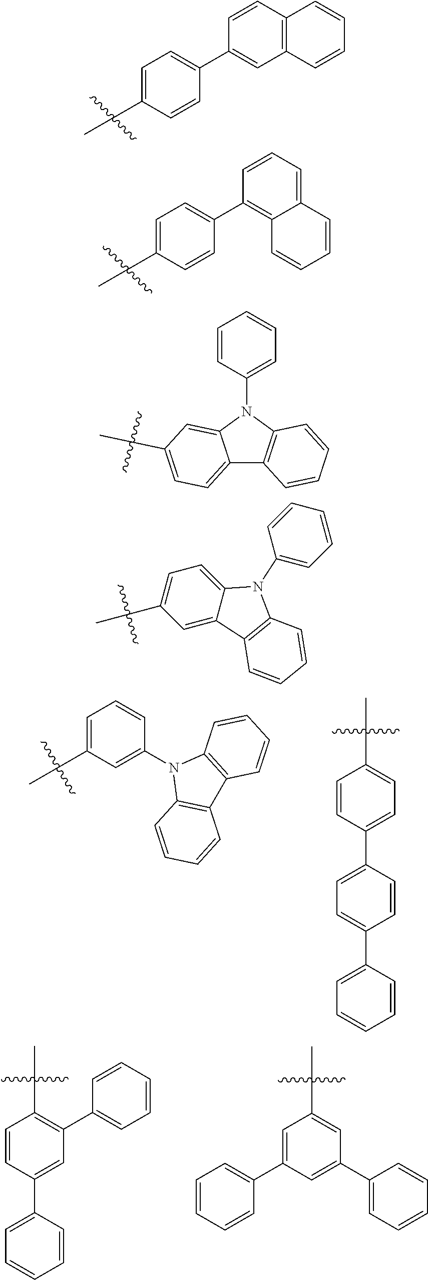

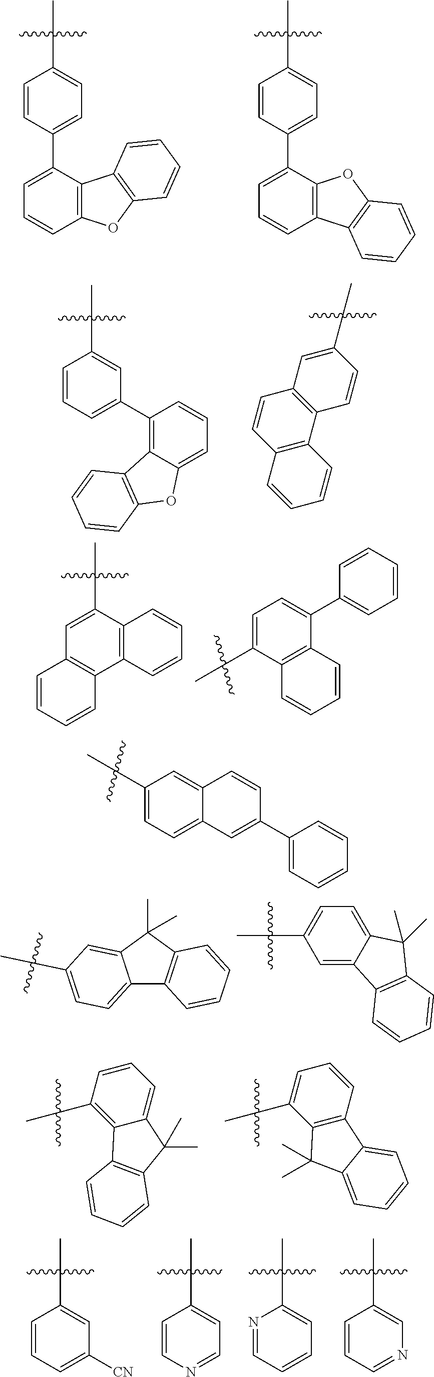

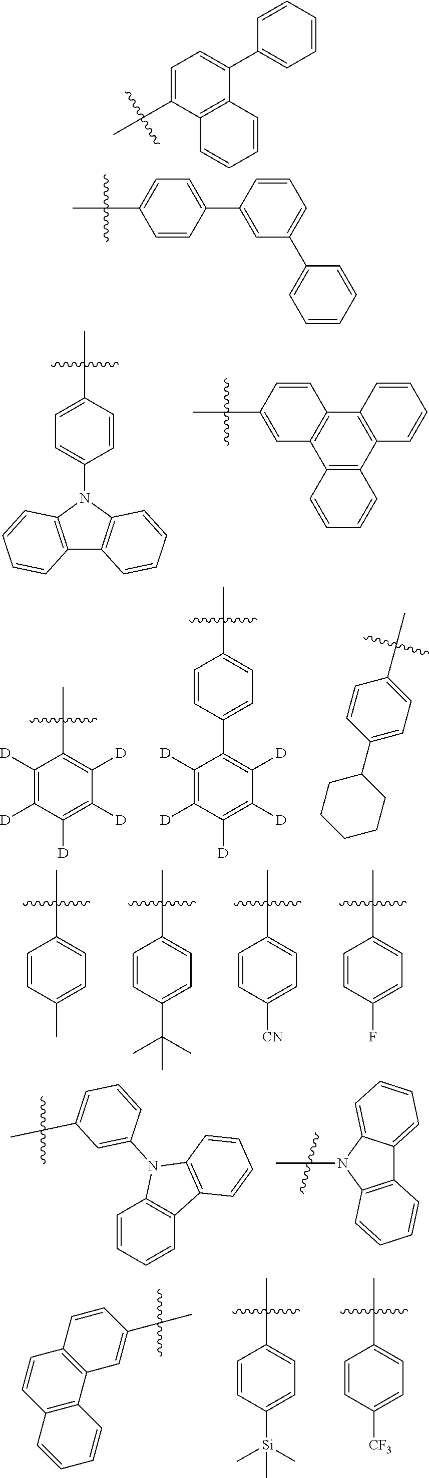

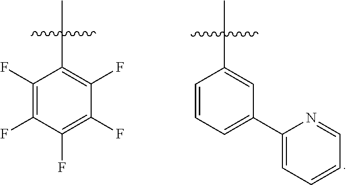 [SMILES: *c1c(F)c(F)c(F)c(F)c1F ; *c1cccc(-c2ccccn2)c1]

In some embodiments of the disclosure,

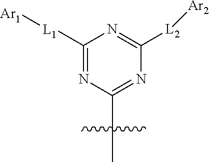

is selected from the following structures:

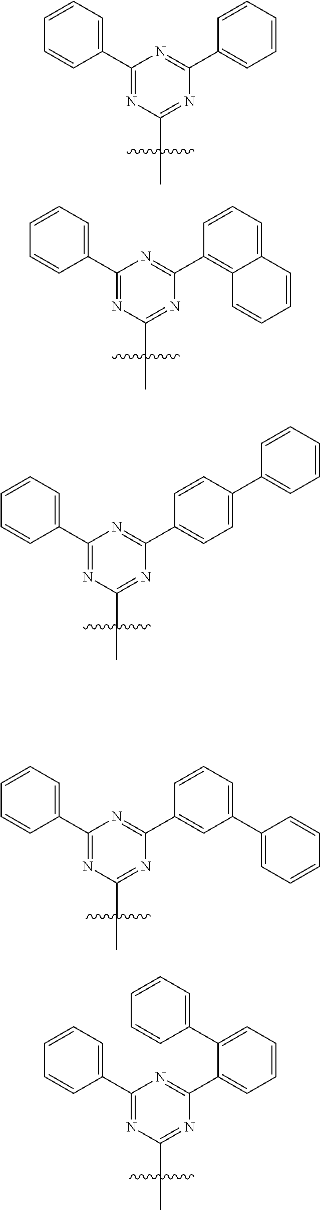 [SMILES: *c1nc(-c2ccccc2)nc(-c2ccccc2)n1 ; *c1nc(-c2ccccc2)nc(-c2cccc3ccccc23)n1 ; *c1nc(-c2ccccc2)nc(-c2ccc(-c3ccccc3)cc2)n1 ; *c1nc(-c2ccccc2)nc(-c2cccc(-c3ccccc3)c2)n1 ; *c1nc(-c2ccccc2)nc(-c2ccccc2-c2ccccc2)n1]

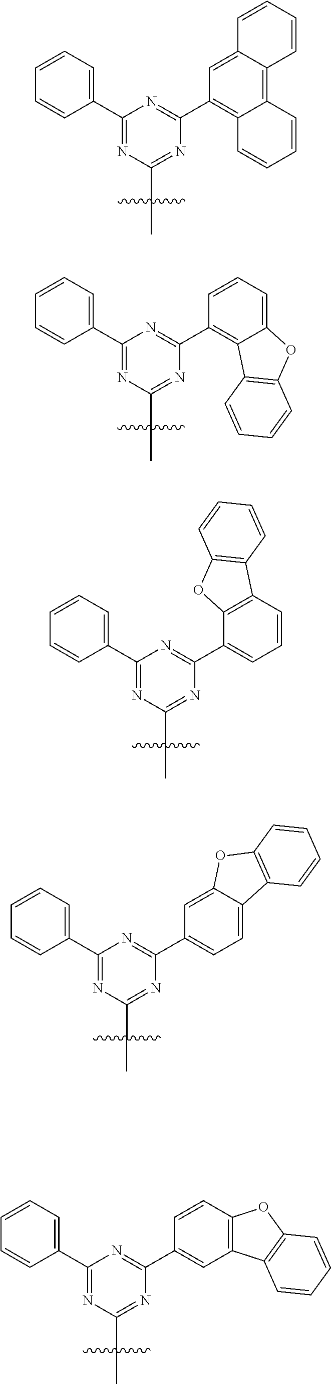 [SMILES: *c1nc(-c2ccccc2)nc(-c2cc3ccccc3c3ccccc23)n1 ; *c1nc(-c2ccccc2)nc(-c2cccc3oc4ccccc4c23)n1 ; *c1nc(-c2ccccc2)nc(-c2cccc3c2oc2ccccc23)n1 ; *c1nc(-c2ccccc2)nc(-c2ccc3c(c2)oc2ccccc23)n1 ; *c1nc(-c2ccccc2)nc(-c2ccc3oc4ccccc4c3c2)n1]

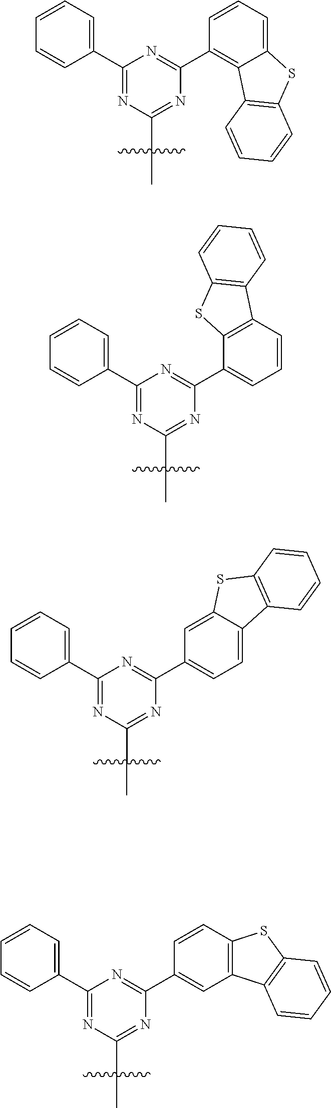 [SMILES: *c1nc(-c2ccccc2)nc(-c2cccc3sc4ccccc4c23)n1 ; *c1nc(-c2ccccc2)nc(-c2cccc3c2sc2ccccc23)n1 ; *c1nc(-c2ccccc2)nc(-c2ccc3c(c2)sc2ccccc23)n1 ; *c1nc(-c2ccccc2)nc(-c2ccc3sc4ccccc4c3c2)n1]

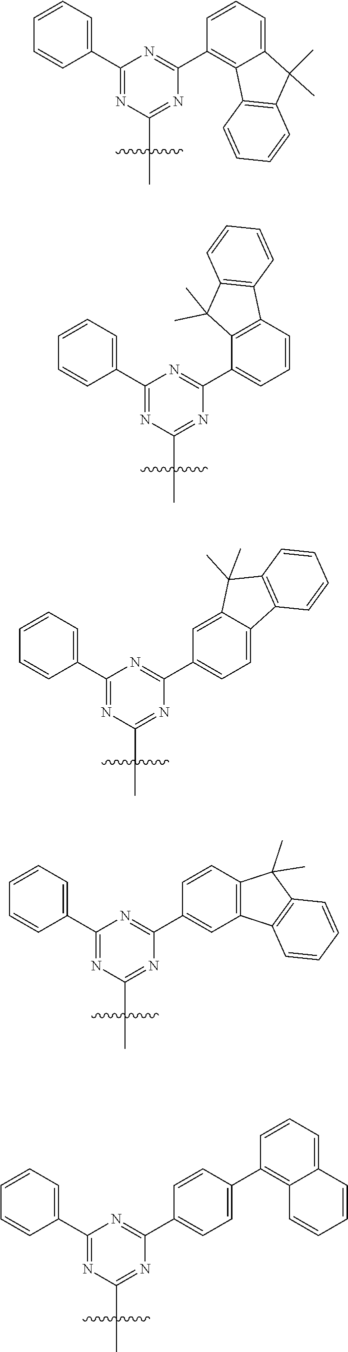 [SMILES: *c1nc(-c2ccccc2)nc(-c2cccc3c2-c2ccccc2C3(C)C)n1 ; *c1nc(-c2ccccc2)nc(-c2cccc3c2C(C)(C)c2ccccc2-3)n1 ; *c1nc(-c2ccccc2)nc(-c2ccc3c(c2)C(C)(C)c2ccccc2-3)n1 ; *c1nc(-c2ccccc2)nc(-c2ccc3c(c2)-c2ccccc2C3(C)C)n1 ; *c1nc(-c2ccccc2)nc(-c2ccc(-c3cccc4ccccc34)cc2)n1]

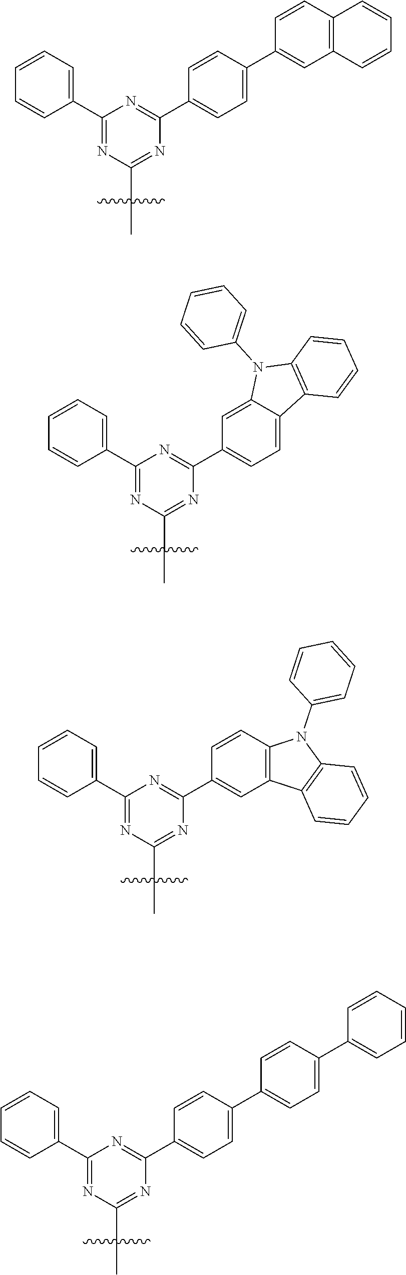 [SMILES: *c1nc(-c2ccccc2)nc(-c2ccc(-c3ccc4ccccc4c3)cc2)n1 ; *c1nc(-c2ccccc2)nc(-c2ccc3c4ccccc4n(-c4ccccc4)c3c2)n1 ; *c1nc(-c2ccccc2)nc(-c2ccc3c(c2)c2ccccc2n3-c2ccccc2)n1 ; *c1nc(-c2ccccc2)nc(-c2ccc(-c3ccc(-c4ccccc4)cc3)cc2)n1]

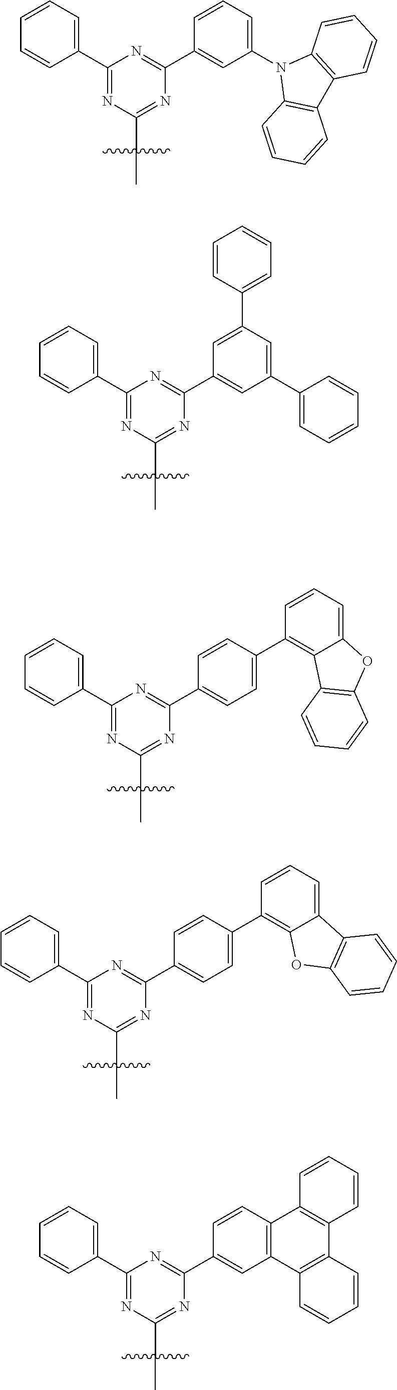 [SMILES: *c1nc(-c2ccccc2)nc(-c2cccc(-n3c4ccccc4c4ccccc43)c2)n1 ; *c1nc(-c2ccccc2)nc(-c2cc(-c3ccccc3)cc(-c3ccccc3)c2)n1 ; *c1nc(-c2ccccc2)nc(-c2ccc(-c3cccc4oc5ccccc5c34)cc2)n1 ; *c1nc(-c2ccccc2)nc(-c2ccc(-c3cccc4c3oc3ccccc34)cc2)n1 ; *c1nc(-c2ccccc2)nc(-c2ccc3c4ccccc4c4ccccc4c3c2)n1]

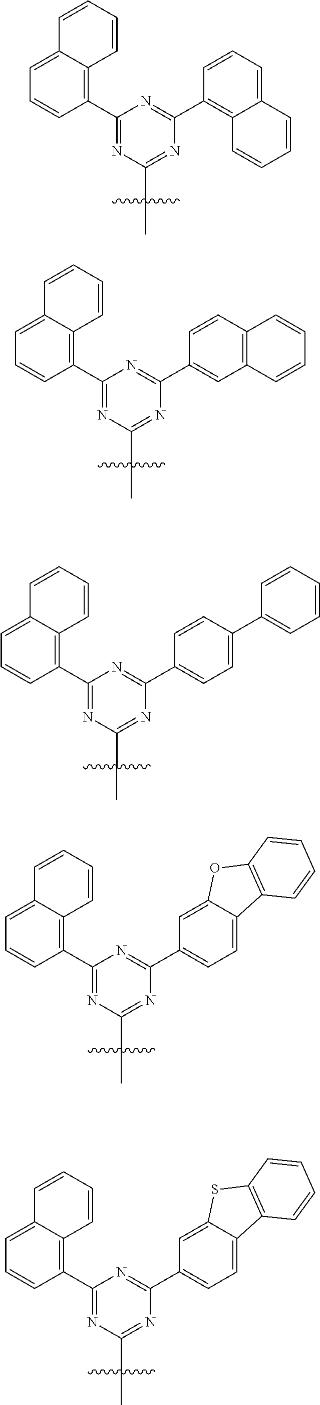 [SMILES: *c1nc(-c2cccc3ccccc23)nc(-c2cccc3ccccc23)n1 ; *c1nc(-c2ccc3ccccc3c2)nc(-c2cccc3ccccc23)n1 ; *c1nc(-c2ccc(-c3ccccc3)cc2)nc(-c2cccc3ccccc23)n1 ; *c1nc(-c2ccc3c(c2)oc2ccccc23)nc(-c2cccc3ccccc23)n1 ; *c1nc(-c2ccc3c(c2)sc2ccccc23)nc(-c2cccc3ccccc23)n1]

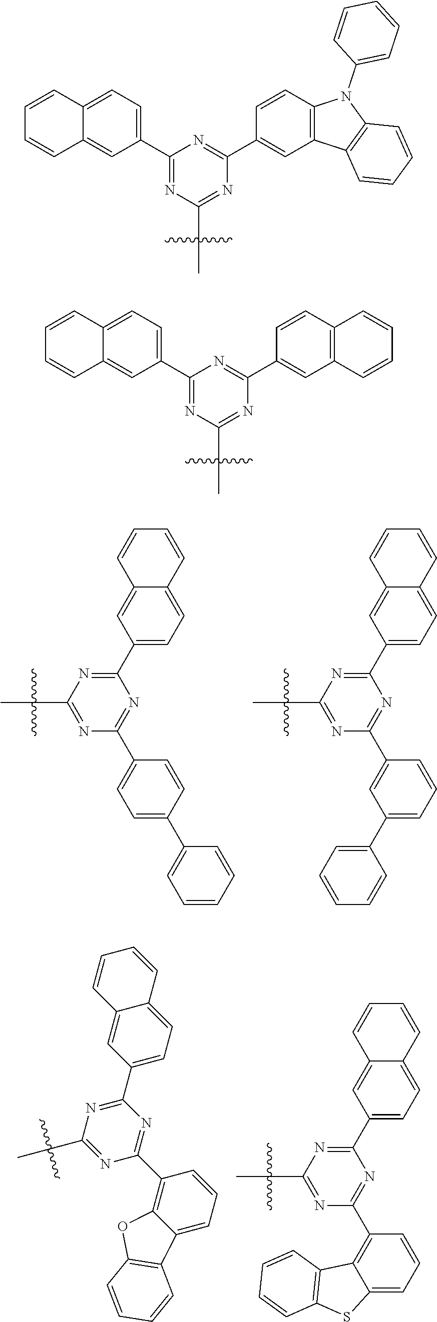 [SMILES: *c1nc(-c2ccc3ccccc3c2)nc(-c2ccc3c(c2)c2ccccc2n3-c2ccccc2)n1 ; *c1nc(-c2ccc3ccccc3c2)nc(-c2ccc3ccccc3c2)n1 ; *c1nc(-c2ccc(-c3ccccc3)cc2)nc(-c2ccc3ccccc3c2)n1 ; *c1nc(-c2cccc(-c3ccccc3)c2)nc(-c2ccc3ccccc3c2)n1 ; *c1nc(-c2ccc3ccccc3c2)nc(-c2cccc3c2oc2ccccc23)n1 ; *c1nc(-c2ccc3ccccc3c2)nc(-c2cccc3sc4ccccc4c23)n1]

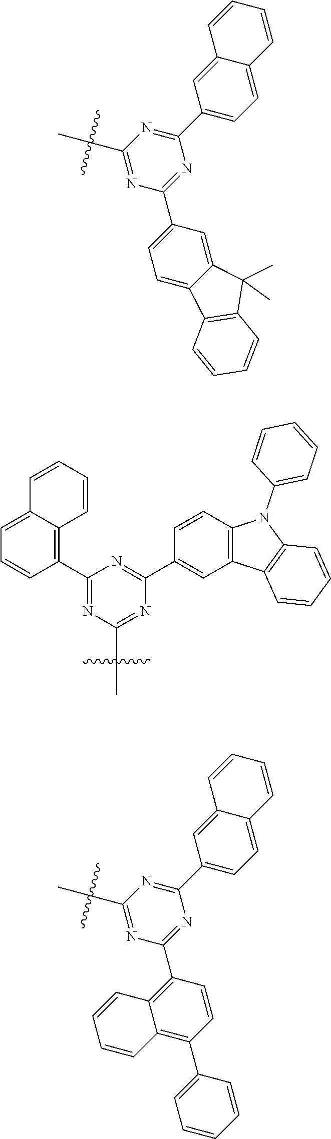 [SMILES: *c1nc(-c2ccc3c(c2)C(C)(C)c2ccccc2-3)nc(-c2ccc3ccccc3c2)n1 ; *c1nc(-c2ccc3c(c2)c2ccccc2n3-c2ccccc2)nc(-c2cccc3ccccc23)n1 ; *c1nc(-c2ccc3ccccc3c2)nc(-c2ccc(-c3ccccc3)c3ccccc23)n1]

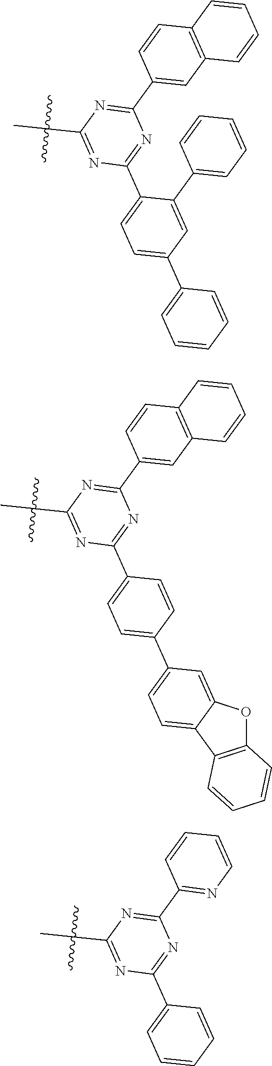

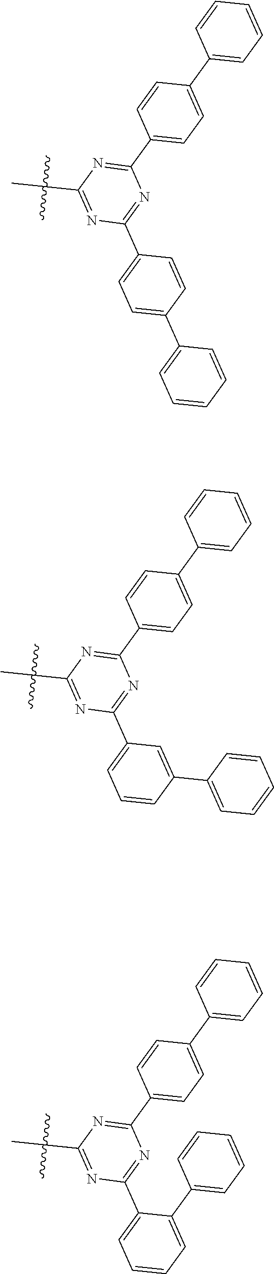 [SMILES: *c1nc(-c2ccc(-c3ccccc3)cc2)nc(-c2ccc(-c3ccccc3)cc2)n1 ; *c1nc(-c2ccc(-c3ccccc3)cc2)nc(-c2cccc(-c3ccccc3)c2)n1 ; *c1nc(-c2ccc(-c3ccccc3)cc2)nc(-c2ccccc2-c2ccccc2)n1]

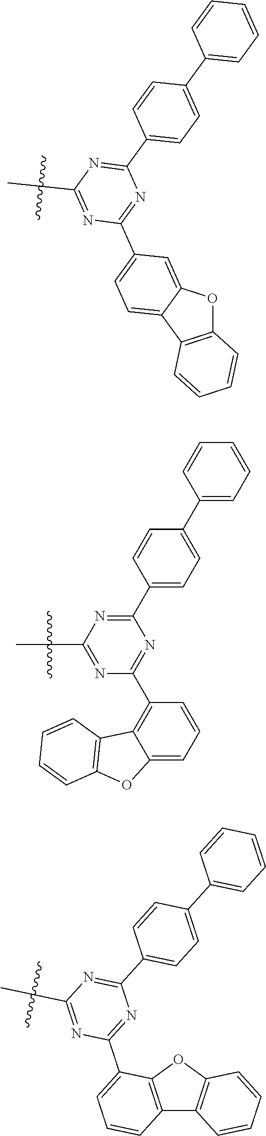

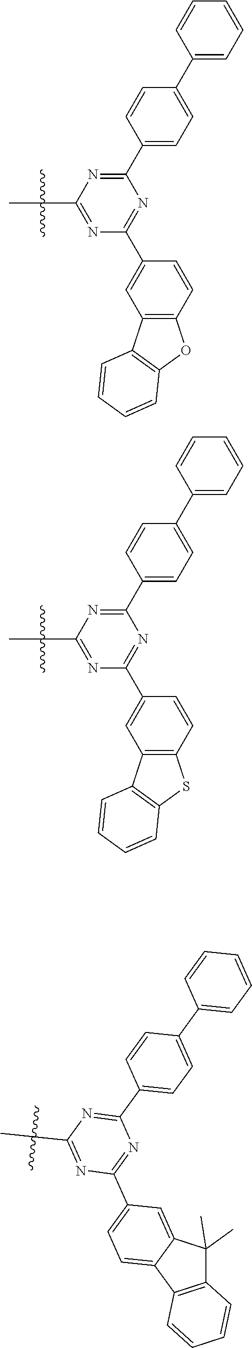 [SMILES: *c1nc(-c2ccc(-c3ccccc3)cc2)nc(-c2ccc3oc4ccccc4c3c2)n1 ; *c1nc(-c2ccc(-c3ccccc3)cc2)nc(-c2ccc3sc4ccccc4c3c2)n1 ; *c1nc(-c2ccc(-c3ccccc3)cc2)nc(-c2ccc3c(c2)C(C)(C)c2ccccc2-3)n1]

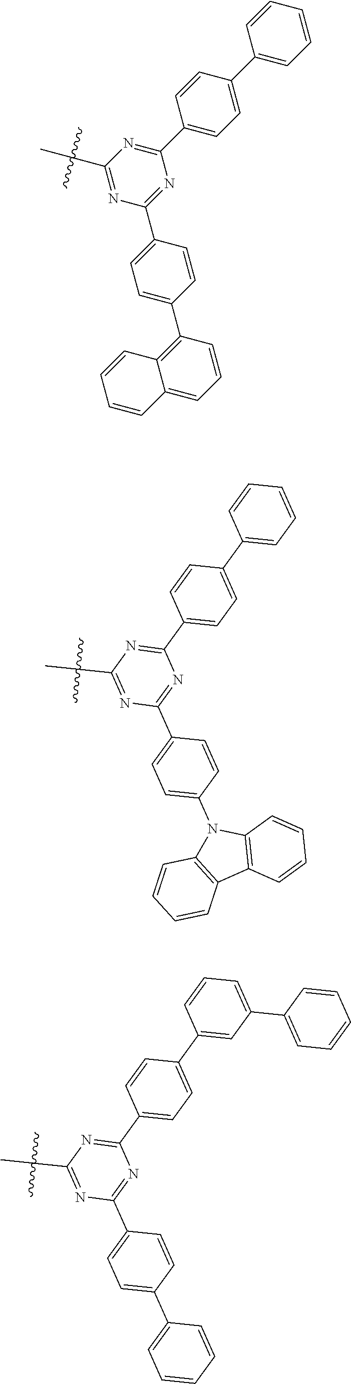 [SMILES: *c1nc(-c2ccc(-c3ccccc3)cc2)nc(-c2ccc(-c3cccc4ccccc34)cc2)n1 ; *c1nc(-c2ccc(-c3ccccc3)cc2)nc(-c2ccc(-n3c4ccccc4c4ccccc43)cc2)n1 ; *c1nc(-c2ccc(-c3ccccc3)cc2)nc(-c2ccc(-c3cccc(-c4ccccc4)c3)cc2)n1]

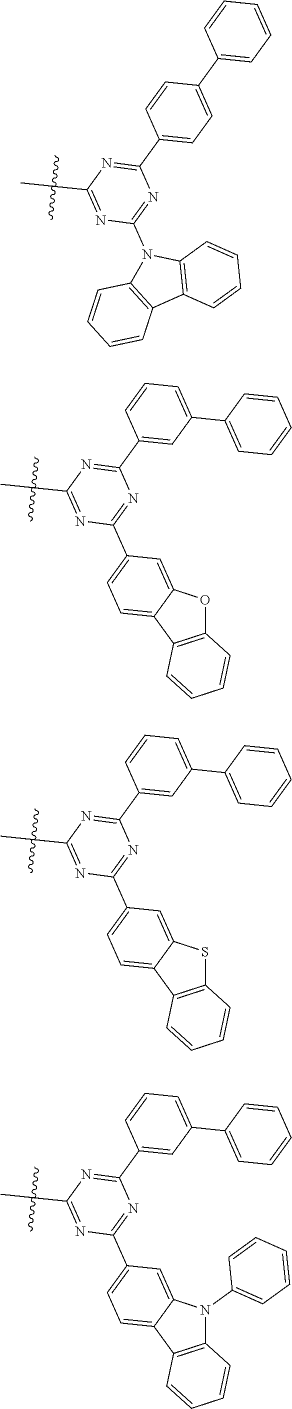

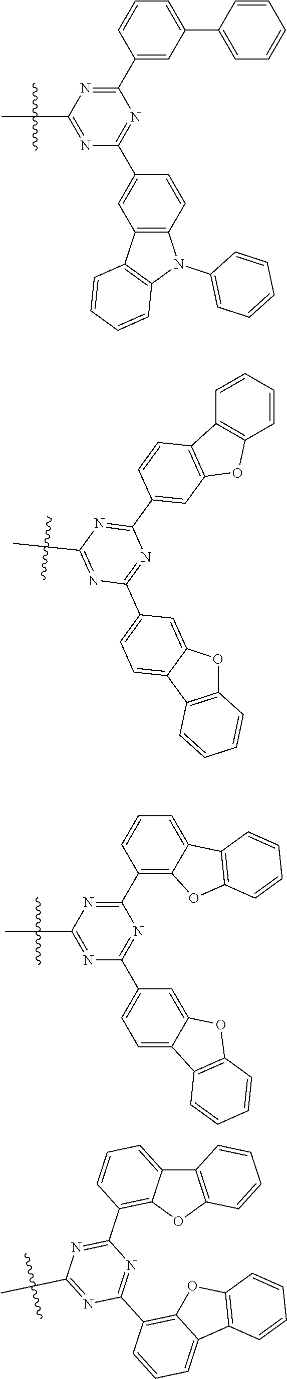

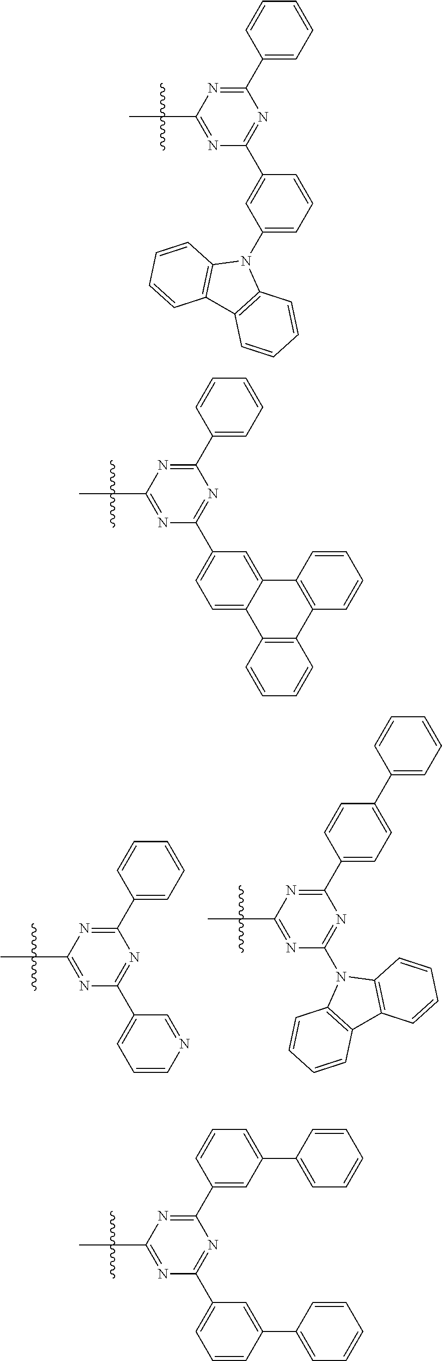

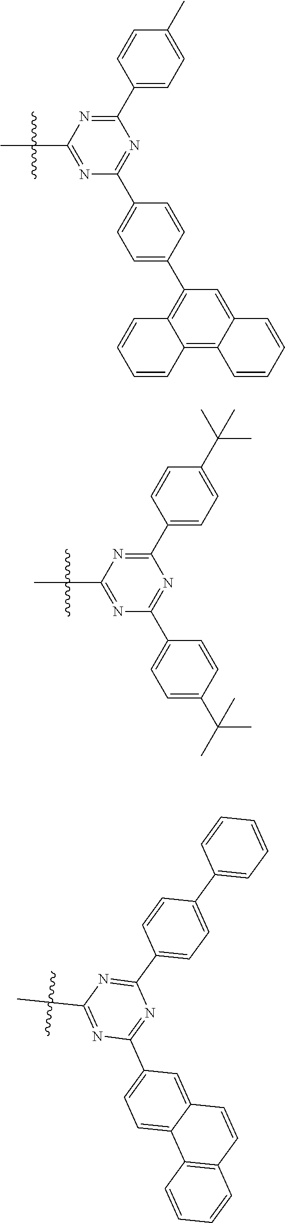

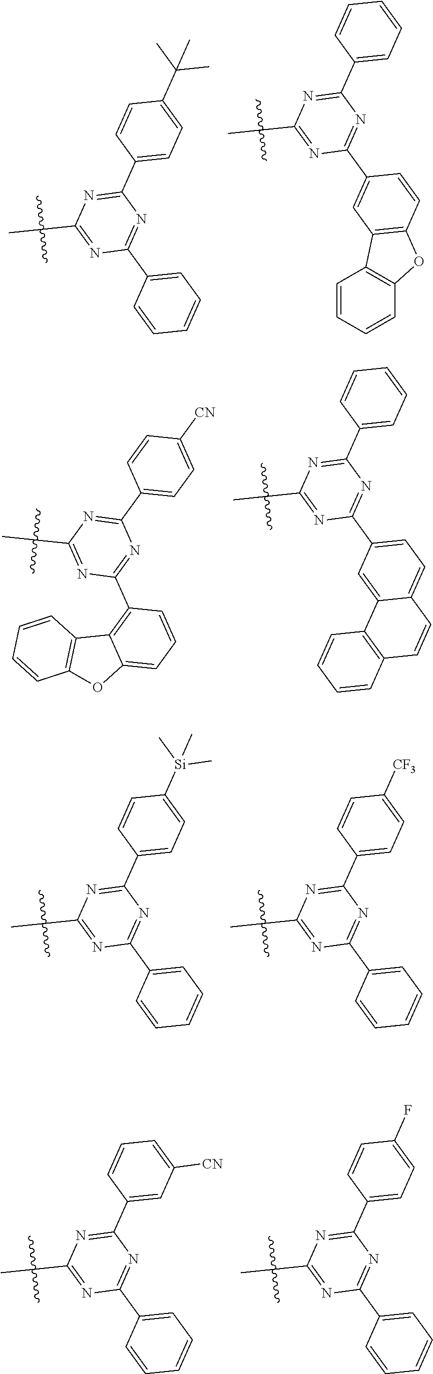

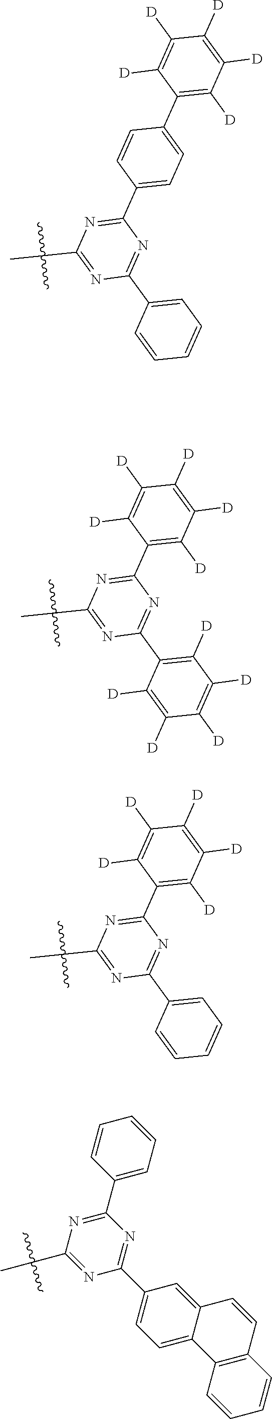 [SMILES: *c1nc(-c2ccccc2)nc(-c2ccc(-c3c([2H])c([2H])c([2H])c([2H])c3[2H])cc2)n1 ; *c1nc(-c2c([2H])c([2H])c([2H])c([2H])c2[2H])nc(-c2c([2H])c([2H])c([2H])c([2H])c2[2H])n1 ; *c1nc(-c2ccccc2)nc(-c2c([2H])c([2H])c([2H])c([2H])c2[2H])n1 ; *c1nc(-c2ccccc2)nc(-c2ccc3c(ccc4ccccc43)c2)n1]

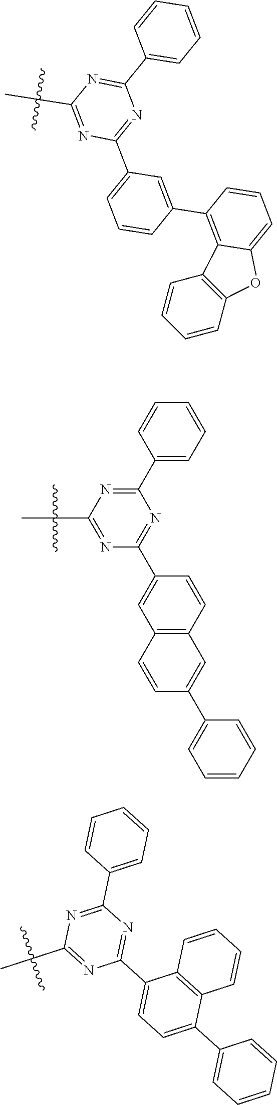 [SMILES: *c1nc(-c2ccccc2)nc(-c2cccc(-c3cccc4oc5ccccc5c34)c2)n1 ; *c1nc(-c2ccccc2)nc(-c2ccc3cc(-c4ccccc4)ccc3c2)n1 ; *c1nc(-c2ccccc2)nc(-c2ccc(-c3ccccc3)c3ccccc23)n1]

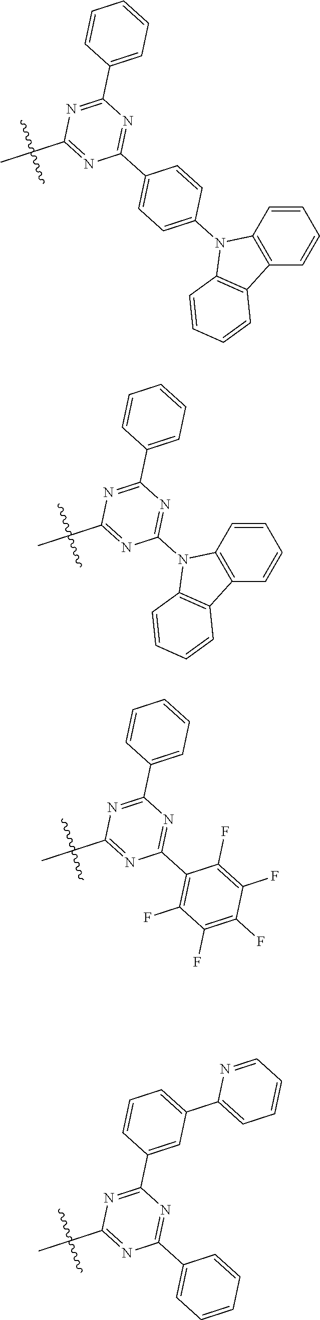

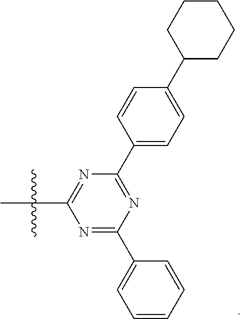 [SMILES: *c1nc(-c2ccccc2)nc(-c2ccc(C3CCCCC3)cc2)n1]

In some embodiments of the disclosure, R₄ and R₅ are the same or different, and are respectively and independently selected from methyl and substituted or unsubstituted phenyl.

Optionally, substituent(s) in R₄ and R₅ are the same or different, and are respectively and independently selected from deuterium, fluorine, cyano, methyl, ethyl, n-propyl, isopropyl, tert-butyl and phenyl.

In some more specific embodiments of the disclosure, R₄ and R₅ are both methyl.

In some embodiments of the disclosure, R₁ is selected from the group represented by the formula 2 or the group consisting of:

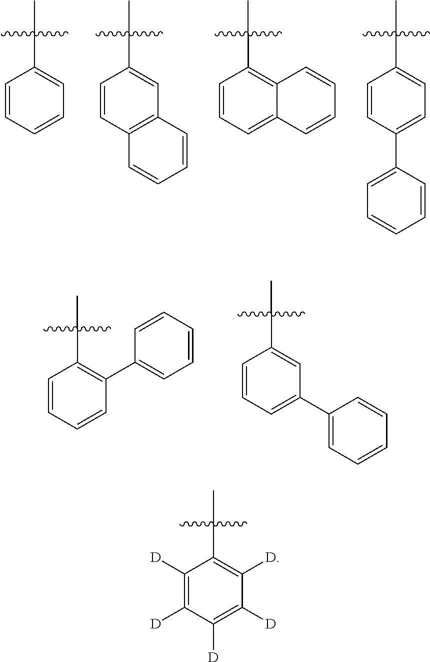

In some embodiments of the disclosure, R₆ is selected from the group represented by the formula 2 or the group consisting of:

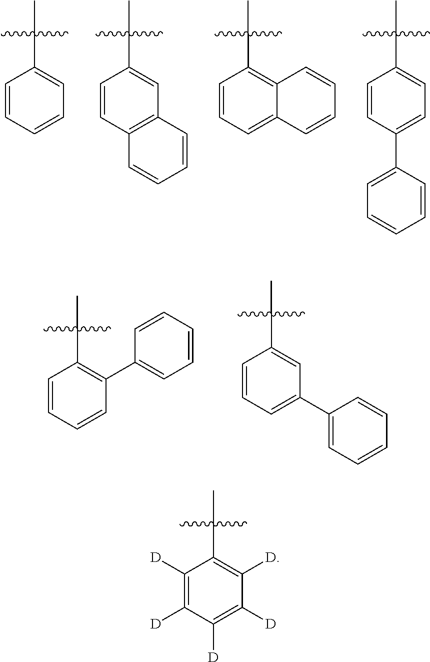

In some embodiments of the disclosure, R₁ and R₆ are the same or different, and are respectively and independently selected from substituted or unsubstituted phenyl, substituted or unsubstituted naphthyl, substituted or unsubstituted biphenyl and the group represented by the formula 2;
R₂ and R₃ are the same or different, and are respectively and independently selected from hydrogen and the group represented by the formula 2;
and one and only one of R₁, R₆, R₂ and R₃ is the group represented by the formula 2; and
optionally, substituent(s) in R₁ and R₆ are the same or different, and are respectively and independently selected from deuterium, fluorine, cyano, methyl, ethyl, n-propyl, isopropyl, tert-butyl and phenyl.

In some specific embodiments of the disclosure, R₁ is selected from substituted or unsubstituted phenyl, substituted or unsubstituted naphthyl, substituted or unsubstituted biphenyl and the group represented by the formula 2; and
substituent(s) in R₁ are the same or different, and are respectively and independently selected from deuterium, fluorine, cyano, methyl, ethyl, n-propyl, isopropyl, tert-butyl and phenyl.

In some embodiments of the disclosure, R₆ is selected from substituted or unsubstituted phenyl, substituted or unsubstituted naphthyl, substituted or unsubstituted biphenyl, and the group represented by the formula 2; and
substituent(s) in R₆ are the same or different, and are respectively and independently selected from deuterium, fluorine, cyano, methyl, ethyl, n-propyl, isopropyl, tert-butyl and phenyl.

R₂ is selected from hydrogen or the group represented by the formula 2.

R₃ is selected from hydrogen or the group represented by the formula 2.

In some embodiments of the disclosure, one and only one of R₁, R₆, R₂ and R₃ is the group represented by the formula 2.

In some more specific embodiments of the disclosure, X is O or S, and R₂ and R₃ are the same or different, and are respectively and independently selected from hydrogen and the group represented by the formula 2;
R₆ is selected from substituted or unsubstituted phenyl, substituted or unsubstituted naphthyl, substituted or unsubstituted biphenyl and the group represented by the formula 2;
and one and only one of R₁, R₂ and R₃ is the group represented by the formula 2; and
substituent(s) in R₆ are the same or different, and are respectively and independently selected from deuterium, fluorine, cyano, methyl, ethyl, n-propyl, isopropyl, tert-butyl and phenyl.

In some embodiments of the disclosure, the organic compound is selected from the group consisting of the following compounds:

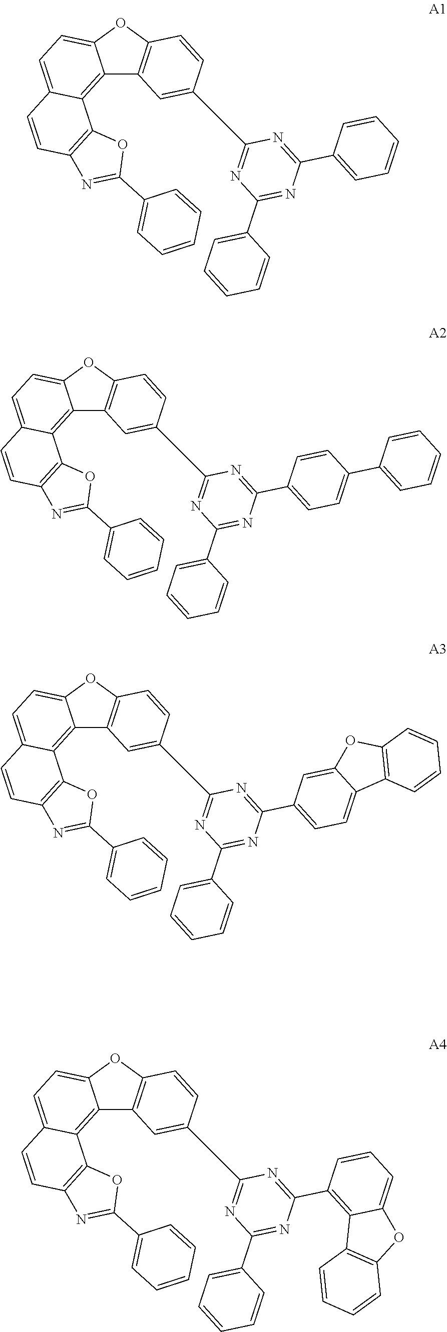 [SMILES: c1ccc(-c2nc(-c3ccccc3)nc(-c3ccc4oc5ccc6ccc7nc(-c8ccccc8)oc7c6c5c4c3)n2)cc1 ; c1ccc(-c2ccc(-c3nc(-c4ccccc4)nc(-c4ccc5oc6ccc7ccc8nc(-c9ccccc9)oc8c7c6c5c4)n3)cc2)cc1 ; c1ccc(-c2nc(-c3ccc4c(c3)oc3ccccc34)nc(-c3ccc4oc5ccc6ccc7nc(-c8ccccc8)oc7c6c5c4c3)n2)cc1 ; c1ccc(-c2nc(-c3ccc4oc5ccc6ccc7nc(-c8ccccc8)oc7c6c5c4c3)nc(-c3cccc4oc5ccccc5c34)n2)cc1]

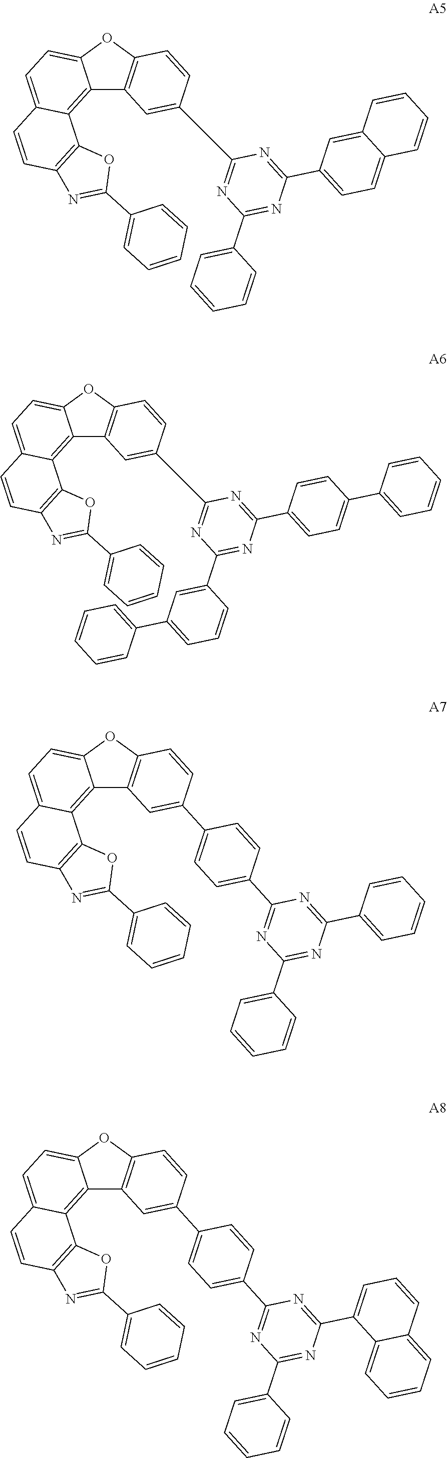 [SMILES: c1ccc(-c2nc(-c3ccc4ccccc4c3)nc(-c3ccc4oc5ccc6ccc7nc(-c8ccccc8)oc7c6c5c4c3)n2)cc1 ; c1ccc(-c2ccc(-c3nc(-c4cccc(-c5ccccc5)c4)nc(-c4ccc5oc6ccc7ccc8nc(-c9ccccc9)oc8c7c6c5c4)n3)cc2)cc1 ; c1ccc(-c2nc(-c3ccccc3)nc(-c3ccc(-c4ccc5oc6ccc7ccc8nc(-c9ccccc9)oc8c7c6c5c4)cc3)n2)cc1 ; c1ccc(-c2nc(-c3ccc(-c4ccc5oc6ccc7ccc8nc(-c9ccccc9)oc8c7c6c5c4)cc3)nc(-c3cccc4ccccc34)n2)cc1]

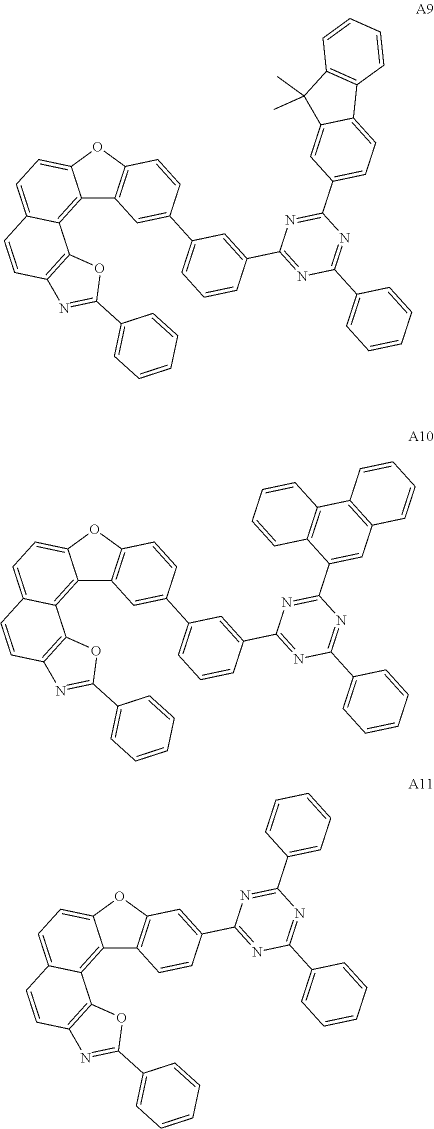 [SMILES: CC1(C)c2ccccc2-c2ccc(-c3nc(-c4ccccc4)nc(-c4cccc(-c5ccc6oc7ccc8ccc9nc(-c%10ccccc%10)oc9c8c7c6c5)c4)n3)cc21 ; c1ccc(-c2nc(-c3cccc(-c4ccc5oc6ccc7ccc8nc(-c9ccccc9)oc8c7c6c5c4)c3)nc(-c3cc4ccccc4c4ccccc34)n2)cc1 ; c1ccc(-c2nc(-c3ccccc3)nc(-c3ccc4c(c3)oc3ccc5ccc6nc(-c7ccccc7)oc6c5c34)n2)cc1]

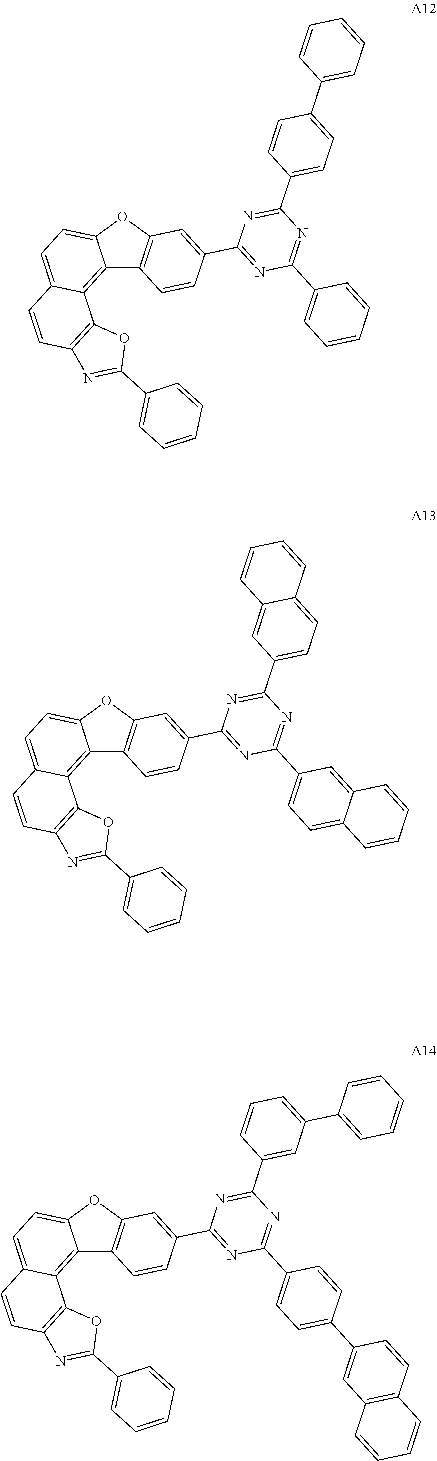 [SMILES: c1ccc(-c2ccc(-c3nc(-c4ccccc4)nc(-c4ccc5c(c4)oc4ccc6ccc7nc(-c8ccccc8)oc7c6c45)n3)cc2)cc1 ; c1ccc(-c2nc3ccc4ccc5oc6cc(-c7nc(-c8ccc9ccccc9c8)nc(-c8ccc9ccccc9c8)n7)ccc6c5c4c3o2)cc1 ; c1ccc(-c2cccc(-c3nc(-c4ccc(-c5ccc6ccccc6c5)cc4)nc(-c4ccc5c(c4)oc4ccc6ccc7nc(-c8ccccc8)oc7c6c45)n3)c2)cc1]

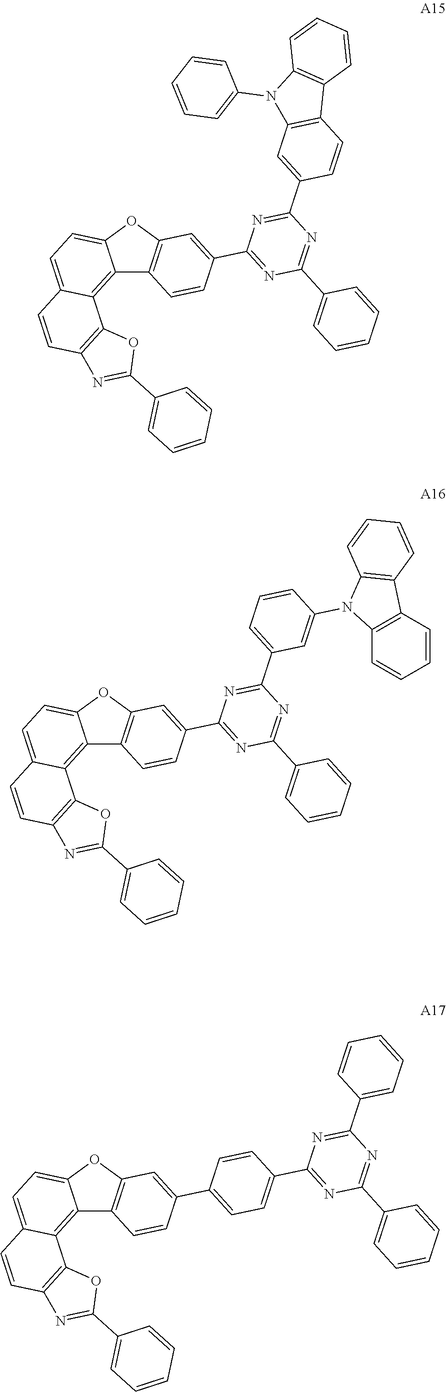 [SMILES: c1ccc(-c2nc(-c3ccc4c(c3)oc3ccc5ccc6nc(-c7ccccc7)oc6c5c34)nc(-c3ccc4c5ccccc5n(-c5ccccc5)c4c3)n2)cc1 ; c1ccc(-c2nc(-c3cccc(-n4c5ccccc5c5ccccc54)c3)nc(-c3ccc4c(c3)oc3ccc5ccc6nc(-c7ccccc7)oc6c5c34)n2)cc1 ; c1ccc(-c2nc(-c3ccccc3)nc(-c3ccc(-c4ccc5c(c4)oc4ccc6ccc7nc(-c8ccccc8)oc7c6c45)cc3)n2)cc1]

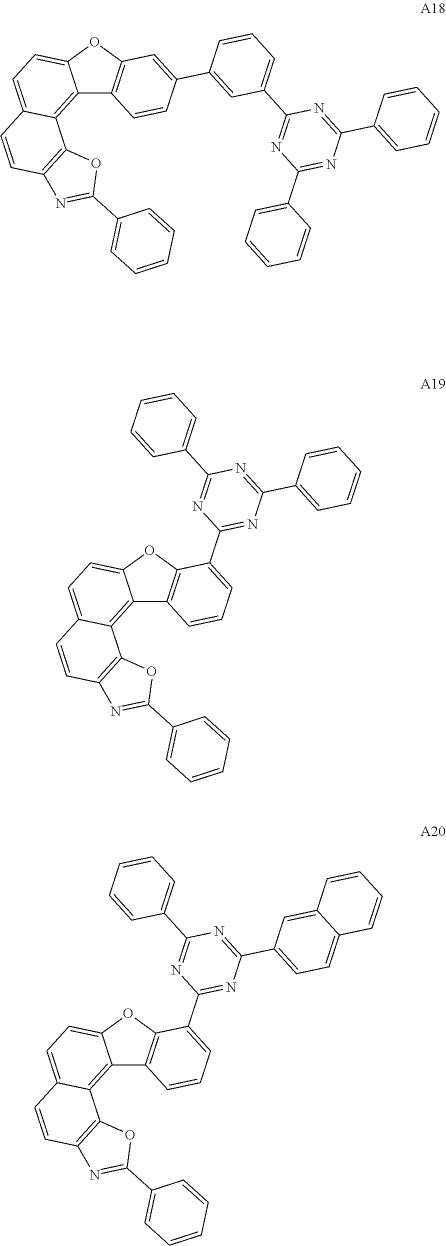 [SMILES: c1ccc(-c2nc(-c3ccccc3)nc(-c3cccc(-c4ccc5c(c4)oc4ccc6ccc7nc(-c8ccccc8)oc7c6c45)c3)n2)cc1 ; c1ccc(-c2nc(-c3ccccc3)nc(-c3cccc4c3oc3ccc5ccc6nc(-c7ccccc7)oc6c5c34)n2)cc1 ; c1ccc(-c2nc(-c3ccc4ccccc4c3)nc(-c3cccc4c3oc3ccc5ccc6nc(-c7ccccc7)oc6c5c34)n2)cc1]

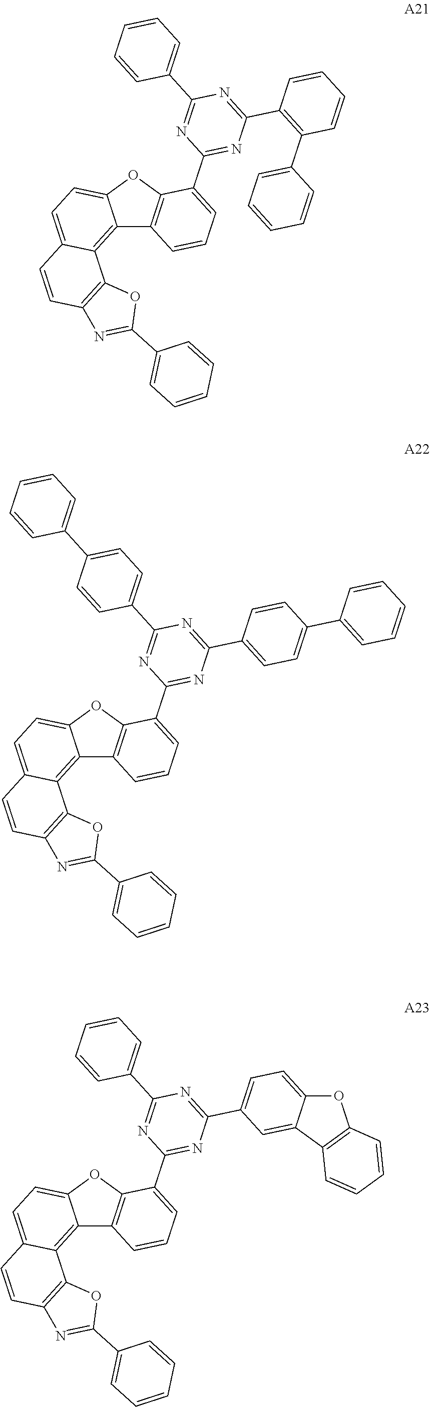 [SMILES: c1ccc(-c2nc(-c3ccccc3-c3ccccc3)nc(-c3cccc4c3oc3ccc5ccc6nc(-c7ccccc7)oc6c5c34)n2)cc1 ; c1ccc(-c2ccc(-c3nc(-c4ccc(-c5ccccc5)cc4)nc(-c4cccc5c4oc4ccc6ccc7nc(-c8ccccc8)oc7c6c45)n3)cc2)cc1 ; c1ccc(-c2nc(-c3ccc4oc5ccccc5c4c3)nc(-c3cccc4c3oc3ccc5ccc6nc(-c7ccccc7)oc6c5c34)n2)cc1]

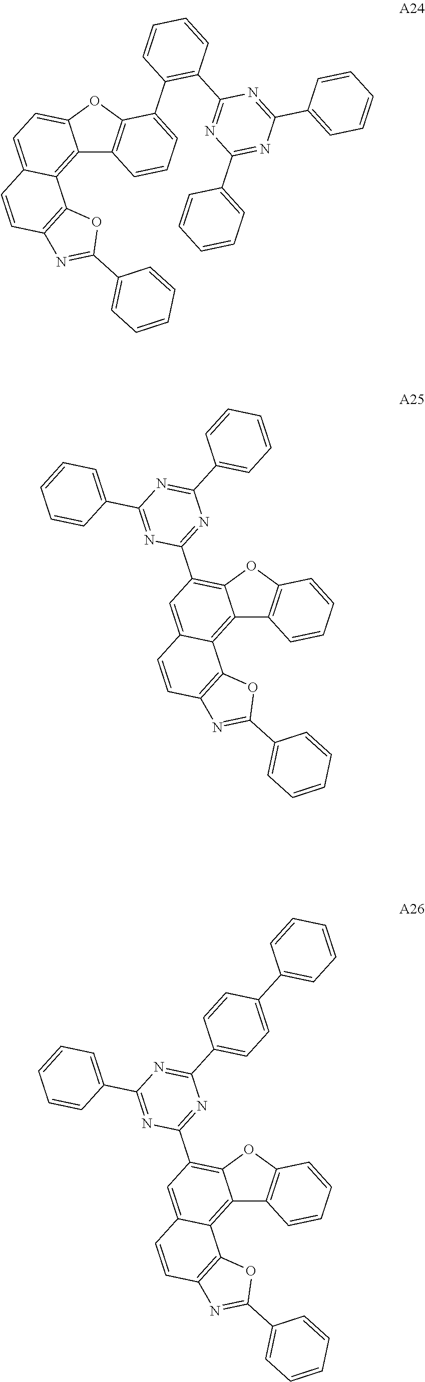 [SMILES: c1ccc(-c2nc(-c3ccccc3)nc(-c3ccccc3-c3cccc4c3oc3ccc5ccc6nc(-c7ccccc7)oc6c5c34)n2)cc1 ; c1ccc(-c2nc(-c3ccccc3)nc(-c3cc4ccc5nc(-c6ccccc6)oc5c4c4c3oc3ccccc34)n2)cc1 ; c1ccc(-c2ccc(-c3nc(-c4ccccc4)nc(-c4cc5ccc6nc(-c7ccccc7)oc6c5c5c4oc4ccccc45)n3)cc2)cc1]

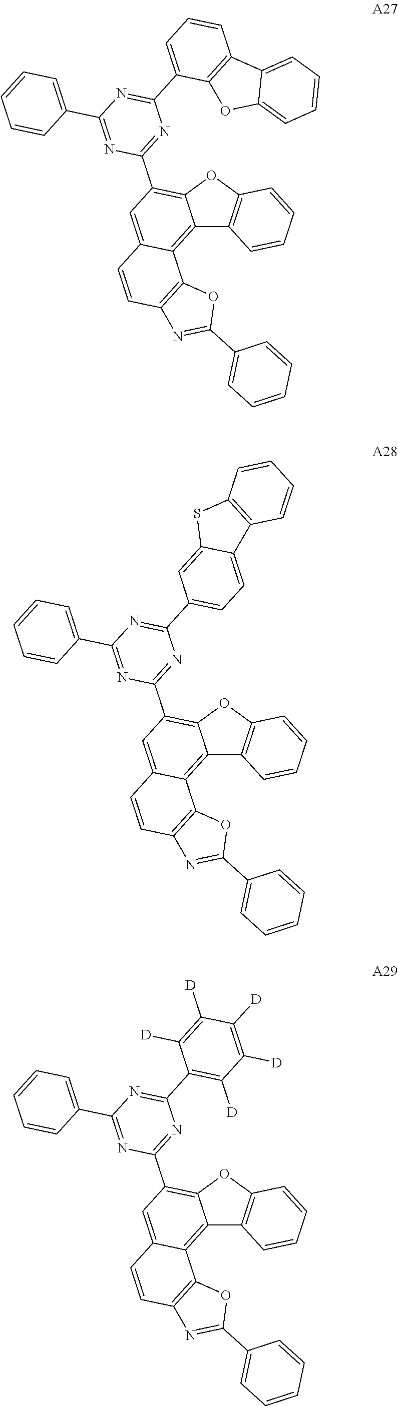 [SMILES: c1ccc(-c2nc(-c3cccc4c3oc3ccccc34)nc(-c3cc4ccc5nc(-c6ccccc6)oc5c4c4c3oc3ccccc34)n2)cc1 ; c1ccc(-c2nc(-c3ccc4c(c3)sc3ccccc34)nc(-c3cc4ccc5nc(-c6ccccc6)oc5c4c4c3oc3ccccc34)n2)cc1 ; [2H]c1c([2H])c([2H])c(-c2nc(-c3ccccc3)nc(-c3cc4ccc5nc(-c6ccccc6)oc5c4c4c3oc3ccccc34)n2)c([2H])c1[2H]]

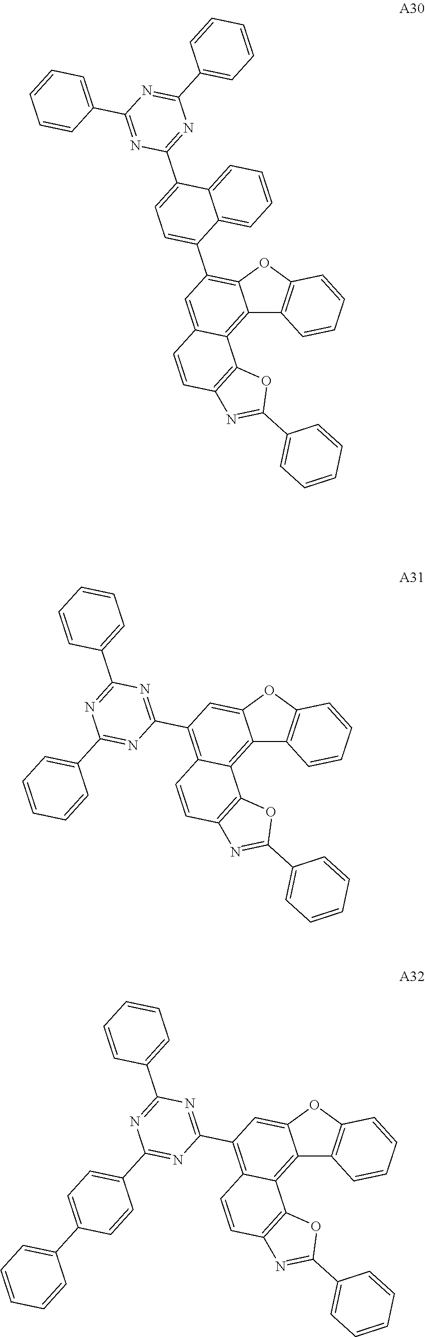 [SMILES: c1ccc(-c2nc(-c3ccccc3)nc(-c3ccc(-c4cc5ccc6nc(-c7ccccc7)oc6c5c5c4oc4ccccc45)c4ccccc34)n2)cc1 ; c1ccc(-c2nc(-c3ccccc3)nc(-c3cc4oc5ccccc5c4c4c3ccc3nc(-c5ccccc5)oc34)n2)cc1 ; c1ccc(-c2ccc(-c3nc(-c4ccccc4)nc(-c4cc5oc6ccccc6c5c5c4ccc4nc(-c6ccccc6)oc45)n3)cc2)cc1]

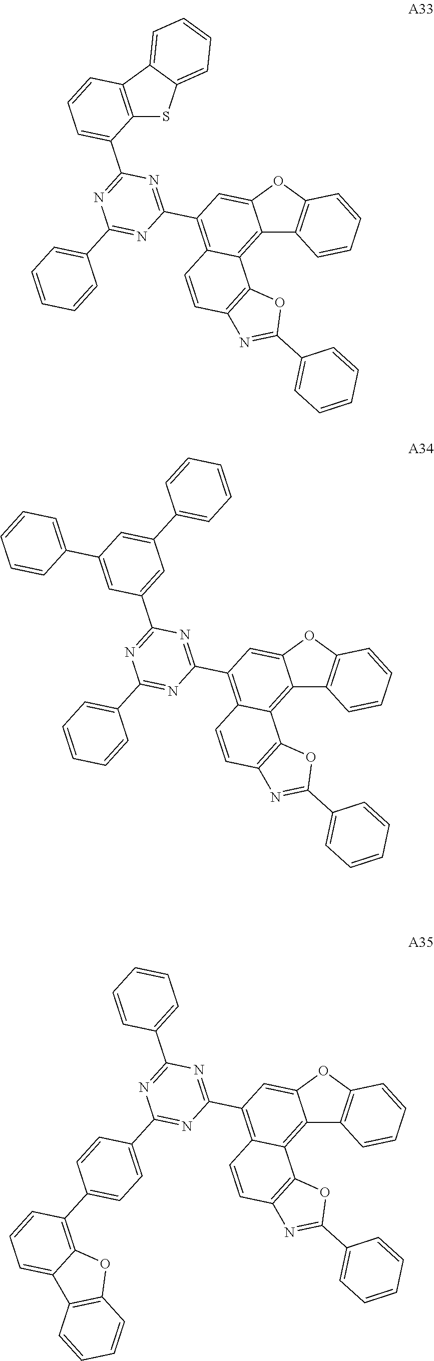 [SMILES: c1ccc(-c2nc(-c3cc4oc5ccccc5c4c4c3ccc3nc(-c5ccccc5)oc34)nc(-c3cccc4c3sc3ccccc34)n2)cc1 ; c1ccc(-c2cc(-c3ccccc3)cc(-c3nc(-c4ccccc4)nc(-c4cc5oc6ccccc6c5c5c4ccc4nc(-c6ccccc6)oc45)n3)c2)cc1 ; c1ccc(-c2nc(-c3ccc(-c4cccc5c4oc4ccccc45)cc3)nc(-c3cc4oc5ccccc5c4c4c3ccc3nc(-c5ccccc5)oc34)n2)cc1]

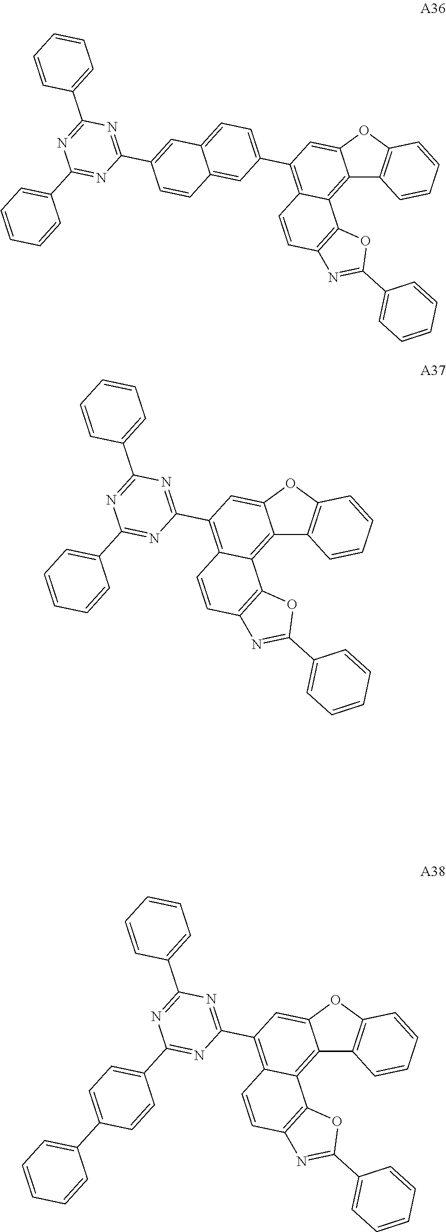 [SMILES: c1ccc(-c2nc(-c3ccccc3)nc(-c3ccc4cc(-c5cc6oc7ccccc7c6c6c5ccc5nc(-c7ccccc7)oc56)ccc4c3)n2)cc1 ; c1ccc(-c2nc(-c3ccccc3)nc(-c3cc4oc5ccccc5c4c4c3ccc3nc(-c5ccccc5)oc34)n2)cc1 ; c1ccc(-c2ccc(-c3nc(-c4ccccc4)nc(-c4cc5oc6ccccc6c5c5c4ccc4nc(-c6ccccc6)oc45)n3)cc2)cc1]

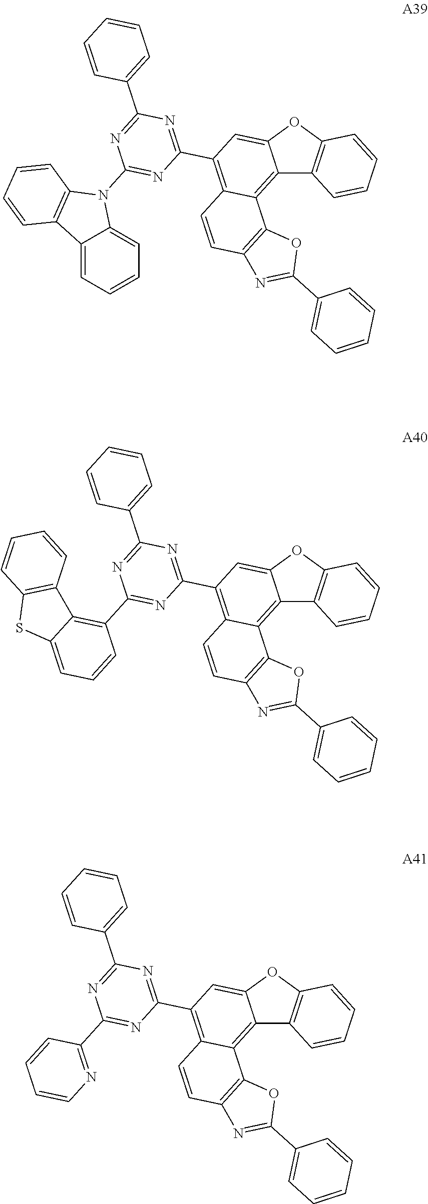 [SMILES: c1ccc(-c2nc(-c3cc4oc5ccccc5c4c4c3ccc3nc(-c5ccccc5)oc34)nc(-n3c4ccccc4c4ccccc43)n2)cc1 ; c1ccc(-c2nc(-c3cc4oc5ccccc5c4c4c3ccc3nc(-c5ccccc5)oc34)nc(-c3cccc4sc5ccccc5c34)n2)cc1 ; c1ccc(-c2nc(-c3ccccn3)nc(-c3cc4oc5ccccc5c4c4c3ccc3nc(-c5ccccc5)oc34)n2)cc1]

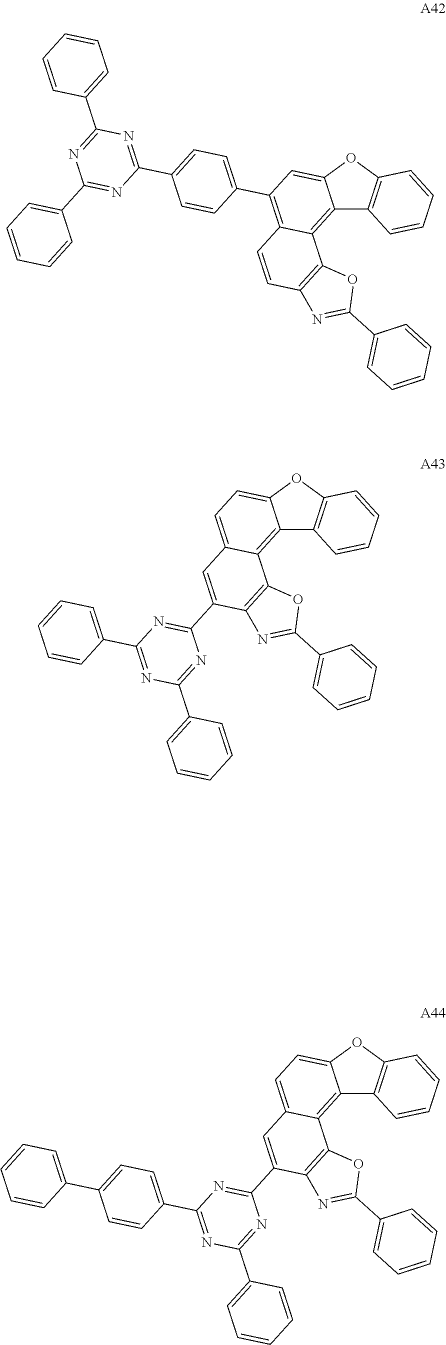 [SMILES: c1ccc(-c2nc(-c3ccccc3)nc(-c3ccc(-c4cc5oc6ccccc6c5c5c4ccc4nc(-c6ccccc6)oc45)cc3)n2)cc1 ; c1ccc(-c2nc(-c3ccccc3)nc(-c3cc4ccc5oc6ccccc6c5c4c4oc(-c5ccccc5)nc34)n2)cc1 ; c1ccc(-c2ccc(-c3nc(-c4ccccc4)nc(-c4cc5ccc6oc7ccccc7c6c5c5oc(-c6ccccc6)nc45)n3)cc2)cc1]

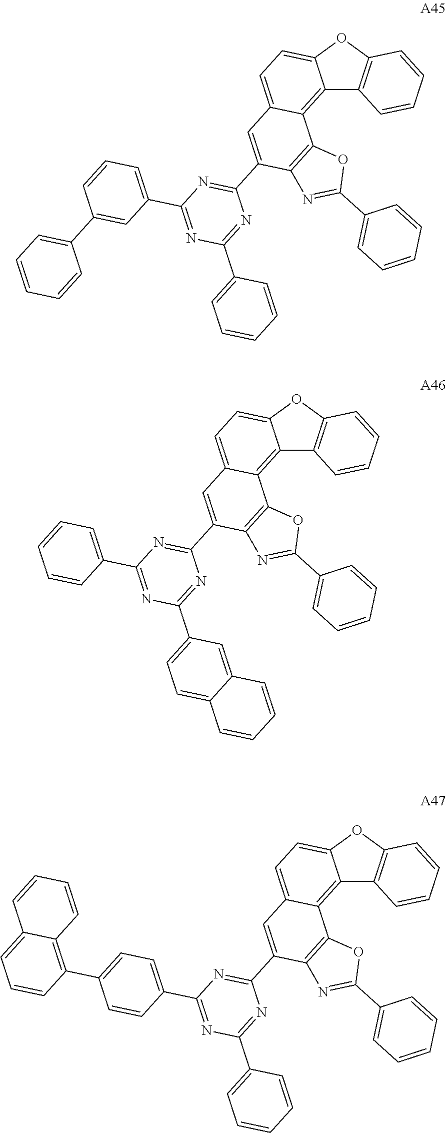 [SMILES: c1ccc(-c2cccc(-c3nc(-c4ccccc4)nc(-c4cc5ccc6oc7ccccc7c6c5c5oc(-c6ccccc6)nc45)n3)c2)cc1 ; c1ccc(-c2nc(-c3ccc4ccccc4c3)nc(-c3cc4ccc5oc6ccccc6c5c4c4oc(-c5ccccc5)nc34)n2)cc1 ; c1ccc(-c2nc(-c3ccc(-c4cccc5ccccc45)cc3)nc(-c3cc4ccc5oc6ccccc6c5c4c4oc(-c5ccccc5)nc34)n2)cc1]

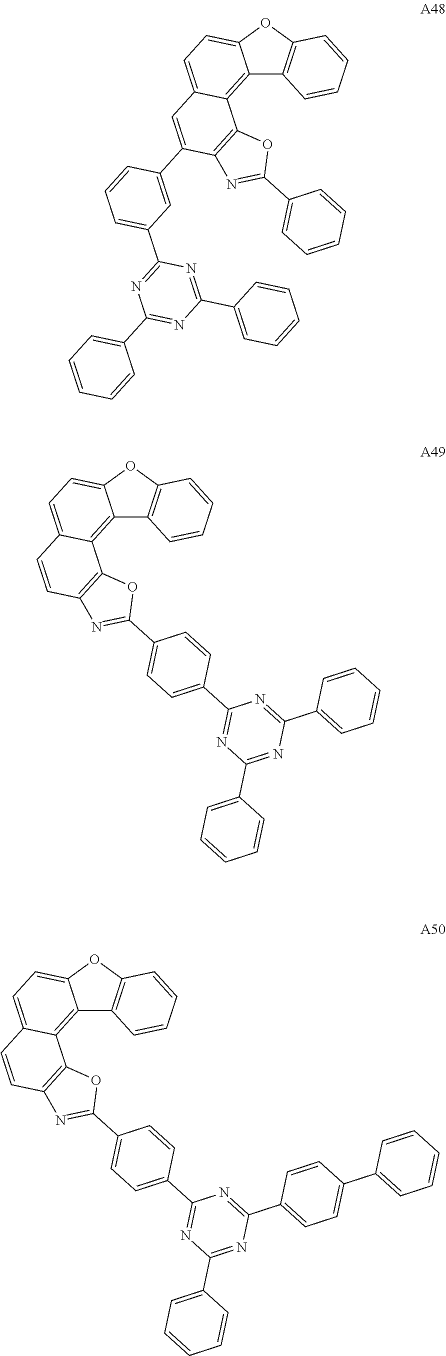 [SMILES: c1ccc(-c2nc(-c3ccccc3)nc(-c3cccc(-c4cc5ccc6oc7ccccc7c6c5c5oc(-c6ccccc6)nc45)c3)n2)cc1 ; c1ccc(-c2nc(-c3ccccc3)nc(-c3ccc(-c4nc5ccc6ccc7oc8ccccc8c7c6c5o4)cc3)n2)cc1 ; c1ccc(-c2ccc(-c3nc(-c4ccccc4)nc(-c4ccc(-c5nc6ccc7ccc8oc9ccccc9c8c7c6o5)cc4)n3)cc2)cc1]

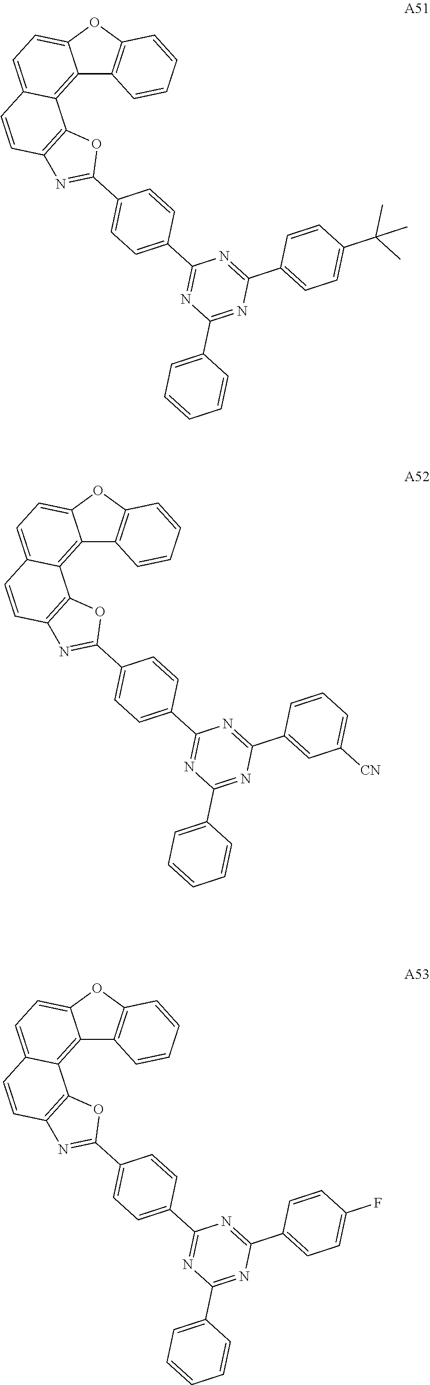 [SMILES: CC(C)(C)c1ccc(-c2nc(-c3ccccc3)nc(-c3ccc(-c4nc5ccc6ccc7oc8ccccc8c7c6c5o4)cc3)n2)cc1 ; N#Cc1cccc(-c2nc(-c3ccccc3)nc(-c3ccc(-c4nc5ccc6ccc7oc8ccccc8c7c6c5o4)cc3)n2)c1 ; Fc1ccc(-c2nc(-c3ccccc3)nc(-c3ccc(-c4nc5ccc6ccc7oc8ccccc8c7c6c5o4)cc3)n2)cc1]

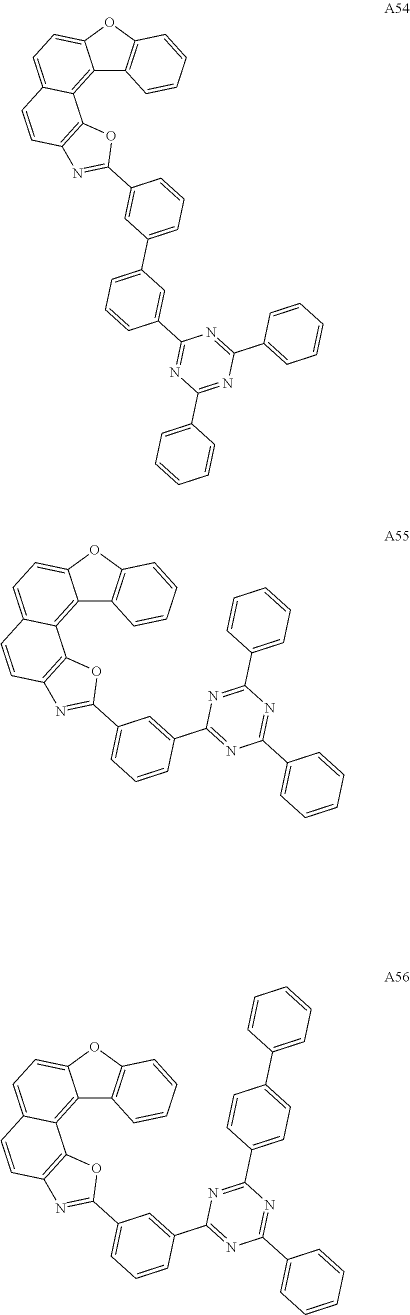 [SMILES: c1ccc(-c2nc(-c3ccccc3)nc(-c3cccc(-c4cccc(-c5nc6ccc7ccc8oc9ccccc9c8c7c6o5)c4)c3)n2)cc1 ; c1ccc(-c2nc(-c3ccccc3)nc(-c3cccc(-c4nc5ccc6ccc7oc8ccccc8c7c6c5o4)c3)n2)cc1 ; c1ccc(-c2ccc(-c3nc(-c4ccccc4)nc(-c4cccc(-c5nc6ccc7ccc8oc9ccccc9c8c7c6o5)c4)n3)cc2)cc1]

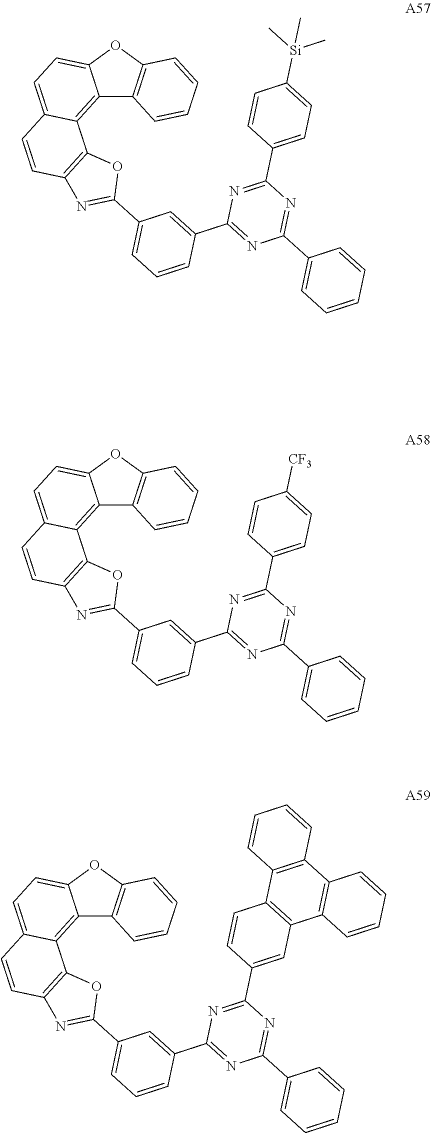 [SMILES: C[Si](C)(C)c1ccc(-c2nc(-c3ccccc3)nc(-c3cccc(-c4nc5ccc6ccc7oc8ccccc8c7c6c5o4)c3)n2)cc1 ; FC(F)(F)c1ccc(-c2nc(-c3ccccc3)nc(-c3cccc(-c4nc5ccc6ccc7oc8ccccc8c7c6c5o4)c3)n2)cc1 ; c1ccc(-c2nc(-c3cccc(-c4nc5ccc6ccc7oc8ccccc8c7c6c5o4)c3)nc(-c3ccc4c5ccccc5c5ccccc5c4c3)n2)cc1]

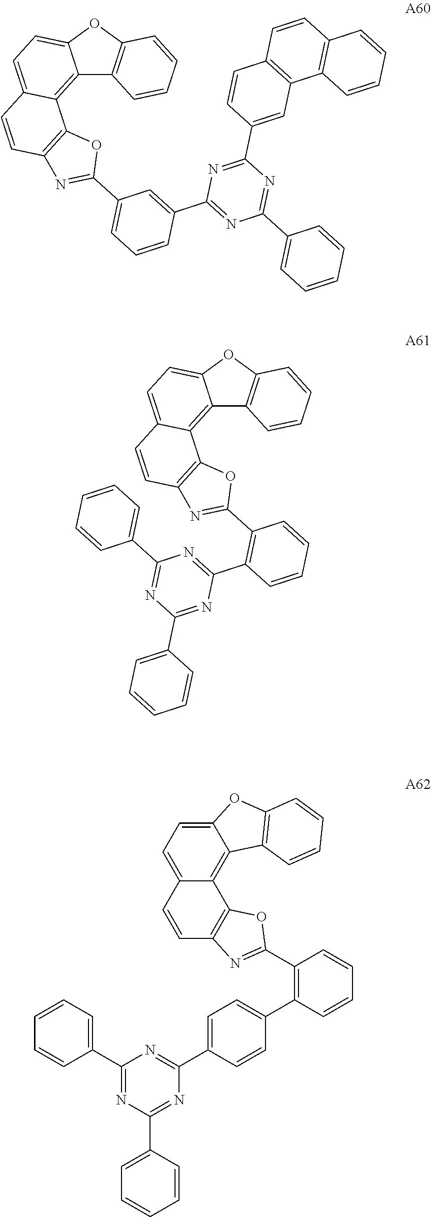 [SMILES: c1ccc(-c2nc(-c3cccc(-c4nc5ccc6ccc7oc8ccccc8c7c6c5o4)c3)nc(-c3ccc4ccc5ccccc5c4c3)n2)cc1 ; c1ccc(-c2nc(-c3ccccc3)nc(-c3ccccc3-c3nc4ccc5ccc6oc7ccccc7c6c5c4o3)n2)cc1 ; c1ccc(-c2nc(-c3ccccc3)nc(-c3ccc(-c4ccccc4-c4nc5ccc6ccc7oc8ccccc8c7c6c5o4)cc3)n2)cc1]

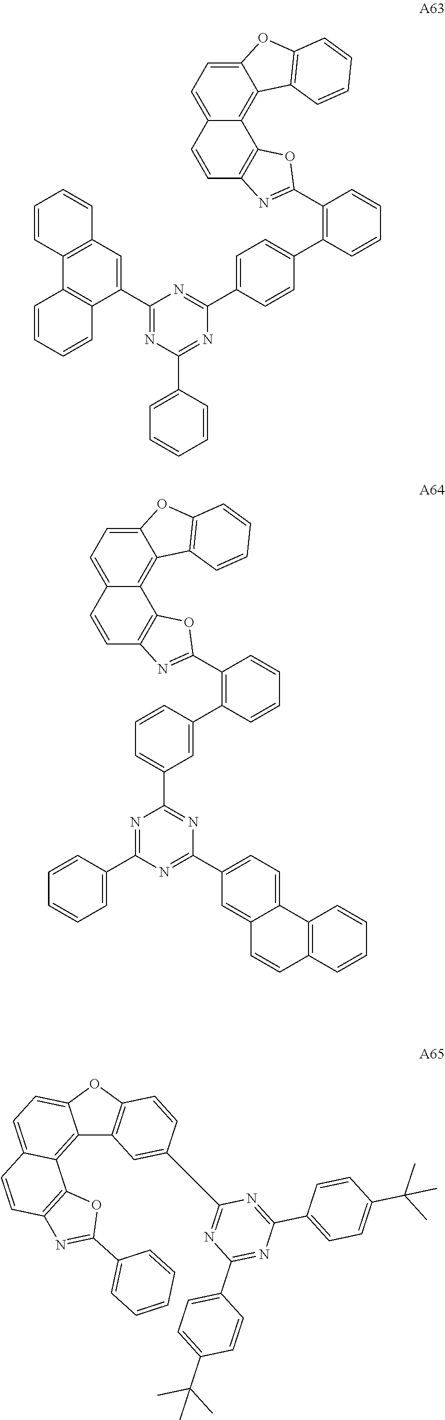 [SMILES: c1ccc(-c2nc(-c3ccc(-c4ccccc4-c4nc5ccc6ccc7oc8ccccc8c7c6c5o4)cc3)nc(-c3cc4ccccc4c4ccccc34)n2)cc1 ; c1ccc(-c2nc(-c3cccc(-c4ccccc4-c4nc5ccc6ccc7oc8ccccc8c7c6c5o4)c3)nc(-c3ccc4c(ccc5ccccc54)c3)n2)cc1 ; CC(C)(C)c1ccc(-c2nc(-c3ccc(C(C)(C)C)cc3)nc(-c3ccc4oc5ccc6ccc7nc(-c8ccccc8)oc7c6c5c4c3)n2)cc1]

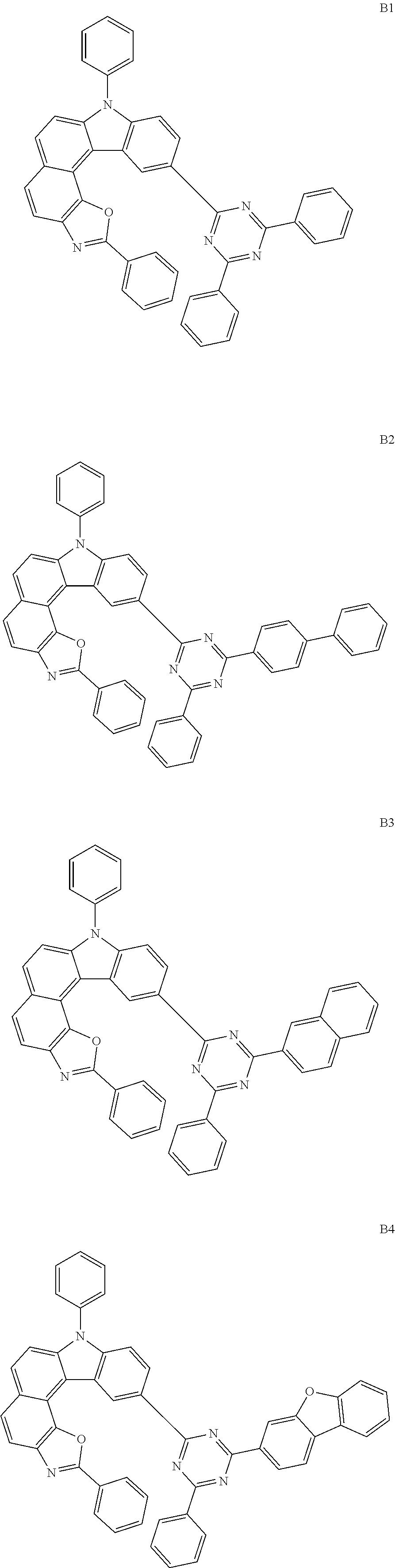 [SMILES: c1ccc(-c2nc(-c3ccccc3)nc(-c3ccc4c(c3)c3c5c(ccc6nc(-c7ccccc7)oc65)ccc3n4-c3ccccc3)n2)cc1 ; c1ccc(-c2ccc(-c3nc(-c4ccccc4)nc(-c4ccc5c(c4)c4c6c(ccc7nc(-c8ccccc8)oc76)ccc4n5-c4ccccc4)n3)cc2)cc1 ; c1ccc(-c2nc(-c3ccc4ccccc4c3)nc(-c3ccc4c(c3)c3c5c(ccc6nc(-c7ccccc7)oc65)ccc3n4-c3ccccc3)n2)cc1 ; c1ccc(-c2nc(-c3ccc4c(c3)oc3ccccc34)nc(-c3ccc4c(c3)c3c5c(ccc6nc(-c7ccccc7)oc65)ccc3n4-c3ccccc3)n2)cc1]

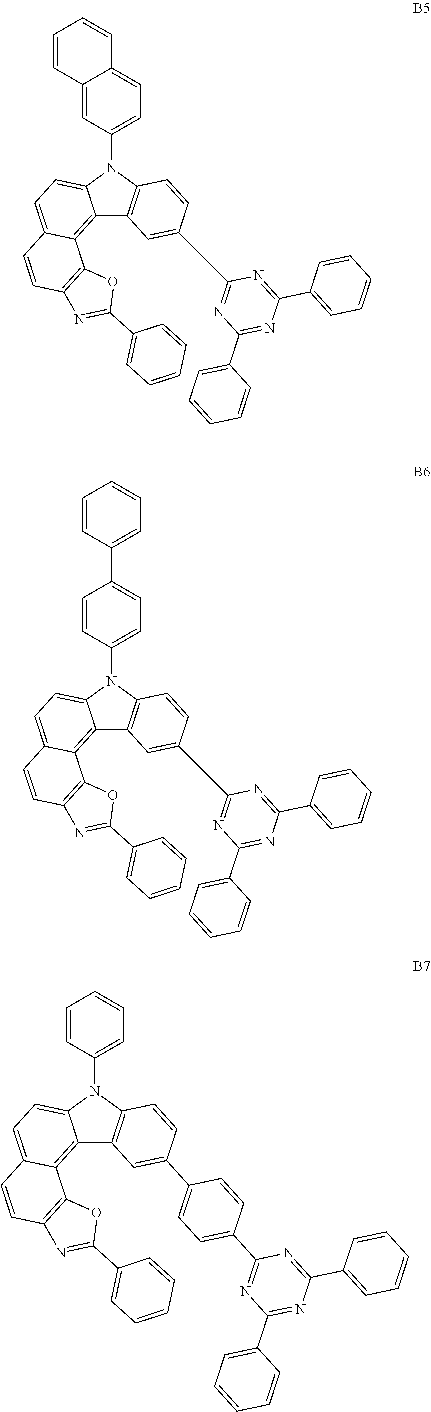 [SMILES: c1ccc(-c2nc(-c3ccccc3)nc(-c3ccc4c(c3)c3c5c(ccc6nc(-c7ccccc7)oc65)ccc3n4-c3ccc4ccccc4c3)n2)cc1 ; c1ccc(-c2ccc(-n3c4ccc(-c5nc(-c6ccccc6)nc(-c6ccccc6)n5)cc4c4c5c(ccc6nc(-c7ccccc7)oc65)ccc43)cc2)cc1 ; c1ccc(-c2nc(-c3ccccc3)nc(-c3ccc(-c4ccc5c(c4)c4c6c(ccc7nc(-c8ccccc8)oc76)ccc4n5-c4ccccc4)cc3)n2)cc1]

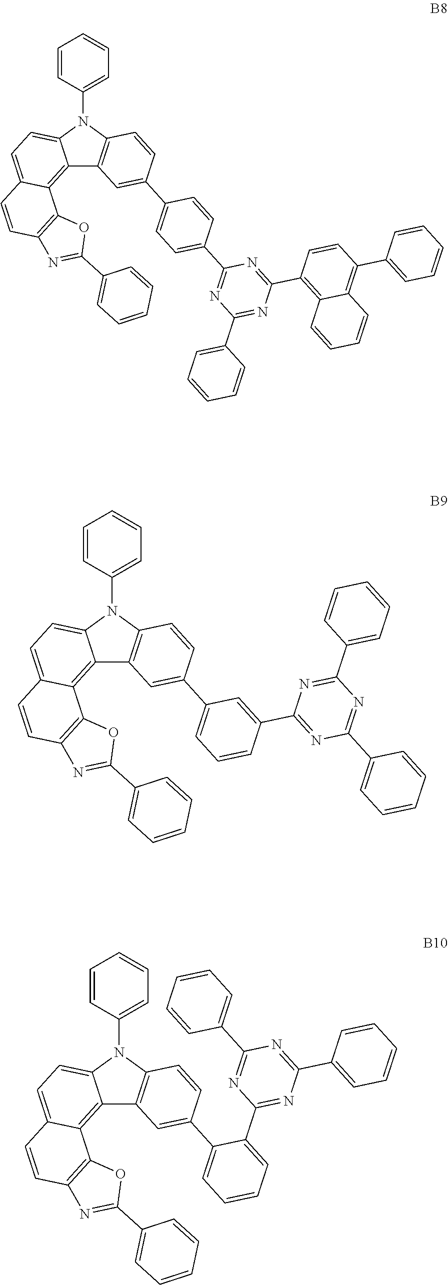 [SMILES: c1ccc(-c2nc(-c3ccc(-c4ccc5c(c4)c4c6c(ccc7nc(-c8ccccc8)oc76)ccc4n5-c4ccccc4)cc3)nc(-c3ccc(-c4ccccc4)c4ccccc34)n2)cc1 ; c1ccc(-c2nc(-c3ccccc3)nc(-c3cccc(-c4ccc5c(c4)c4c6c(ccc7nc(-c8ccccc8)oc76)ccc4n5-c4ccccc4)c3)n2)cc1 ; c1ccc(-c2nc(-c3ccccc3)nc(-c3ccccc3-c3ccc4c(c3)c3c5c(ccc6nc(-c7ccccc7)oc65)ccc3n4-c3ccccc3)n2)cc1]

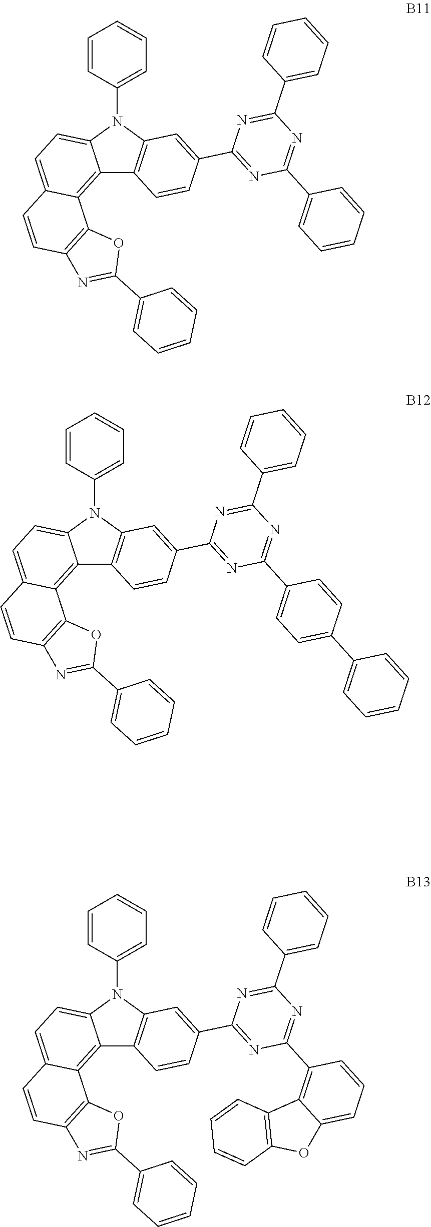 [SMILES: c1ccc(-c2nc(-c3ccccc3)nc(-c3ccc4c5c6c(ccc7nc(-c8ccccc8)oc76)ccc5n(-c5ccccc5)c4c3)n2)cc1 ; c1ccc(-c2ccc(-c3nc(-c4ccccc4)nc(-c4ccc5c6c7c(ccc8nc(-c9ccccc9)oc87)ccc6n(-c6ccccc6)c5c4)n3)cc2)cc1 ; c1ccc(-c2nc(-c3ccc4c5c6c(ccc7nc(-c8ccccc8)oc76)ccc5n(-c5ccccc5)c4c3)nc(-c3cccc4oc5ccccc5c34)n2)cc1]

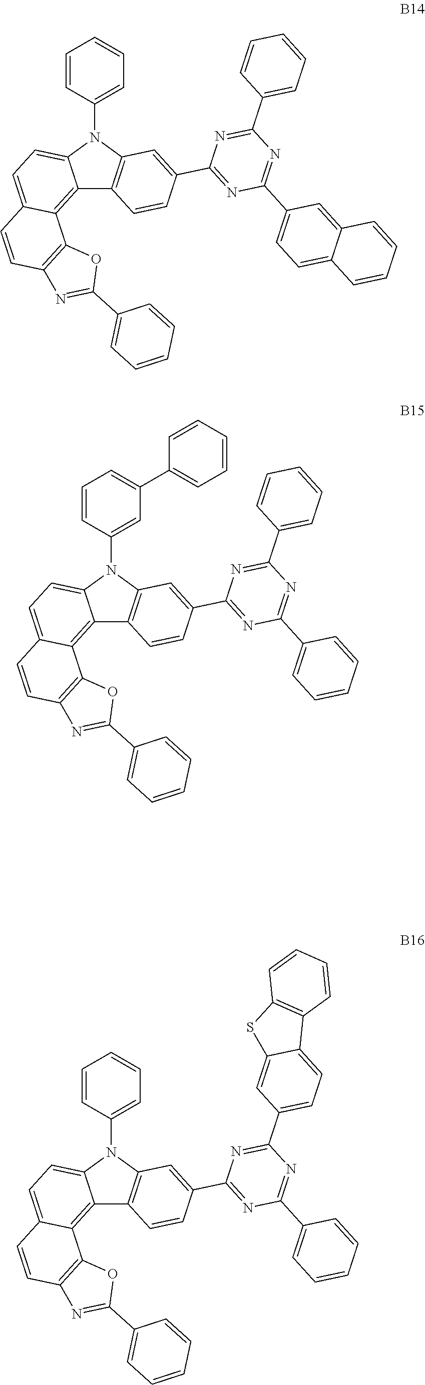 [SMILES: c1ccc(-c2nc(-c3ccc4ccccc4c3)nc(-c3ccc4c5c6c(ccc7nc(-c8ccccc8)oc76)ccc5n(-c5ccccc5)c4c3)n2)cc1 ; c1ccc(-c2cccc(-n3c4cc(-c5nc(-c6ccccc6)nc(-c6ccccc6)n5)ccc4c4c5c(ccc6nc(-c7ccccc7)oc65)ccc43)c2)cc1 ; c1ccc(-c2nc(-c3ccc4c(c3)sc3ccccc34)nc(-c3ccc4c5c6c(ccc7nc(-c8ccccc8)oc76)ccc5n(-c5ccccc5)c4c3)n2)cc1]

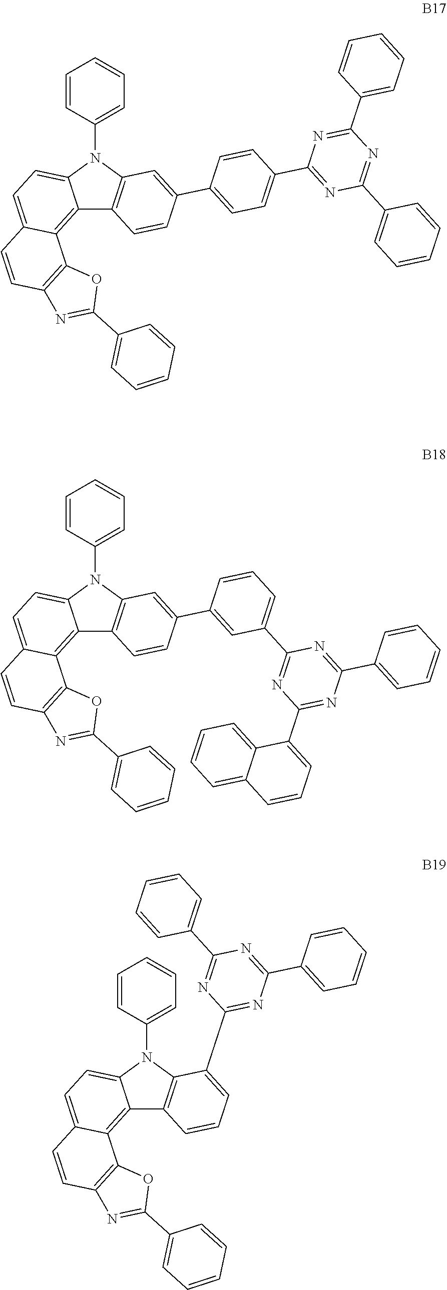 [SMILES: c1ccc(-c2nc(-c3ccccc3)nc(-c3ccc(-c4ccc5c6c7c(ccc8nc(-c9ccccc9)oc87)ccc6n(-c6ccccc6)c5c4)cc3)n2)cc1 ; c1ccc(-c2nc(-c3cccc(-c4ccc5c6c7c(ccc8nc(-c9ccccc9)oc87)ccc6n(-c6ccccc6)c5c4)c3)nc(-c3cccc4ccccc34)n2)cc1 ; c1ccc(-c2nc(-c3ccccc3)nc(-c3cccc4c5c6c(ccc7nc(-c8ccccc8)oc76)ccc5n(-c5ccccc5)c34)n2)cc1]

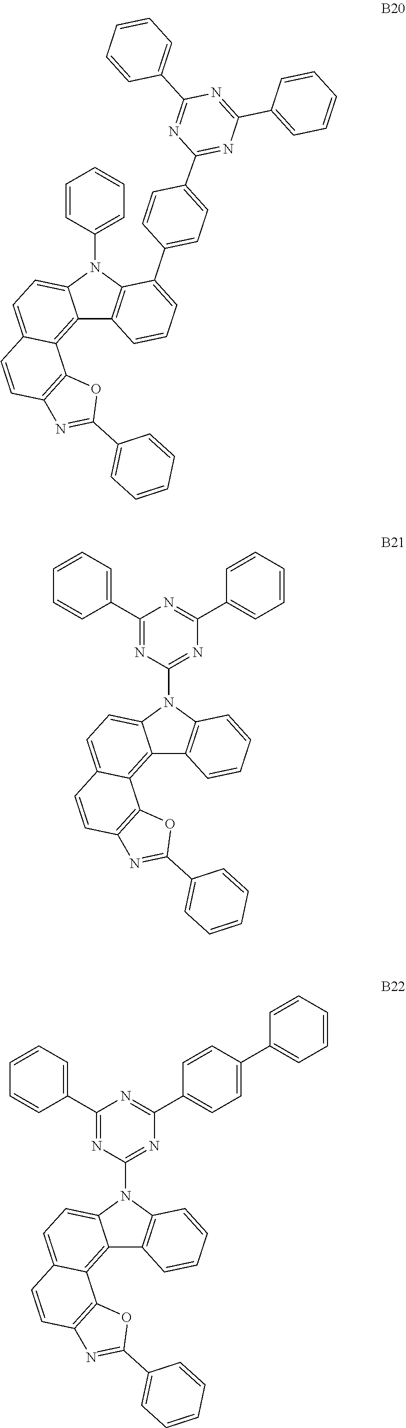 [SMILES: c1ccc(-c2nc(-c3ccccc3)nc(-c3ccc(-c4cccc5c6c7c(ccc8nc(-c9ccccc9)oc87)ccc6n(-c6ccccc6)c45)cc3)n2)cc1 ; c1ccc(-c2nc(-c3ccccc3)nc(-n3c4ccccc4c4c5c(ccc6nc(-c7ccccc7)oc65)ccc43)n2)cc1 ; c1ccc(-c2ccc(-c3nc(-c4ccccc4)nc(-n4c5ccccc5c5c6c(ccc7nc(-c8ccccc8)oc76)ccc54)n3)cc2)cc1]

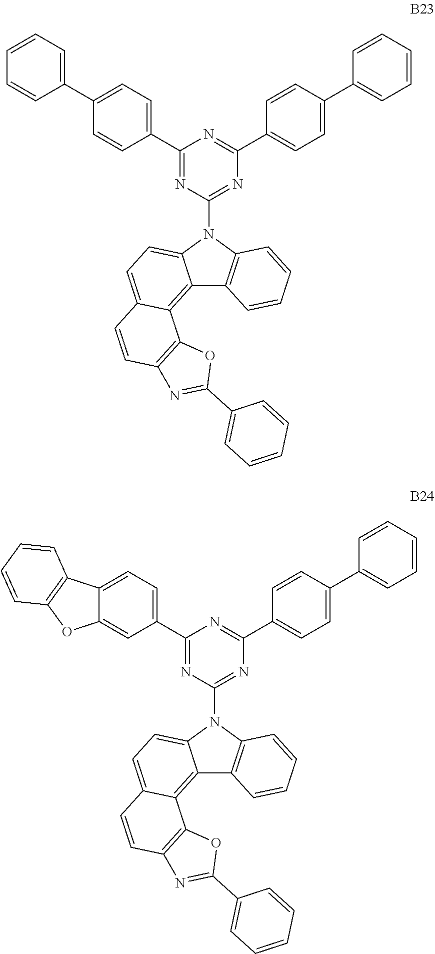 [SMILES: c1ccc(-c2ccc(-c3nc(-c4ccc(-c5ccccc5)cc4)nc(-n4c5ccccc5c5c6c(ccc7nc(-c8ccccc8)oc76)ccc54)n3)cc2)cc1 ; c1ccc(-c2ccc(-c3nc(-c4ccc5c(c4)oc4ccccc45)nc(-n4c5ccccc5c5c6c(ccc7nc(-c8ccccc8)oc76)ccc54)n3)cc2)cc1]

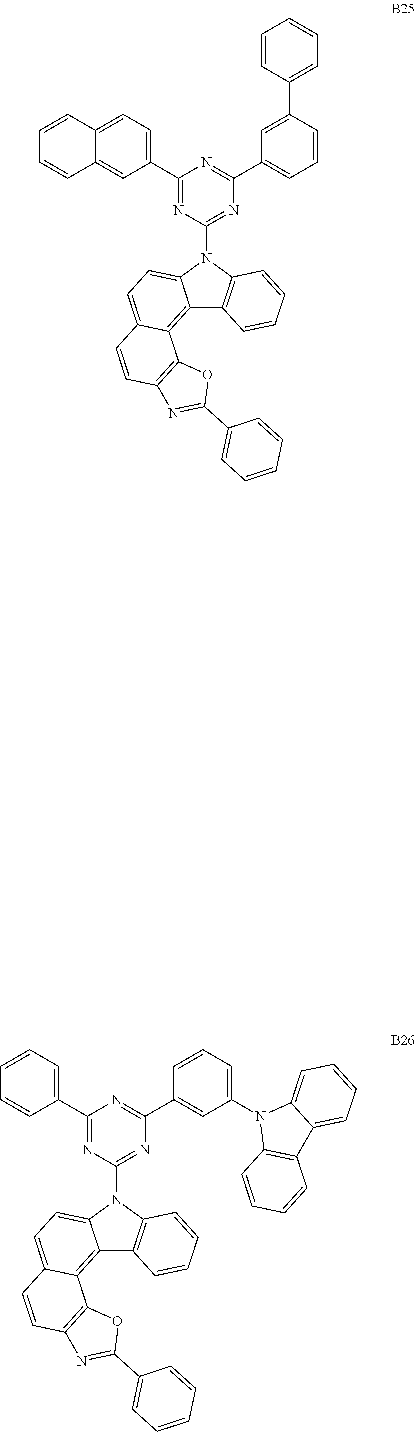 [SMILES: c1ccc(-c2cccc(-c3nc(-c4ccc5ccccc5c4)nc(-n4c5ccccc5c5c6c(ccc7nc(-c8ccccc8)oc76)ccc54)n3)c2)cc1 ; c1ccc(-c2nc(-c3cccc(-n4c5ccccc5c5ccccc54)c3)nc(-n3c4ccccc4c4c5c(ccc6nc(-c7ccccc7)oc65)ccc43)n2)cc1]

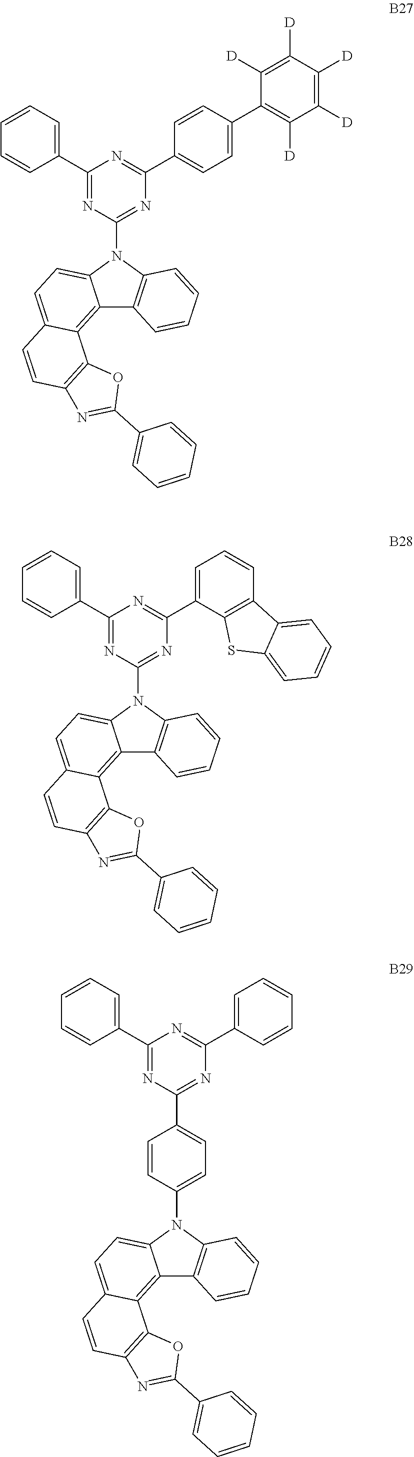 [SMILES: [2H]c1c([2H])c([2H])c(-c2ccc(-c3nc(-c4ccccc4)nc(-n4c5ccccc5c5c6c(ccc7nc(-c8ccccc8)oc76)ccc54)n3)cc2)c([2H])c1[2H] ; c1ccc(-c2nc(-c3cccc4c3sc3ccccc34)nc(-n3c4ccccc4c4c5c(ccc6nc(-c7ccccc7)oc65)ccc43)n2)cc1 ; c1ccc(-c2nc(-c3ccccc3)nc(-c3ccc(-n4c5ccccc5c5c6c(ccc7nc(-c8ccccc8)oc76)ccc54)cc3)n2)cc1]

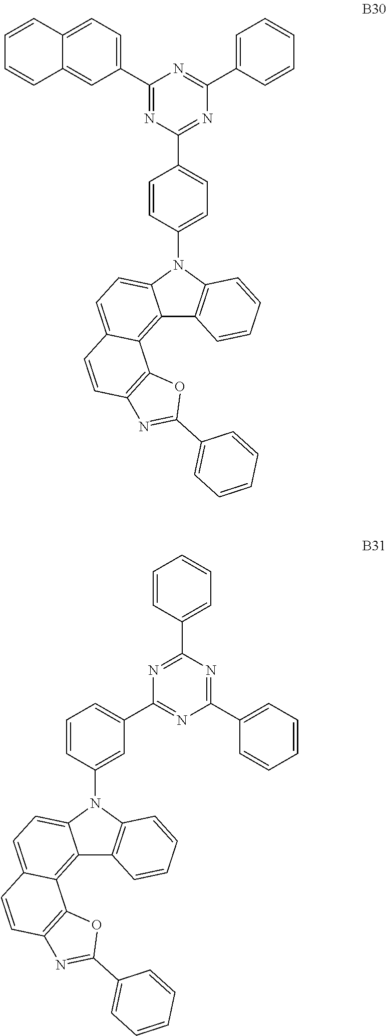 [SMILES: c1ccc(-c2nc(-c3ccc(-n4c5ccccc5c5c6c(ccc7nc(-c8ccccc8)oc76)ccc54)cc3)nc(-c3ccc4ccccc4c3)n2)cc1 ; c1ccc(-c2nc(-c3ccccc3)nc(-c3cccc(-n4c5ccccc5c5c6c(ccc7nc(-c8ccccc8)oc76)ccc54)c3)n2)cc1]

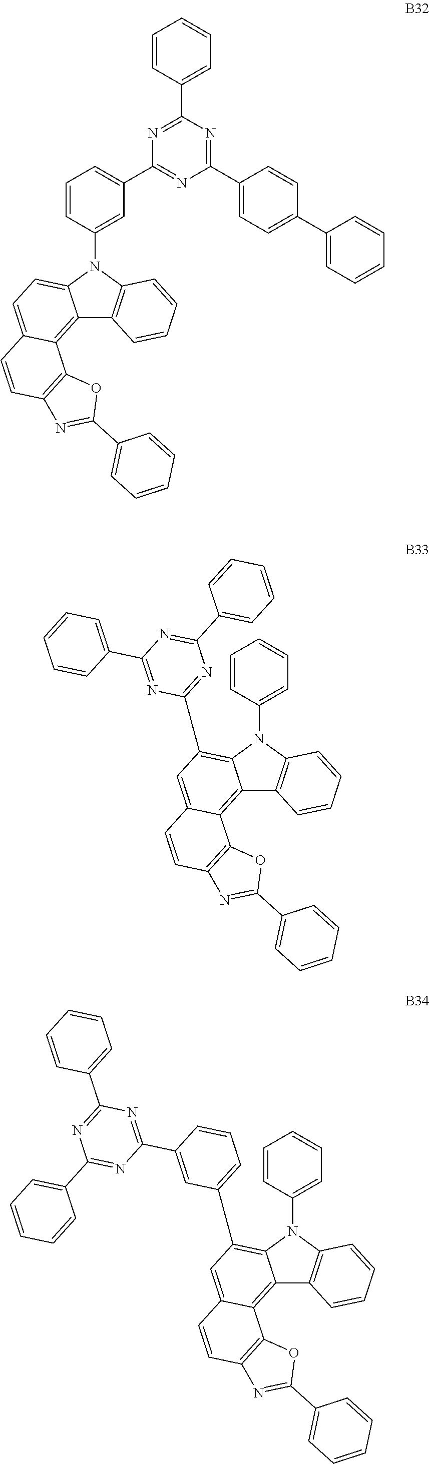 [SMILES: c1ccc(-c2ccc(-c3nc(-c4ccccc4)nc(-c4cccc(-n5c6ccccc6c6c7c(ccc8nc(-c9ccccc9)oc87)ccc65)c4)n3)cc2)cc1 ; c1ccc(-c2nc(-c3ccccc3)nc(-c3cc4ccc5nc(-c6ccccc6)oc5c4c4c5ccccc5n(-c5ccccc5)c34)n2)cc1 ; c1ccc(-c2nc(-c3ccccc3)nc(-c3cccc(-c4cc5ccc6nc(-c7ccccc7)oc6c5c5c6ccccc6n(-c6ccccc6)c45)c3)n2)cc1]

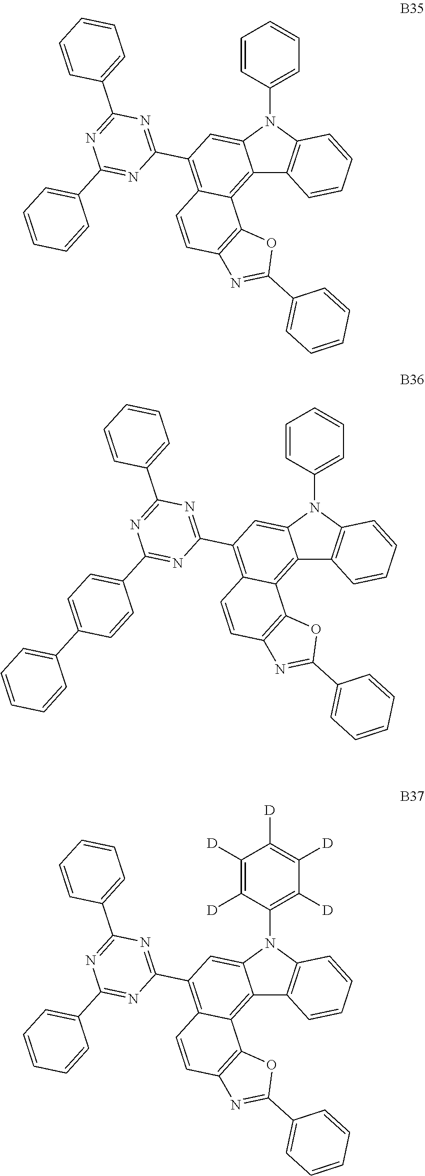 [SMILES: c1ccc(-c2nc(-c3ccccc3)nc(-c3cc4c(c5ccccc5n4-c4ccccc4)c4c3ccc3nc(-c5ccccc5)oc34)n2)cc1 ; c1ccc(-c2ccc(-c3nc(-c4ccccc4)nc(-c4cc5c(c6ccccc6n5-c5ccccc5)c5c4ccc4nc(-c6ccccc6)oc45)n3)cc2)cc1 ; [2H]c1c([2H])c([2H])c(-n2c3ccccc3c3c4c(ccc5nc(-c6ccccc6)oc54)c(-c4nc(-c5ccccc5)nc(-c5ccccc5)n4)cc32)c([2H])c1[2H]]

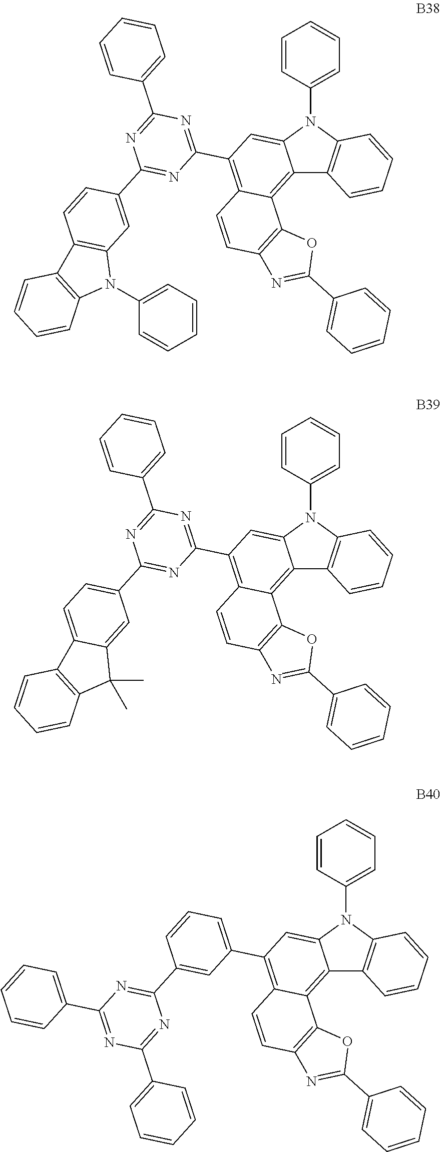 [SMILES: c1ccc(-c2nc(-c3ccc4c5ccccc5n(-c5ccccc5)c4c3)nc(-c3cc4c(c5ccccc5n4-c4ccccc4)c4c3ccc3nc(-c5ccccc5)oc34)n2)cc1 ; CC1(C)c2ccccc2-c2ccc(-c3nc(-c4ccccc4)nc(-c4cc5c(c6ccccc6n5-c5ccccc5)c5c4ccc4nc(-c6ccccc6)oc45)n3)cc21 ; c1ccc(-c2nc(-c3ccccc3)nc(-c3cccc(-c4cc5c(c6ccccc6n5-c5ccccc5)c5c4ccc4nc(-c6ccccc6)oc45)c3)n2)cc1]

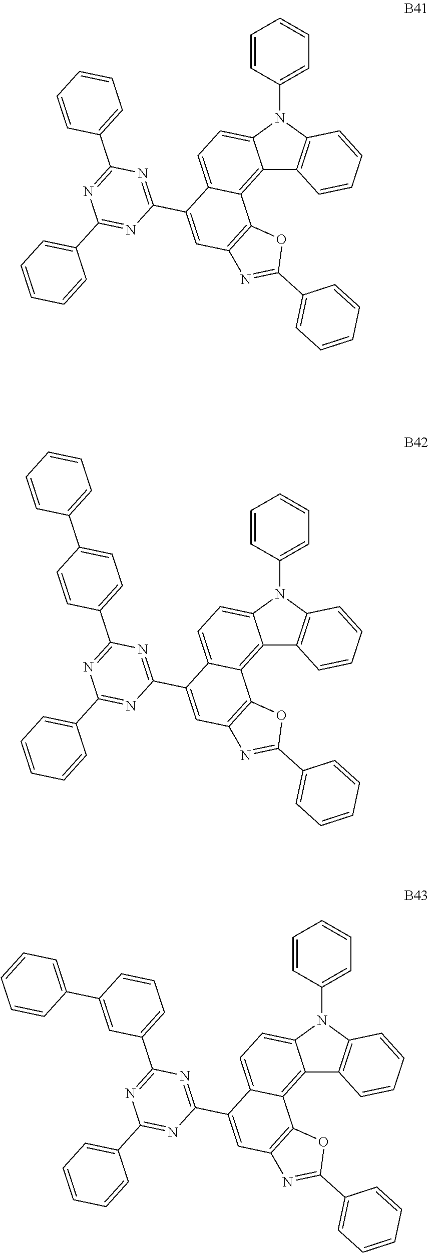 [SMILES: c1ccc(-c2nc(-c3ccccc3)nc(-c3cc4nc(-c5ccccc5)oc4c4c3ccc3c4c4ccccc4n3-c3ccccc3)n2)cc1 ; c1ccc(-c2ccc(-c3nc(-c4ccccc4)nc(-c4cc5nc(-c6ccccc6)oc5c5c4ccc4c5c5ccccc5n4-c4ccccc4)n3)cc2)cc1 ; c1ccc(-c2cccc(-c3nc(-c4ccccc4)nc(-c4cc5nc(-c6ccccc6)oc5c5c4ccc4c5c5ccccc5n4-c4ccccc4)n3)c2)cc1]

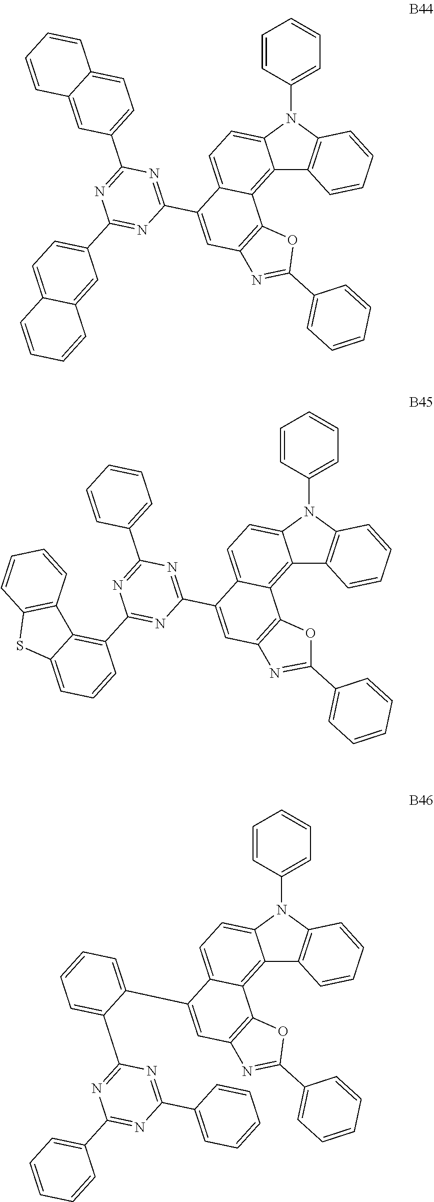 [SMILES: c1ccc(-c2nc3cc(-c4nc(-c5ccc6ccccc6c5)nc(-c5ccc6ccccc6c5)n4)c4ccc5c(c6ccccc6n5-c5ccccc5)c4c3o2)cc1 ; c1ccc(-c2nc(-c3cc4nc(-c5ccccc5)oc4c4c3ccc3c4c4ccccc4n3-c3ccccc3)nc(-c3cccc4sc5ccccc5c34)n2)cc1 ; c1ccc(-c2nc(-c3ccccc3)nc(-c3ccccc3-c3cc4nc(-c5ccccc5)oc4c4c3ccc3c4c4ccccc4n3-c3ccccc3)n2)cc1]

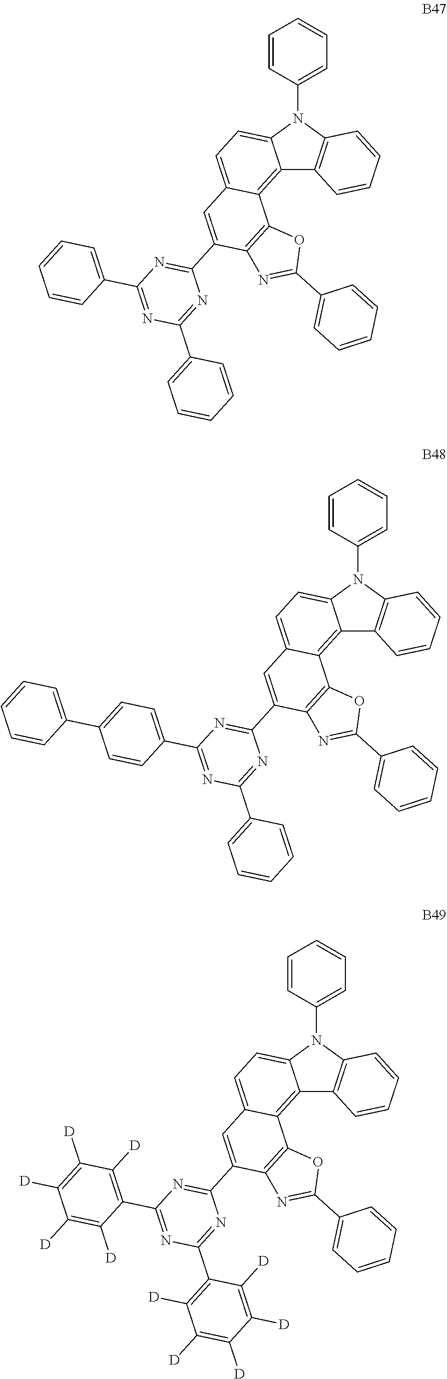 [SMILES: c1ccc(-c2nc(-c3ccccc3)nc(-c3cc4ccc5c(c6ccccc6n5-c5ccccc5)c4c4oc(-c5ccccc5)nc34)n2)cc1 ; c1ccc(-c2ccc(-c3nc(-c4ccccc4)nc(-c4cc5ccc6c(c7ccccc7n6-c6ccccc6)c5c5oc(-c6ccccc6)nc45)n3)cc2)cc1 ; [2H]c1c([2H])c([2H])c(-c2nc(-c3c([2H])c([2H])c([2H])c([2H])c3[2H])nc(-c3cc4ccc5c(c6ccccc6n5-c5ccccc5)c4c4oc(-c5ccccc5)nc34)n2)c([2H])c1[2H]]

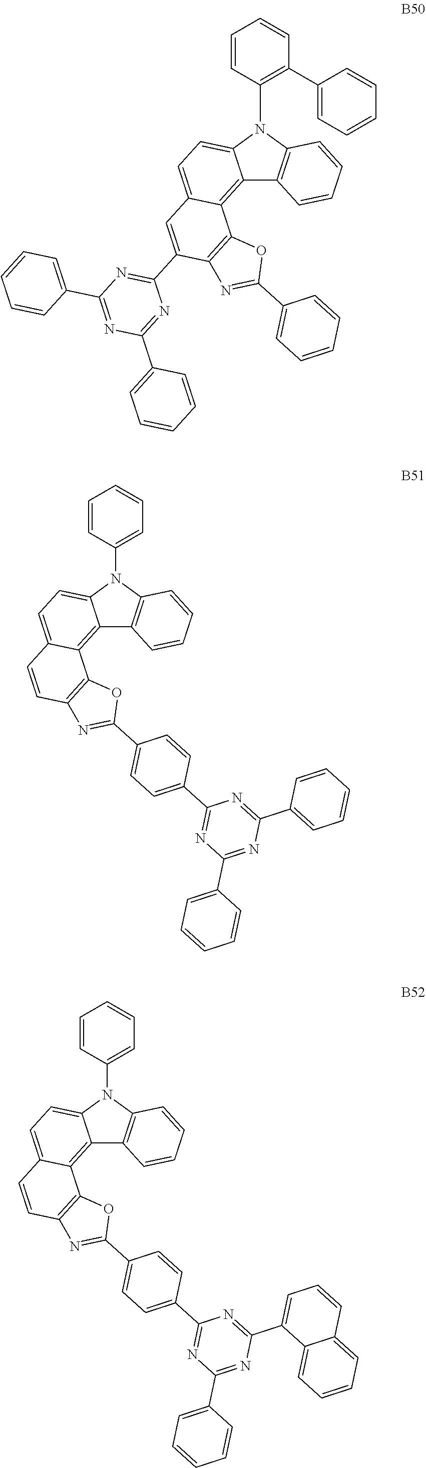 [SMILES: c1ccc(-c2nc(-c3ccccc3)nc(-c3cc4ccc5c(c6ccccc6n5-c5ccccc5-c5ccccc5)c4c4oc(-c5ccccc5)nc34)n2)cc1 ; c1ccc(-c2nc(-c3ccccc3)nc(-c3ccc(-c4nc5ccc6ccc7c(c8ccccc8n7-c7ccccc7)c6c5o4)cc3)n2)cc1 ; c1ccc(-c2nc(-c3ccc(-c4nc5ccc6ccc7c(c8ccccc8n7-c7ccccc7)c6c5o4)cc3)nc(-c3cccc4ccccc34)n2)cc1]

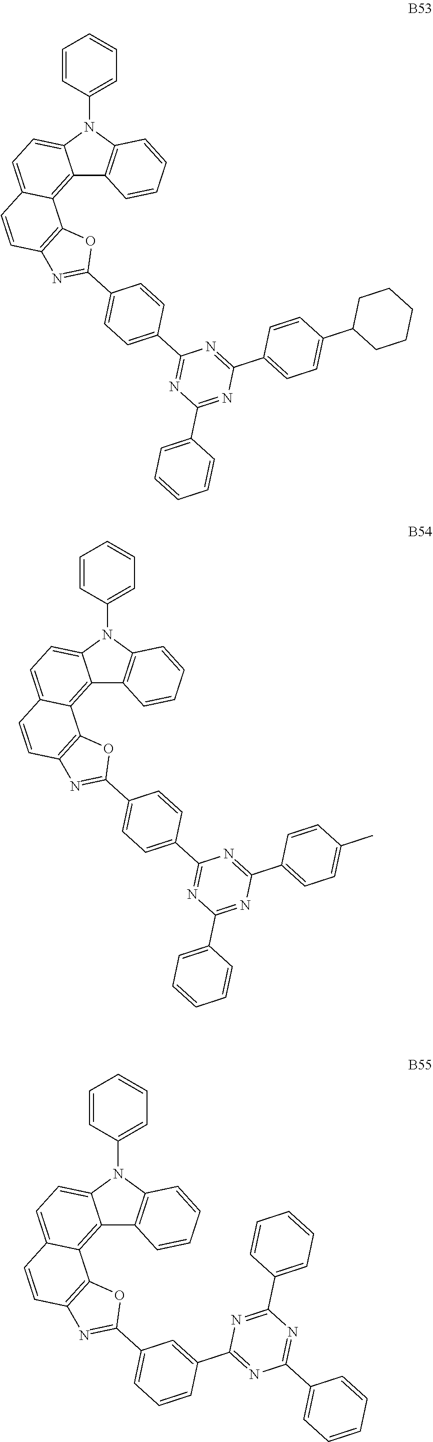 [SMILES: c1ccc(-c2nc(-c3ccc(-c4nc5ccc6ccc7c(c8ccccc8n7-c7ccccc7)c6c5o4)cc3)nc(-c3ccc(C4CCCCC4)cc3)n2)cc1 ; Cc1ccc(-c2nc(-c3ccccc3)nc(-c3ccc(-c4nc5ccc6ccc7c(c8ccccc8n7-c7ccccc7)c6c5o4)cc3)n2)cc1 ; c1ccc(-c2nc(-c3ccccc3)nc(-c3cccc(-c4nc5ccc6ccc7c(c8ccccc8n7-c7ccccc7)c6c5o4)c3)n2)cc1]

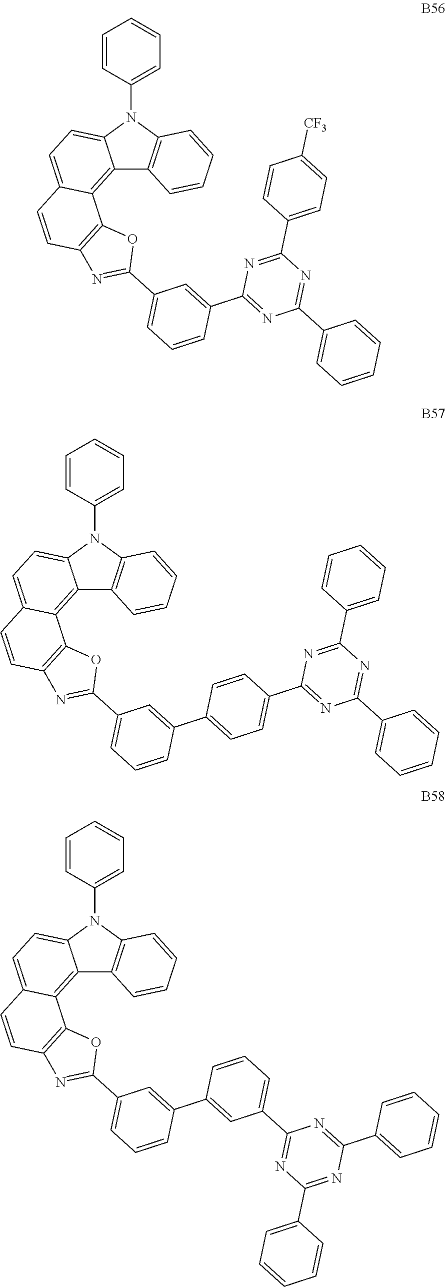 [SMILES: FC(F)(F)c1ccc(-c2nc(-c3ccccc3)nc(-c3cccc(-c4nc5ccc6ccc7c(c8ccccc8n7-c7ccccc7)c6c5o4)c3)n2)cc1 ; c1ccc(-c2nc(-c3ccccc3)nc(-c3ccc(-c4cccc(-c5nc6ccc7ccc8c(c9ccccc9n8-c8ccccc8)c7c6o5)c4)cc3)n2)cc1 ; c1ccc(-c2nc(-c3ccccc3)nc(-c3cccc(-c4cccc(-c5nc6ccc7ccc8c(c9ccccc9n8-c8ccccc8)c7c6o5)c4)c3)n2)cc1]

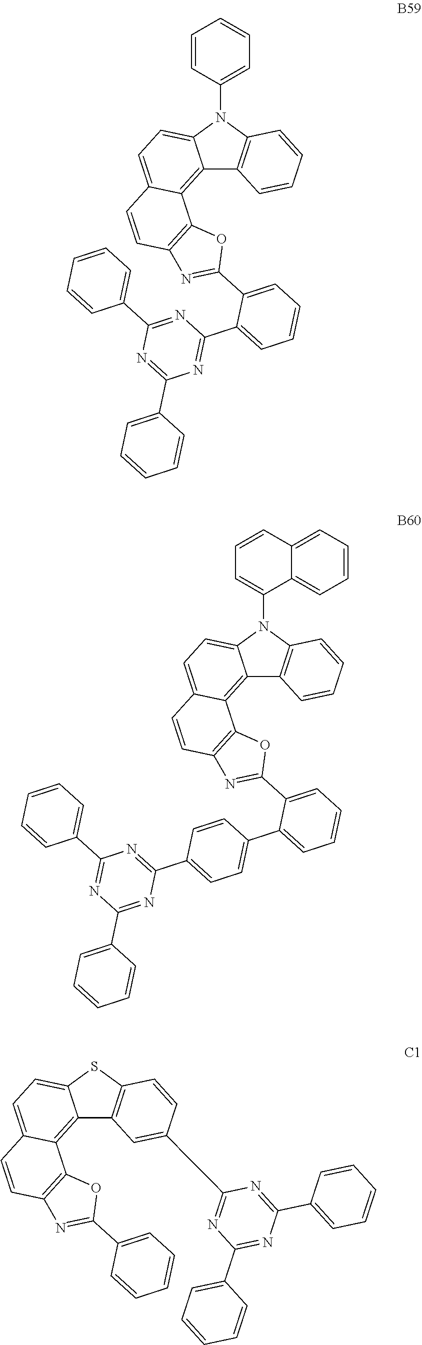 [SMILES: c1ccc(-c2nc(-c3ccccc3)nc(-c3ccccc3-c3nc4ccc5ccc6c(c7ccccc7n6-c6ccccc6)c5c4o3)n2)cc1 ; c1ccc(-c2nc(-c3ccccc3)nc(-c3ccc(-c4ccccc4-c4nc5ccc6ccc7c(c8ccccc8n7-c7cccc8ccccc78)c6c5o4)cc3)n2)cc1 ; c1ccc(-c2nc(-c3ccccc3)nc(-c3ccc4sc5ccc6ccc7nc(-c8ccccc8)oc7c6c5c4c3)n2)cc1]

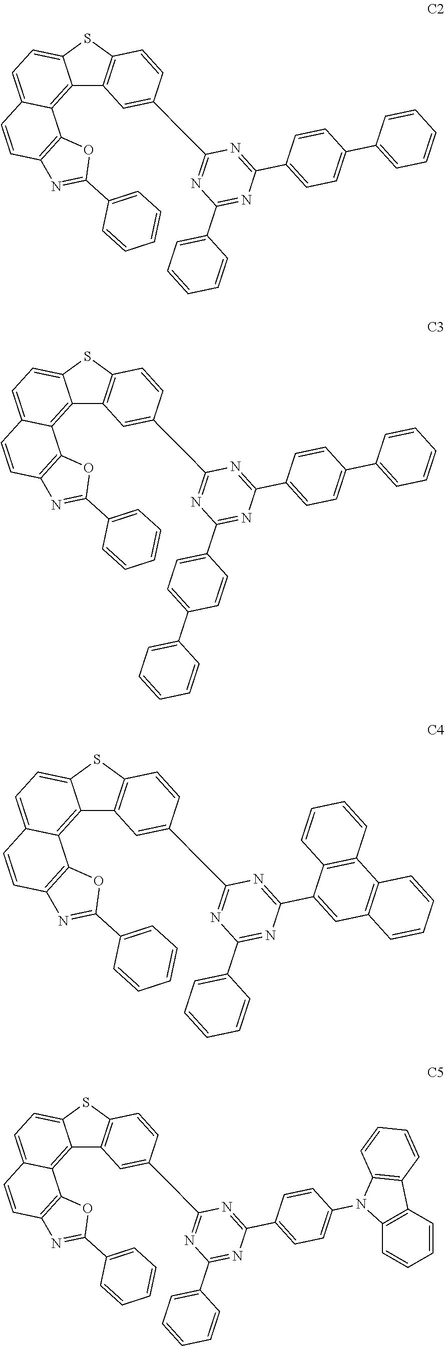 [SMILES: c1ccc(-c2ccc(-c3nc(-c4ccccc4)nc(-c4ccc5sc6ccc7ccc8nc(-c9ccccc9)oc8c7c6c5c4)n3)cc2)cc1 ; c1ccc(-c2ccc(-c3nc(-c4ccc(-c5ccccc5)cc4)nc(-c4ccc5sc6ccc7ccc8nc(-c9ccccc9)oc8c7c6c5c4)n3)cc2)cc1 ; c1ccc(-c2nc(-c3ccc4sc5ccc6ccc7nc(-c8ccccc8)oc7c6c5c4c3)nc(-c3cc4ccccc4c4ccccc34)n2)cc1 ; c1ccc(-c2nc(-c3ccc(-n4c5ccccc5c5ccccc54)cc3)nc(-c3ccc4sc5ccc6ccc7nc(-c8ccccc8)oc7c6c5c4c3)n2)cc1]

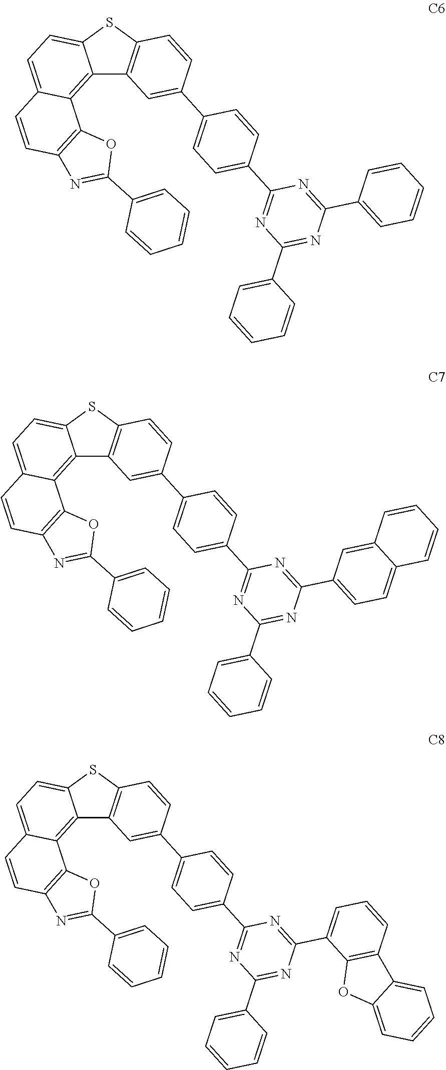 [SMILES: c1ccc(-c2nc(-c3ccccc3)nc(-c3ccc(-c4ccc5sc6ccc7ccc8nc(-c9ccccc9)oc8c7c6c5c4)cc3)n2)cc1 ; c1ccc(-c2nc(-c3ccc(-c4ccc5sc6ccc7ccc8nc(-c9ccccc9)oc8c7c6c5c4)cc3)nc(-c3ccc4ccccc4c3)n2)cc1 ; c1ccc(-c2nc(-c3ccc(-c4ccc5sc6ccc7ccc8nc(-c9ccccc9)oc8c7c6c5c4)cc3)nc(-c3cccc4c3oc3ccccc34)n2)cc1]

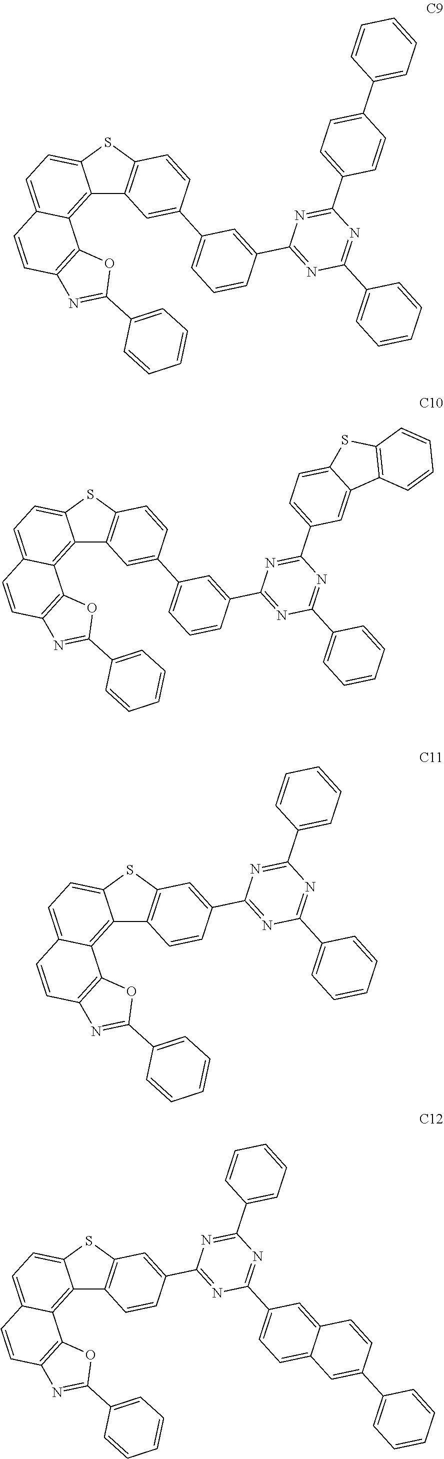 [SMILES: c1ccc(-c2ccc(-c3nc(-c4ccccc4)nc(-c4cccc(-c5ccc6sc7ccc8ccc9nc(-c%10ccccc%10)oc9c8c7c6c5)c4)n3)cc2)cc1 ; c1ccc(-c2nc(-c3cccc(-c4ccc5sc6ccc7ccc8nc(-c9ccccc9)oc8c7c6c5c4)c3)nc(-c3ccc4sc5ccccc5c4c3)n2)cc1 ; c1ccc(-c2nc(-c3ccccc3)nc(-c3ccc4c(c3)sc3ccc5ccc6nc(-c7ccccc7)oc6c5c34)n2)cc1 ; c1ccc(-c2ccc3cc(-c4nc(-c5ccccc5)nc(-c5ccc6c(c5)sc5ccc7ccc8nc(-c9ccccc9)oc8c7c56)n4)ccc3c2)cc1]

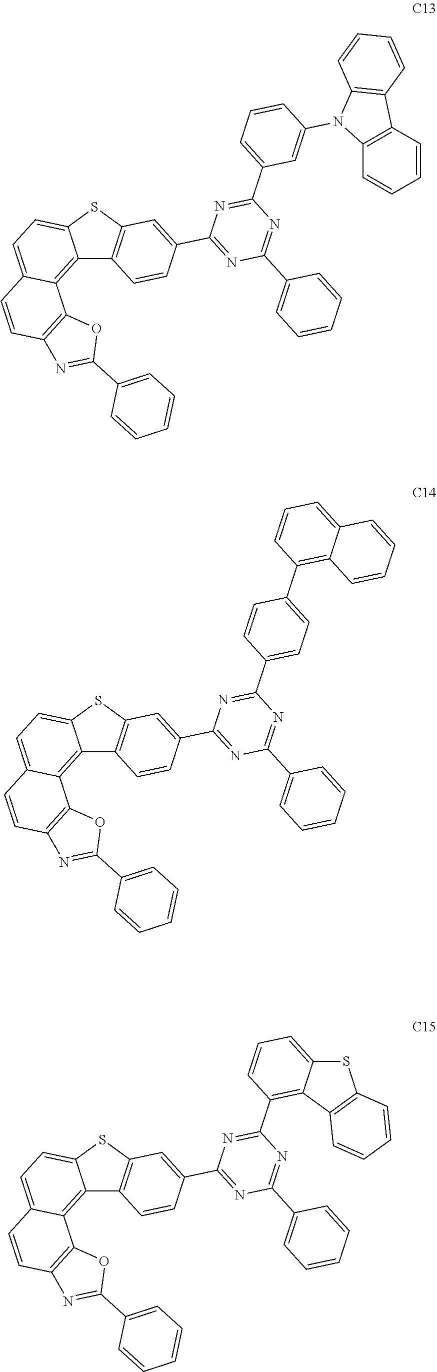 [SMILES: c1ccc(-c2nc(-c3cccc(-n4c5ccccc5c5ccccc54)c3)nc(-c3ccc4c(c3)sc3ccc5ccc6nc(-c7ccccc7)oc6c5c34)n2)cc1 ; c1ccc(-c2nc(-c3ccc(-c4cccc5ccccc45)cc3)nc(-c3ccc4c(c3)sc3ccc5ccc6nc(-c7ccccc7)oc6c5c34)n2)cc1 ; c1ccc(-c2nc(-c3ccc4c(c3)sc3ccc5ccc6nc(-c7ccccc7)oc6c5c34)nc(-c3cccc4sc5ccccc5c34)n2)cc1]

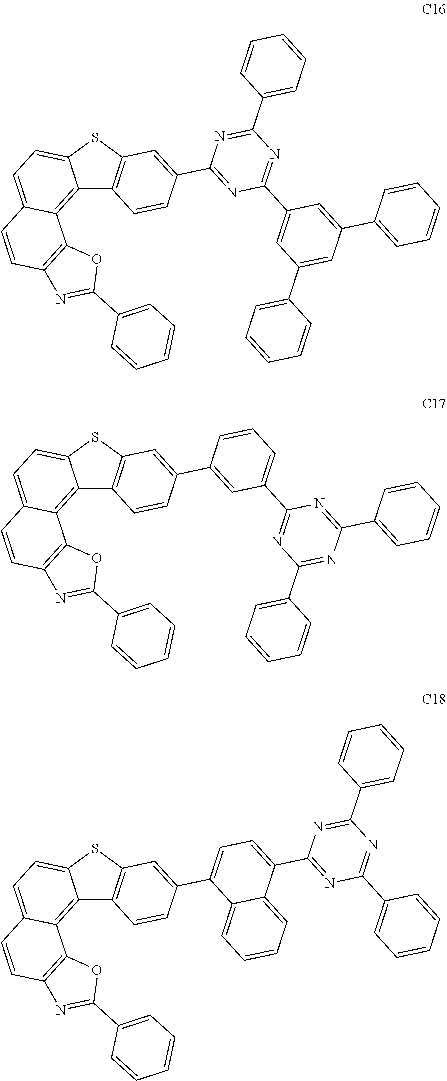 [SMILES: c1ccc(-c2cc(-c3ccccc3)cc(-c3nc(-c4ccccc4)nc(-c4ccc5c(c4)sc4ccc6ccc7nc(-c8ccccc8)oc7c6c45)n3)c2)cc1 ; c1ccc(-c2nc(-c3ccccc3)nc(-c3cccc(-c4ccc5c(c4)sc4ccc6ccc7nc(-c8ccccc8)oc7c6c45)c3)n2)cc1 ; c1ccc(-c2nc(-c3ccccc3)nc(-c3ccc(-c4ccc5c(c4)sc4ccc6ccc7nc(-c8ccccc8)oc7c6c45)c4ccccc34)n2)cc1]

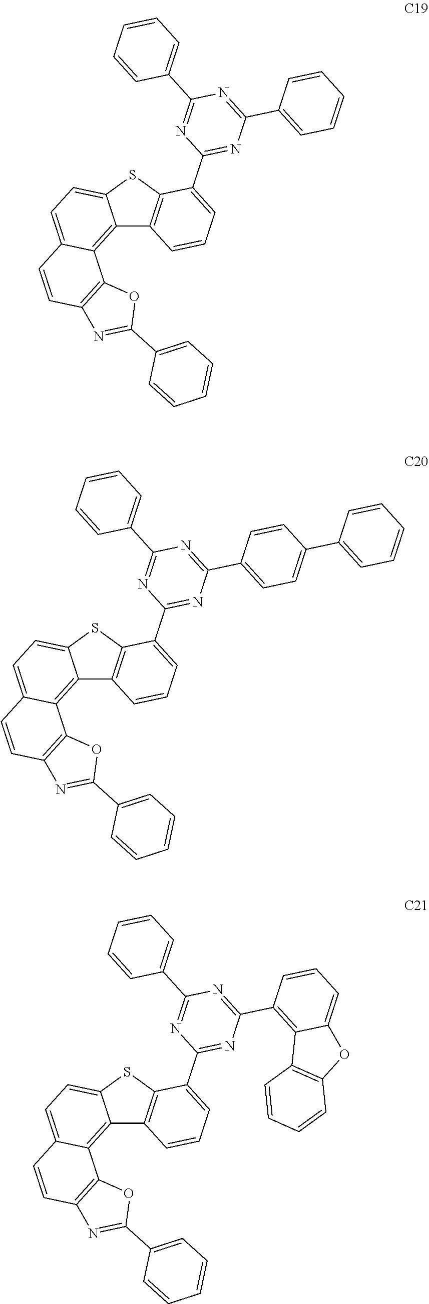 [SMILES: c1ccc(-c2nc(-c3ccccc3)nc(-c3cccc4c3sc3ccc5ccc6nc(-c7ccccc7)oc6c5c34)n2)cc1 ; c1ccc(-c2ccc(-c3nc(-c4ccccc4)nc(-c4cccc5c4sc4ccc6ccc7nc(-c8ccccc8)oc7c6c45)n3)cc2)cc1 ; c1ccc(-c2nc(-c3cccc4c3sc3ccc5ccc6nc(-c7ccccc7)oc6c5c34)nc(-c3cccc4oc5ccccc5c34)n2)cc1]

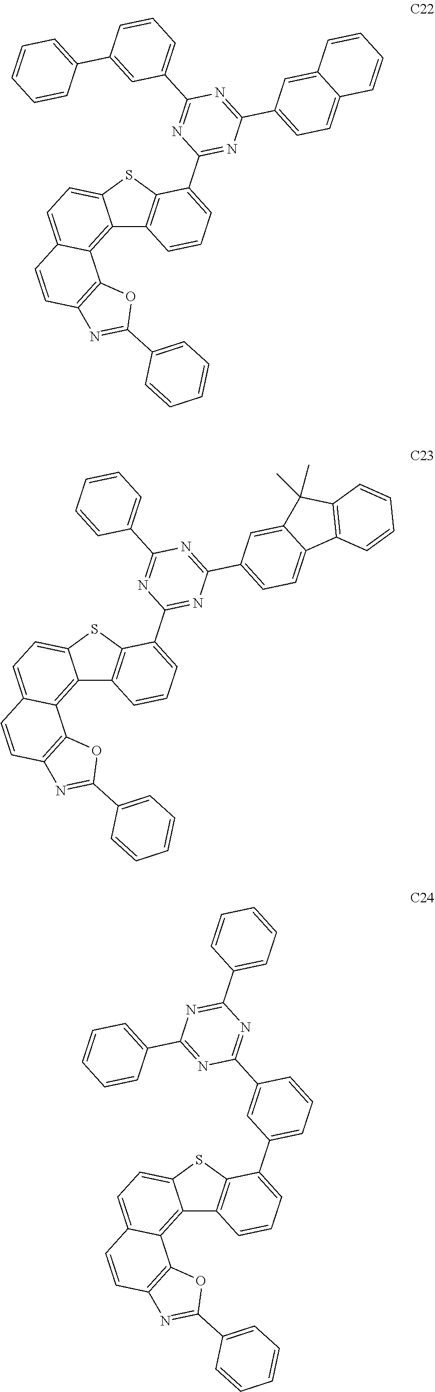 [SMILES: c1ccc(-c2cccc(-c3nc(-c4ccc5ccccc5c4)nc(-c4cccc5c4sc4ccc6ccc7nc(-c8ccccc8)oc7c6c45)n3)c2)cc1 ; CC1(C)c2ccccc2-c2ccc(-c3nc(-c4ccccc4)nc(-c4cccc5c4sc4ccc6ccc7nc(-c8ccccc8)oc7c6c45)n3)cc21 ; c1ccc(-c2nc(-c3ccccc3)nc(-c3cccc(-c4cccc5c4sc4ccc6ccc7nc(-c8ccccc8)oc7c6c45)c3)n2)cc1]

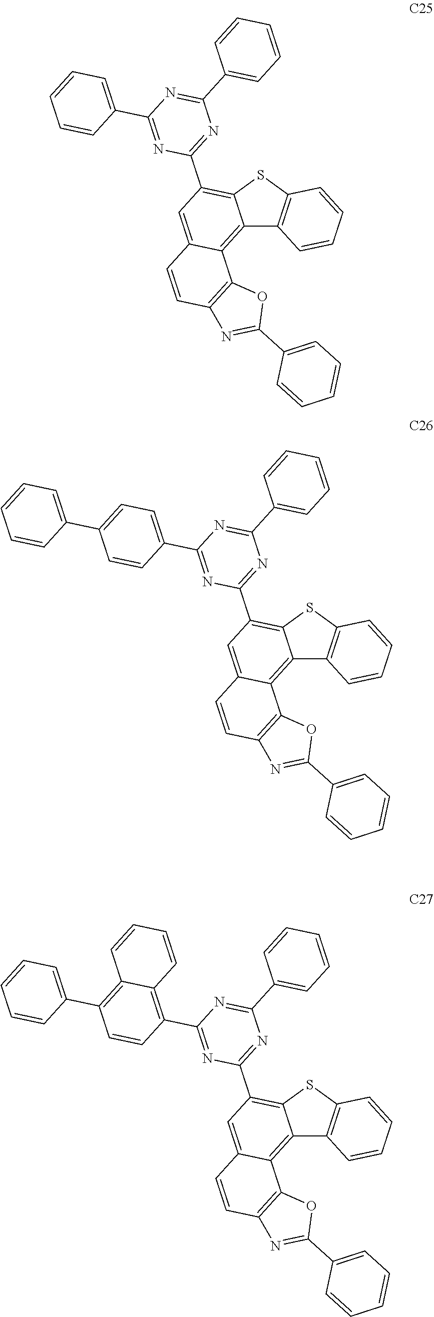 [SMILES: c1ccc(-c2nc(-c3ccccc3)nc(-c3cc4ccc5nc(-c6ccccc6)oc5c4c4c3sc3ccccc34)n2)cc1 ; c1ccc(-c2ccc(-c3nc(-c4ccccc4)nc(-c4cc5ccc6nc(-c7ccccc7)oc6c5c5c4sc4ccccc45)n3)cc2)cc1 ; c1ccc(-c2nc(-c3ccc(-c4ccccc4)c4ccccc34)nc(-c3cc4ccc5nc(-c6ccccc6)oc5c4c4c3sc3ccccc34)n2)cc1]

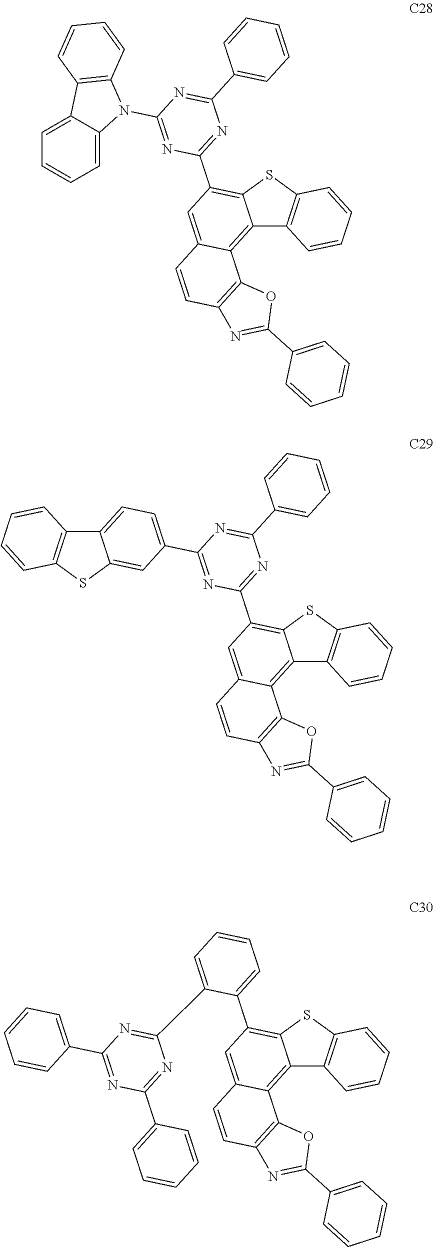 [SMILES: c1ccc(-c2nc(-c3cc4ccc5nc(-c6ccccc6)oc5c4c4c3sc3ccccc34)nc(-n3c4ccccc4c4ccccc43)n2)cc1 ; c1ccc(-c2nc(-c3ccc4c(c3)sc3ccccc34)nc(-c3cc4ccc5nc(-c6ccccc6)oc5c4c4c3sc3ccccc34)n2)cc1 ; c1ccc(-c2nc(-c3ccccc3)nc(-c3ccccc3-c3cc4ccc5nc(-c6ccccc6)oc5c4c4c3sc3ccccc34)n2)cc1]

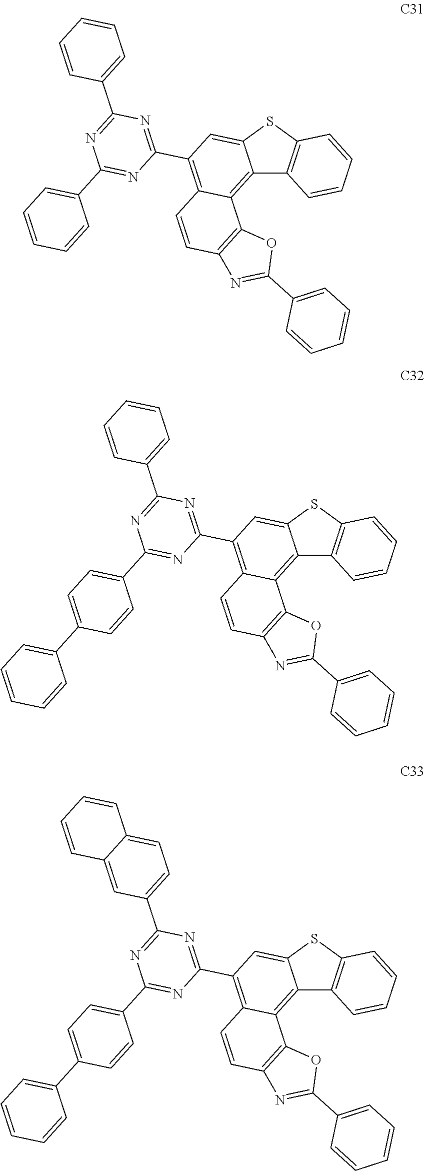 [SMILES: c1ccc(-c2nc(-c3ccccc3)nc(-c3cc4sc5ccccc5c4c4c3ccc3nc(-c5ccccc5)oc34)n2)cc1 ; c1ccc(-c2ccc(-c3nc(-c4ccccc4)nc(-c4cc5sc6ccccc6c5c5c4ccc4nc(-c6ccccc6)oc45)n3)cc2)cc1 ; c1ccc(-c2ccc(-c3nc(-c4ccc5ccccc5c4)nc(-c4cc5sc6ccccc6c5c5c4ccc4nc(-c6ccccc6)oc45)n3)cc2)cc1]

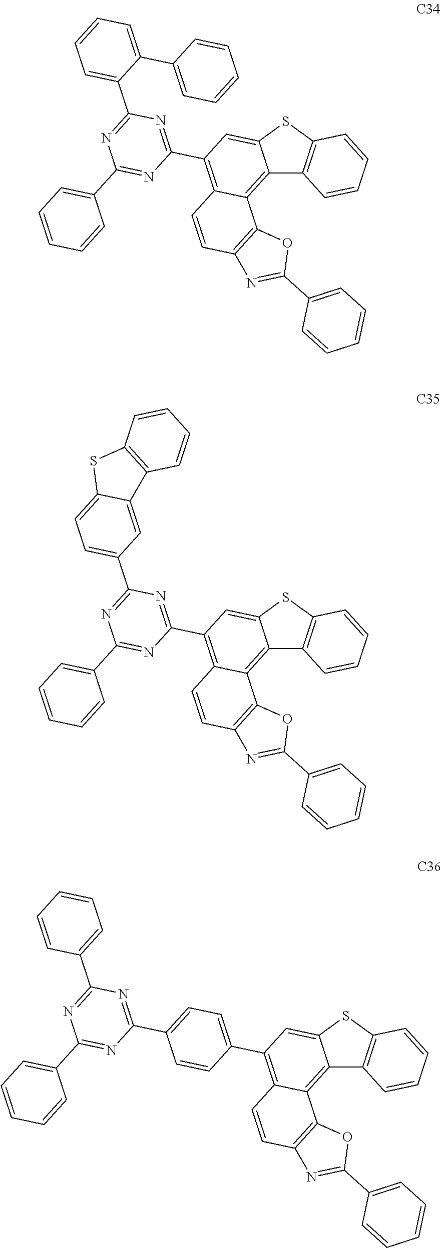 [SMILES: c1ccc(-c2nc(-c3ccccc3-c3ccccc3)nc(-c3cc4sc5ccccc5c4c4c3ccc3nc(-c5ccccc5)oc34)n2)cc1 ; c1ccc(-c2nc(-c3ccc4sc5ccccc5c4c3)nc(-c3cc4sc5ccccc5c4c4c3ccc3nc(-c5ccccc5)oc34)n2)cc1 ; c1ccc(-c2nc(-c3ccccc3)nc(-c3ccc(-c4cc5sc6ccccc6c5c5c4ccc4nc(-c6ccccc6)oc45)cc3)n2)cc1]

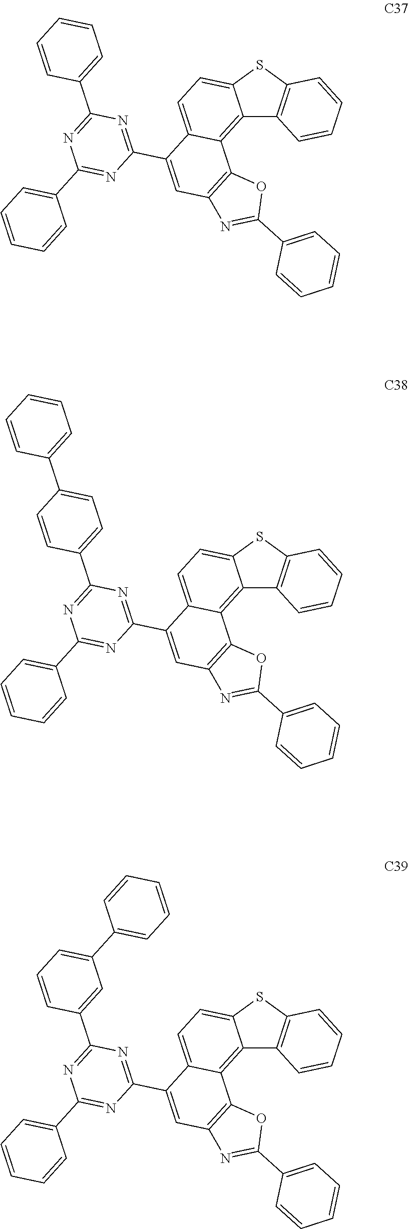 [SMILES: c1ccc(-c2nc(-c3ccccc3)nc(-c3cc4nc(-c5ccccc5)oc4c4c3ccc3sc5ccccc5c34)n2)cc1 ; c1ccc(-c2ccc(-c3nc(-c4ccccc4)nc(-c4cc5nc(-c6ccccc6)oc5c5c4ccc4sc6ccccc6c45)n3)cc2)cc1 ; c1ccc(-c2cccc(-c3nc(-c4ccccc4)nc(-c4cc5nc(-c6ccccc6)oc5c5c4ccc4sc6ccccc6c45)n3)c2)cc1]

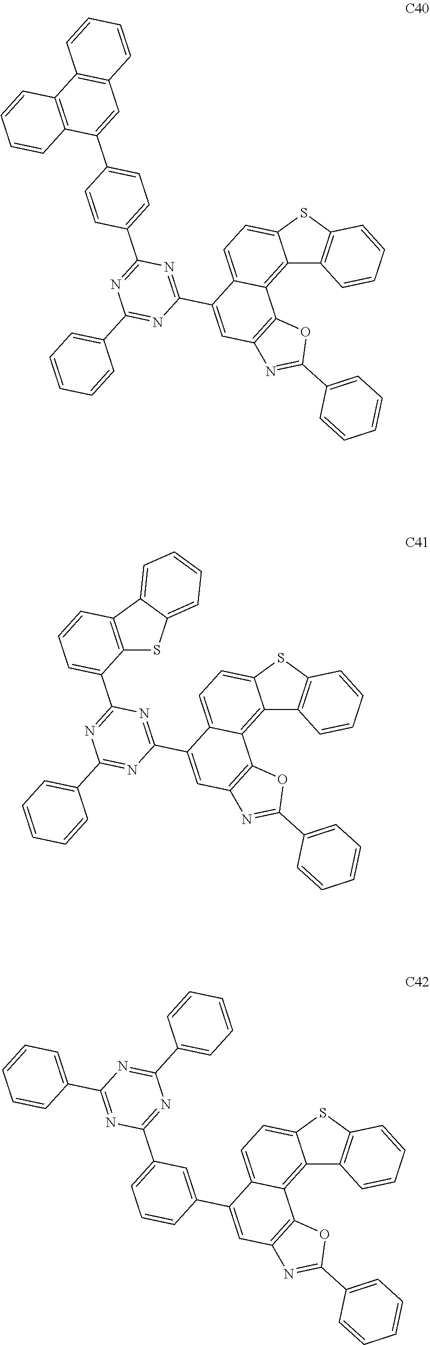 [SMILES: c1ccc(-c2nc(-c3ccc(-c4cc5ccccc5c5ccccc45)cc3)nc(-c3cc4nc(-c5ccccc5)oc4c4c3ccc3sc5ccccc5c34)n2)cc1 ; c1ccc(-c2nc(-c3cc4nc(-c5ccccc5)oc4c4c3ccc3sc5ccccc5c34)nc(-c3cccc4c3sc3ccccc34)n2)cc1 ; c1ccc(-c2nc(-c3ccccc3)nc(-c3cccc(-c4cc5nc(-c6ccccc6)oc5c5c4ccc4sc6ccccc6c45)c3)n2)cc1]

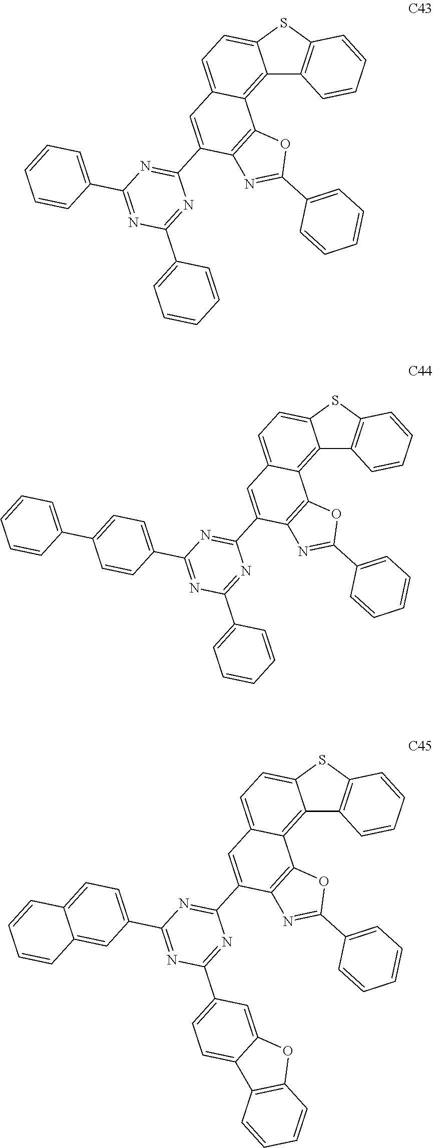 [SMILES: c1ccc(-c2nc(-c3ccccc3)nc(-c3cc4ccc5sc6ccccc6c5c4c4oc(-c5ccccc5)nc34)n2)cc1 ; c1ccc(-c2ccc(-c3nc(-c4ccccc4)nc(-c4cc5ccc6sc7ccccc7c6c5c5oc(-c6ccccc6)nc45)n3)cc2)cc1 ; c1ccc(-c2nc3c(-c4nc(-c5ccc6ccccc6c5)nc(-c5ccc6c(c5)oc5ccccc56)n4)cc4ccc5sc6ccccc6c5c4c3o2)cc1]

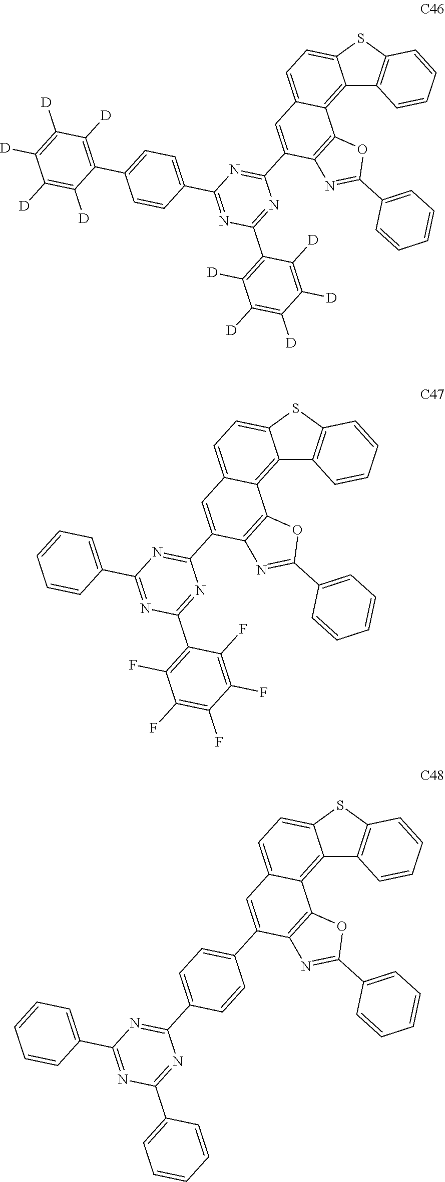 [SMILES: [2H]c1c([2H])c([2H])c(-c2ccc(-c3nc(-c4c([2H])c([2H])c([2H])c([2H])c4[2H])nc(-c4cc5ccc6sc7ccccc7c6c5c5oc(-c6ccccc6)nc45)n3)cc2)c([2H])c1[2H] ; Fc1c(F)c(F)c(-c2nc(-c3ccccc3)nc(-c3cc4ccc5sc6ccccc6c5c4c4oc(-c5ccccc5)nc34)n2)c(F)c1F ; c1ccc(-c2nc(-c3ccccc3)nc(-c3ccc(-c4cc5ccc6sc7ccccc7c6c5c5oc(-c6ccccc6)nc45)cc3)n2)cc1]

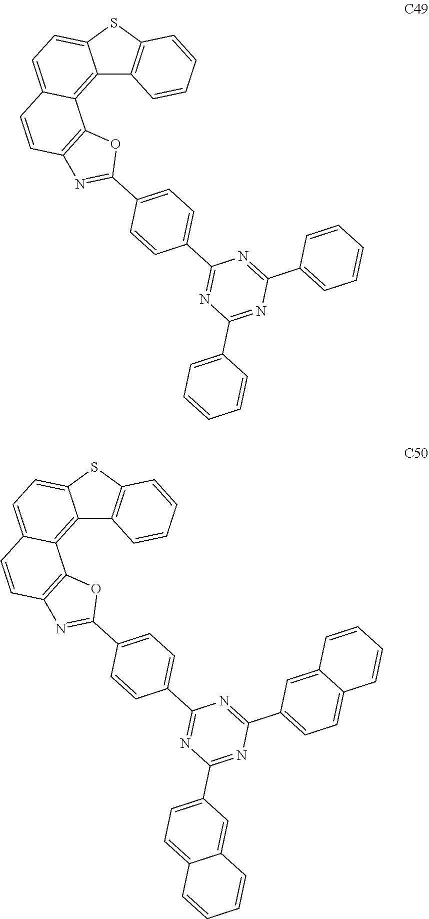 [SMILES: c1ccc(-c2nc(-c3ccccc3)nc(-c3ccc(-c4nc5ccc6ccc7sc8ccccc8c7c6c5o4)cc3)n2)cc1 ; c1ccc2cc(-c3nc(-c4ccc(-c5nc6ccc7ccc8sc9ccccc9c8c7c6o5)cc4)nc(-c4ccc5ccccc5c4)n3)ccc2c1]

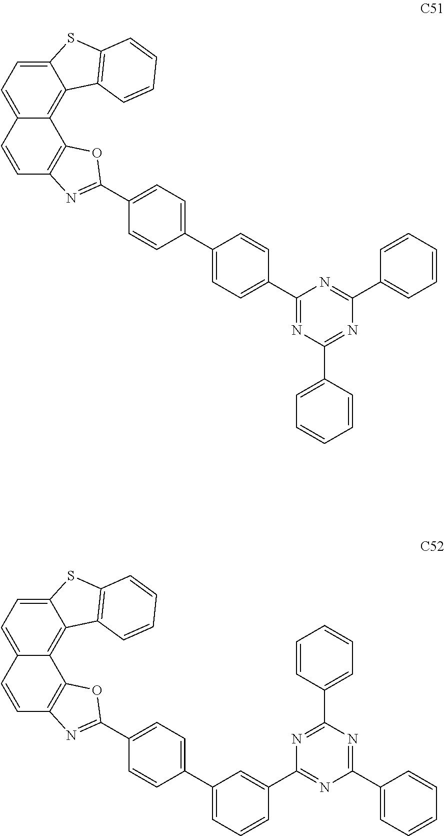 [SMILES: c1ccc(-c2nc(-c3ccccc3)nc(-c3ccc(-c4ccc(-c5nc6ccc7ccc8sc9ccccc9c8c7c6o5)cc4)cc3)n2)cc1 ; c1ccc(-c2nc(-c3ccccc3)nc(-c3cccc(-c4ccc(-c5nc6ccc7ccc8sc9ccccc9c8c7c6o5)cc4)c3)n2)cc1]

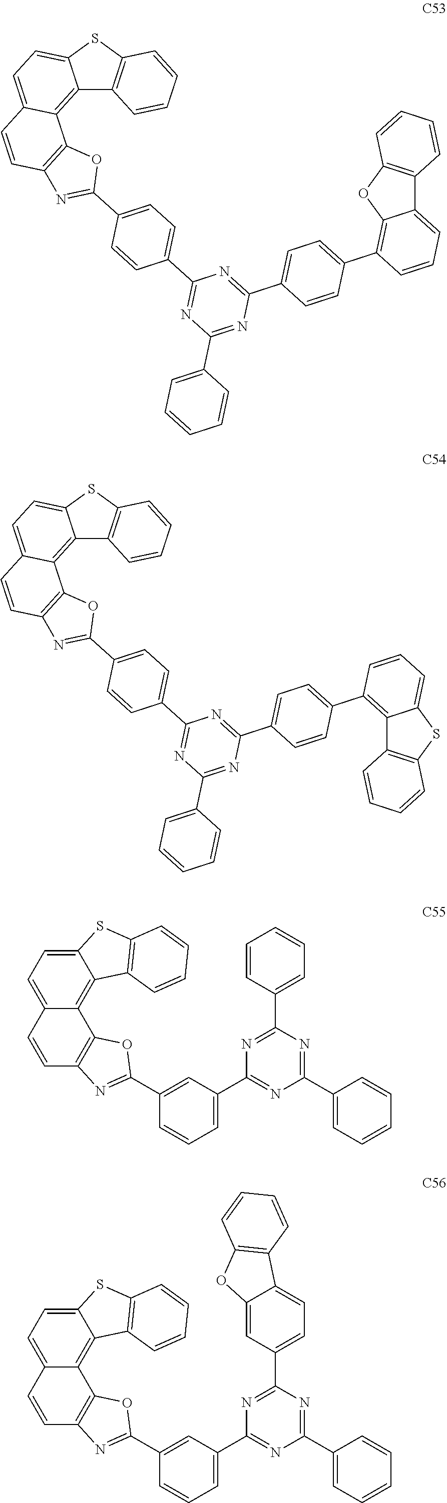 [SMILES: c1ccc(-c2nc(-c3ccc(-c4nc5ccc6ccc7sc8ccccc8c7c6c5o4)cc3)nc(-c3ccc(-c4cccc5c4oc4ccccc45)cc3)n2)cc1 ; c1ccc(-c2nc(-c3ccc(-c4nc5ccc6ccc7sc8ccccc8c7c6c5o4)cc3)nc(-c3ccc(-c4cccc5sc6ccccc6c45)cc3)n2)cc1 ; c1ccc(-c2nc(-c3ccccc3)nc(-c3cccc(-c4nc5ccc6ccc7sc8ccccc8c7c6c5o4)c3)n2)cc1 ; c1ccc(-c2nc(-c3cccc(-c4nc5ccc6ccc7sc8ccccc8c7c6c5o4)c3)nc(-c3ccc4c(c3)oc3ccccc34)n2)cc1]

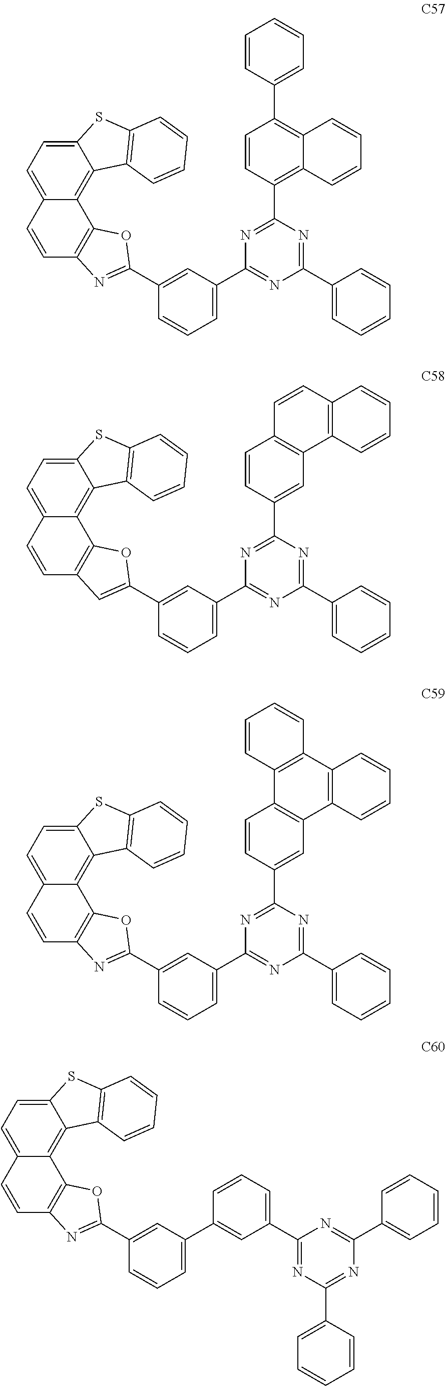 [SMILES: c1ccc(-c2nc(-c3cccc(-c4nc5ccc6ccc7sc8ccccc8c7c6c5o4)c3)nc(-c3ccc(-c4ccccc4)c4ccccc34)n2)cc1 ; c1ccc(-c2nc(-c3cccc(-c4cc5ccc6ccc7sc8ccccc8c7c6c5o4)c3)nc(-c3ccc4ccc5ccccc5c4c3)n2)cc1 ; c1ccc(-c2nc(-c3cccc(-c4nc5ccc6ccc7sc8ccccc8c7c6c5o4)c3)nc(-c3ccc4c5ccccc5c5ccccc5c4c3)n2)cc1 ; c1ccc(-c2nc(-c3ccccc3)nc(-c3cccc(-c4cccc(-c5nc6ccc7ccc8sc9ccccc9c8c7c6o5)c4)c3)n2)cc1]

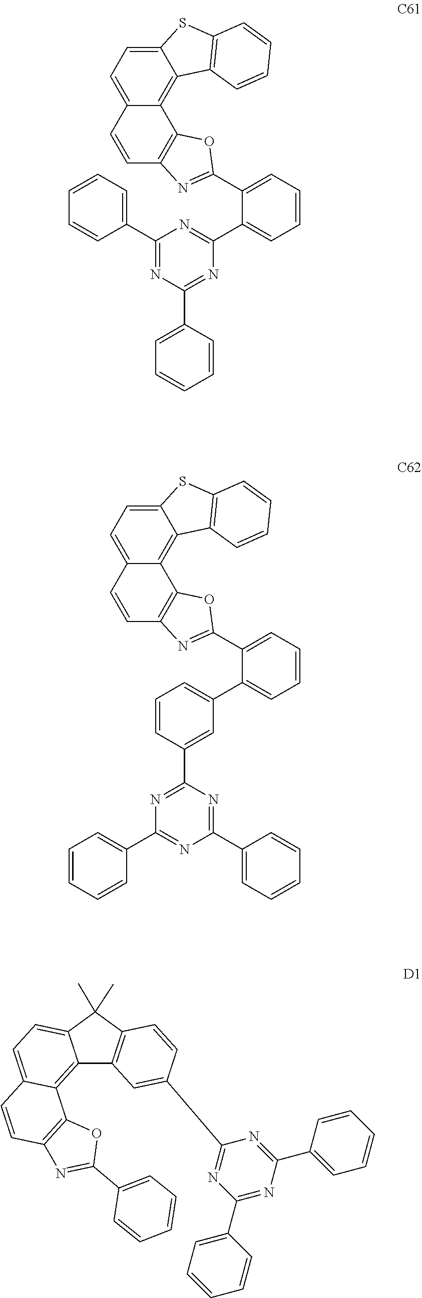 [SMILES: c1ccc(-c2nc(-c3ccccc3)nc(-c3ccccc3-c3nc4ccc5ccc6sc7ccccc7c6c5c4o3)n2)cc1 ; c1ccc(-c2nc(-c3ccccc3)nc(-c3cccc(-c4ccccc4-c4nc5ccc6ccc7sc8ccccc8c7c6c5o4)c3)n2)cc1 ; CC1(C)c2ccc(-c3nc(-c4ccccc4)nc(-c4ccccc4)n3)cc2-c2c1ccc1ccc3nc(-c4ccccc4)oc3c21]

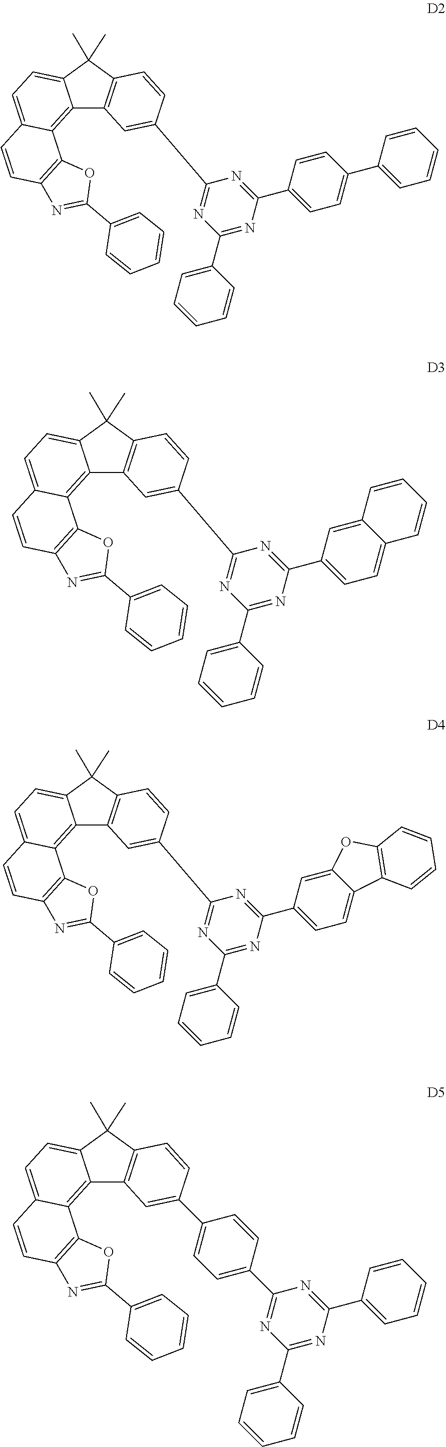 [SMILES: CC1(C)c2ccc(-c3nc(-c4ccccc4)nc(-c4ccc(-c5ccccc5)cc4)n3)cc2-c2c1ccc1ccc3nc(-c4ccccc4)oc3c21 ; CC1(C)c2ccc(-c3nc(-c4ccccc4)nc(-c4ccc5ccccc5c4)n3)cc2-c2c1ccc1ccc3nc(-c4ccccc4)oc3c21 ; CC1(C)c2ccc(-c3nc(-c4ccccc4)nc(-c4ccc5c(c4)oc4ccccc45)n3)cc2-c2c1ccc1ccc3nc(-c4ccccc4)oc3c21 ; CC1(C)c2ccc(-c3ccc(-c4nc(-c5ccccc5)nc(-c5ccccc5)n4)cc3)cc2-c2c1ccc1ccc3nc(-c4ccccc4)oc3c21]

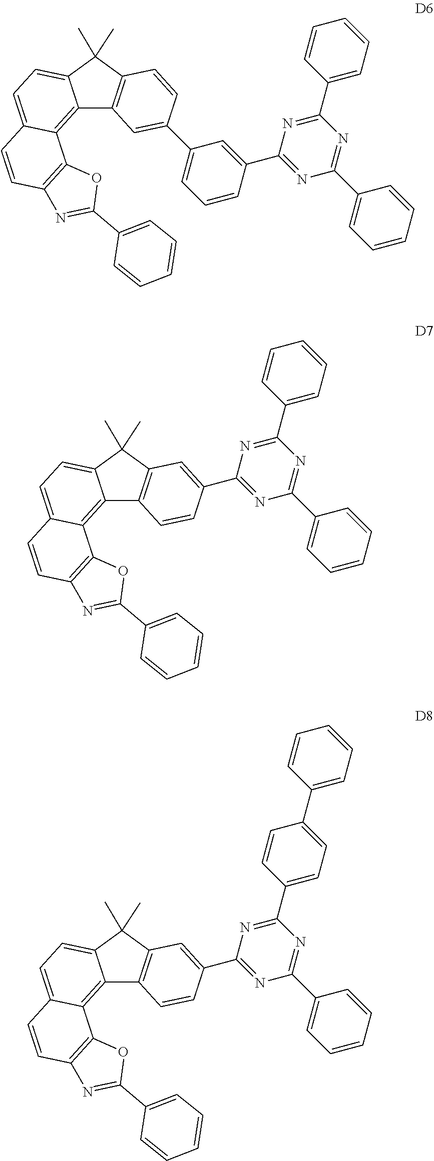 [SMILES: CC1(C)c2ccc(-c3cccc(-c4nc(-c5ccccc5)nc(-c5ccccc5)n4)c3)cc2-c2c1ccc1ccc3nc(-c4ccccc4)oc3c21 ; CC1(C)c2cc(-c3nc(-c4ccccc4)nc(-c4ccccc4)n3)ccc2-c2c1ccc1ccc3nc(-c4ccccc4)oc3c21 ; CC1(C)c2cc(-c3nc(-c4ccccc4)nc(-c4ccc(-c5ccccc5)cc4)n3)ccc2-c2c1ccc1ccc3nc(-c4ccccc4)oc3c21]

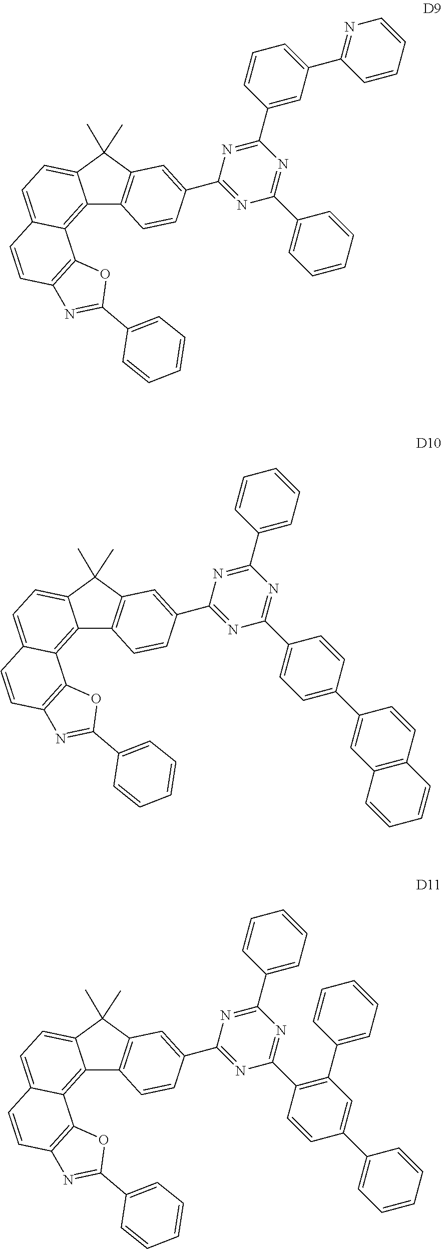 [SMILES: CC1(C)c2cc(-c3nc(-c4ccccc4)nc(-c4cccc(-c5ccccn5)c4)n3)ccc2-c2c1ccc1ccc3nc(-c4ccccc4)oc3c21 ; CC1(C)c2cc(-c3nc(-c4ccccc4)nc(-c4ccc(-c5ccc6ccccc6c5)cc4)n3)ccc2-c2c1ccc1ccc3nc(-c4ccccc4)oc3c21 ; CC1(C)c2cc(-c3nc(-c4ccccc4)nc(-c4ccc(-c5ccccc5)cc4-c4ccccc4)n3)ccc2-c2c1ccc1ccc3nc(-c4ccccc4)oc3c21]

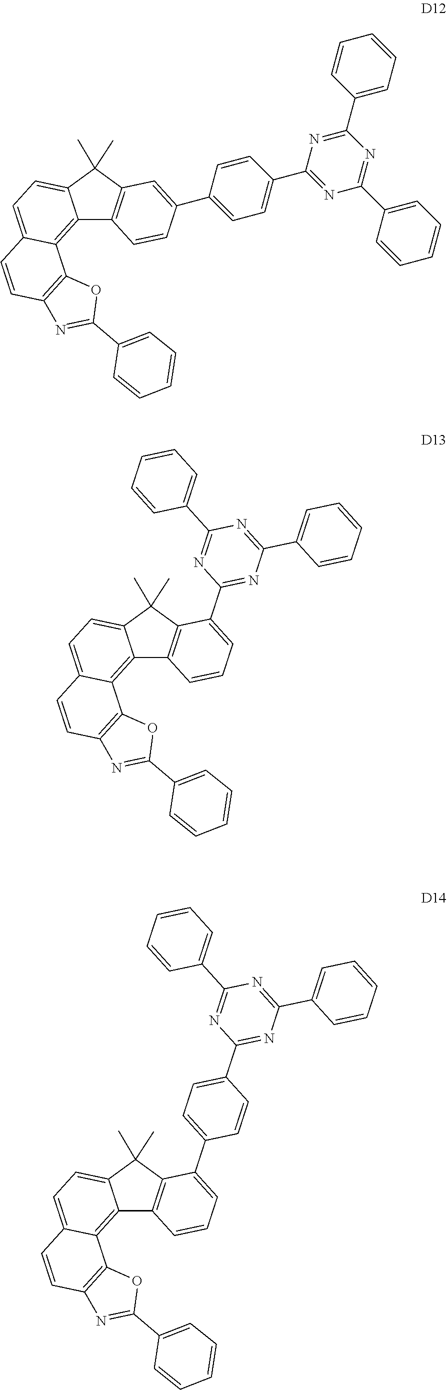 [SMILES: CC1(C)c2cc(-c3ccc(-c4nc(-c5ccccc5)nc(-c5ccccc5)n4)cc3)ccc2-c2c1ccc1ccc3nc(-c4ccccc4)oc3c21 ; CC1(C)c2ccc3ccc4nc(-c5ccccc5)oc4c3c2-c2cccc(-c3nc(-c4ccccc4)nc(-c4ccccc4)n3)c21 ; CC1(C)c2ccc3ccc4nc(-c5ccccc5)oc4c3c2-c2cccc(-c3ccc(-c4nc(-c5ccccc5)nc(-c5ccccc5)n4)cc3)c21]

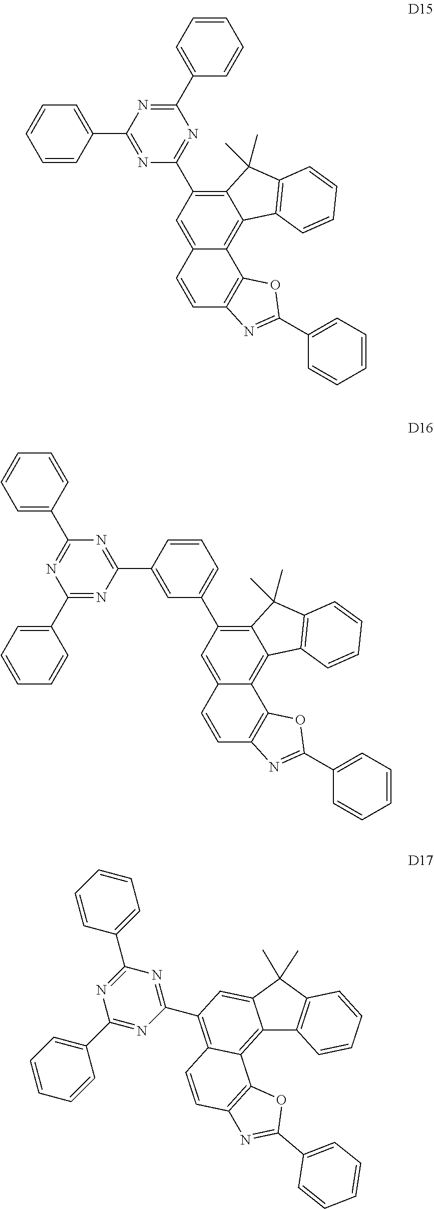 [SMILES: CC1(C)c2ccccc2-c2c1c(-c1nc(-c3ccccc3)nc(-c3ccccc3)n1)cc1ccc3nc(-c4ccccc4)oc3c21 ; CC1(C)c2ccccc2-c2c1c(-c1cccc(-c3nc(-c4ccccc4)nc(-c4ccccc4)n3)c1)cc1ccc3nc(-c4ccccc4)oc3c21 ; CC1(C)c2ccccc2-c2c1cc(-c1nc(-c3ccccc3)nc(-c3ccccc3)n1)c1ccc3nc(-c4ccccc4)oc3c21]

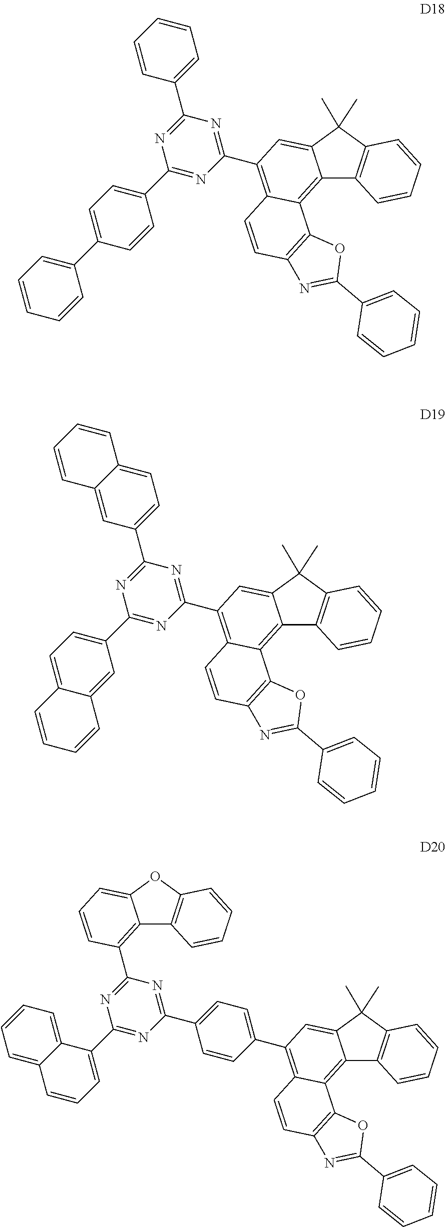 [SMILES: CC1(C)c2ccccc2-c2c1cc(-c1nc(-c3ccccc3)nc(-c3ccc(-c4ccccc4)cc3)n1)c1ccc3nc(-c4ccccc4)oc3c21 ; CC1(C)c2ccccc2-c2c1cc(-c1nc(-c3ccc4ccccc4c3)nc(-c3ccc4ccccc4c3)n1)c1ccc3nc(-c4ccccc4)oc3c21 ; CC1(C)c2ccccc2-c2c1cc(-c1ccc(-c3nc(-c4cccc5ccccc45)nc(-c4cccc5oc6ccccc6c45)n3)cc1)c1ccc3nc(-c4ccccc4)oc3c21]

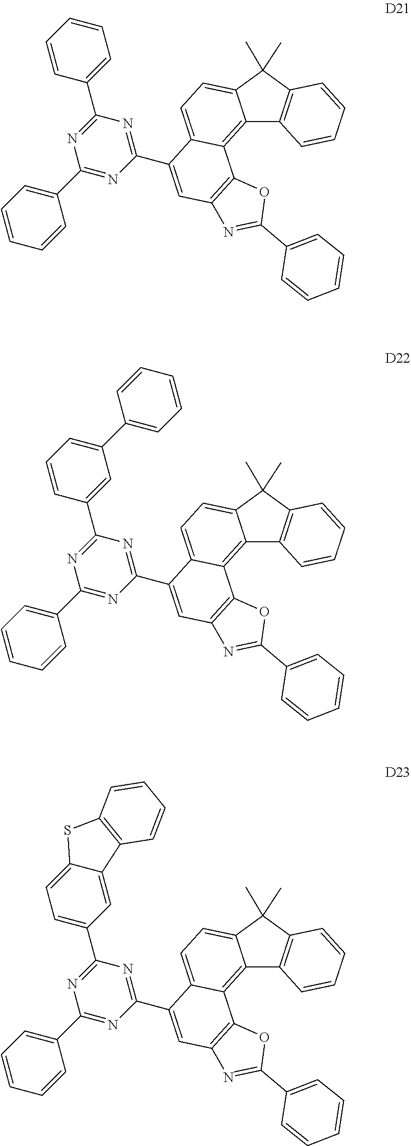 [SMILES: CC1(C)c2ccccc2-c2c1ccc1c(-c3nc(-c4ccccc4)nc(-c4ccccc4)n3)cc3nc(-c4ccccc4)oc3c21 ; CC1(C)c2ccccc2-c2c1ccc1c(-c3nc(-c4ccccc4)nc(-c4cccc(-c5ccccc5)c4)n3)cc3nc(-c4ccccc4)oc3c21 ; CC1(C)c2ccccc2-c2c1ccc1c(-c3nc(-c4ccccc4)nc(-c4ccc5sc6ccccc6c5c4)n3)cc3nc(-c4ccccc4)oc3c21]

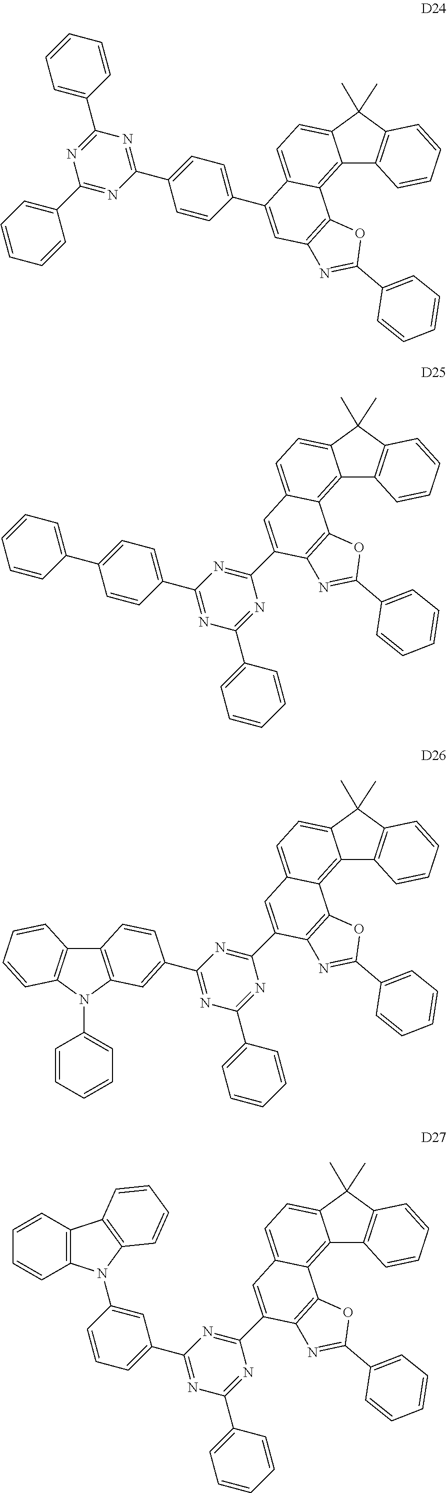 [SMILES: CC1(C)c2ccccc2-c2c1ccc1c(-c3ccc(-c4nc(-c5ccccc5)nc(-c5ccccc5)n4)cc3)cc3nc(-c4ccccc4)oc3c21 ; CC1(C)c2ccccc2-c2c1ccc1cc(-c3nc(-c4ccccc4)nc(-c4ccc(-c5ccccc5)cc4)n3)c3nc(-c4ccccc4)oc3c21 ; CC1(C)c2ccccc2-c2c1ccc1cc(-c3nc(-c4ccccc4)nc(-c4ccc5c6ccccc6n(-c6ccccc6)c5c4)n3)c3nc(-c4ccccc4)oc3c21 ; CC1(C)c2ccccc2-c2c1ccc1cc(-c3nc(-c4ccccc4)nc(-c4cccc(-n5c6ccccc6c6ccccc65)c4)n3)c3nc(-c4ccccc4)oc3c21]

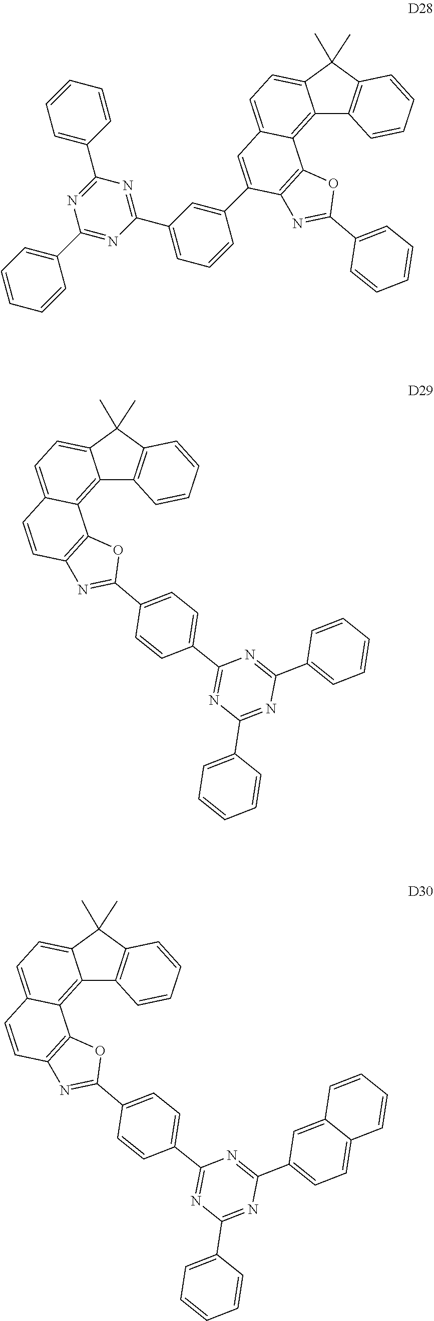 [SMILES: CC1(C)c2ccccc2-c2c1ccc1cc(-c3cccc(-c4nc(-c5ccccc5)nc(-c5ccccc5)n4)c3)c3nc(-c4ccccc4)oc3c21 ; CC1(C)c2ccccc2-c2c1ccc1ccc3nc(-c4ccc(-c5nc(-c6ccccc6)nc(-c6ccccc6)n5)cc4)oc3c21 ; CC1(C)c2ccccc2-c2c1ccc1ccc3nc(-c4ccc(-c5nc(-c6ccccc6)nc(-c6ccc7ccccc7c6)n5)cc4)oc3c21]

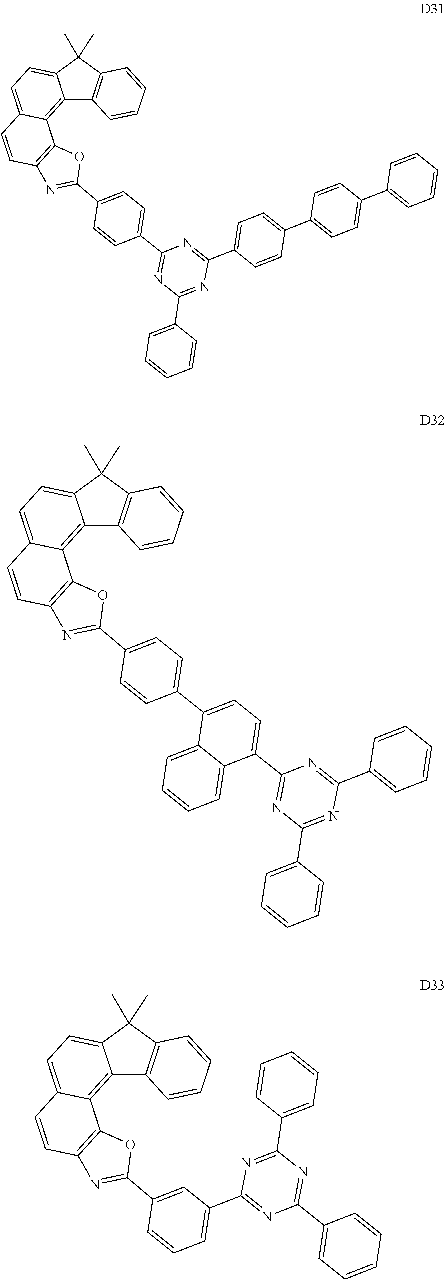 [SMILES: CC1(C)c2ccccc2-c2c1ccc1ccc3nc(-c4ccc(-c5nc(-c6ccccc6)nc(-c6ccc(-c7ccc(-c8ccccc8)cc7)cc6)n5)cc4)oc3c21 ; CC1(C)c2ccccc2-c2c1ccc1ccc3nc(-c4ccc(-c5ccc(-c6nc(-c7ccccc7)nc(-c7ccccc7)n6)c6ccccc56)cc4)oc3c21 ; CC1(C)c2ccccc2-c2c1ccc1ccc3nc(-c4cccc(-c5nc(-c6ccccc6)nc(-c6ccccc6)n5)c4)oc3c21]

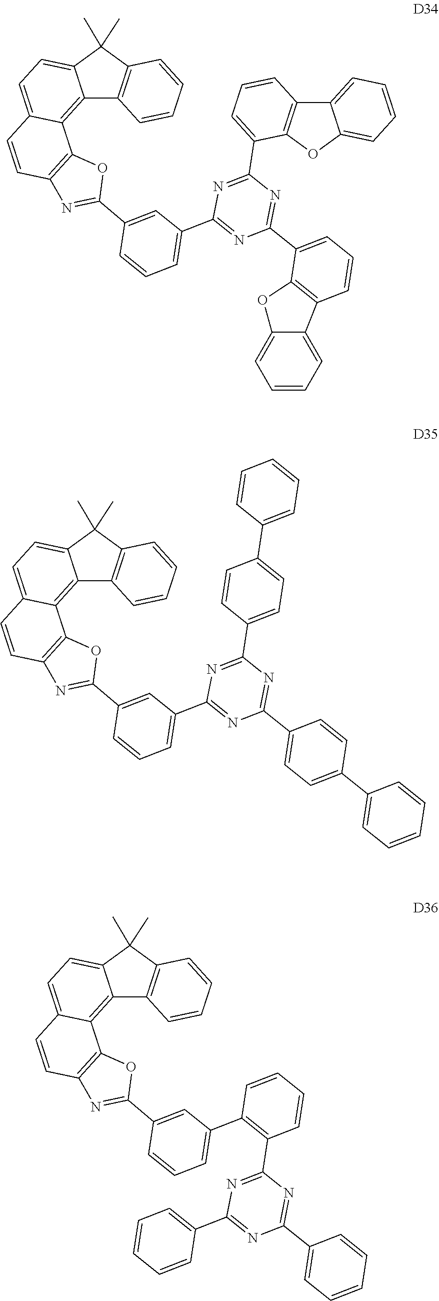 [SMILES: CC1(C)c2ccccc2-c2c1ccc1ccc3nc(-c4cccc(-c5nc(-c6cccc7c6oc6ccccc67)nc(-c6cccc7c6oc6ccccc67)n5)c4)oc3c21 ; CC1(C)c2ccccc2-c2c1ccc1ccc3nc(-c4cccc(-c5nc(-c6ccc(-c7ccccc7)cc6)nc(-c6ccc(-c7ccccc7)cc6)n5)c4)oc3c21 ; CC1(C)c2ccccc2-c2c1ccc1ccc3nc(-c4cccc(-c5ccccc5-c5nc(-c6ccccc6)nc(-c6ccccc6)n5)c4)oc3c21]

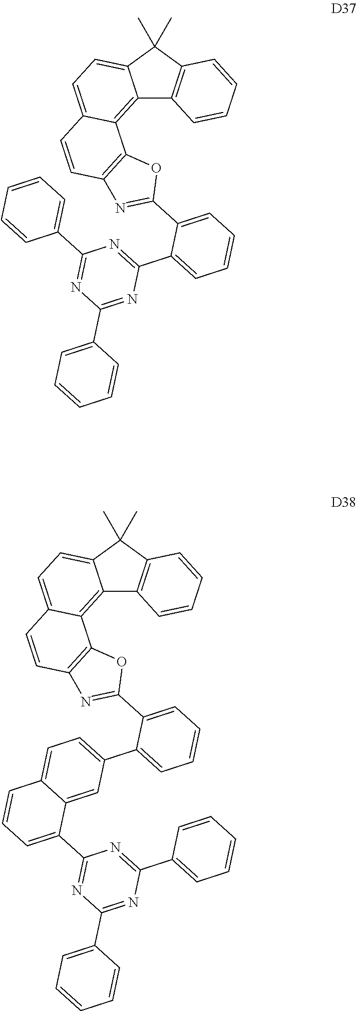 [SMILES: CC1(C)c2ccccc2-c2c1ccc1ccc3nc(-c4ccccc4-c4nc(-c5ccccc5)nc(-c5ccccc5)n4)oc3c21 ; CC1(C)c2ccccc2-c2c1ccc1ccc3nc(-c4ccccc4-c4ccc5cccc(-c6nc(-c7ccccc7)nc(-c7ccccc7)n6)c5c4)oc3c21]

In a second aspect of the disclosure, the disclosure provides an organic electroluminescent device, including an anode and a cathode which are oppositely disposed, and a functional layer disposed between the anode and the cathode, wherein the functional layer includes the organic compound of the disclosure.

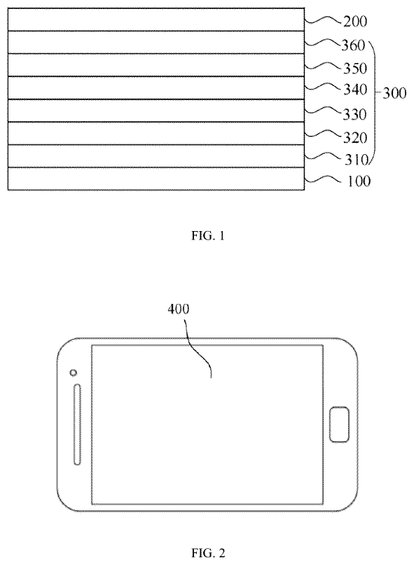

In some embodiments of the disclosure, the organic electroluminescent device is a red organic electroluminescent device. As shown in FIG. 1, the organic electroluminescent device may include an anode 100, a first hole transport layer 320, a second hole transport layer 330, an organic light-emitting layer 340, an electron transport layer 350, an electron injection layer 360, and a cathode 200 which are sequentially stacked.

Optionally, the anode 100 includes the following anode materials which are optionally materials having a large work function that facilitate hole injection into the functional layer. Specific examples of the anode materials include metals such as nickel, platinum, vanadium, chromium, copper, zinc, and gold, or an alloy of them; metal oxides such as zinc oxide, indium oxide, indium tin oxide (ITO) and indium zinc oxide (IZO); combined metals and oxides, such as ZnO:Al or SnO₂:Sb; or a conductive polymer such as poly(3-methylthiophene), poly[3,4-(ethylene-1,2-dioxy)thiophene](PEDT), polypyrrole, and polyaniline, but are not limited to this. It preferably includes a transparent electrode containing indium tin oxide (ITO) as the anode.

Optionally, the first hole transport layer 320 and the second hole transport layer 330 include one or more hole transport materials, and the hole transport materials may be selected from a carbazole multimer, carbazole connected triarylamine compounds, or other types of compounds, which can be selected by those skilled in the art with reference to the prior art, and are not specially limited in the disclosure. In some embodiments of the disclosure, the first hole transport layer 320 is made of HT-20 and the second hole transport layer 330 is made of HT-21.

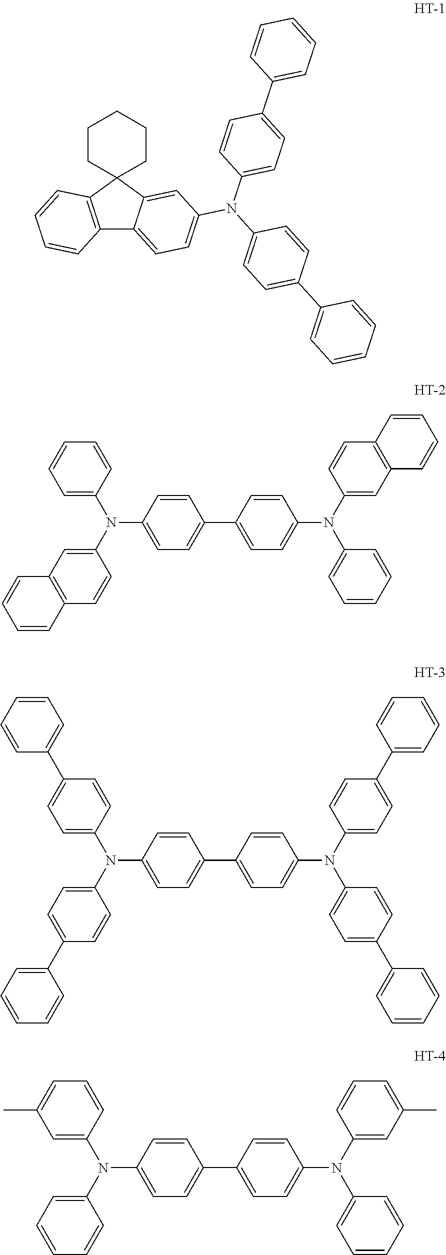

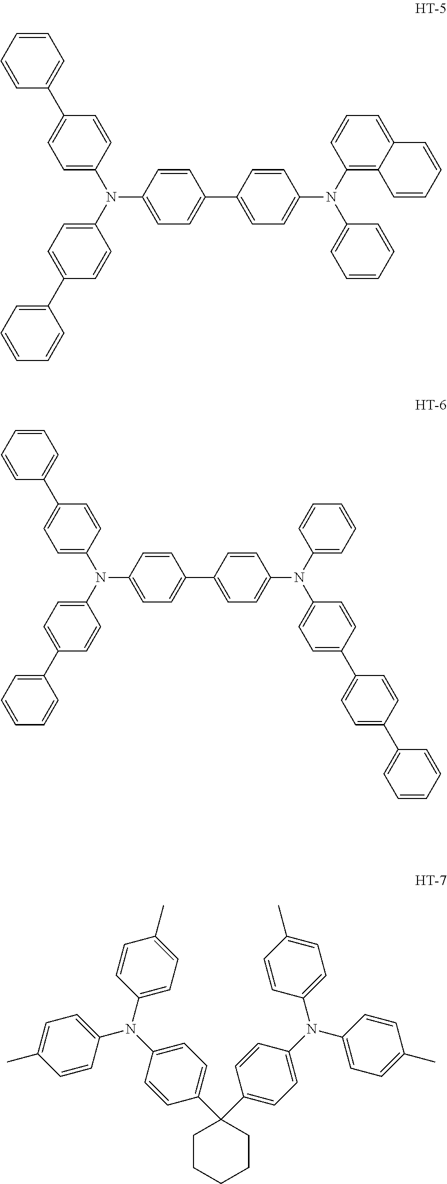

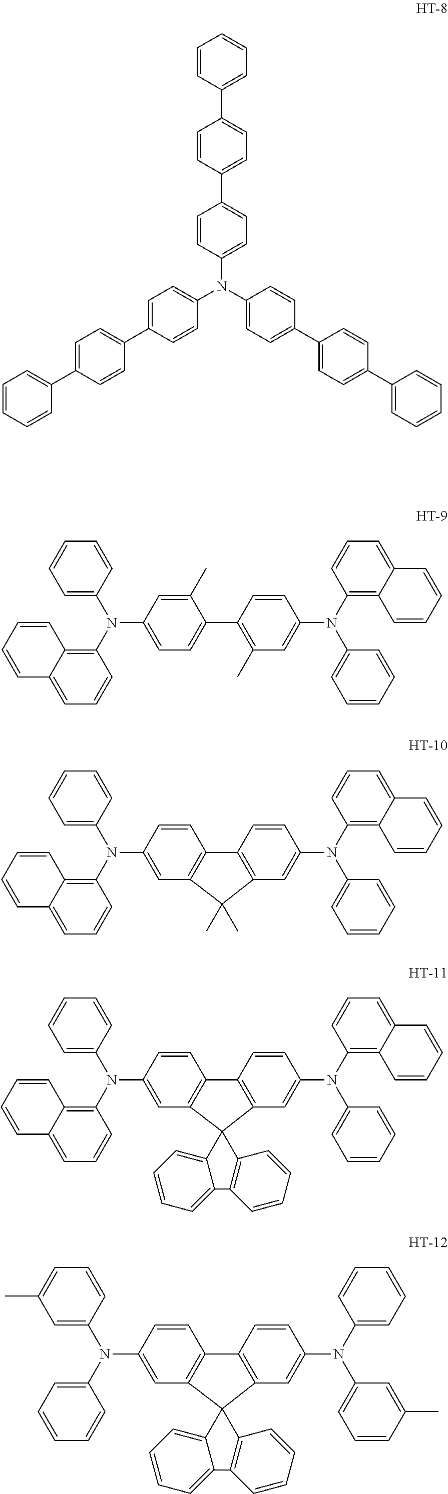

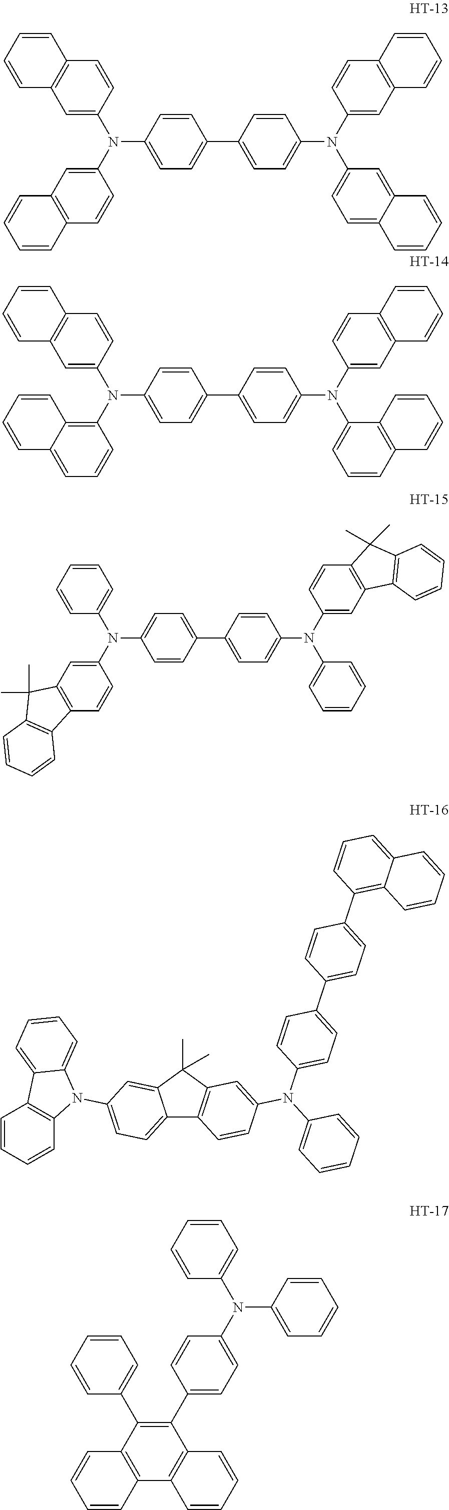

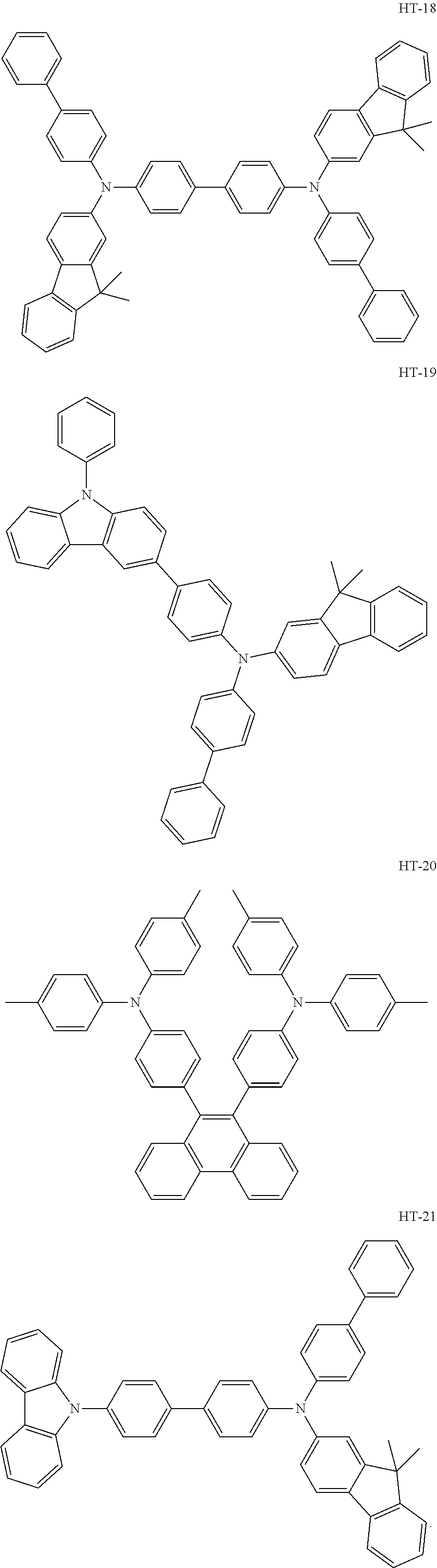

Optionally, the hole injection layer 310 may be further disposed between the anode 100 and the first hole transport layer 320 to enhance the ability to inject holes into the first hole transport layer 320. The hole injection layer 310 can be made of a benzidine derivative, a starburst arylamine compound, a phthalocyanine derivative or other materials, which is not specially limited in the disclosure. A material of the hole injection layer 310 may be selected from, for example, the following compounds or any combination of them;

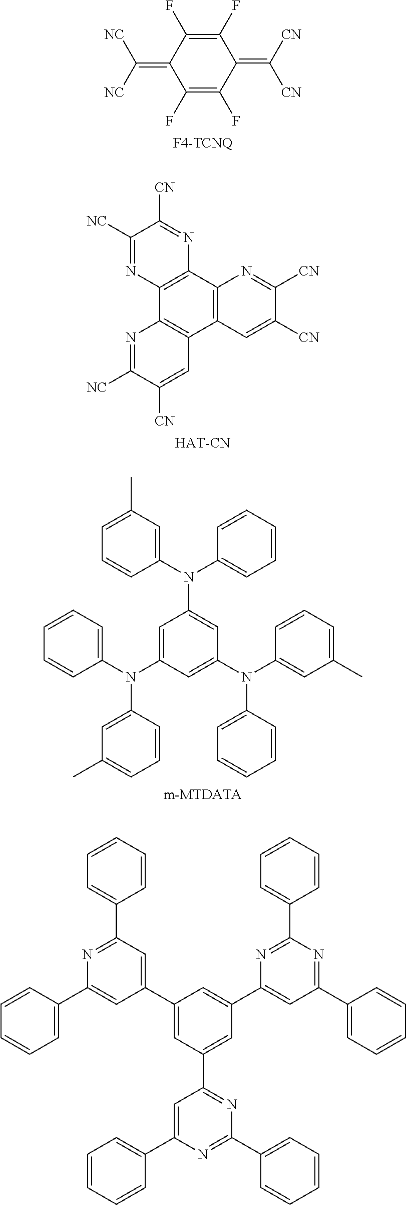 [SMILES: N#CC(C#N)=c1c(F)c(F)c(=C(C#N)C#N)c(F)c1F ; N#Cc1cc2c3cc(C#N)c(C#N)nc3c3nc(C#N)c(C#N)nc3c2nc1C#N ; Cc1cccc(N(c2ccccc2)c2cc(N(c3ccccc3)c3cccc(C)c3)cc(N(c3ccccc3)c3cccc(C)c3)c2)c1 ; c1ccc(-c2cc(-c3cc(-c4cc(-c5ccccc5)nc(-c5ccccc5)n4)cc(-c4cc(-c5ccccc5)nc(-c5ccccc5)n4)c3)cc(-c3ccccc3)n2)cc1]

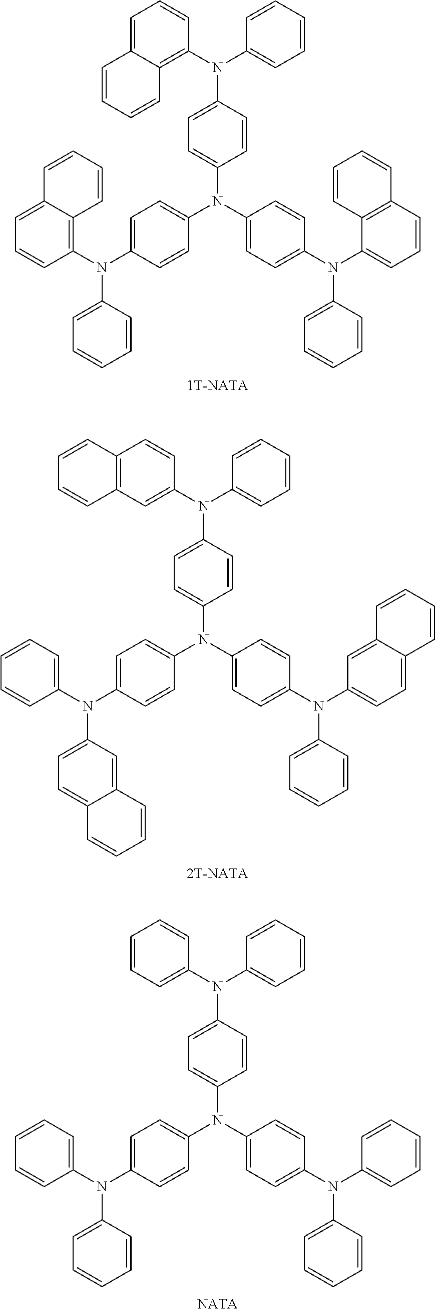

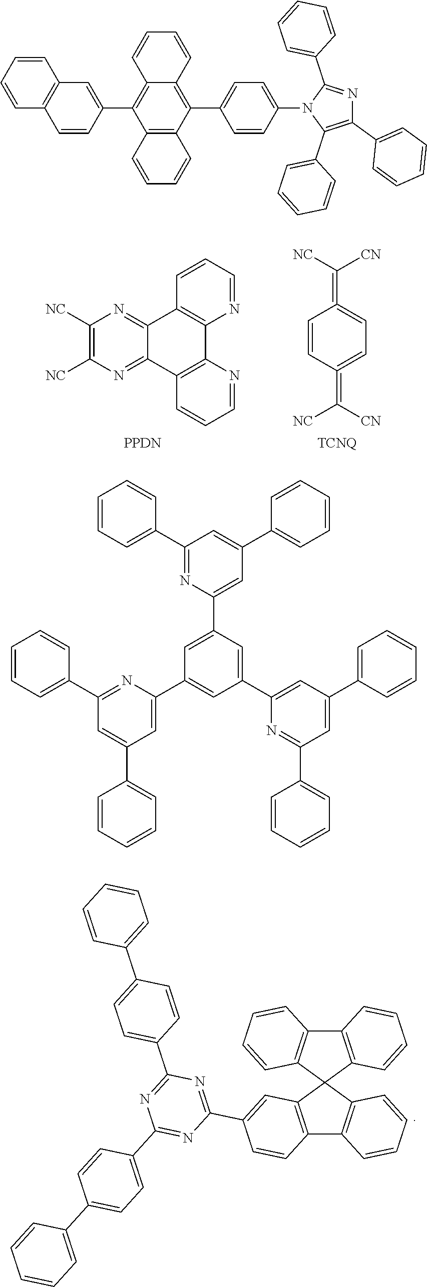

In some embodiments of the disclosure, the hole injection layer 310 is composed of HAT-CN.

Optionally, the organic light-emitting layer 340 may be composed of a single light-emitting layer material, and may also include a host material and a doping material. Optionally, the organic light-emitting layer 340 is composed of the host material and the doping material, holes injected into the organic light-emitting layer 340 and electrons injected into the organic light-emitting layer 340 can be recombined in the organic light-emitting layer 340 to form excitons, the excitons transfer energy to the host material, the host material transfers energy to the doping material, and then the doping material can emit light.

The host material of the organic light-emitting layer 340 may be a metal chelated compound, a distyryl derivative, an aromatic amine derivative, a dibenzofuran derivative or other types of materials, which is not specially limited in the disclosure.

In one embodiment of the disclosure, the organic light-emitting layer 340 includes the organic compound of the disclosure.

Optionally, the organic compound of the disclosure is used as a host material (an electron-type host material) of the organic light-emitting layer 340.

In some embodiments of the disclosure, a hole-type host material of the organic light-emitting layer 340 is

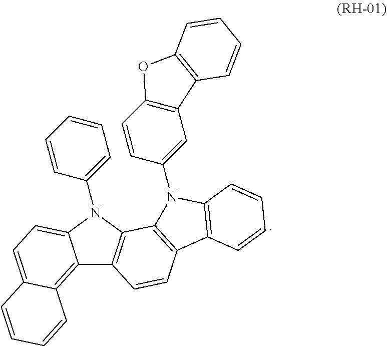 [SMILES: c1ccc(-n2c3ccc4ccccc4c3c3ccc4c5ccccc5n(-c5ccc6oc7ccccc7c6c5)c4c32)cc1]

The guest material of the organic light-emitting layer 340 may be a compound having a condensed aryl ring or its derivative, a compound having a heteroaryl ring or its derivative, an aromatic amine derivative, or other materials, which is not particularly limited in the disclosure. The guest material is also referred to as a doping material or a dopant. Specific examples of red phosphorescent dopants for red organic electroluminescent devices include, but are not limited to,

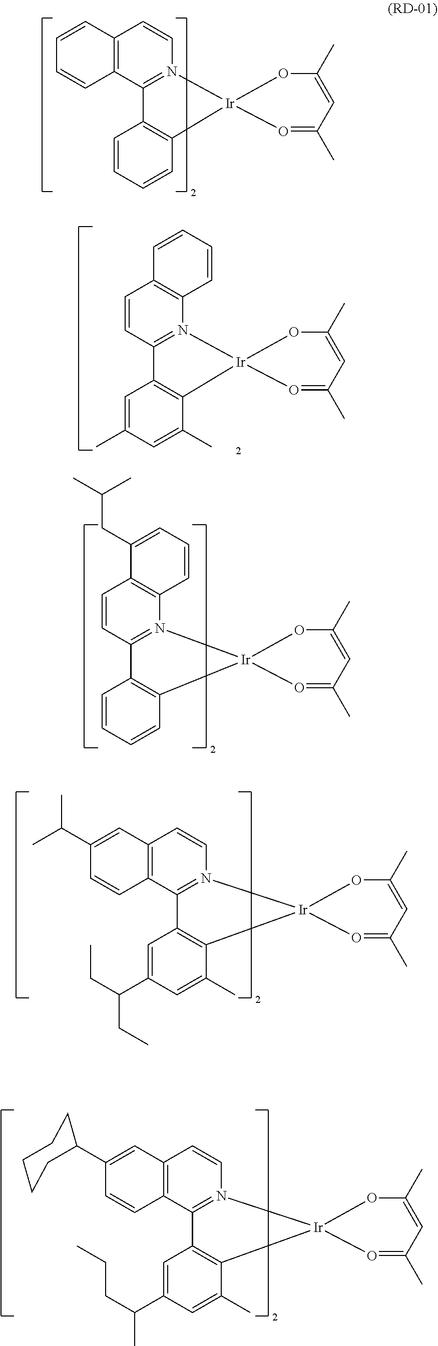

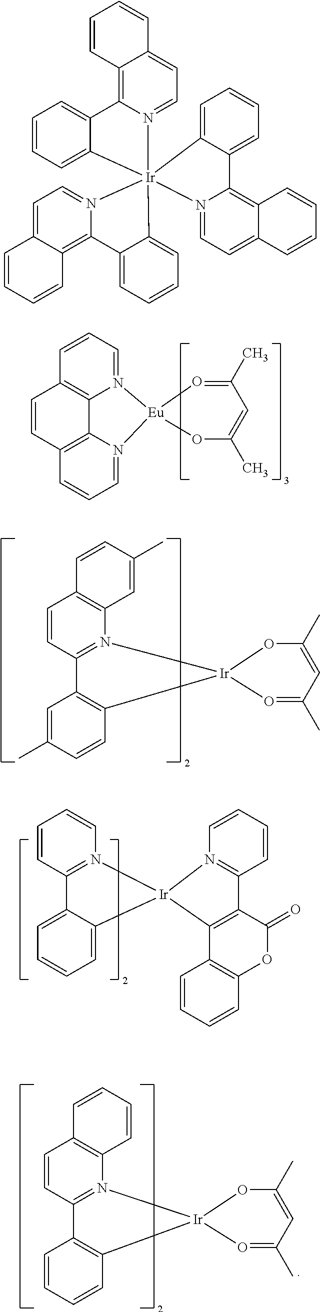

In one more specific embodiment, the host material of the organic light-emitting layer 340 includes the organic compound of the disclosure and RH-P, and the guest material is RD-01.

The electron transport layer 350 may be of a single-layer structure or a multi-layer structure, which may include one or more electron transport materials, and the electron transport materials may be selected from, but are not limited to, ET-01, LiQ, a benzimidazole derivative, an oxadiazole derivative, a quinoxaline derivative, or other electron transport materials, which are not particularly limited in the disclosure. A material of the electron transport layer 350 includes, but is not limited to, the following compounds:

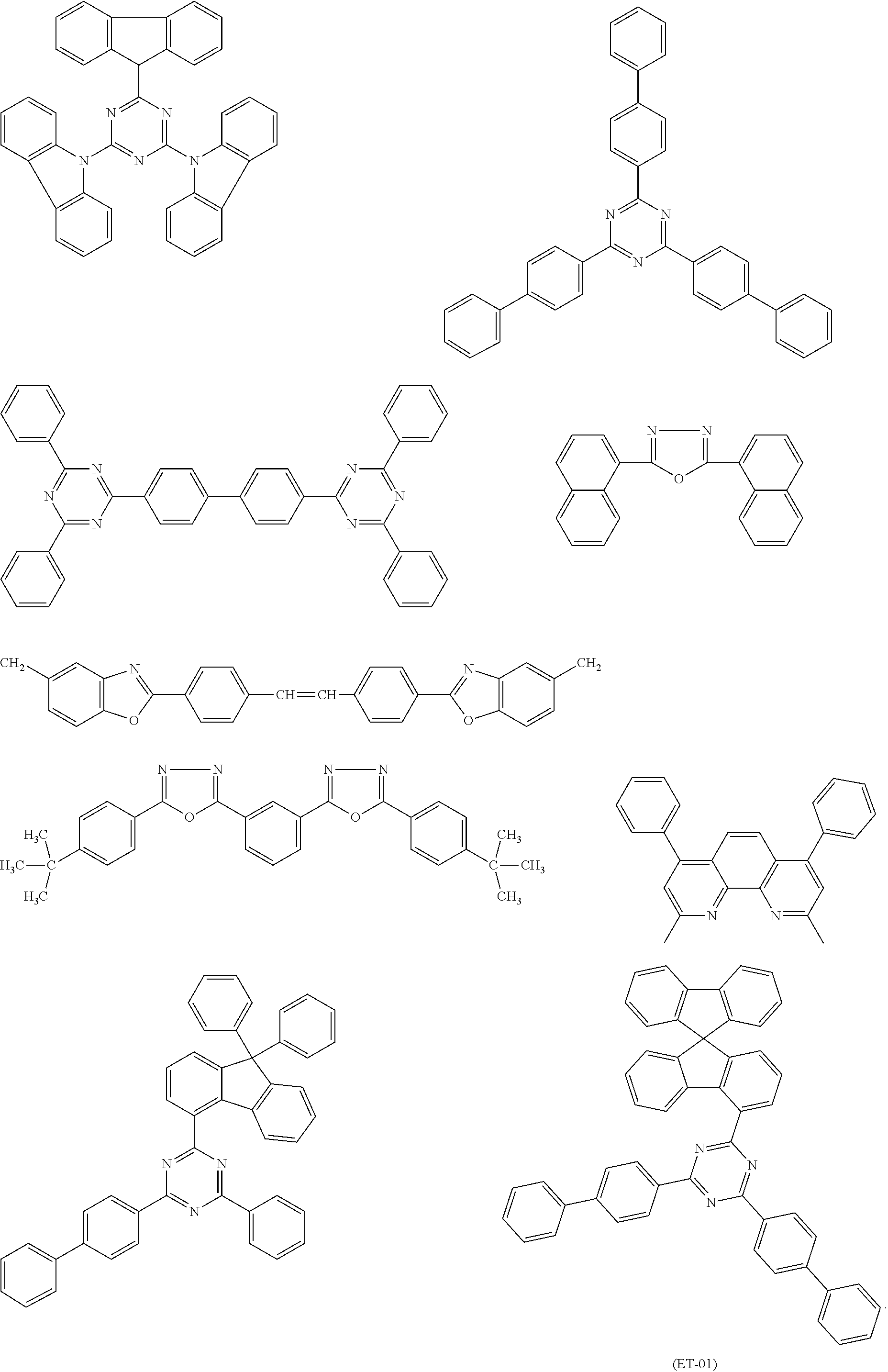

In some embodiments of the disclosure, the electron transport layer 350 consists of ET-01 and LiQ.

In the disclosure, the cathode 200 may include a cathode material, which is a material with a small work function that facilitates electron injection into the functional layer. Specific examples of the cathode material include, but are not limited to, metals such as magnesium, calcium, sodium, potassium, titanium, indium, yttrium, lithium, gadolinium, aluminum, silver, tin, and lead, or an alloy of them; or a multilayer material such as LiF/Al, Liq/Al, LiO₂/Al, LiF/Ca, LiF/Al, and BaF₂/Ca. Optionally, a metal electrode including magnesium and silver as the cathode is included.

In some embodiments of the disclosure, the electron injection layer 360 may include ytterbium (Yb).

A third aspect of the disclosure provides an electronic apparatus, including the organic electroluminescent device according to the second aspect of the disclosure.

According to one embodiment, as shown in FIG. 2, the electronic apparatus provided is an electronic apparatus 400 including the organic electroluminescent device described above. The electronic apparatus 400 may be, for example, a display device, a lighting device, an optical communication device, or other type of electronic devices, and may include, for example, but is not limited to, a computer screen, a mobile phone screen, a television, electronic paper, an emergency lighting lamp, an optical module, and the like.

A synthesis method of the organic compound of the disclosure is specifically described below in combination with the synthesis examples, but the disclosure is not limited in any way accordingly.

Compounds of which synthesis methods were not mentioned in the disclosure were all commercially available raw material products.

The synthesis method of the organic compound provided is not particularly limited in the disclosure, and those skilled in the art can determine a proper synthesis method according to the organic compound provided by the disclosure in combination with a preparation method provided in the synthesis examples. All the organic compounds provided by the disclosure can be obtained by those skilled in the art according to these exemplary preparation methods, and all specific preparation methods for preparing the organic compound are no longer detailed, which should not be understood by those skilled in the art as limiting the disclosure.

### SYNTHESIS EXAMPLES

Those skilled in the art will recognize that chemical reactions described in the disclosure may be used to suitably prepare a number of organic compounds of the disclosure, and that other methods for preparing the compounds of the disclosure are considered to be within the scope of the disclosure. For example, the synthesis of those non-exemplified compounds according to the disclosure can be successfully accomplished by those skilled in the art by modification methods such as appropriately protecting interfering groups, by utilizing other known reagents other than those described in the disclosure, or by making some conventional modification of reaction conditions. Compounds of which synthesis methods were not mentioned in the disclosure were all commercially available raw material products.

### Synthesis of Intermediate a1

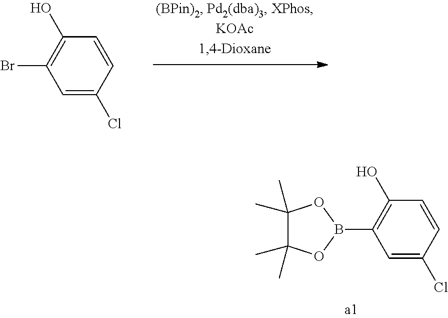 [SMILES: Oc1ccc(Cl)cc1Br ; CC1(C)OB(c2cc(Cl)ccc2O)OC1(C)C]

2-Bromo-4-chlorophenol (25.0 g; 120.5 mmol), bis(pinacolato)diboron (30.6 g; 120.5 mmol), tris(dibenzylideneacetone)dipalladium (1.1 g; 1.2 mmol), 2-dicyclohexylphosphino-2′,4′,6′-triisopropylbiphenyl (1.1 g; 2.4 mmol), potassium acetate (17.7 g; 180.8 mmol) and 1,4-dioxane (250 mL) were added into a round bottom flask under nitrogen protection, and a reaction was carried out under stirring at 90° C.-95° C. for 48 h. The reaction solution was cooled to room temperature, dichloromethane (250 mL) and deionized water (500 mL) were added into the reaction solution, liquid separation was performed, an organic phase was washed with water and dried over anhydrous magnesium sulfate, and a solvent was removed under reduced pressure; and the obtained crude product was purified by silica gel column chromatography using dichloromethane/n-heptane as a solvent to obtain an intermediate a1 (16.1 g; yield: 52%) as a white solid.

Referring to the synthesis method of the intermediate a1, intermediates shown in Table 1 below were synthesized by using a reactant A instead of 2-bromo-4-chlorophenol:

TABLE 1

| Inter- | | | Yield |
|---|---|---|---|
| mediate | Reactant A | Structure | (%) |
| a1 | 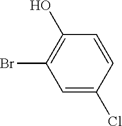 | 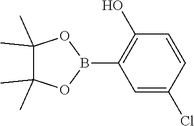 [SMILES: CC1(C)OB(c2cc(Cl)ccc2O)OC1(C)C] | 52 |
| a2 | 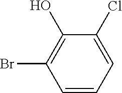 | 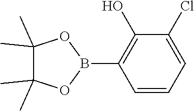 [SMILES: CC1(C)OB(c2cccc(Cl)c2O)OC1(C)C] | 65 |
| a3 | 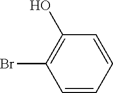 | 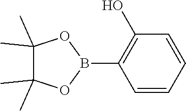 [SMILES: CC1(C)OB(c2ccccc2O)OC1(C)C] | 70 |
| a4 | 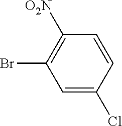 [SMILES: O=[N+]([O-])c1ccc(Cl)cc1Br] | 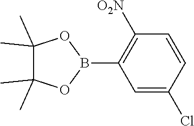 [SMILES: CC1(C)OB(c2cc(Cl)ccc2[N+](=O)[O-])OC1(C)C] | 48 |
| a5 | 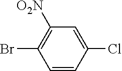 [SMILES: O=[N+]([O-])c1cc(Cl)ccc1Br] | 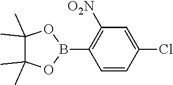 [SMILES: CC1(C)OB(c2ccc(Cl)cc2[N+](=O)[O-])OC1(C)C] | 40 |
| a6 | 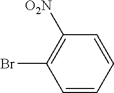 | 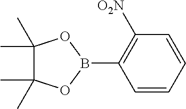 [SMILES: CC1(C)OB(c2ccccc2[N+](=O)[O-])OC1(C)C] | 53 |
| a7 | 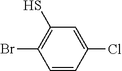 | 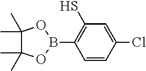 [SMILES: CC1(C)OB(c2ccc(Cl)cc2S)OC1(C)C] | 56 |
| a8 | 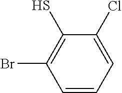 | 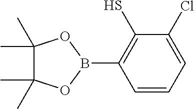 [SMILES: CC1(C)OB(c2cccc(Cl)c2S)OC1(C)C] | 53 |
| a9 | 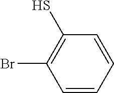 | 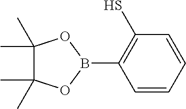 [SMILES: CC1(C)OB(c2ccccc2S)OC1(C)C] | 67 |
| a10 | 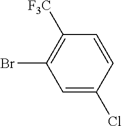 [SMILES: FC(F)(F)c1ccc(Cl)cc1Br] | 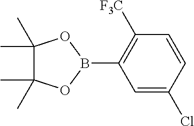 [SMILES: CC1(C)OB(c2cc(Cl)ccc2C(F)(F)F)OC1(C)C] | 55 |
| a11 | 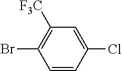 [SMILES: FC(F)(F)c1cc(Cl)ccc1Br] | 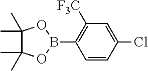 [SMILES: CC1(C)OB(c2ccc(Cl)cc2C(F)(F)F)OC1(C)C] | 48 |
| a12 | 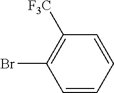 [SMILES: FC(F)(F)c1ccccc1Br] | 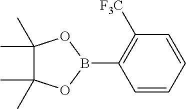 [SMILES: CC1(C)OB(c2ccccc2C(F)(F)F)OC1(C)C] | 57 |

### Synthesis of Intermediate b1

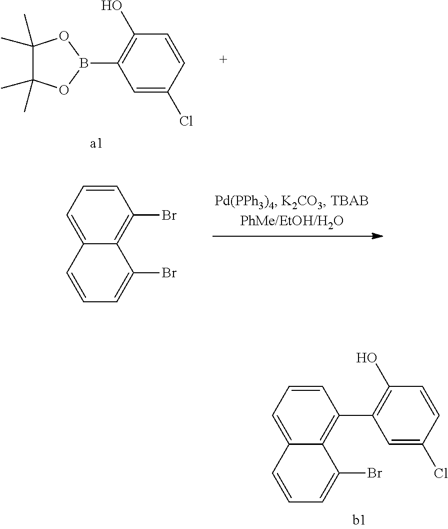 [SMILES: CC1(C)OB(c2cc(Cl)ccc2O)OC1(C)C ; Brc1cccc2cccc(Br)c12 ; Oc1ccc(Cl)cc1-c1cccc2cccc(Br)c12]

The intermediate a1 (16.1 g; 63.4 mmol), 1,8-dibromonaphthalene (18.1 g; 63.4 mmol), tetrakis(triphenylphosphine)palladium (1.5 g; 1.3 mmol), potassium carbonate (17.5 g; 126.7 mmol), tetrabutylammonium bromide (4.1 g; 12.7 mmol), toluene (120 mL), ethanol (30 mL) and deionized water (30 mL) were added into a round bottom flask under nitrogen protection, and heated to 75° C.-80° C., and a reaction was carried out under stirring for 24 h. The reaction solution was cooled to room temperature, deionized water (200 mL) was added, liquid separation was performed, an organic phase was washed with water and dried over anhydrous magnesium sulfate, and a solvent was removed under reduced pressure; and the obtained crude product was purified by silica gel column chromatography using a dichloromethane/n-heptane solvent system to obtain an intermediate b1 (15.5 g; yield: 73%) as a light yellow oily substance.

Referring to the synthesis method of the intermediate b1, intermediates shown in Table 2 below were synthesized by using a reactant C instead of the intermediate a1, and using a reactant B instead of 1,8-dibromonaphthalene:

TABLE 2

| | | | | Yield |
|---|---|---|---|---|
| Intermediate | Reactant C | Reactant B | Structure | (%) |
| b1 | 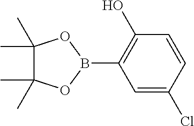 [SMILES: CC1(C)OB(c2cc(Cl)ccc2O)OC1(C)C] | 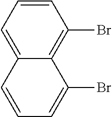 [SMILES: Brc1cccc2cccc(Br)c12] | 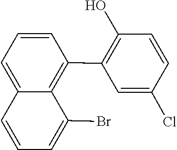 [SMILES: Oc1ccc(Cl)cc1-c1cccc2cccc(Br)c12] | 73 |
| b2 | 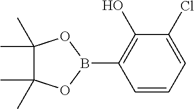 [SMILES: CC1(C)OB(c2cccc(Cl)c2O)OC1(C)C] | 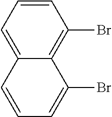 [SMILES: Brc1cccc2cccc(Br)c12] | 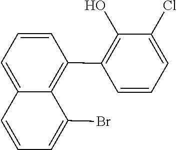 [SMILES: Oc1c(Cl)cccc1-c1cccc2cccc(Br)c12] | 75 |
| b3 | 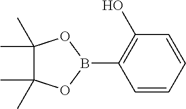 [SMILES: CC1(C)OB(c2ccccc2O)OC1(C)C] | 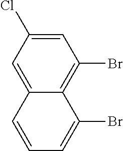 [SMILES: Clc1cc(Br)c2c(Br)cccc2c1] | 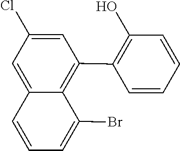 [SMILES: Oc1ccccc1-c1cc(Cl)cc2cccc(Br)c12] | 33 |
| b4 | 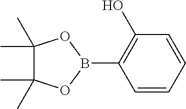 [SMILES: CC1(C)OB(c2ccccc2O)OC1(C)C] | 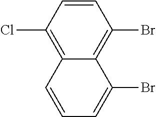 [SMILES: Clc1ccc(Br)c2c(Br)cccc12] | 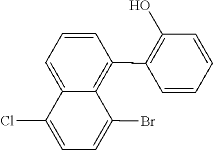 [SMILES: Oc1ccccc1-c1cccc2c(Cl)ccc(Br)c12] | 37 |
| b5 | 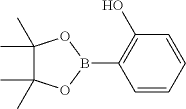 [SMILES: CC1(C)OB(c2ccccc2O)OC1(C)C] | 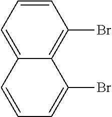 [SMILES: Brc1cccc2cccc(Br)c12] | 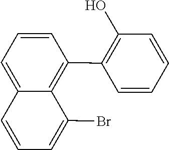 [SMILES: Oc1ccccc1-c1cccc2cccc(Br)c12] | 72 |
| b6 | 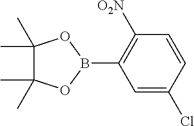 [SMILES: CC1(C)OB(c2cc(Cl)ccc2[N+](=O)[O-])OC1(C)C] | 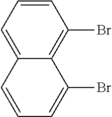 [SMILES: Brc1cccc2cccc(Br)c12] | 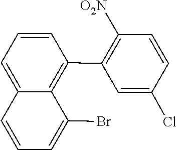 [SMILES: O=[N+]([O-])c1ccc(Cl)cc1-c1cccc2cccc(Br)c12] | 69 |
| b7 | 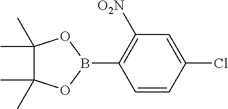 [SMILES: CC1(C)OB(c2ccc(Cl)cc2[N+](=O)[O-])OC1(C)C] | 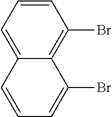 [SMILES: Brc1cccc2cccc(Br)c12] | 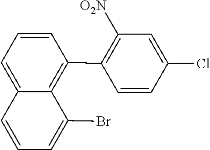 [SMILES: O=[N+]([O-])c1cc(Cl)ccc1-c1cccc2cccc(Br)c12] | 70 |
| b8 | 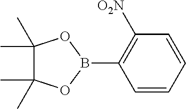 [SMILES: CC1(C)OB(c2ccccc2[N+](=O)[O-])OC1(C)C] | 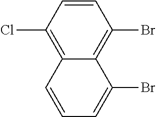 [SMILES: Clc1ccc(Br)c2c(Br)cccc12] | 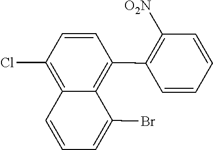 [SMILES: O=[N+]([O-])c1ccccc1-c1ccc(Cl)c2cccc(Br)c12] | 29 |
| b9 | 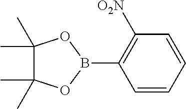 [SMILES: CC1(C)OB(c2ccccc2[N+](=O)[O-])OC1(C)C] | 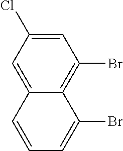 [SMILES: Clc1cc(Br)c2c(Br)cccc2c1] | 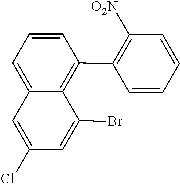 [SMILES: O=[N+]([O-])c1ccccc1-c1cccc2cc(Cl)cc(Br)c12] | 31 |
| b10 | 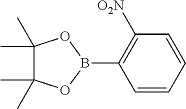 [SMILES: CC1(C)OB(c2ccccc2[N+](=O)[O-])OC1(C)C] | 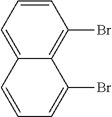 [SMILES: Brc1cccc2cccc(Br)c12] | 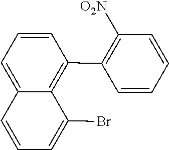 [SMILES: O=[N+]([O-])c1ccccc1-c1cccc2cccc(Br)c12] | 60 |
| b11 | 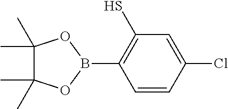 [SMILES: CC1(C)OB(c2ccc(Cl)cc2S)OC1(C)C] | 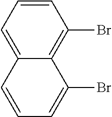 [SMILES: Brc1cccc2cccc(Br)c12] | 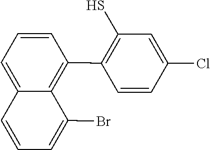 [SMILES: Sc1cc(Cl)ccc1-c1cccc2cccc(Br)c12] | 70 |
| b12 | 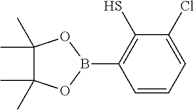 [SMILES: CC1(C)OB(c2cccc(Cl)c2S)OC1(C)C] | 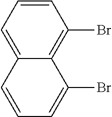 [SMILES: Brc1cccc2cccc(Br)c12] | 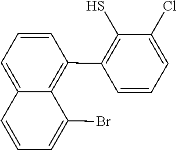 [SMILES: Sc1c(Cl)cccc1-c1cccc2cccc(Br)c12] | 69 |
| b13 | 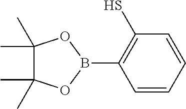 [SMILES: CC1(C)OB(c2ccccc2S)OC1(C)C] | 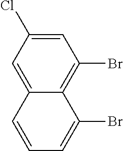 [SMILES: Clc1cc(Br)c2c(Br)cccc2c1] | 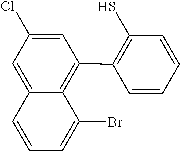 [SMILES: Sc1ccccc1-c1cc(Cl)cc2cccc(Br)c12] | 25 |
| b14 | 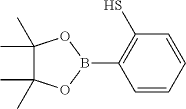 [SMILES: CC1(C)OB(c2ccccc2S)OC1(C)C] | 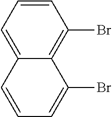 [SMILES: Brc1cccc2cccc(Br)c12] | 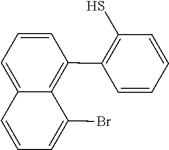 [SMILES: Sc1ccccc1-c1cccc2cccc(Br)c12] | 64 |
| b15 | 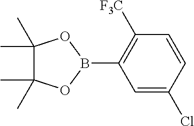 [SMILES: CC1(C)OB(c2cc(Cl)ccc2C(F)(F)F)OC1(C)C] | 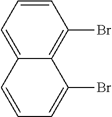 [SMILES: Brc1cccc2cccc(Br)c12] | 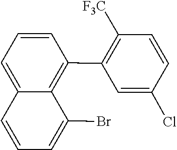 [SMILES: FC(F)(F)c1ccc(Cl)cc1-c1cccc2cccc(Br)c12] | 71 |
| b16 | 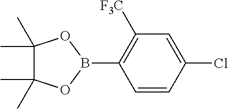 [SMILES: CC1(C)OB(c2ccc(Cl)cc2C(F)(F)F)OC1(C)C] | 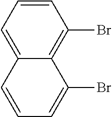 [SMILES: Brc1cccc2cccc(Br)c12] | 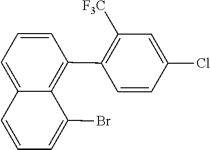 [SMILES: FC(F)(F)c1cc(Cl)ccc1-c1cccc2cccc(Br)c12] | 64 |
| b17 | 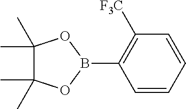 [SMILES: CC1(C)OB(c2ccccc2C(F)(F)F)OC1(C)C] | 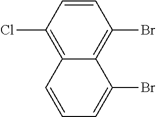 [SMILES: Clc1ccc(Br)c2c(Br)cccc12] | 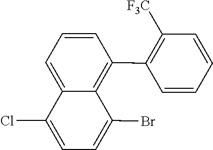 [SMILES: FC(F)(F)c1ccccc1-c1cccc2c(Cl)ccc(Br)c12] | 36 |

### Synthesis of Intermediate c1

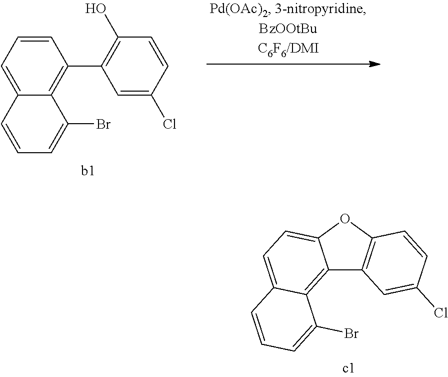 [SMILES: Oc1ccc(Cl)cc1-c1cccc2cccc(Br)c12 ; Clc1ccc2oc3ccc4cccc(Br)c4c3c2c1]

The intermediate b1 (15.5 g; 46.5 mmol), palladium acetate (1.0 g; 4.6 mmol), 3-nitropyridine (0.6 g; 4.6 mmol), hexafluorobenzene (60 mL), 1,3-dimethyl-2-imidazolidinone (50 mL), and tert-butyl peroxybenzoate (18.0 g; 92.9 mmol) were added into a round bottom flask under nitrogen protection, and heated to 90° C.-95° C., and a reaction was carried out for 12 h. The reaction was stopped, and the reaction solution was cooled to room temperature, dichloromethane (100 mL) and deionized water (200 mL) were added into the reaction solution, liquid separation was performed, an organic phase was washed with water and dried over anhydrous magnesium sulfate, and a solvent was removed under reduced pressure; and the obtained crude product was purified by silica gel column chromatography using dichloromethane/n-heptane as a solvent to obtain an intermediate c1 (8.0 g; yield: 52%) as a white solid.

Referring to the synthesis method of the intermediate c1, intermediates shown in Table 3 below were synthesized by using a reactant I instead of the intermediate b1:

TABLE 3

| | | | Yield |
|---|---|---|---|
| Intermediate | Reactant I | Structure | (%) |
| c1 | 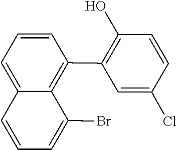 [SMILES: Oc1ccc(Cl)cc1-c1cccc2cccc(Br)c12] | 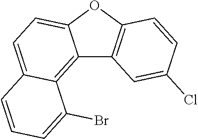 [SMILES: Clc1ccc2oc3ccc4cccc(Br)c4c3c2c1] | 52 |
| c2 | 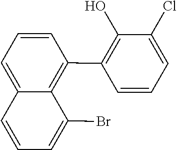 [SMILES: Oc1c(Cl)cccc1-c1cccc2cccc(Br)c12] | 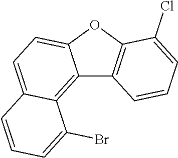 [SMILES: Clc1cccc2c1oc1ccc3cccc(Br)c3c12] | 42 |
| c3 | 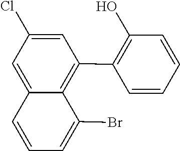 [SMILES: Oc1ccccc1-c1cc(Cl)cc2cccc(Br)c12] | 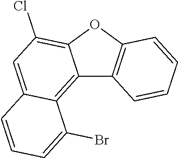 [SMILES: Clc1cc2cccc(Br)c2c2c1oc1ccccc12] | 38 |
| c4 | 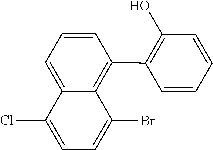 [SMILES: Oc1ccccc1-c1cccc2c(Cl)ccc(Br)c12] | 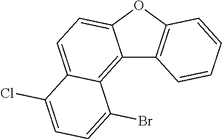 [SMILES: Clc1ccc(Br)c2c1ccc1oc3ccccc3c12] | 57 |
| c5 | 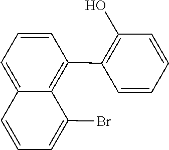 [SMILES: Oc1ccccc1-c1cccc2cccc(Br)c12] | 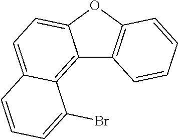 [SMILES: Brc1cccc2ccc3oc4ccccc4c3c12] | 49 |

### Synthesis of Intermediate c6

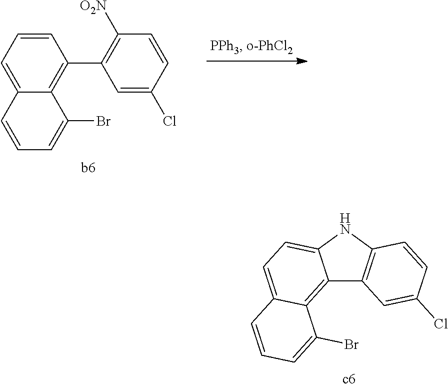 [SMILES: O=[N+]([O-])c1ccc(Cl)cc1-c1cccc2cccc(Br)c12 ; Clc1ccc2[nH]c3ccc4cccc(Br)c4c3c2c1]

The intermediate b6 (15.0 g; 41.4 mmol), triphenylphosphine (27.1 g; 103.4 mmol), and o-dichlorobenzene (150 mL) were added into a round bottom flask under nitrogen protection, and heated to 175° C.-180° C. under stirring, and a reaction was carried out for 48 h. The reaction solution was cooled to room temperature, deionized water (200 mL) was added, liquid separation was performed, an organic phase was washed with water and dried over anhydrous magnesium sulfate, and a solvent was removed under reduced pressure at high temperature; and the obtained crude product was purified by silica gel column chromatography using a dichloromethane/n-heptane system to obtain an intermediate c6 (9.2 g; yield: 67%) as a white solid.

Referring to the synthesis method of the intermediate c6, intermediates shown in Table 4 below were synthesized by using a reactant E instead of the intermediate b6:

TABLE 4

| Intermediate | Reactant E | Structure | Yield (%) |
|---|---|---|---|
| c6 | 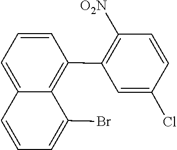 [SMILES: O=[N+]([O-])c1ccc(Cl)cc1-c1cccc2cccc(Br)c12] | 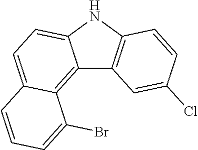 [SMILES: Clc1ccc2[nH]c3ccc4cccc(Br)c4c3c2c1] | 67 |
| c7 | 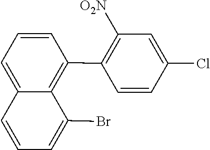 [SMILES: O=[N+]([O-])c1cc(Cl)ccc1-c1cccc2cccc(Br)c12] | 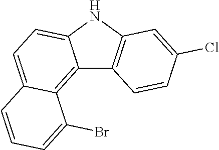 [SMILES: Clc1ccc2c(c1)[nH]c1ccc3cccc(Br)c3c12] | 61 |
| c8 | 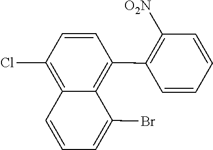 [SMILES: O=[N+]([O-])c1ccccc1-c1ccc(Cl)c2cccc(Br)c12] | 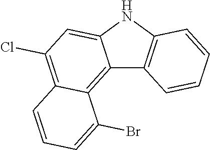 [SMILES: Clc1cc2[nH]c3ccccc3c2c2c(Br)cccc12] | 57 |
| c9 | 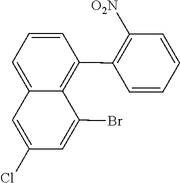 [SMILES: O=[N+]([O-])c1ccccc1-c1cccc2cc(Cl)cc(Br)c12] | 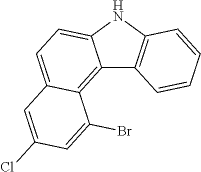 [SMILES: Clc1cc(Br)c2c(ccc3[nH]c4ccccc4c32)c1] | 60 |
| c10 | 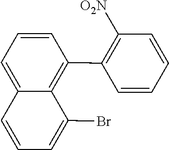 [SMILES: O=[N+]([O-])c1ccccc1-c1cccc2cccc(Br)c12] | 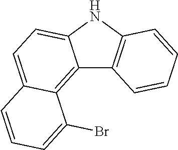 [SMILES: Brc1cccc2ccc3[nH]c4ccccc4c3c12] | 66 |

### Synthesis of Intermediate c11

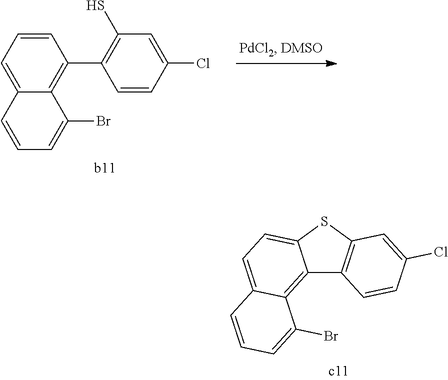 [SMILES: Sc1cc(Cl)ccc1-c1cccc2cccc(Br)c12 ; Clc1ccc2c(c1)sc1ccc3cccc(Br)c3c12]

The intermediate b11 (8.5 g; 24.4 mmol), palladium chloride (0.2 g; 1.2 mmol) and dimethylsulfoxide (80 mL) were added to a round bottom flask under nitrogen protection, and a reaction was carried out under stirring at 140° C.-145° C. for 24 h. The reaction was cooled to room temperature, dichloromethane (100 mL) and deionized water (150 mL) were added into the reaction solution, liquid separation was performed, an organic phase was washed with water and dried over anhydrous magnesium sulfate, and a solvent was removed under reduced pressure; and the obtained crude product was purified by silica gel column chromatography using a dichloromethane/n-heptane system to obtain an intermediate c11 (6.0 g; yield: 71%) as a white solid.

Referring to the synthesis method of the intermediate c11, intermediates shown in Table 5 below were synthesized by using a reactant G instead of the intermediate b11:

TABLE 5

| Intermediate | Reactant G | Structure | Yield (%) |
|---|---|---|---|
| c11 | 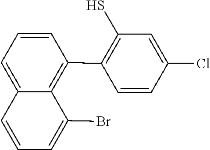 [SMILES: Sc1cc(Cl)ccc1-c1cccc2cccc(Br)c12] | 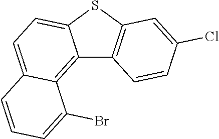 [SMILES: Clc1ccc2c(c1)sc1ccc3cccc(Br)c3c12] | 71 |
| c12 | 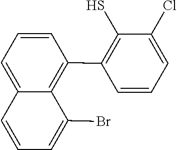 [SMILES: Sc1c(Cl)cccc1-c1cccc2cccc(Br)c12] | 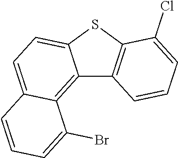 [SMILES: Clc1cccc2c1sc1ccc3cccc(Br)c3c12] | 69 |
| c13 | 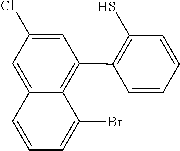 [SMILES: Sc1ccccc1-c1cc(Cl)cc2cccc(Br)c12] | 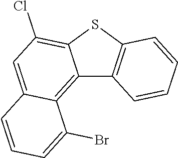 [SMILES: Clc1cc2cccc(Br)c2c2c1sc1ccccc12] | 65 |
| c14 | 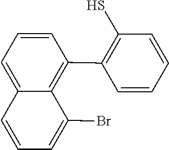 [SMILES: Sc1ccccc1-c1cccc2cccc(Br)c12] | 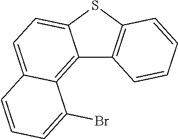 [SMILES: Brc1cccc2ccc3sc4ccccc4c3c12] | 75 |

### Synthesis of Intermediate c15

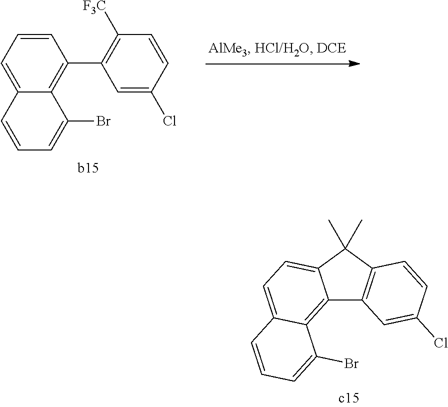 [SMILES: FC(F)(F)c1ccc(Cl)cc1-c1cccc2cccc(Br)c12 ; CC1(C)c2ccc(Cl)cc2-c2c1ccc1cccc(Br)c21]

The intermediate b15 (18.0 g; 46.7 mmol) and dichloroethane (150 mL) were added into a round bottom flask under nitrogen protection, a solution of trimethylaluminum in n-hexane (116.7 mL, 233.4 mmol, 2M) was slowly added dropwise with stirring at 20° C.-25° C., and a reaction was continued to be carried out at 20° C.-25° C. for 2 h after the dropwise addition was complete; hydrochloric acid (25.5 g; 700.2 mmol) in water was added into the reaction solution, extraction was carried out by using diethyl ether, organic phases were combined, and dried, and a solvent was removed under reduced pressure; and a crude product was purified by silica gel chromatography using dichloromethane/n-heptane as a solvent system to obtain an intermediate c15 (7.5 g; yield: 45%) as a white solid.

Referring to the synthesis method of the intermediate c15, intermediates shown in Table 6 below were synthesized by using a reactant L instead of the intermediate b15:

TABLE 6

| Intermediate | Reactant L | Structure | Yield (%) |
|---|---|---|---|
| c15 | 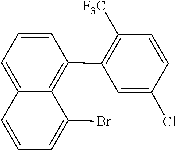 [SMILES: FC(F)(F)c1ccc(Cl)cc1-c1cccc2cccc(Br)c12] | 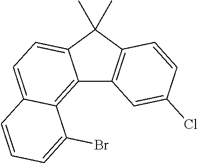 [SMILES: CC1(C)c2ccc(Cl)cc2-c2c1ccc1cccc(Br)c21] | 45 |
| c16 | 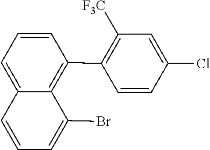 [SMILES: FC(F)(F)c1cc(Cl)ccc1-c1cccc2cccc(Br)c12] | 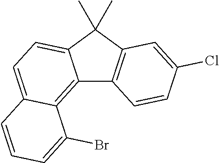 [SMILES: CC1(C)c2cc(Cl)ccc2-c2c1ccc1cccc(Br)c21] | 49 |
| c17 | 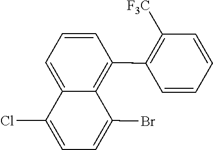 [SMILES: FC(F)(F)c1ccccc1-c1cccc2c(Cl)ccc(Br)c12] | 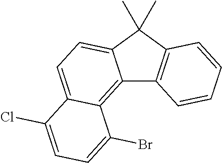 [SMILES: CC1(C)c2ccccc2-c2c1ccc1c(Cl)ccc(Br)c21] | 40 |

### Synthesis of Intermediate d1

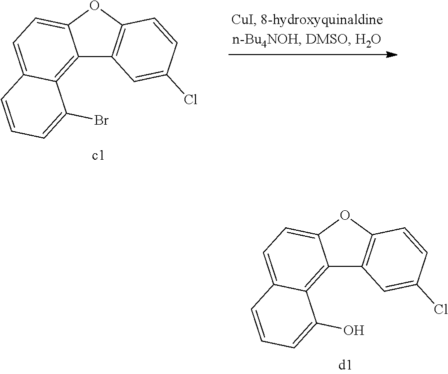 [SMILES: Clc1ccc2oc3ccc4cccc(Br)c4c3c2c1 ; Oc1cccc2ccc3oc4ccc(Cl)cc4c3c12]

The intermediate c1 (7.9 g; 23.8 mol), cuprous iodide (0.5 g; 2.4 mmol), 8-hydroxyquinaldine (0.8 g; 4.8 mmol), tetrabutylammonium hydroxide (18.5 g; 71.5 mmol), dimethylsulfoxide (80 mL) and deionized water (120 mL) were added into a round bottom flask under nitrogen protection, and heated under stirring to 125° C.-130° C., and a reaction was carried out for 24 h. The reaction was cooled to room temperature, dichloromethane (150 mL) and deionized water (200 mL) were added into the reaction solution, liquid separation was performed, an organic phase was washed with water and dried over anhydrous magnesium sulfate, and a solvent was removed under reduced pressure; and the obtained crude product was purified by silica gel column chromatography using a dichloromethane/n-heptane system to obtain an intermediate d1 (5.1 g; yield: 80%) as a white solid.

Referring to the synthesis method of the intermediate d1, intermediates shown in Table 7 below were synthesized by using a reactant M instead of the intermediate c1:

TABLE 7

| Intermediate | Reactant M | Structure | Yield (%) |
|---|---|---|---|
| d1 | 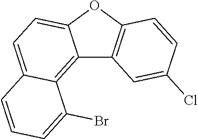 [SMILES: Clc1ccc2oc3ccc4cccc(Br)c4c3c2c1] | 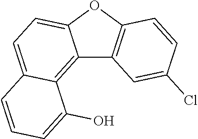 [SMILES: Oc1cccc2ccc3oc4ccc(Cl)cc4c3c12] | 80 |
| d2 | 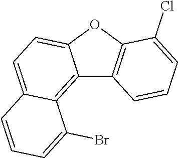 [SMILES: Clc1cccc2c1oc1ccc3cccc(Br)c3c12] | 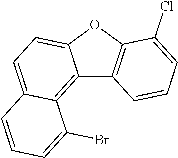 [SMILES: Clc1cccc2c1oc1ccc3cccc(Br)c3c12] | 74 |
| d3 | 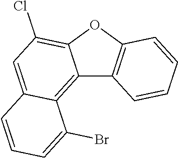 [SMILES: Clc1cc2cccc(Br)c2c2c1oc1ccccc12] | 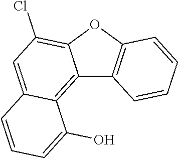 [SMILES: Oc1cccc2cc(Cl)c3oc4ccccc4c3c12] | 71 |
| d4 | 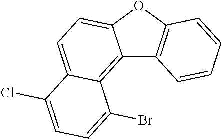 [SMILES: Clc1ccc(Br)c2c1ccc1oc3ccccc3c12] | 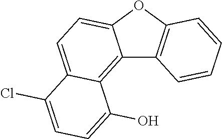 [SMILES: Oc1ccc(Cl)c2ccc3oc4ccccc4c3c12] | 77 |
| d5 | 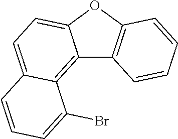 [SMILES: Brc1cccc2ccc3oc4ccccc4c3c12] | 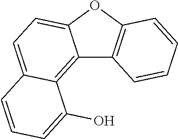 [SMILES: Oc1cccc2ccc3oc4ccccc4c3c12] | 83 |
| d6 | 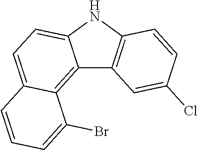 [SMILES: Clc1ccc2[nH]c3ccc4cccc(Br)c4c3c2c1] | 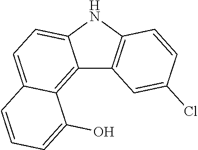 [SMILES: Oc1cccc2ccc3[nH]c4ccc(Cl)cc4c3c12] | 73 |
| d7 | 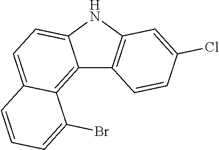 [SMILES: Clc1ccc2c(c1)[nH]c1ccc3cccc(Br)c3c12] | 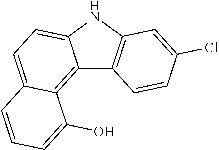 [SMILES: Oc1cccc2ccc3[nH]c4cc(Cl)ccc4c3c12] | 78 |
| d8 | 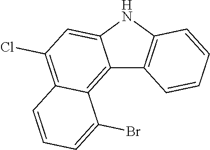 [SMILES: Clc1cc2[nH]c3ccccc3c2c2c(Br)cccc12] | 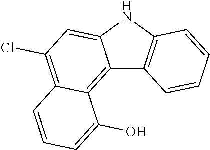 [SMILES: Oc1cccc2c(Cl)cc3[nH]c4ccccc4c3c12] | 76 |
| d9 | 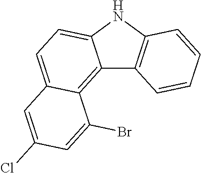 [SMILES: Clc1cc(Br)c2c(ccc3[nH]c4ccccc4c32)c1] | 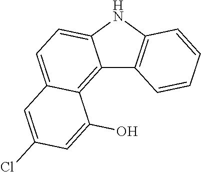 [SMILES: Oc1cc(Cl)cc2ccc3[nH]c4ccccc4c3c12] | 75 |
| d10 | 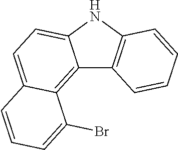 [SMILES: Brc1cccc2ccc3[nH]c4ccccc4c3c12] | 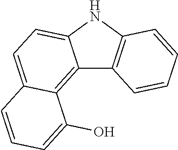 [SMILES: Oc1cccc2ccc3[nH]c4ccccc4c3c12] | 72 |
| d11 | 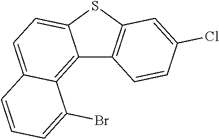 [SMILES: Clc1ccc2c(c1)sc1ccc3cccc(Br)c3c12] | 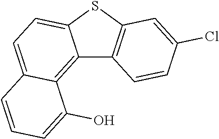 [SMILES: Oc1cccc2ccc3sc4cc(Cl)ccc4c3c12] | 69 |
| d12 | 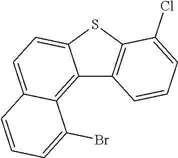 [SMILES: Clc1cccc2c1sc1ccc3cccc(Br)c3c12] | 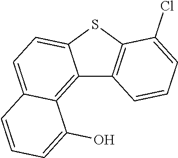 [SMILES: Oc1cccc2ccc3sc4c(Cl)cccc4c3c12] | 64 |
| d13 | 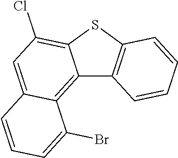 [SMILES: Clc1cc2cccc(Br)c2c2c1sc1ccccc12] | 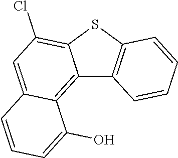 [SMILES: Oc1cccc2cc(Cl)c3sc4ccccc4c3c12] | 76 |
| d14 | 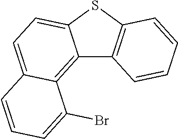 [SMILES: Brc1cccc2ccc3sc4ccccc4c3c12] | 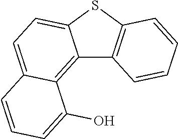 [SMILES: Oc1cccc2ccc3sc4ccccc4c3c12] | 70 |
| d15 | 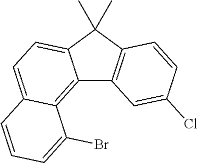 [SMILES: CC1(C)c2ccc(Cl)cc2-c2c1ccc1cccc(Br)c21] | 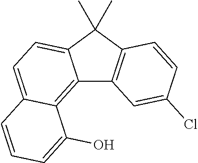 [SMILES: CC1(C)c2ccc(Cl)cc2-c2c1ccc1cccc(O)c21] | 60 |
| d16 | 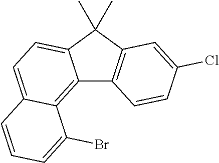 [SMILES: CC1(C)c2cc(Cl)ccc2-c2c1ccc1cccc(Br)c21] | 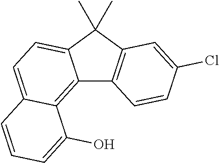 [SMILES: CC1(C)c2cc(Cl)ccc2-c2c1ccc1cccc(O)c21] | 70 |
| d17 | 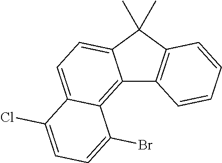 [SMILES: CC1(C)c2ccccc2-c2c1ccc1c(Cl)ccc(Br)c21] | 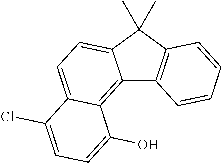 [SMILES: CC1(C)c2ccccc2-c2c1ccc1c(Cl)ccc(O)c21] | 74 |

### Synthesis of Intermediate e1

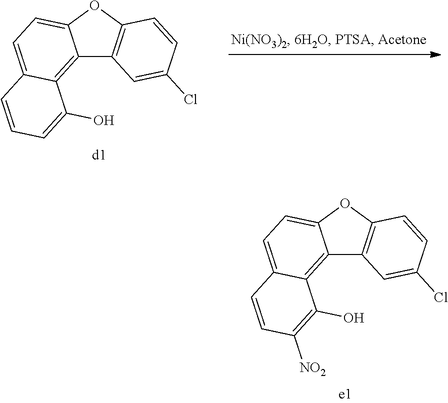 [SMILES: Oc1cccc2ccc3oc4ccc(Cl)cc4c3c12 ; O=[N+]([O-])c1ccc2ccc3oc4ccc(Cl)cc4c3c2c1O]

The intermediate d1 (5.0 g; 18.6 mmol), nickel nitrate hexahydrate (5.4 g; 18.6 mmol), p-toluenesulfonic acid (0.04 g; 0.2 mmol) and acetone (100 mL) were added into a round bottom flask under nitrogen protection, and a reaction was carried out under stirring at 20° C.-25° C. for 2 h. The reaction was stopped, dichloromethane (100 mL) and deionized water (150 mL) were added into the reaction solution, liquid separation was performed, an organic phase was washed with water and dried over anhydrous magnesium sulfate, and a solvent was removed under reduced pressure; and the obtained crude product was purified by silica gel column chromatography using a dichloromethane/n-heptane system to obtain an intermediate e1 (4.7 g; yield: 81%) as a white solid.

Referring to the synthesis method of the intermediate e1, intermediates shown in Table 8 below were synthesized by using a reactant N instead of the intermediate d1:

TABLE 8

| Intermediate | Reactant N | Structure | Yield (%) |
|---|---|---|---|
| e1 | 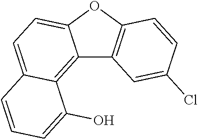 [SMILES: Oc1cccc2ccc3oc4ccc(Cl)cc4c3c12] | 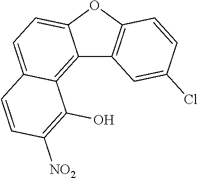 [SMILES: O=[N+]([O-])c1ccc2ccc3oc4ccc(Cl)cc4c3c2c1O] | 84 |
| e2 | 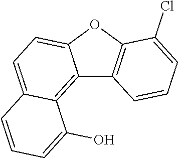 [SMILES: Oc1cccc2ccc3oc4c(Cl)cccc4c3c12] | 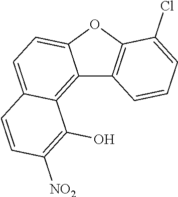 [SMILES: O=[N+]([O-])c1ccc2ccc3oc4c(Cl)cccc4c3c2c1O] | 81 |
| e3 | 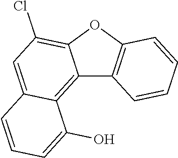 [SMILES: Oc1cccc2cc(Cl)c3oc4ccccc4c3c12] | 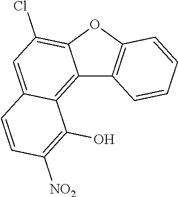 [SMILES: O=[N+]([O-])c1ccc2cc(Cl)c3oc4ccccc4c3c2c1O] | 88 |
| e4 | 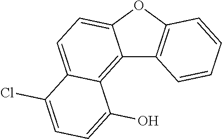 [SMILES: Oc1ccc(Cl)c2ccc3oc4ccccc4c3c12] | 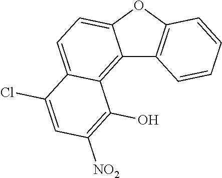 [SMILES: O=[N+]([O-])c1cc(Cl)c2ccc3oc4ccccc4c3c2c1O] | 80 |
| e5 | 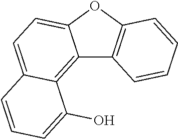 [SMILES: Oc1cccc2ccc3oc4ccccc4c3c12] | 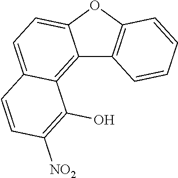 [SMILES: O=[N+]([O-])c1ccc2ccc3oc4ccccc4c3c2c1O] | 76 |
| e6 | 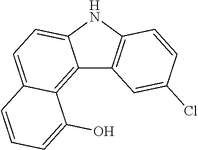 [SMILES: Oc1cccc2ccc3[nH]c4ccc(Cl)cc4c3c12] | 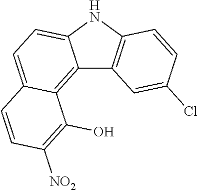 [SMILES: O=[N+]([O-])c1ccc2ccc3[nH]c4ccc(Cl)cc4c3c2c1O] | 75 |
| e7 | 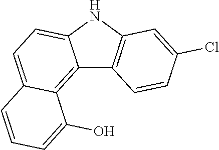 [SMILES: Oc1cccc2ccc3[nH]c4cc(Cl)ccc4c3c12] | 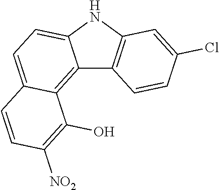 [SMILES: O=[N+]([O-])c1ccc2ccc3[nH]c4cc(Cl)ccc4c3c2c1O] | 76 |
| e8 | 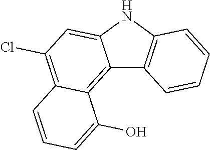 [SMILES: Oc1cccc2c(Cl)cc3[nH]c4ccccc4c3c12] | 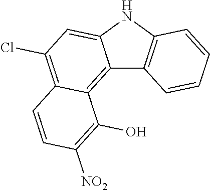 [SMILES: O=[N+]([O-])c1ccc2c(Cl)cc3[nH]c4ccccc4c3c2c1O] | 87 |
| e9 | 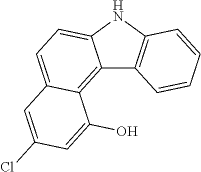 [SMILES: Oc1cc(Cl)cc2ccc3[nH]c4ccccc4c3c12] | 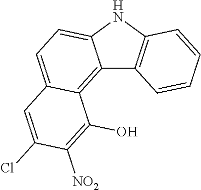 [SMILES: O=[N+]([O-])c1c(Cl)cc2ccc3[nH]c4ccccc4c3c2c1O] | 68 |
| e10 | 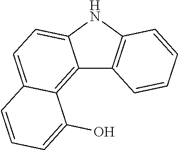 [SMILES: Oc1cccc2ccc3[nH]c4ccccc4c3c12] | 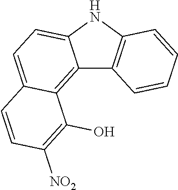 [SMILES: O=[N+]([O-])c1ccc2ccc3[nH]c4ccccc4c3c2c1O] | 89 |
| e11 | 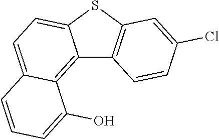 [SMILES: Oc1cccc2ccc3sc4cc(Cl)ccc4c3c12] | 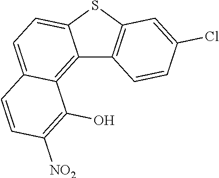 [SMILES: O=[N+]([O-])c1ccc2ccc3sc4cc(Cl)ccc4c3c2c1O] | 82 |
| e12 | 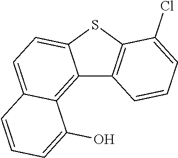 [SMILES: Oc1cccc2ccc3sc4c(Cl)cccc4c3c12] | 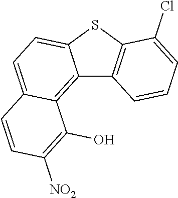 [SMILES: O=[N+]([O-])c1ccc2ccc3sc4c(Cl)cccc4c3c2c1O] | 75 |
| e13 | 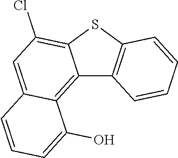 [SMILES: Oc1cccc2cc(Cl)c3sc4ccccc4c3c12] | 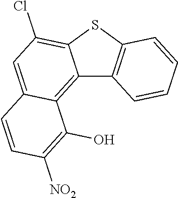 [SMILES: O=[N+]([O-])c1ccc2cc(Cl)c3sc4ccccc4c3c2c1O] | 86 |
| e14 | 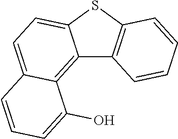 [SMILES: Oc1cccc2ccc3sc4ccccc4c3c12] | 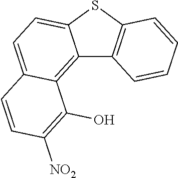 [SMILES: O=[N+]([O-])c1ccc2ccc3sc4ccccc4c3c2c1O] | 87 |
| e15 | 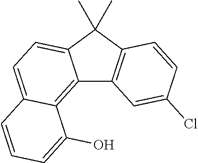 [SMILES: CC1(C)c2ccc(Cl)cc2-c2c1ccc1cccc(O)c21] | 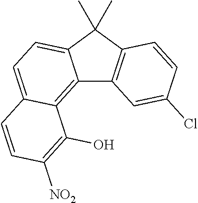 [SMILES: CC1(C)c2ccc(Cl)cc2-c2c1ccc1ccc([N+](=O)[O-])c(O)c21] | 83 |
| e16 | 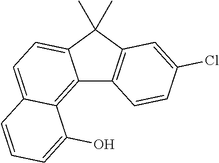 [SMILES: CC1(C)c2cc(Cl)ccc2-c2c1ccc1cccc(O)c21] | 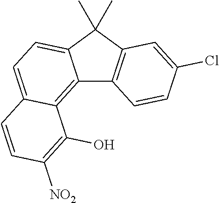 [SMILES: CC1(C)c2cc(Cl)ccc2-c2c1ccc1ccc([N+](=O)[O-])c(O)c21] | 80 |
| e17 | 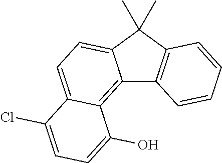 [SMILES: CC1(C)c2ccccc2-c2c1ccc1c(Cl)ccc(O)c21] | 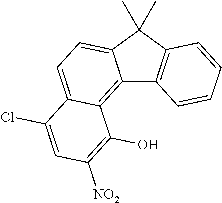 [SMILES: CC1(C)c2ccccc2-c2c1ccc1c(Cl)cc([N+](=O)[O-])c(O)c21] | 87 |

### Synthesis of intermediate o1-c1

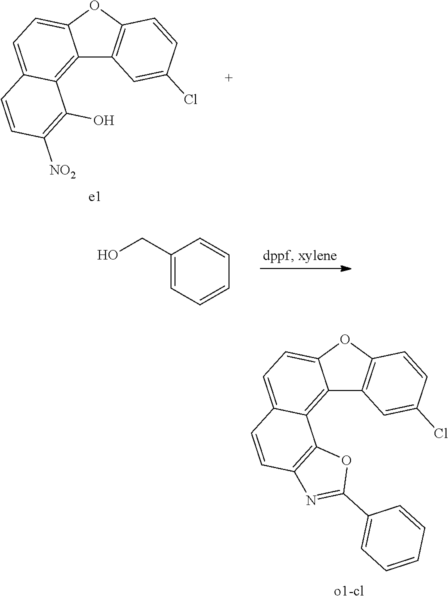 [SMILES: O=[N+]([O-])c1ccc2ccc3oc4ccc(Cl)cc4c3c2c1O ; OCc1ccccc1 ; Clc1ccc2oc3ccc4ccc5nc(-c6ccccc6)oc5c4c3c2c1]

The intermediate e1 (4.7 g; 15.0 mmol), benzyl alcohol (1.9 g, 18.0 mmol), 1,1′-bis(diphenylphosphino)ferrocene (0.2 g; 0.4 mmol) and xylene (50 mL) were added into a round-bottom flask under nitrogen protection, heated to 130° C.-135° C. under stirring, and a reaction was carried out under reflux for 48 h. The reaction was cooled to room temperature, toluene (50 mL) and deionized water (100 mL) were added into the reaction solution, and organic phases were combined, dried over anhydrous magnesium sulfate, filtered, and concentrated; and a crude product was purified by silica gel column chromatography using a dichloromethane/n-heptane system to obtain an intermediate o1-c1 (3.5 g; yield: 63%) as a white solid.

Referring to the synthesis method of the intermediate o1-c1, intermediates shown in Table 9 below were synthesized by using a reactant P instead of the intermediate e1, and using a reactant Q instead of benzyl alcohol:

TABLE 9

| Intermediate | Reactant P | Reactant Q | Structure | Yield (%) |
|---|---|---|---|---|
| 01-cl | 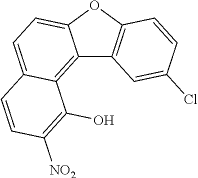 [SMILES: O=[N+]([O-])c1ccc2ccc3oc4ccc(Cl)cc4c3c2c1O] | 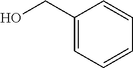 | 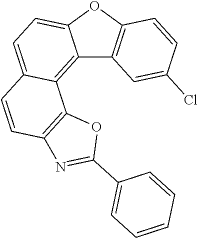 [SMILES: Clc1ccc2oc3ccc4ccc5nc(-c6ccccc6)oc5c4c3c2c1] | 63 |
| 02-cl | 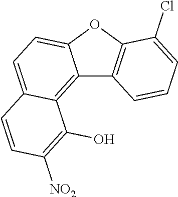 [SMILES: O=[N+]([O-])c1ccc2ccc3oc4c(Cl)cccc4c3c2c1O] | 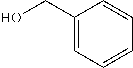 | 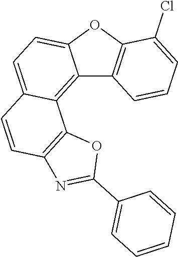 [SMILES: Clc1cccc2c1oc1ccc3ccc4nc(-c5ccccc5)oc4c3c12] | 60 |
| 03-cl | 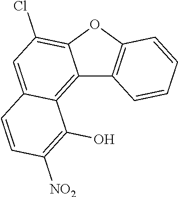 [SMILES: O=[N+]([O-])c1ccc2cc(Cl)c3oc4ccccc4c3c2c1O] | 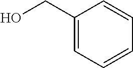 | 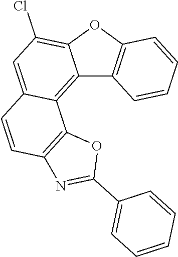 [SMILES: Clc1cc2ccc3nc(-c4ccccc4)oc3c2c2c1oc1ccccc12] | 56 |
| 04-cl | 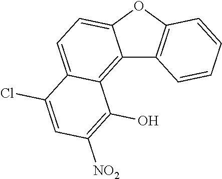 [SMILES: O=[N+]([O-])c1cc(Cl)c2ccc3oc4ccccc4c3c2c1O] | 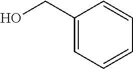 | 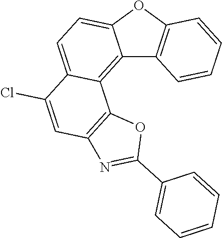 [SMILES: Clc1cc2nc(-c3ccccc3)oc2c2c1ccc1oc3ccccc3c12] | 60 |
| 05-cl | 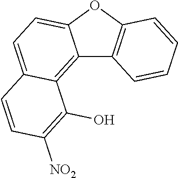 [SMILES: O=[N+]([O-])c1ccc2ccc3oc4ccccc4c3c2c1O] | 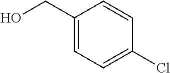 | 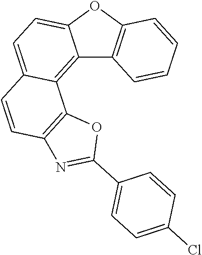 [SMILES: Clc1ccc(-c2nc3ccc4ccc5oc6ccccc6c5c4c3o2)cc1] | 65 |
| n0-h | 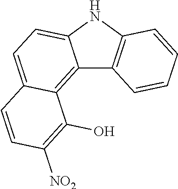 [SMILES: O=[N+]([O-])c1ccc2ccc3[nH]c4ccccc4c3c2c1O] | 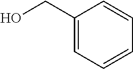 | 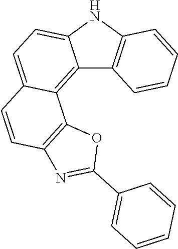 [SMILES: c1ccc(-c2nc3ccc4ccc5[nH]c6ccccc6c5c4c3o2)cc1] | 58 |
| n1-h | 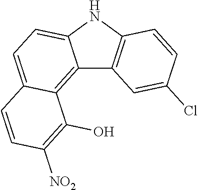 [SMILES: O=[N+]([O-])c1ccc2ccc3[nH]c4ccc(Cl)cc4c3c2c1O] | 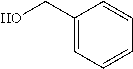 | 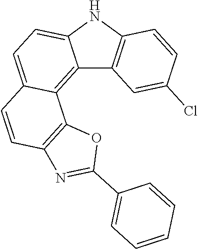 [SMILES: Clc1ccc2[nH]c3ccc4ccc5nc(-c6ccccc6)oc5c4c3c2c1] | 50 |
| n2-h | 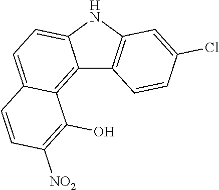 [SMILES: O=[N+]([O-])c1ccc2ccc3[nH]c4cc(Cl)ccc4c3c2c1O] | 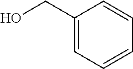 | 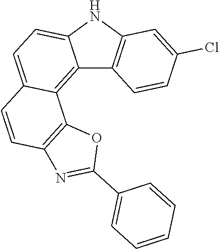 [SMILES: Clc1ccc2c(c1)[nH]c1ccc3ccc4nc(-c5ccccc5)oc4c3c12] | 62 |
| n3-h | 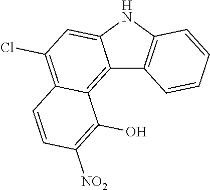 [SMILES: O=[N+]([O-])c1ccc2c(Cl)cc3[nH]c4ccccc4c3c2c1O] | 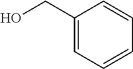 | 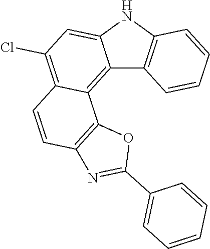 [SMILES: Clc1cc2[nH]c3ccccc3c2c2c1ccc1nc(-c3ccccc3)oc12] | 45 |
| n4-h | 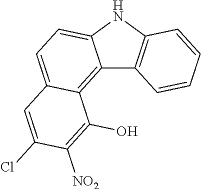 [SMILES: O=[N+]([O-])c1c(Cl)cc2ccc3[nH]c4ccccc4c3c2c1O] | 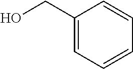 | 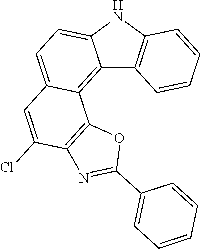 [SMILES: Clc1cc2ccc3[nH]c4ccccc4c3c2c2oc(-c3ccccc3)nc12] | 63 |
| n5-h | 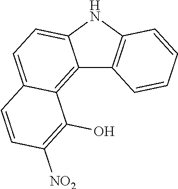 [SMILES: O=[N+]([O-])c1ccc2ccc3[nH]c4ccccc4c3c2c1O] | 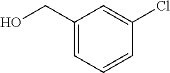 | 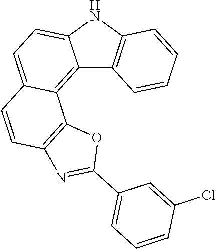 [SMILES: Clc1cccc(-c2nc3ccc4ccc5[nH]c6ccccc6c5c4c3o2)c1] | 54 |
| s1-cl | 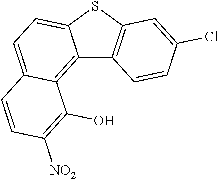 [SMILES: O=[N+]([O-])c1ccc2ccc3sc4cc(Cl)ccc4c3c2c1O] | 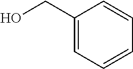 | 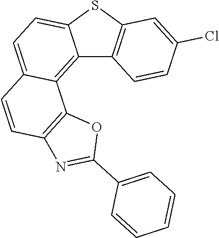 [SMILES: Clc1ccc2c(c1)sc1ccc3ccc4nc(-c5ccccc5)oc4c3c12] | 61 |
| s2-cl | 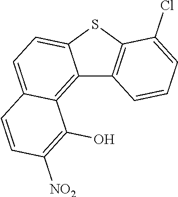 [SMILES: O=[N+]([O-])c1ccc2ccc3sc4c(Cl)cccc4c3c2c1O] | 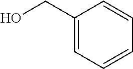 | 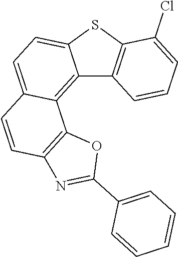 [SMILES: Clc1cccc2c1sc1ccc3ccc4nc(-c5ccccc5)oc4c3c12] | 54 |
| s3-cl | 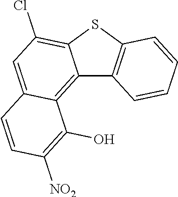 [SMILES: O=[N+]([O-])c1ccc2cc(Cl)c3sc4ccccc4c3c2c1O] | 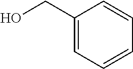 | 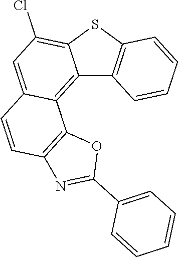 [SMILES: Clc1cc2ccc3nc(-c4ccccc4)oc3c2c2c1sc1ccccc12] | 67 |
| s4-cl | 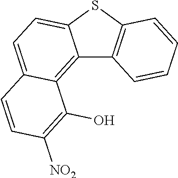 [SMILES: O=[N+]([O-])c1ccc2ccc3sc4ccccc4c3c2c1O] | 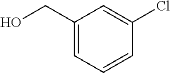 | 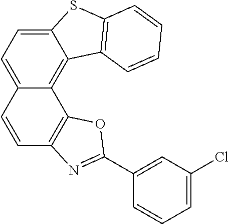 [SMILES: Clc1cccc(-c2nc3ccc4ccc5sc6ccccc6c5c4c3o2)c1] | 45 |
| c1-cl | 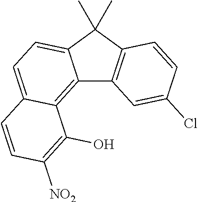 [SMILES: CC1(C)c2ccc(Cl)cc2-c2c1ccc1ccc([N+](=O)[O-])c(O)c21] | 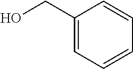 | 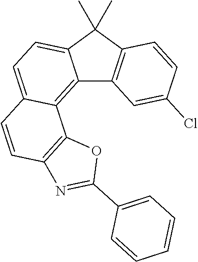 [SMILES: CC1(C)c2ccc(Cl)cc2-c2c1ccc1ccc3nc(-c4ccccc4)oc3c21] | 53 |
| c2-cl | 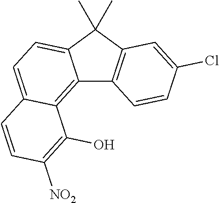 [SMILES: CC1(C)c2cc(Cl)ccc2-c2c1ccc1ccc([N+](=O)[O-])c(O)c21] | 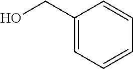 | 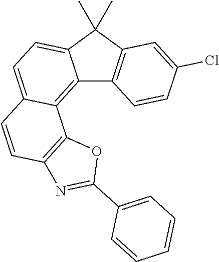 [SMILES: CC1(C)c2cc(Cl)ccc2-c2c1ccc1ccc3nc(-c4ccccc4)oc3c21] | 57 |
| c3-cl | 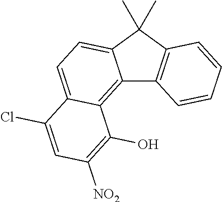 [SMILES: CC1(C)c2ccccc2-c2c1ccc1c(Cl)cc([N+](=O)[O-])c(O)c21] | 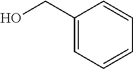 | 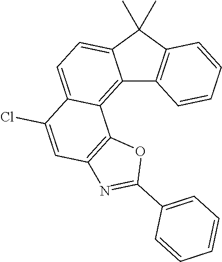 [SMILES: CC1(C)c2ccccc2-c2c1ccc1c(Cl)cc3nc(-c4ccccc4)oc3c21] | 52 |

### Synthesis of Intermediate n1-c1

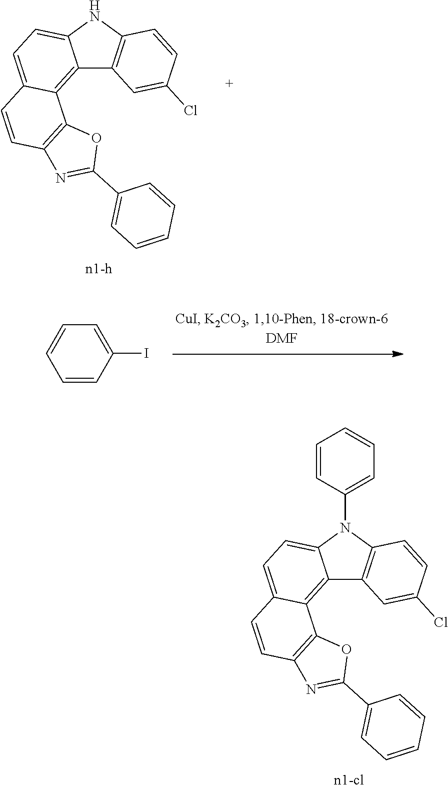 [SMILES: Clc1ccc2[nH]c3ccc4ccc5nc(-c6ccccc6)oc5c4c3c2c1 ; Ic1ccccc1 ; Clc1ccc2c(c1)c1c3c(ccc4nc(-c5ccccc5)oc43)ccc1n2-c1ccccc1]

The intermediate n1-h (5.5 g; 14.9 mmol), iodobenzene (3.3 g; 16.4 mmol), cuprous iodide (0.6 g; 3.0 mmol), anhydrous potassium carbonate (4.5 g; 32.8 mmol), 1,10-phenanthroline (1.1 g; 6.0 mmol), 18-crown-6 (0.8 g; 3.0 mmol) and dimethylformamide (50 mL) were added into a round bottom flask under nitrogen protection, heated to 135° C.-140° C. under stirring, and a reaction was carried out for 24 h. The reaction was stopped, the reaction solution was cooled to room temperature, deionized water (100 mL) and dichloromethane (100 mL) were added, liquid separation was performed, an organic phase was washed with a large amount of water, and dried over anhydrous magnesium sulfate, and a solvent was removed under reduced pressure; and the obtained crude product was purified by silica gel column chromatography using a dichloromethane/n-heptane solvent system, and then purified by recrystallization using a dichloromethane/n-heptane mixed solvent to obtain an intermediate n1-c1 (5.1 g; yield: 77%) as a white solid.

Referring to the synthesis method of the intermediate n1-c1, intermediates/compounds shown in Table 10 below were synthesized by using a reactant R instead of the intermediate n1-h, and using a reactant U instead of iodobenzene:

TABLE 10

| Inter- | | | | |
|---|---|---|---|---|
| mediate/ | | | | Yield |
| compound | Reactant R | Reactant U | Structure | (%) |
| n1-cl | 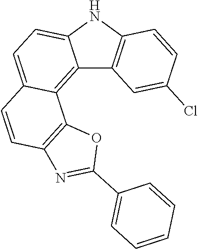 [SMILES: Clc1ccc2[nH]c3ccc4ccc5nc(-c6ccccc6)oc5c4c3c2c1] | 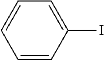 | 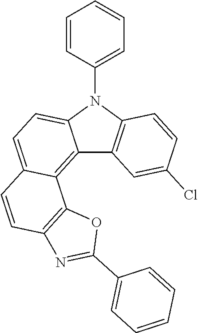 [SMILES: Clc1ccc2c(c1)c1c3c(ccc4nc(-c5ccccc5)oc43)ccc1n2-c1ccccc1] | 77 |
| n2-cl | 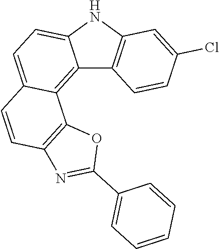 [SMILES: Clc1ccc2c(c1)[nH]c1ccc3ccc4nc(-c5ccccc5)oc4c3c12] | 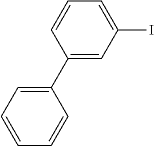 [SMILES: Ic1cccc(-c2ccccc2)c1] | 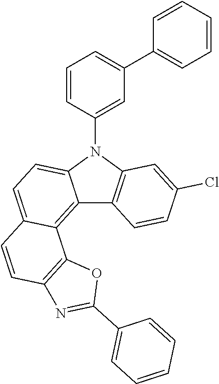 [SMILES: Clc1ccc2c3c4c(ccc5nc(-c6ccccc6)oc54)ccc3n(-c3cccc(-c4ccccc4)c3)c2c1] | 74 |
| n3-cl | 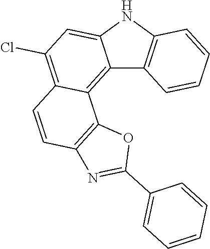 [SMILES: Clc1cc2[nH]c3ccccc3c2c2c1ccc1nc(-c3ccccc3)oc12] | 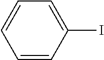 | 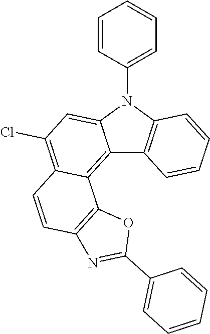 [SMILES: Clc1cc2c(c3ccccc3n2-c2ccccc2)c2c1ccc1nc(-c3ccccc3)oc12] | 81 |
| n4-cl | 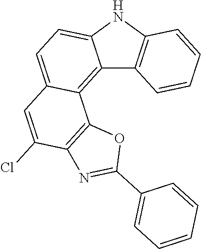 [SMILES: Clc1cc2ccc3[nH]c4ccccc4c3c2c2oc(-c3ccccc3)nc12] | 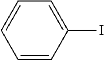 | 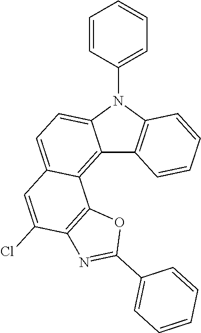 [SMILES: Clc1cc2ccc3c(c4ccccc4n3-c3ccccc3)c2c2oc(-c3ccccc3)nc12] | 75 |
| n5-cl | 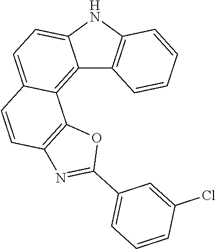 [SMILES: Clc1cccc(-c2nc3ccc4ccc5[nH]c6ccccc6c5c4c3o2)c1] | 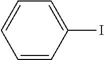 | 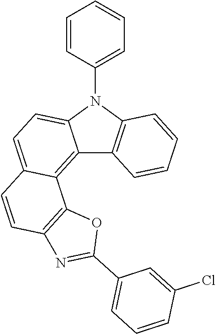 [SMILES: Clc1cccc(-c2nc3ccc4ccc5c(c6ccccc6n5-c5ccccc5)c4c3o2)c1] | 72 |
| B31 | 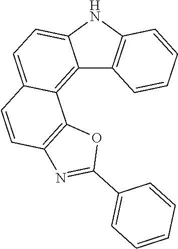 [SMILES: c1ccc(-c2nc3ccc4ccc5[nH]c6ccccc6c5c4c3o2)cc1] | 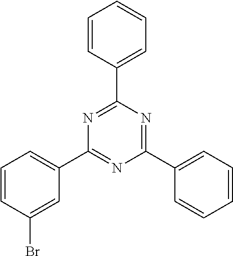 [SMILES: Brc1cccc(-c2nc(-c3ccccc3)nc(-c3ccccc3)n2)c1] | 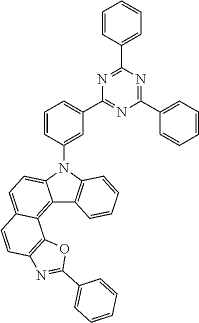 [SMILES: c1ccc(-c2nc(-c3ccccc3)nc(-c3cccc(-n4c5ccccc5c5c6c(ccc7nc(-c8ccccc8)oc76)ccc54)c3)n2)cc1] | 54 |

### Synthesis of compound B24

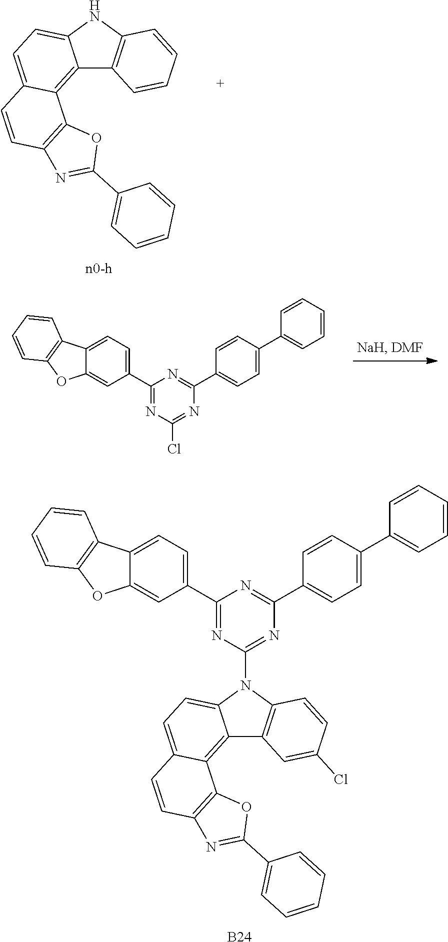 [SMILES: c1ccc(-c2nc3ccc4ccc5[nH]c6ccccc6c5c4c3o2)cc1 ; Clc1nc(-c2ccc(-c3ccccc3)cc2)nc(-c2ccc3c(c2)oc2ccccc23)n1 ; Clc1ccc2c(c1)c1c3c(ccc4nc(-c5ccccc5)oc43)ccc1n2-c1nc(-c2ccc(-c3ccccc3)cc2)nc(-c2ccc3c(c2)oc2ccccc23)n1 ; "B24"]

The intermediate n0-h (3.0 g; 9.0 mmol), 2-chloro-4-(biphenyl-4-yl)-6-(dibenzofuran-3-yl)-1,3,5-triazine (5.8 g; 13.5 mmol) and N,N-dimethylformlamide (30 mL) were added into a round bottom flask, the temperature was cooled to −5° C. to 0° C. with stirring under nitrogen protection, sodium hydride (0.3 g; 10.8 mmol) was added into the reaction, a reaction was carried out under stirring at −5° C. to 0° C. for 30 min, then the temperature was heated to 20° C. to 25° C., and a reaction was carried out for 16 h. The reaction was stopped, the reaction solution was washed with water, and liquid separation was performed, an organic phase was dried over anhydrous magnesium sulfate, and a solvent was removed under reduced pressure to obtain a crude product; and the crude product was purified by silica gel column chromatography using dichloromethane/n-heptane as an eluent, and then purified by recrystallization using a toluene/n-heptane solvent system to obtain a compound B24 (4.0 g; yield: 61%) as a white solid.

### Synthesis of Intermediate o1-bo

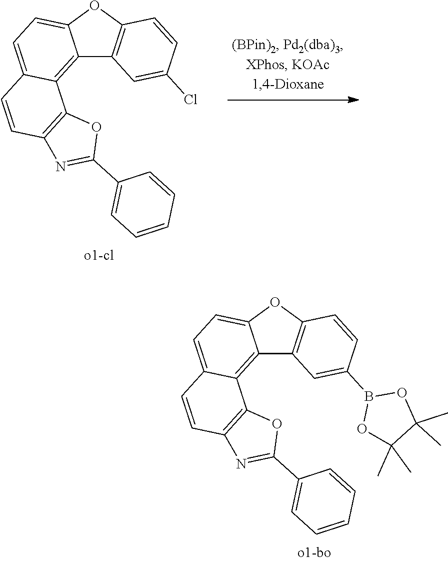 [SMILES: Clc1ccc2oc3ccc4ccc5nc(-c6ccccc6)oc5c4c3c2c1 ; CC1(C)OB(c2ccc3oc4ccc5ccc6nc(-c7ccccc7)oc6c5c4c3c2)OC1(C)C]

The intermediate o1-c1 (3.5 g; 9.5 mmol), bis(pinacolato)diboron (3.6 g; 14.2 mmol), tris(dibenzylideneacetone)dipalladium (0.2 g; 0.2 mmol), 2-dicyclohexylphosphino-2′,4′,6′-triisopropylbiphenyl (0.2 g; 0.4 mmol), potassium acetate (1.4 g; 14.2 mmol) and 1,4-dioxane (30 mL) were added into a round-bottom flask under nitrogen protection, and a reaction was carried out under stirring at 100° C.-105° C. for 24 h. The reaction was cooled to room temperature, dichloromethane (50 mL) and deionized water (50 mL) were added into the reaction solution, liquid separation was performed, an organic phase was washed with water and dried over anhydrous magnesium sulfate, and a solvent was removed under reduced pressure; and the obtained crude was purified by silica gel column chromatography using dichloromethane/n-heptane as a solvent to obtain an intermediate o1-bo (3.1 g; yield: 71%) as a white solid.

Referring to the synthesis method of the intermediate o1-bo, intermediates shown in Table 11 below were synthesized by using a reactant V instead of the intermediate o1-c1:

TABLE 11

| Intermediate | Reactant V | Structure | Yield (%) |
|---|---|---|---|
| 01-bo | 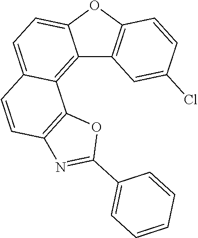 [SMILES: Clc1ccc2oc3ccc4ccc5nc(-c6ccccc6)oc5c4c3c2c1] | 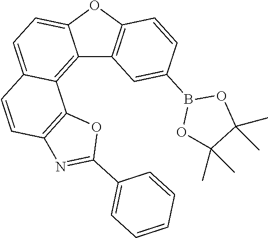 [SMILES: CC1(C)OB(c2ccc3oc4ccc5ccc6nc(-c7ccccc7)oc6c5c4c3c2)OC1(C)C] | 71 |
| 02-bo | 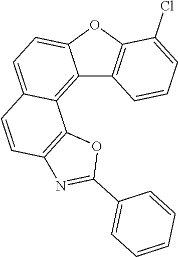 [SMILES: Clc1cccc2c1oc1ccc3ccc4nc(-c5ccccc5)oc4c3c12] | 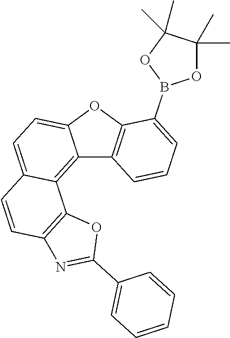 [SMILES: CC1(C)OB(c2cccc3c2oc2ccc4ccc5nc(-c6ccccc6)oc5c4c23)OC1(C)C] | 79 |
| 03-bo | 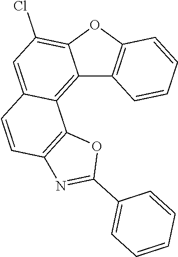 [SMILES: Clc1cc2ccc3nc(-c4ccccc4)oc3c2c2c1oc1ccccc12] | 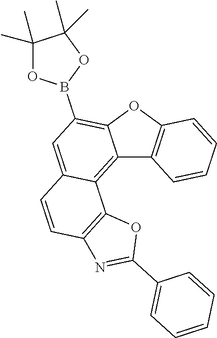 [SMILES: CC1(C)OB(c2cc3ccc4nc(-c5ccccc5)oc4c3c3c2oc2ccccc23)OC1(C)C] | 73 |
| 04-bo | 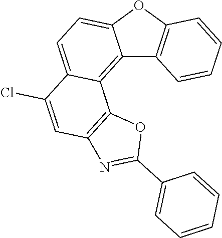 [SMILES: Clc1cc2nc(-c3ccccc3)oc2c2c1ccc1oc3ccccc3c12] | 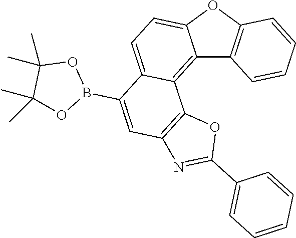 [SMILES: CC1(C)OB(c2cc3nc(-c4ccccc4)oc3c3c2ccc2oc4ccccc4c23)OC1(C)C] | 65 |
| 05-bo | 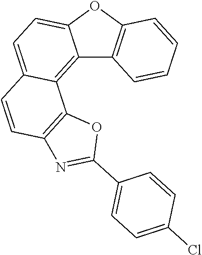 [SMILES: Clc1ccc(-c2nc3ccc4ccc5oc6ccccc6c5c4c3o2)cc1] | 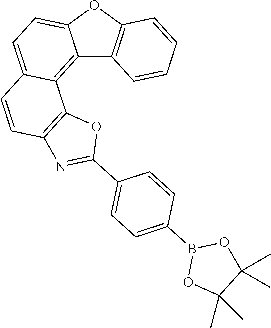 [SMILES: CC1(C)OB(c2ccc(-c3nc4ccc5ccc6oc7ccccc7c6c5c4o3)cc2)OC1(C)C] | 74 |
| n1-bo | 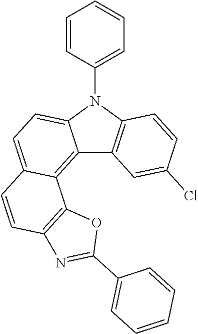 [SMILES: Clc1ccc2c(c1)c1c3c(ccc4nc(-c5ccccc5)oc43)ccc1n2-c1ccccc1] | 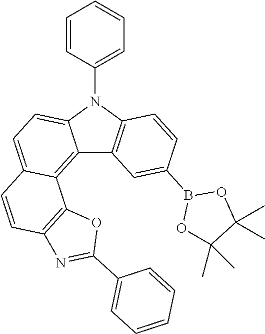 [SMILES: CC1(C)OB(c2ccc3c(c2)c2c4c(ccc5nc(-c6ccccc6)oc54)ccc2n3-c2ccccc2)OC1(C)C] | 58 |
| n2-bo | 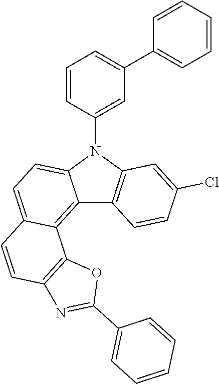 [SMILES: Clc1ccc2c3c4c(ccc5nc(-c6ccccc6)oc54)ccc3n(-c3cccc(-c4ccccc4)c3)c2c1] | 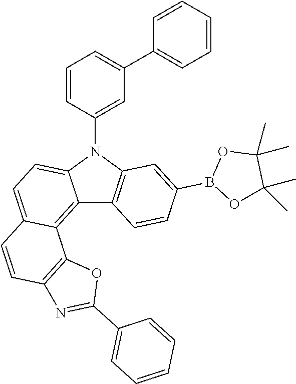 [SMILES: CC1(C)OB(c2ccc3c4c5c(ccc6nc(-c7ccccc7)oc65)ccc4n(-c4cccc(-c5ccccc5)c4)c3c2)OC1(C)C] | 75 |
| n3-bo | 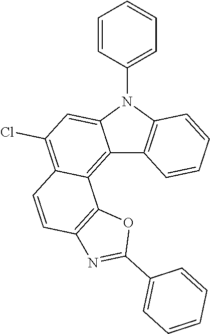 [SMILES: Clc1cc2c(c3ccccc3n2-c2ccccc2)c2c1ccc1nc(-c3ccccc3)oc12] | 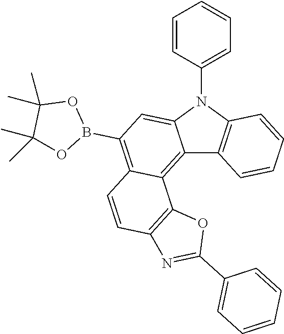 [SMILES: CC1(C)OB(c2cc3c(c4ccccc4n3-c3ccccc3)c3c2ccc2nc(-c4ccccc4)oc23)OC1(C)C] | 66 |
| n4-bo | 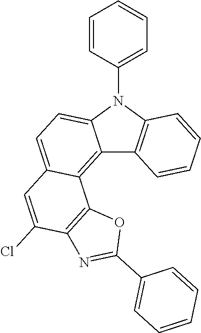 [SMILES: Clc1cc2ccc3c(c4ccccc4n3-c3ccccc3)c2c2oc(-c3ccccc3)nc12] | 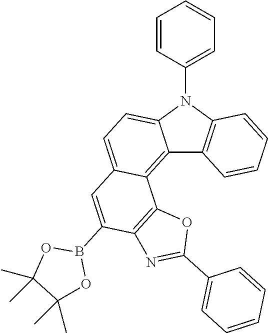 [SMILES: CC1(C)OB(c2cc3ccc4c(c5ccccc5n4-c4ccccc4)c3c3oc(-c4ccccc4)nc23)OC1(C)C] | 61 |
| n5-bo | 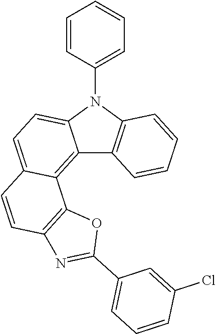 [SMILES: Clc1cccc(-c2nc3ccc4ccc5c(c6ccccc6n5-c5ccccc5)c4c3o2)c1] | 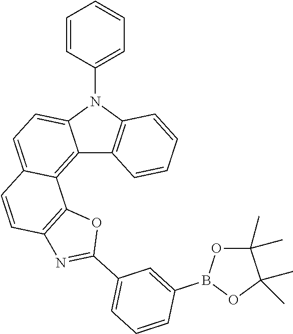 [SMILES: CC1(C)OB(c2cccc(-c3nc4ccc5ccc6c(c7ccccc7n6-c6ccccc6)c5c4o3)c2)OC1(C)C] | 77 |
| s1-bo | 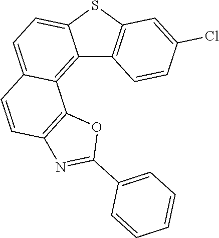 [SMILES: Clc1ccc2c(c1)sc1ccc3ccc4nc(-c5ccccc5)oc4c3c12] | 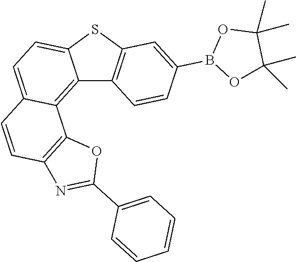 [SMILES: CC1(C)OB(c2ccc3c(c2)sc2ccc4ccc5nc(-c6ccccc6)oc5c4c23)OC1(C)C] | 56 |
| s2-bo | 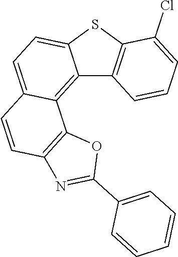 [SMILES: Clc1cccc2c1sc1ccc3ccc4nc(-c5ccccc5)oc4c3c12] | 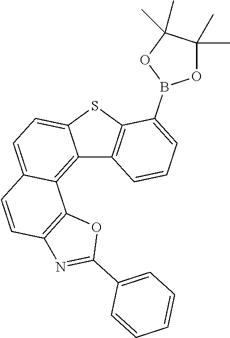 [SMILES: CC1(C)OB(c2cccc3c2sc2ccc4ccc5nc(-c6ccccc6)oc5c4c23)OC1(C)C] | 68 |
| s3-bo | 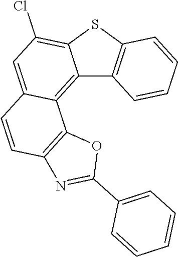 [SMILES: Clc1cc2ccc3nc(-c4ccccc4)oc3c2c2c1sc1ccccc12] | 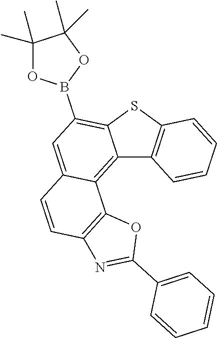 [SMILES: CC1(C)OB(c2cc3ccc4nc(-c5ccccc5)oc4c3c3c2sc2ccccc23)OC1(C)C] | 57 |
| s4-bo | 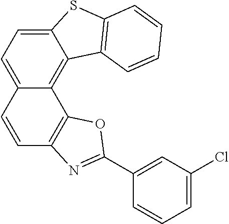 [SMILES: Clc1cccc(-c2nc3ccc4ccc5sc6ccccc6c5c4c3o2)c1] | 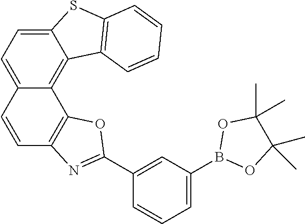 [SMILES: CC1(C)OB(c2cccc(-c3nc4ccc5ccc6sc7ccccc7c6c5c4o3)c2)OC1(C)C] | 70 |
| c1-bo | 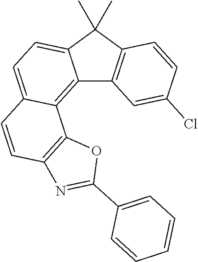 [SMILES: CC1(C)c2ccc(Cl)cc2-c2c1ccc1ccc3nc(-c4ccccc4)oc3c21] | 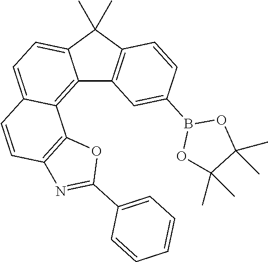 [SMILES: CC1(C)c2ccc(B3OC(C)(C)C(C)(C)O3)cc2-c2c1ccc1ccc3nc(-c4ccccc4)oc3c21] | 44 |
| c2-bo | 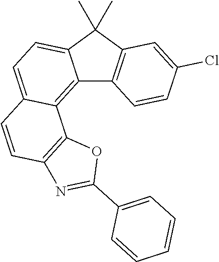 [SMILES: CC1(C)c2cc(Cl)ccc2-c2c1ccc1ccc3nc(-c4ccccc4)oc3c21] | 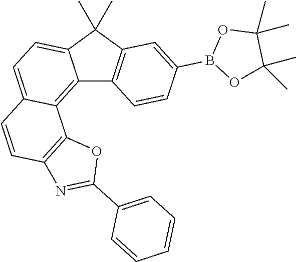 [SMILES: CC1(C)c2cc(B3OC(C)(C)C(C)(C)O3)ccc2-c2c1ccc1ccc3nc(-c4ccccc4)oc3c21] | 52 |
| c3-bo | 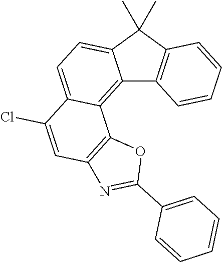 [SMILES: CC1(C)c2ccccc2-c2c1ccc1c(Cl)cc3nc(-c4ccccc4)oc3c21] | 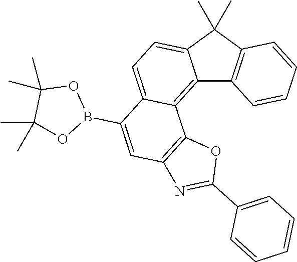 [SMILES: CC1(C)c2ccccc2-c2c1ccc1c(B3OC(C)(C)C(C)(C)O3)cc3nc(-c4ccccc4)oc3c21] | 65 |

### Synthesis of compound A2

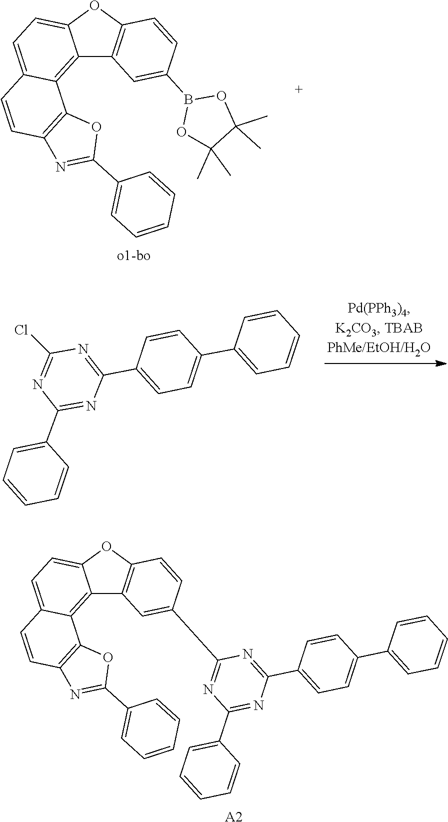

The intermediate o1-bo (3.0 g; 6.5 mmol), 2-chloro-4-(biphenyl-4-yl)-6-phenyl-1,3,5-triazine (2.3 g; 6.8 mmol), tetrakis(triphenylphosphine)palladium (0.2 g; 0.1 mmol), potassium carbonate (1.8 g; 13.0 mmol), tetrabutylammonium bromide (0.4 g; 1.3 mmol), toluene (24 mL), ethanol (6 mL) and deionized water (6 mL) were added into a round bottom flask under nitrogen protection, heated to 75° C.-80° C., and a reaction was carried out under stirring for 24 h. The reaction solution was cooled to room temperature, deionized water (50 mL) was added, liquid separation was performed, an organic phase was washed with water and dried over anhydrous magnesium sulfate, and a solvent was removed under reduced pressure; and the obtained crude product was purified by silica gel column chromatography using a dichloromethane/n-heptane solvent system, and then purified by recrystallization using a toluene/n-heptane solvent system to obtain a compound A2 (2.3 g; yield: 55%) as a white solid.

Referring to the synthesis method of the compound A2, compounds shown in Table 12 below were synthesized by using a reactant Y instead of the intermediate o1-bo, and using a reactant Z instead of 2-chloro-4-(biphenyl-4-yl)-6-phenyl-1,3,5-triazine:

TABLE 12

| Compound | Reactant Y | Reactant Z |
|---|---|---|
| A9 | 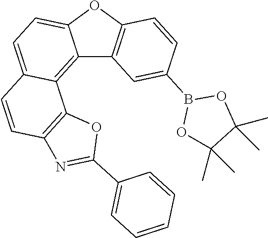 [SMILES: CC1(C)OB(c2ccc3oc4ccc5ccc6nc(-c7ccccc7)oc6c5c4c3c2)OC1(C)C] | 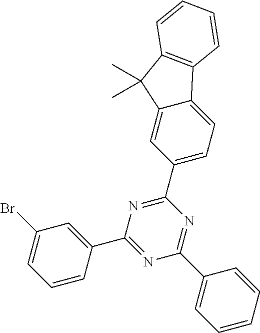 [SMILES: CC1(C)c2ccccc2-c2ccc(-c3nc(-c4ccccc4)nc(-c4cccc(Br)c4)n3)cc21] |
| A20 | 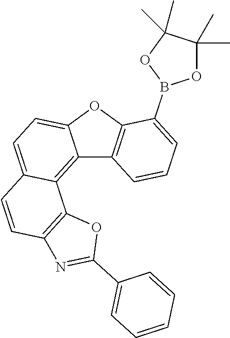 [SMILES: CC1(C)OB(c2cccc3c2oc2ccc4ccc5nc(-c6ccccc6)oc5c4c23)OC1(C)C] | 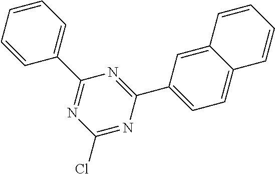 [SMILES: Clc1nc(-c2ccccc2)nc(-c2ccc3ccccc3c2)n1] |
| A27 | 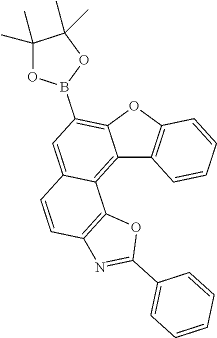 [SMILES: CC1(C)OB(c2cc3ccc4nc(-c5ccccc5)oc4c3c3c2oc2ccccc23)OC1(C)C] | 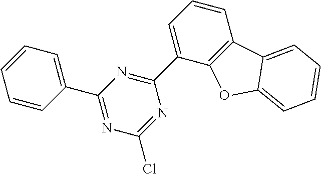 [SMILES: Clc1nc(-c2ccccc2)nc(-c2cccc3c2oc2ccccc23)n1] |
| A42 | 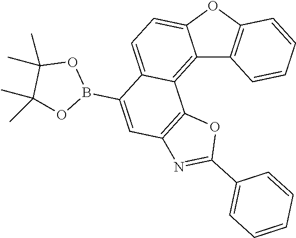 [SMILES: CC1(C)OB(c2cc3nc(-c4ccccc4)oc3c3c2ccc2oc4ccccc4c23)OC1(C)C] | 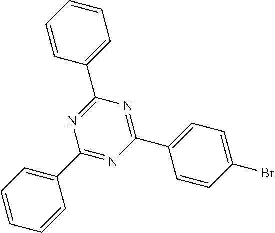 [SMILES: Brc1ccc(-c2nc(-c3ccccc3)nc(-c3ccccc3)n2)cc1] |
| A52 | 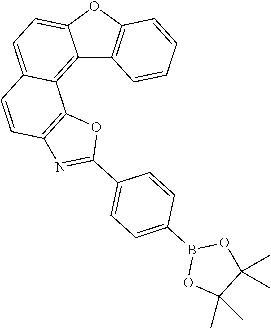 [SMILES: CC1(C)OB(c2ccc(-c3nc4ccc5ccc6oc7ccccc7c6c5c4o3)cc2)OC1(C)C] | 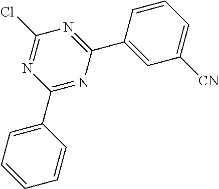 [SMILES: N#Cc1cccc(-c2nc(Cl)nc(-c3ccccc3)n2)c1] |
| B7 | 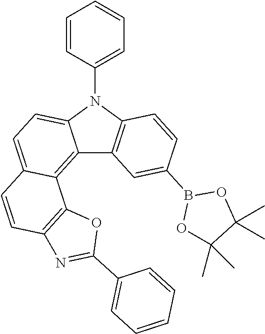 [SMILES: CC1(C)OB(c2ccc3c(c2)c2c4c(ccc5nc(-c6ccccc6)oc54)ccc2n3-c2ccccc2)OC1(C)C] | 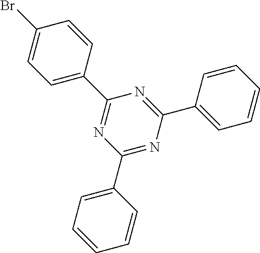 [SMILES: Brc1ccc(-c2nc(-c3ccccc3)nc(-c3ccccc3)n2)cc1] |
| B15 | 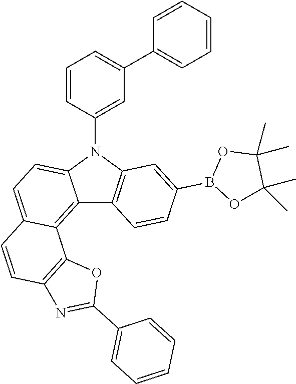 [SMILES: CC1(C)OB(c2ccc3c4c5c(ccc6nc(-c7ccccc7)oc65)ccc4n(-c4cccc(-c5ccccc5)c4)c3c2)OC1(C)C] | 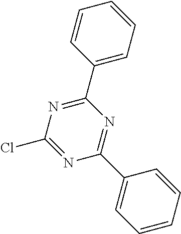 [SMILES: Clc1nc(-c2ccccc2)nc(-c2ccccc2)n1] |
| B38 | 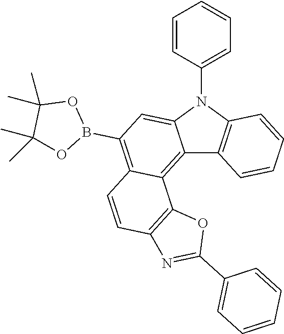 [SMILES: CC1(C)OB(c2cc3c(c4ccccc4n3-c3ccccc3)c3c2ccc2nc(-c4ccccc4)oc23)OC1(C)C] | 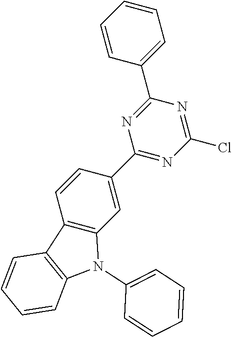 [SMILES: Clc1nc(-c2ccccc2)nc(-c2ccc3c4ccccc4n(-c4ccccc4)c3c2)n1] |
| B49 | 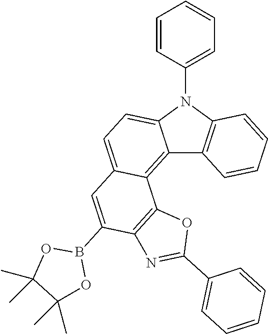 [SMILES: CC1(C)OB(c2cc3ccc4c(c5ccccc5n4-c4ccccc4)c3c3oc(-c4ccccc4)nc23)OC1(C)C] | 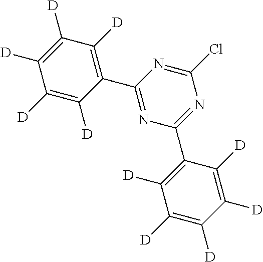 [SMILES: [2H]c1c([2H])c([2H])c(-c2nc(Cl)nc(-c3c([2H])c([2H])c([2H])c([2H])c3[2H])n2)c([2H])c1[2H]] |
| B57 | 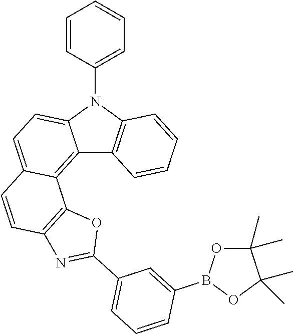 [SMILES: CC1(C)OB(c2cccc(-c3nc4ccc5ccc6c(c7ccccc7n6-c6ccccc6)c5c4o3)c2)OC1(C)C] | 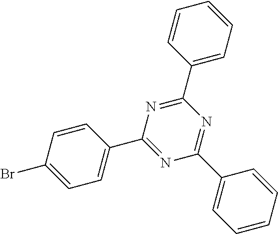 [SMILES: Brc1ccc(-c2nc(-c3ccccc3)nc(-c3ccccc3)n2)cc1] |
| C15 | 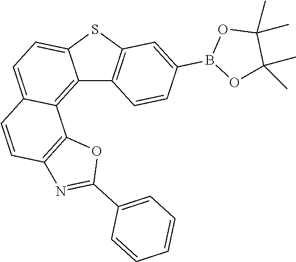 [SMILES: CC1(C)OB(c2ccc3c(c2)sc2ccc4ccc5nc(-c6ccccc6)oc5c4c23)OC1(C)C] | 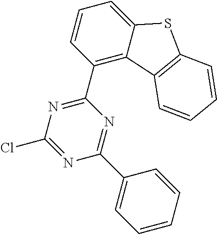 [SMILES: Clc1nc(-c2ccccc2)nc(-c2cccc3sc4ccccc4c23)n1] |
| C21 | 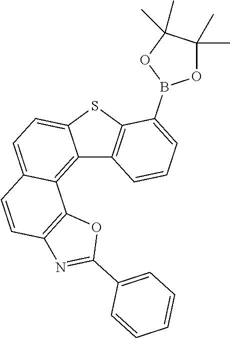 [SMILES: CC1(C)OB(c2cccc3c2sc2ccc4ccc5nc(-c6ccccc6)oc5c4c23)OC1(C)C] | 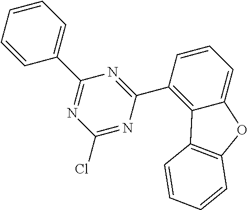 [SMILES: Clc1nc(-c2ccccc2)nc(-c2cccc3oc4ccccc4c23)n1] |
| C28 | 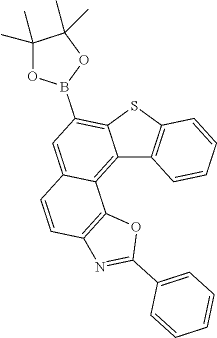 [SMILES: CC1(C)OB(c2cc3ccc4nc(-c5ccccc5)oc4c3c3c2sc2ccccc23)OC1(C)C] | 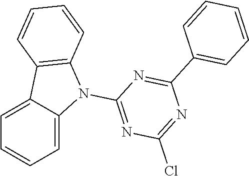 [SMILES: Clc1nc(-c2ccccc2)nc(-n2c3ccccc3c3ccccc32)n1] |
| C59 | 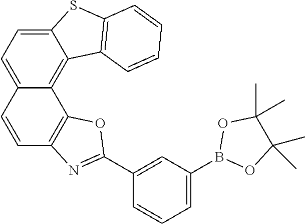 [SMILES: CC1(C)OB(c2cccc(-c3nc4ccc5ccc6sc7ccccc7c6c5c4o3)c2)OC1(C)C] | 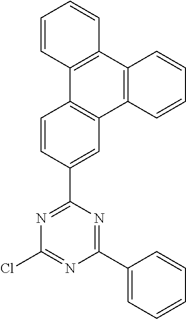 [SMILES: Clc1nc(-c2ccccc2)nc(-c2ccc3c4ccccc4c4ccccc4c3c2)n1] |
| D1 | 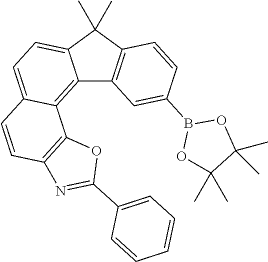 [SMILES: CC1(C)c2ccc(B3OC(C)(C)C(C)(C)O3)cc2-c2c1ccc1ccc3nc(-c4ccccc4)oc3c21] | 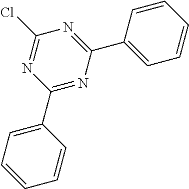 [SMILES: Clc1nc(-c2ccccc2)nc(-c2ccccc2)n1] |
| D9 | 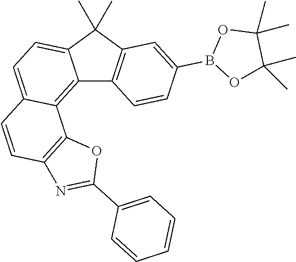 [SMILES: CC1(C)c2cc(B3OC(C)(C)C(C)(C)O3)ccc2-c2c1ccc1ccc3nc(-c4ccccc4)oc3c21] | 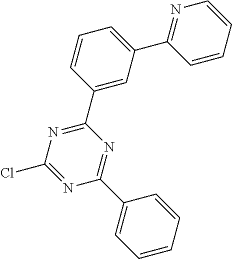 [SMILES: Clc1nc(-c2ccccc2)nc(-c2cccc(-c3ccccn3)c2)n1] |
| D22 | 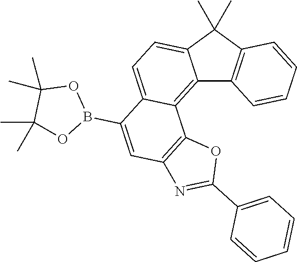 [SMILES: CC1(C)c2ccccc2-c2c1ccc1c(B3OC(C)(C)C(C)(C)O3)cc3nc(-c4ccccc4)oc3c21] | 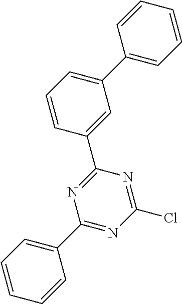 [SMILES: Clc1nc(-c2ccccc2)nc(-c2cccc(-c3ccccc3)c2)n1] |
| A65 | 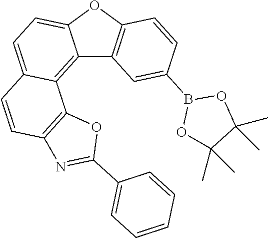 [SMILES: CC1(C)OB(c2ccc3oc4ccc5ccc6nc(-c7ccccc7)oc6c5c4c3c2)OC1(C)C] | 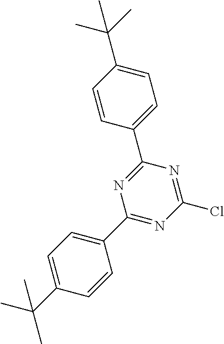 [SMILES: CC(C)(C)c1ccc(-c2nc(Cl)nc(-c3ccc(C(C)(C)C)cc3)n2)cc1] |

| | | Yield |
|---|---|---|
| Compound | Structure | (%) |
| A9 | 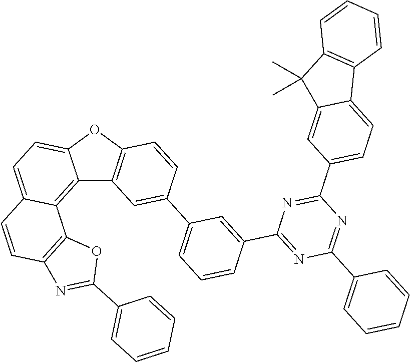 [SMILES: CC1(C)c2ccccc2-c2ccc(-c3nc(-c4ccccc4)nc(-c4cccc(-c5ccc6oc7ccc8ccc9nc(-c%10ccccc%10)oc9c8c7c6c5)c4)n3)cc21] | 61 |
| A20 | 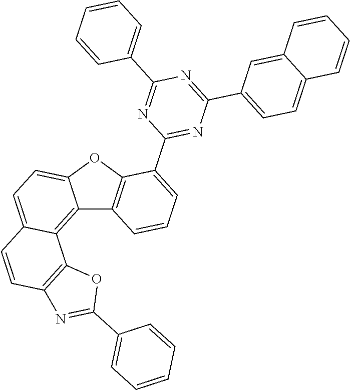 [SMILES: c1ccc(-c2nc(-c3ccc4ccccc4c3)nc(-c3cccc4c3oc3ccc5ccc6nc(-c7ccccc7)oc6c5c34)n2)cc1] | 73 |
| A27 | 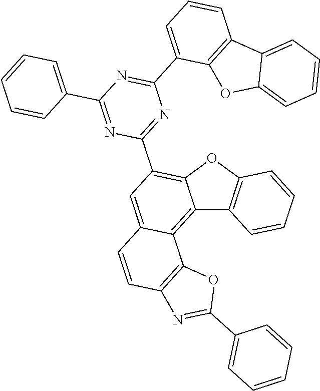 [SMILES: c1ccc(-c2nc(-c3cccc4c3oc3ccccc34)nc(-c3cc4ccc5nc(-c6ccccc6)oc5c4c4c3oc3ccccc34)n2)cc1] | 70 |
| A42 | 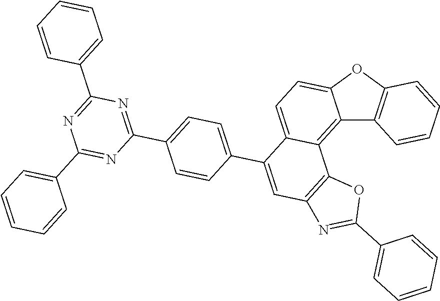 [SMILES: c1ccc(-c2nc(-c3ccccc3)nc(-c3ccc(-c4cc5nc(-c6ccccc6)oc5c5c4ccc4oc6ccccc6c45)cc3)n2)cc1] | 66 |
| A52 | 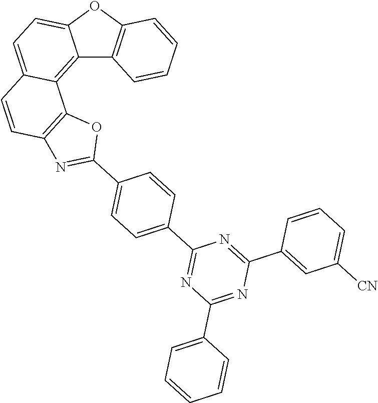 [SMILES: N#Cc1cccc(-c2nc(-c3ccccc3)nc(-c3ccc(-c4nc5ccc6ccc7oc8ccccc8c7c6c5o4)cc3)n2)c1] | 67 |
| B7 | 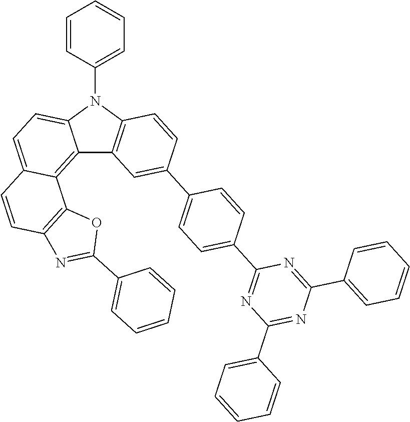 [SMILES: c1ccc(-c2nc(-c3ccccc3)nc(-c3ccc(-c4ccc5c(c4)c4c6c(ccc7nc(-c8ccccc8)oc76)ccc4n5-c4ccccc4)cc3)n2)cc1] | 58 |
| B15 | 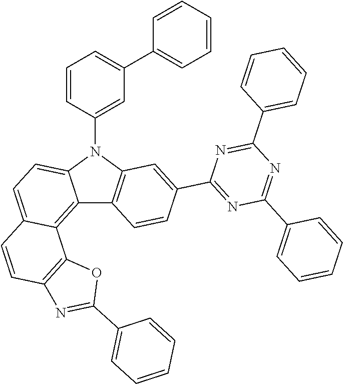 [SMILES: c1ccc(-c2cccc(-n3c4cc(-c5nc(-c6ccccc6)nc(-c6ccccc6)n5)ccc4c4c5c(ccc6nc(-c7ccccc7)oc65)ccc43)c2)cc1] | 73 |
| B38 | 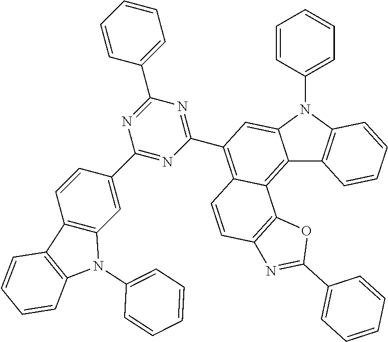 [SMILES: c1ccc(-c2nc(-c3ccc4c5ccccc5n(-c5ccccc5)c4c3)nc(-c3cc4c(c5ccccc5n4-c4ccccc4)c4c3ccc3nc(-c5ccccc5)oc34)n2)cc1] | 63 |
| B49 | 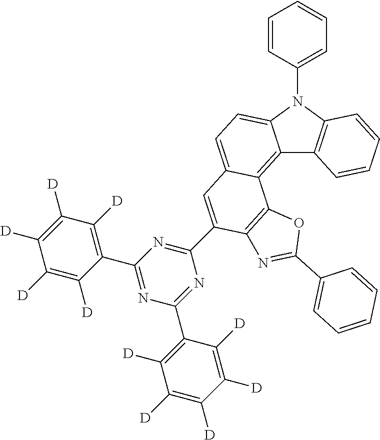 [SMILES: [2H]c1c([2H])c([2H])c(-c2nc(-c3c([2H])c([2H])c([2H])c([2H])c3[2H])nc(-c3cc4ccc5c(c6ccccc6n5-c5ccccc5)c4c4oc(-c5ccccc5)nc34)n2)c([2H])c1[2H]] | 55 |
| B57 | 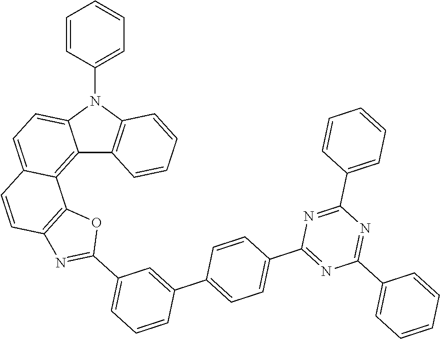 [SMILES: c1ccc(-c2nc(-c3ccccc3)nc(-c3ccc(-c4cccc(-c5nc6ccc7ccc8c(c9ccccc9n8-c8ccccc8)c7c6o5)c4)cc3)n2)cc1] | 58 |
| C15 | 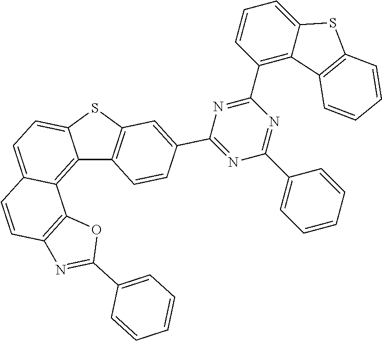 [SMILES: c1ccc(-c2nc(-c3ccc4c(c3)sc3ccc5ccc6nc(-c7ccccc7)oc6c5c34)nc(-c3cccc4sc5ccccc5c34)n2)cc1] | 70 |
| C21 | 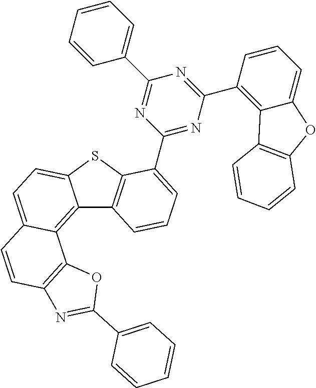 [SMILES: c1ccc(-c2nc(-c3cccc4c3sc3ccc5ccc6nc(-c7ccccc7)oc6c5c34)nc(-c3cccc4oc5ccccc5c34)n2)cc1] | 71 |
| C28 | 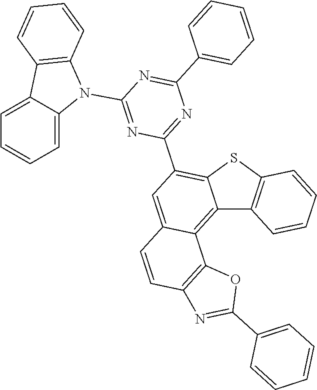 [SMILES: c1ccc(-c2nc(-c3cc4ccc5nc(-c6ccccc6)oc5c4c4c3sc3ccccc34)nc(-n3c4ccccc4c4ccccc43)n2)cc1] | 68 |
| C59 | 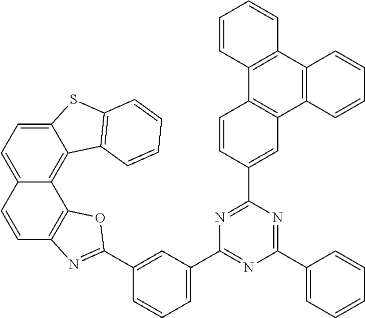 [SMILES: c1ccc(-c2nc(-c3cccc(-c4nc5ccc6ccc7sc8ccccc8c7c6c5o4)c3)nc(-c3ccc4c5ccccc5c5ccccc5c4c3)n2)cc1] | 70 |
| D1 | 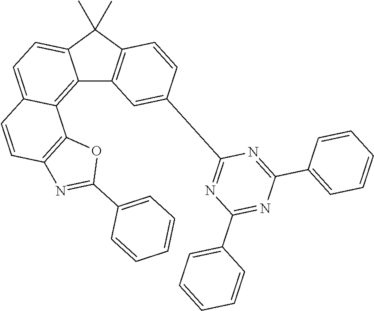 [SMILES: CC1(C)c2ccc(-c3nc(-c4ccccc4)nc(-c4ccccc4)n3)cc2-c2c1ccc1ccc3nc(-c4ccccc4)oc3c21] | 45 |
| D9 | 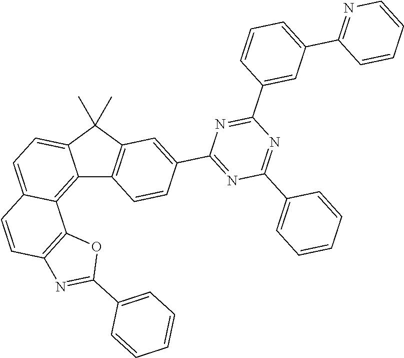 [SMILES: CC1(C)c2cc(-c3nc(-c4ccccc4)nc(-c4cccc(-c5ccccn5)c4)n3)ccc2-c2c1ccc1ccc3nc(-c4ccccc4)oc3c21] | 71 |
| D22 | 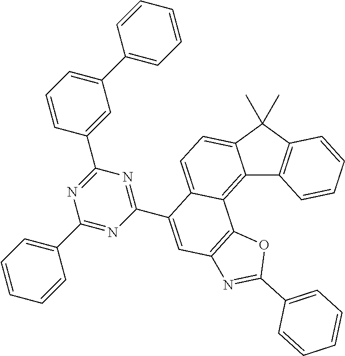 [SMILES: CC1(C)c2ccccc2-c2c1ccc1c(-c3nc(-c4ccccc4)nc(-c4cccc(-c5ccccc5)c4)n3)cc3nc(-c4ccccc4)oc3c21] | 50 |
| A65 | 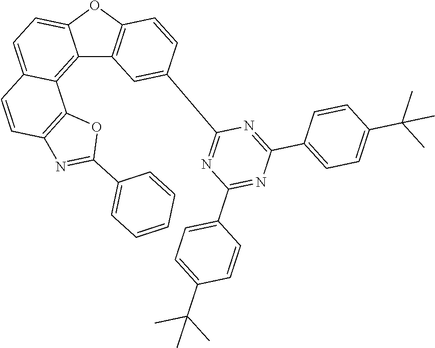 [SMILES: CC(C)(C)c1ccc(-c2nc(-c3ccc(C(C)(C)C)cc3)nc(-c3ccc4oc5ccc6ccc7nc(-c8ccccc8)oc7c6c5c4c3)n2)cc1] | 43 |

Mass spectrum data for some compounds are shown in Table 13 below:

TABLE 13

| Compound | Mass spectrum data | Compound | Mass spectrum data |
|---|---|---|---|
| Compound A2 | m/z = 643.2 (M + H)⁺ | Compound B38 | m/z = 807.3 (M + H)⁺ |
| Compound A9 | m/z = 759.3 (M + H)⁺ | Compound B49 | m/z = 652.3 (M + H)⁺ |
| Compound A20 | m/z = 617.2 (M + H)⁺ | Compound B57 | m/z = 718.3 (M + H)⁺ |
| Compound A27 | m/z = 657.2 (M + H)⁺ | Compound C15 | m/z = 689.1 (M + H)⁺ |
| Compound A42 | m/z = 643.2 (M + H)⁺ | Compound C21 | m/z = 673.2 (M + H)⁺ |
| Compound A52 | m/z = 592.2 (M + H)⁺ | Compound C28 | m/z = 672.2 (M + H)⁺ |
| Compound B7 | m/z = 718.3 (M + H)⁺ | Compound C59 | m/z = 733.2 (M + H)⁺ |
| Compound B15 | m/z = 718.3 (M + H)⁺ | Compound D1 | m/z = 593.2 (M + H)⁺ |
| Compound B24 | m/z = 732.2 (M + H)⁺ | Compound D9 | m/z = 670.3 (M + H)⁺ |
| Compound B31 | m/z = 642.2 (M + H)⁺ | Compound D22 | m/z = 669.3 (M + H)⁺ |
| Compound A65 | m/z = 679.3 (M + H)⁺ | | |

NMR data of some compounds are shown in Table 14 below:

TABLE 14

| Compound | NMR data |
|---|---|
| Compound | ¹H NMR (CD₂Cl₂, 400 MHz) δ ppm: 11.9 (s, 1H), |
| A2 | 8.57 (d, 1H), 8.30 (d, 2H), 8.25 (d, 2H), 7.95 (d, 1H), |
| | 7.91-7.84 (m, 4H), 7.73 (d, 1H), 7.64 (d, 1H), 7.47-7.38 |
| | (m, 7H), 7.25-7.14 (m, 4H), 7.03 (t, 2H). |
| Compound | ¹H NMR (CD₂Cl₂, 400 MHz) δ ppm: 9.55 (d, 1H), |
| B24 | 9.27 (d, 1H), 9.14 (d, 1H), 8.93 (s, 1H), 8.85 (d, 2H), |
| | 8.77 (d, 1H), 8.18 (d, 2H), 8.10-8.04 (m, 2H), 7.98-7.86 |
| | (m, 5H), 7.62-7.39 (m, 13H). |

### Manufacture of Organic Electroluminescent Devices

### Example 1: Manufacture of Red Organic Electroluminescent Device

An anode was pretreated by the following process: surface treatment was performed with UV ozone and O₂:N₂ plasma on an ITO/Ag/ITO substrate with a thickness of 110 Å, 900 Å, and 110 Å in sequence to increase the work function of the anode, and the surface of the ITO/Ag/ITO substrate was cleaned with an organic solvent to remove impurities and oil on the surface of the substrate.

HAT-CN was vacuum evaporated on an experimental substrate (the anode) to form a hole injection layer (HIL) with a thickness of 130 Å, and then HT-20 was vacuum evaporated on the hole injection layer to form a first hole transport layer with a thickness of 1150 Å.

A compound HT-21 was vacuum evaporated on the first hole transport layer to form a second hole transport layer with a thickness of 950 Å.

Next, RH-P, a compound A2 and RD-01 were co-evaporated on the second hole transport layer at an evaporation rate ratio of 47%:47%:6% to form an organic light-emitting layer (EML) with a thickness of 400 Å.

A compound ET-O1 and LiQ were mixed in a weight ratio of 1:1 and evaporated on the organic light-emitting layer to form an electron transport layer (ETL) with a thickness of 340 Å, Yb was evaporated on the electron transport layer to form an electron injection layer (EIL) with a thickness of 10 Å, and then magnesium (Mg) and silver (Ag) were mixed at an evaporation rate of 1:9 and vacuum evaporated on the electron injection layer to form a cathode with a thickness of 120 Å.

In addition, CP-1 was vacuum evaporated on the above cathode to form an organic capping layer with a thickness of 700 Å, thus completing the manufacture of a red organic electroluminescent device.

### Examples 2-21

An organic electroluminescent device was manufactured by the same method as that in Example 1, except that compounds (collectively referred to as a “compound X”) in Table 15 below were used instead of the compound A2 in Example 1 when the organic light-emitting layer was manufactured.

### Comparative Examples 1-2

An organic electroluminescent device was manufactured by the same method as that in Example 1, except that a compound I and a compound II were respectively used instead of the compound A2 in Example 1 when the organic light-emitting layer was manufactured.

When the organic electroluminescent devices in the Examples and Comparative examples were manufactured, the structures of main compounds used are as follows:

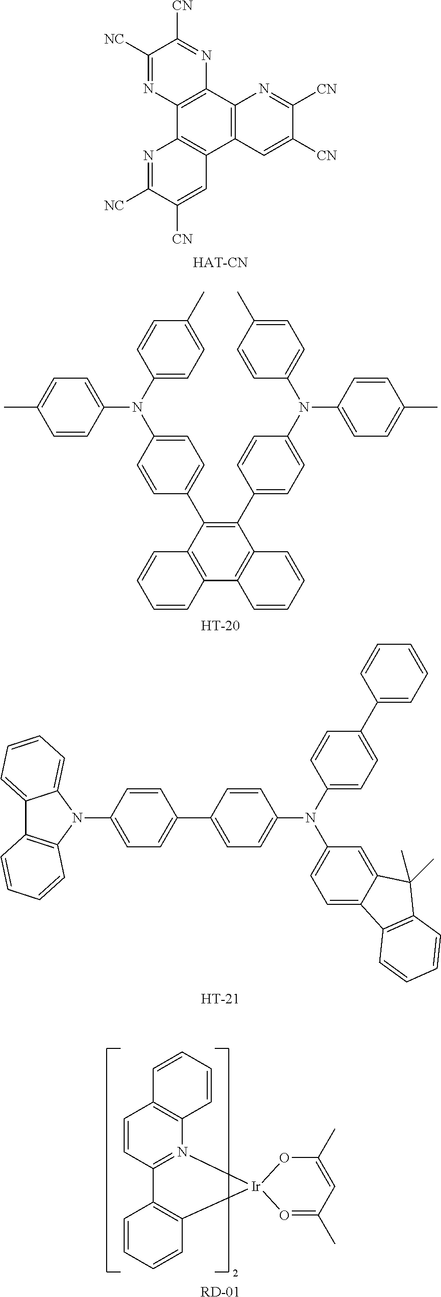

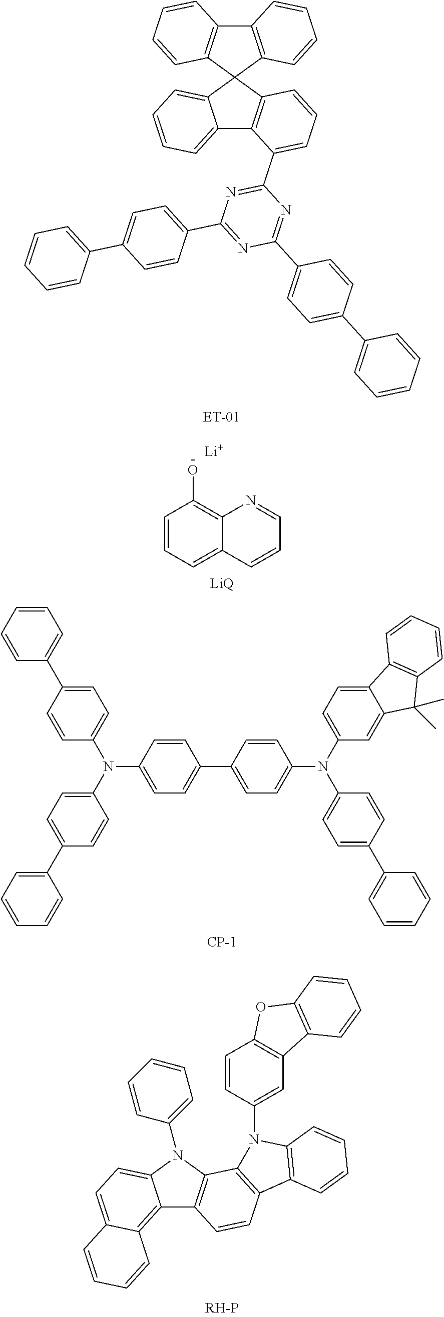

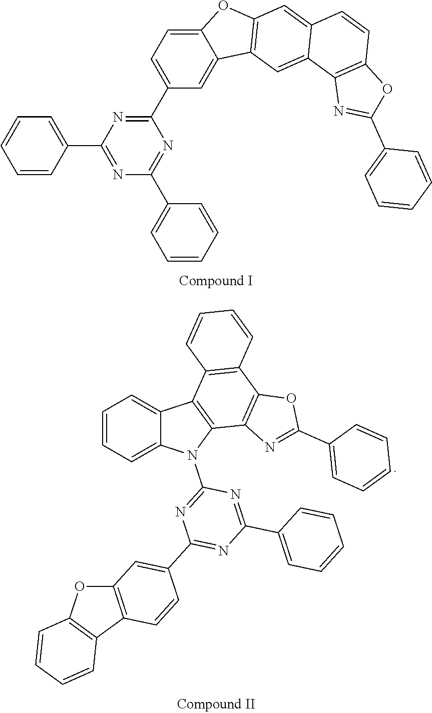 [SMILES: c1ccc(-c2nc(-c3ccccc3)nc(-c3ccc4oc5cc6ccc7oc(-c8ccccc8)nc7c6cc5c4c3)n2)cc1 ; c1ccc(-c2nc(-c3ccc4c(c3)oc3ccccc34)nc(-n3c4ccccc4c4c5ccccc5c5oc(-c6ccccc6)nc5c43)n2)cc1]

The red organic electroluminescent devices manufactured in Examples 1-21 and Comparative examples 1-2 were subjected to performance tests, and specifically, the current-voltage-brightness (IVL) performance of the devices was tested under the condition of 10 mA/cm, and the T95 device service life was tested under the condition of 20 mA/cm², and the test results are shown in Table 15.

TABLE 15

| | | Operating voltage | | | | T95 (h) |
|---|---|---|---|---|---|---|
| No. | Compound X | Volt (V) | Cd/A | CIEx | CIEy | @20 mA/cm² |
| Example 1 | Compound A2 | 3.33 | 65.30 | 0.68 | 0.32 | 514 |
| Example 2 | Compound A9 | 3.39 | 64.90 | 0.68 | 0.32 | 505 |
| Example 3 | Compound A20 | 3.38 | 65.90 | 0.68 | 0.32 | 499 |
| Example 4 | Compound A27 | 3.35 | 65.30 | 0.68 | 0.32 | 523 |
| Example 5 | Compound A42 | 3.36 | 65.40 | 0.68 | 0.32 | 509 |
| Example 6 | Compound A52 | 3.33 | 64.70 | 0.68 | 0.32 | 522 |
| Example 7 | Compound B7 | 3.35 | 65.30 | 0.68 | 0.32 | 471 |
| Example 8 | Compound B15 | 3.35 | 65.10 | 0.68 | 0.32 | 475 |
| Example 9 | Compound B24 | 3.35 | 65.40 | 0.68 | 0.32 | 467 |
| Example 10 | Compound B31 | 3.38 | 65.90 | 0.68 | 0.32 | 476 |
| Example 11 | Compound B38 | 3.33 | 65.30 | 0.68 | 0.32 | 475 |
| Example 12 | Compound B49 | 3.35 | 64.80 | 0.68 | 0.32 | 466 |
| Example 13 | Compound B57 | 3.33 | 64.60 | 0.68 | 0.32 | 473 |
| Example 14 | Compound C15 | 3.34 | 64.80 | 0.68 | 0.32 | 510 |
| Example 15 | Compound C21 | 3.39 | 64.70 | 0.68 | 0.32 | 518 |
| Example 16 | Compound C28 | 3.33 | 65.70 | 0.68 | 0.32 | 517 |
| Example 17 | Compound C59 | 3.36 | 65.60 | 0.68 | 0.32 | 517 |
| Example 18 | Compound D1 | 3.38 | 60.60 | 0.68 | 0.32 | 467 |
| Example 19 | Compound D9 | 3.33 | 60.33 | 0.68 | 0.32 | 471 |
| Example 20 | Compound D22 | 3.33 | 61.10 | 0.68 | 0.32 | 468 |
| Example 21 | Compound A65 | 3.37 | 64.90 | 0.68 | 0.32 | 511 |
| Comparative | Compound I | 3.67 | 53.20 | 0.68 | 0.32 | 415 |
| example 1 | | | | | | |
| Comparative | Compound II | 3.71 | 50.80 | 0.68 | 0.32 | 400 |
| example 2 | | | | | | |

As can be seen from Table 15, Examples 1-21 in which the compound of the disclosure is used as an electron-type host material in a mixed host material of a red light-emitting layer have the advantages that the voltage, current efficiency, and service life of the device are all significantly improved compared with Comparative examples 1 and 2. Specifically, the device efficiency is improved by at least 13.4% and the service life is improved by at least 12.3%.

A core group of the organic compound of the disclosure is formed by fusing a dibenzo five-membered ring and benzoxazole in a specific manner, the core group having a planar ring-like structure. When this core group is bonded to a triazine group, the compound can have a strong carrier transport capability and a high energy transfer capability while maintaining a high value of a first triplet energy level. When the organic compound of the disclosure is used as a host material for a light-emitting layer in a red organic electroluminescent device, the performance of the device can be significantly improved. In particular, the device performance is optimal when the dibenzo five-membered ring is dibenzofuran/dibenzothiophene.

It should be understood that the disclosure is not limited to the precise structures that have been described above and shown in the drawings, and that various modifications and changes can be made without departing from the scope of the disclosure. The scope of the disclosure is limited only by the appended claims.

1. An organic compound, having a structure as shown in a formula 1: 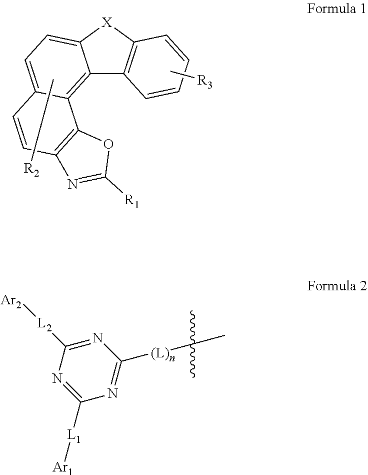
wherein, X is selected from C(R₄R₅), O, S or N(R₆);
R₁ and R₆ are the same or different, and are respectively and independently selected from substituted or unsubstituted aryl with 6 to 30 carbon atoms, substituted or unsubstituted heteroaryl with 3 to 30 carbon atoms, and a group represented by a formula 2;
R₂ and R₃ are the same or different, and are respectively and independently selected from hydrogen, deuterium, cyano, a halogen group, substituted or unsubstituted aryl with 6 to 30 carbon atoms, substituted or unsubstituted heteroaryl with 3 to 30 carbon atoms, and a group represented by the formula 2;
one and only one of R₁, R₆, R₂ and R₃ is the group represented by the formula 2;
R₄ and R₅ are the same or different, and are respectively and independently selected from alkyl with 1 to 10 carbon atoms, substituted or unsubstituted aryl with 6 to 30 carbon atoms, and substituted or unsubstituted heteroaryl with 3 to 30 carbon atoms;
each L, L₁ and L₂ are the same or different, and are respectively and independently selected from a single bond, substituted or unsubstituted arylene with 6 to 30 carbon atoms, and substituted or unsubstituted heteroarylene with 3 to 30 carbon atoms;
n is the number of groups L linked in sequence, and selected from 1, 2 or 3, and when n is greater than 1, any two L are the same or different;
Ar₁ and Ar₂ are the same or different, and are respectively and independently selected from substituted or unsubstituted aryl with 6 to 30 carbon atoms, and substituted or unsubstituted heteroaryl with 3 to 30 carbon atoms; and
substituent(s) in R₁, R₂, R₃, R₄, R₅, R₆, L, L₁, L₂, Ar₁ and Ar₂ are the same or different, and are respectively and independently selected from deuterium, a halogen group, cyano, alkyl with 1 to 10 carbon atoms, cycloalkyl with 3 to 20 carbon atoms, heteroaryl with 3 to 20 carbon atoms, aryl with 6 to 20 carbon atoms, deuteroaryl with 6 to 20 carbon atoms, haloaryl with 6 to 20 carbon atoms, trialkylsilyl with 3 to 12 carbon atoms, haloalkyl with 1 to 10 carbon atoms, and deuteroalkyl with 1 to 10 carbon atoms.

2. The organic compound according to claim 1, wherein substituent(s) in R₁, R₂, R₃, R₄, R₅, R₆, L, L₁, L₂, Ar₁ and Ar₂ are the same or different, and are respectively and independently selected from deuterium, a halogen group, cyano, alkyl with 1 to 10 carbon atoms, cycloalkyl with 3 to 20 carbon atoms, heteroaryl with 3 to 20 carbon atoms, deuteroaryl with 6 to 20 carbon atoms, haloaryl with 6 to 20 carbon atoms, trialkylsilyl with 3 to 12 carbon atoms, haloalkyl with 1 to 10 carbon atoms, and deuteroalkyl with 1 to 10 carbon atoms.

3. The organic compound according to claim 1, having a structure represented by a formula 1-1, a formula 1-2, a formula 1-3, a formula 1-4, a formula 1-5, a formula 1-6, a formula 1-7, a formula 1-8, a formula 1-9, a formula 1-10, a formula 1-11, a formula 1-12, or a formula 1-13:

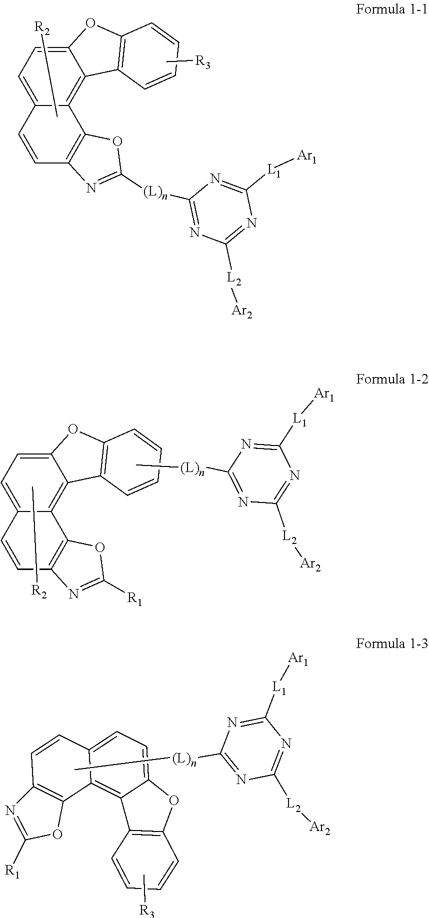

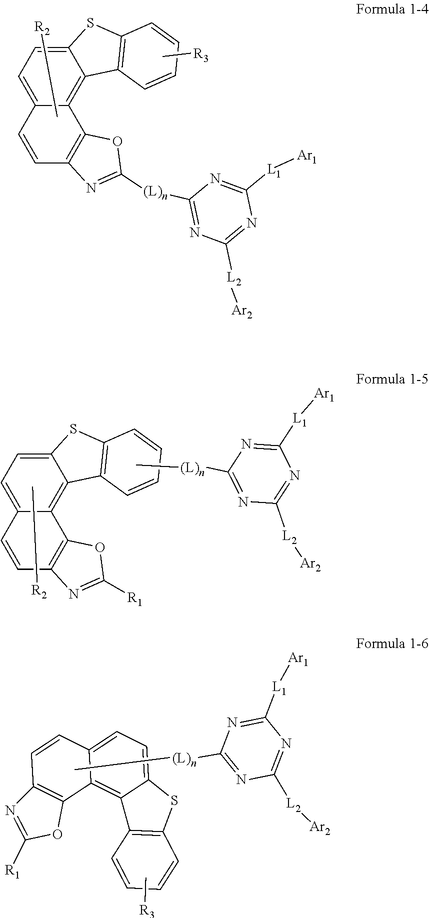

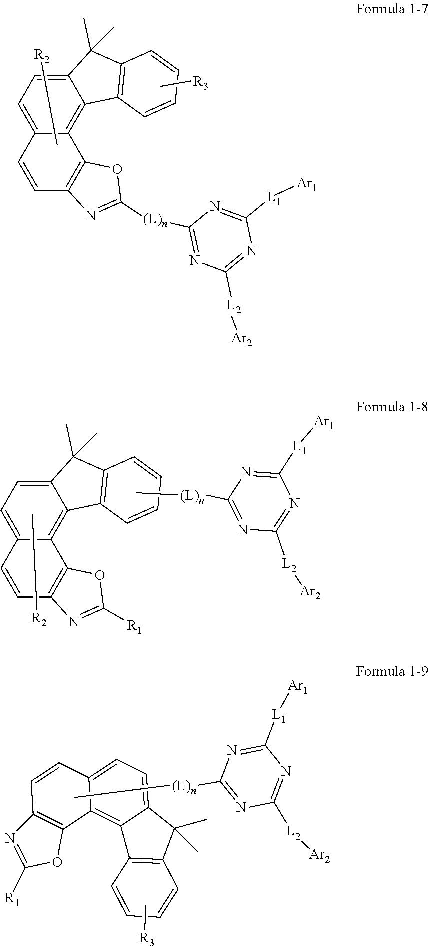

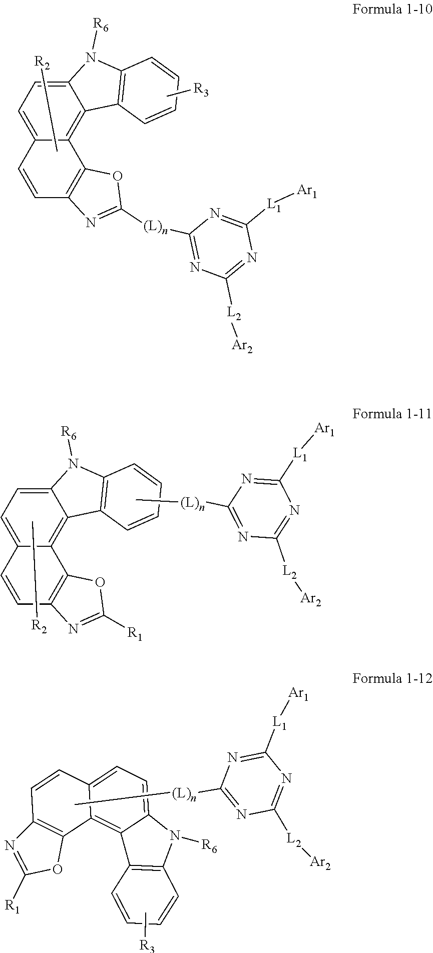

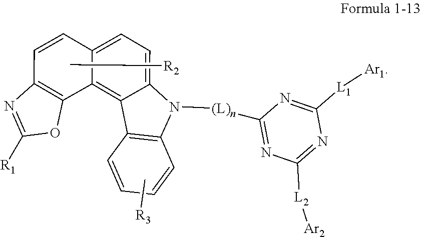

4. The organic compound according to claim 1, wherein each L is the same or different, and is independently selected from a single bond, and substituted or unsubstituted arylene with 6 to 12 carbon atoms; and
optionally, substituent(s) in L are the same or different, and are respectively and independently selected from deuterium, a halogen group, cyano, alkyl with 1 to 5 carbon atoms and phenyl.

5. The organic compound according to claim 1, wherein each L is the same or different, and is independently selected from a single bond, phenylene, naphthylene and biphenylene.

6. The organic compound according to claim 1, wherein 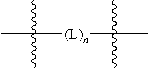
in the formula 2 is selected from a single bond or the group consisting of:

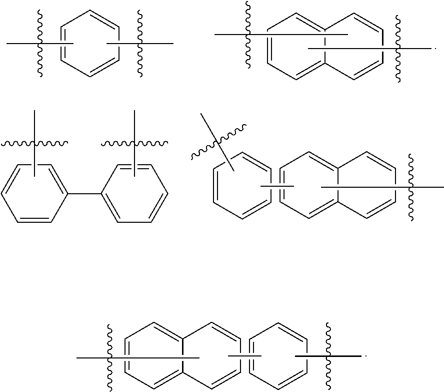

7. The organic compound according to claim 1, wherein L₁ and L₂ are the same or different, and are respectively and independently selected from a single bond, substituted or unsubstituted arylene with 6 to 12 carbon atoms, and substituted or unsubstituted heteroarylene with 12 to 18 carbon atoms; and
optionally, substituent(s) in L₁ and L₂ are the same or different, and are respectively and independently selected from deuterium, a halogen group, cyano, alkyl with 1 to 5 carbon atoms and phenyl.

8. The organic compound according to claim 1, wherein L₁ and L₂ are the same or different, and are respectively and independently selected from a single bond, substituted or unsubstituted phenylene, substituted or unsubstituted naphthylene, substituted or unsubstituted biphenylene, substituted or unsubstituted carbazolylene, substituted or unsubstituted dibenzofurylene, and substituted or unsubstituted dibenzothenylene; and
optionally, substituent(s) in L₁ and L₂ are the same or different, and are respectively and independently selected from deuterium, fluorine, cyano, methyl, ethyl, n-propyl, isopropyl, tert-butyl and phenyl.

9. The organic compound according to claim 1, wherein Ar₁ and Ar₂ are the same or different, and are respectively and independently selected from substituted or unsubstituted aryl with 6 to 20 carbon atoms, and substituted or unsubstituted heteroaryl with 5 to 20 carbon atoms; and
optionally, substituent(s) in Ar₁ and Ar₂ are the same or different, and are respectively and independently selected from deuterium, a halogen group, cyano, alkyl with 1 to 5 carbon atoms, haloalkyl with 1 to 5 carbon atoms, cycloalkyl with 1 to 10 carbon atoms, trimethylsilyl and phenyl.

10. The organic compound according to claim 1, wherein Ar₁ and Ar₂ are the same or different, and are respectively and independently selected from substituted or unsubstituted terphenyl, substituted or unsubstituted phenyl, substituted or unsubstituted naphthyl, substituted or unsubstituted biphenyl, substituted or unsubstituted phenanthryl, substituted or unsubstituted fluorenyl, substituted or unsubstituted triphenylene, substituted or unsubstituted pyridyl, substituted or unsubstituted carbazolyl, substituted or unsubstituted dibenzofuranyl, and substituted or unsubstituted dibenzothienyl; and
optionally, substituent(s) in Ar₁ and Ar₂ are the same or different, and are respectively and independently selected from deuterium, fluorine, cyano, methyl, ethyl, n-propyl, isopropyl, tert-butyl, cyclohexyl, trifluoromethyl, trimethylsilyl and phenyl.

11. The organic compound according to claim 1, wherein R₄ and R₅ are the same or different, and are respectively and independently selected from methyl and substituted or unsubstituted phenyl; and
optionally, substituent(s) in R₄ and R₅ are the same or different, and are respectively and independently selected from deuterium, fluorine, cyano, methyl, ethyl, n-propyl, isopropyl, tert-butyl and phenyl.

12. The organic compound according to claim 1, wherein R₁ and R₆ are the same or different, and are respectively and independently selected from substituted or unsubstituted phenyl, substituted or unsubstituted naphthyl, substituted or unsubstituted biphenyl and the group represented by the formula 2;
R₂ and R₃ are the same or different, and are respectively and independently selected from hydrogen and the group represented by the formula 2;
and one and only one of R₁, R₆, R₂ and R₃ is the group represented by the formula 2; and
optionally, substituent(s) in R₁ and R₆ are the same or different, and are respectively and independently selected from deuterium, fluorine, cyano, methyl, ethyl, n-propyl, isopropyl, tert-butyl and phenyl.

13. The organic compound according to claim 1, wherein the organic compound is selected from the group consisting of the following compounds:

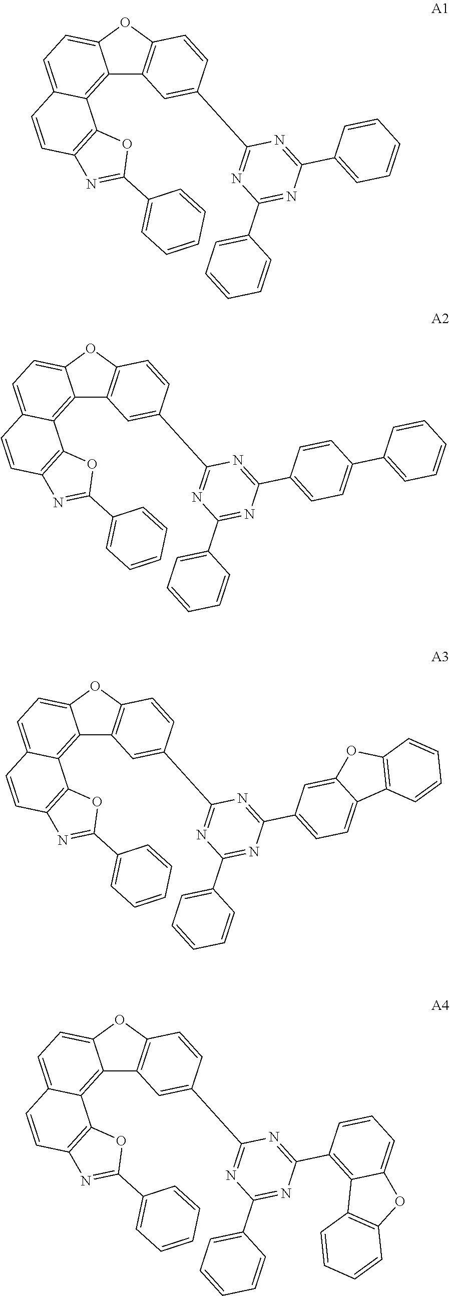 [SMILES: c1ccc(-c2nc(-c3ccccc3)nc(-c3ccc4oc5ccc6ccc7nc(-c8ccccc8)oc7c6c5c4c3)n2)cc1 ; c1ccc(-c2ccc(-c3nc(-c4ccccc4)nc(-c4ccc5oc6ccc7ccc8nc(-c9ccccc9)oc8c7c6c5c4)n3)cc2)cc1 ; c1ccc(-c2nc(-c3ccc4c(c3)oc3ccccc34)nc(-c3ccc4oc5ccc6ccc7nc(-c8ccccc8)oc7c6c5c4c3)n2)cc1 ; c1ccc(-c2nc(-c3ccc4oc5ccc6ccc7nc(-c8ccccc8)oc7c6c5c4c3)nc(-c3cccc4oc5ccccc5c34)n2)cc1]

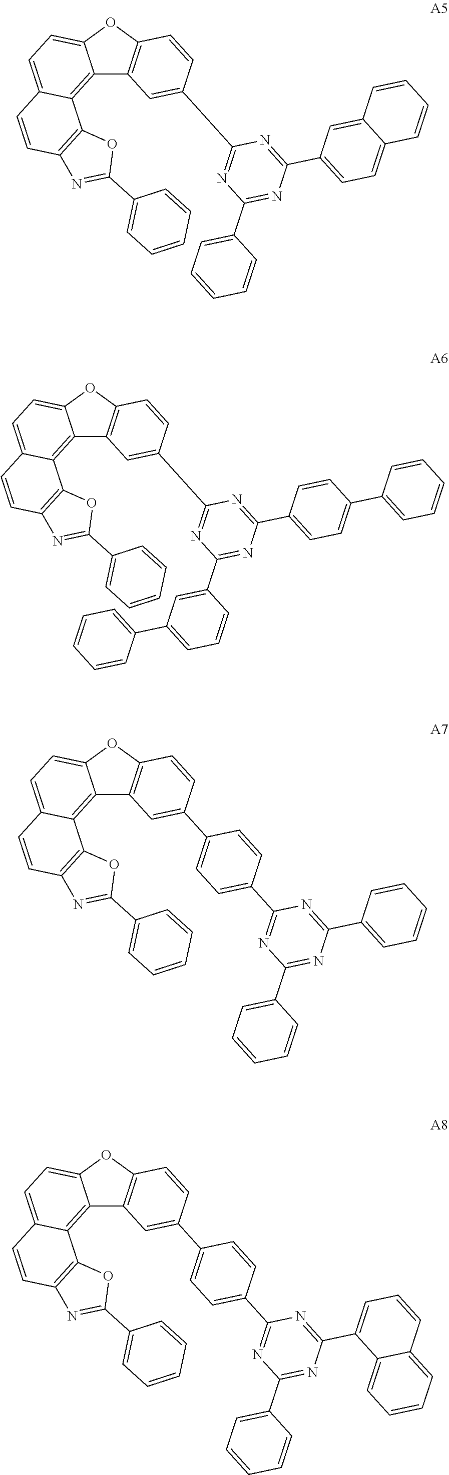 [SMILES: c1ccc(-c2nc(-c3ccc4ccccc4c3)nc(-c3ccc4oc5ccc6ccc7nc(-c8ccccc8)oc7c6c5c4c3)n2)cc1 ; c1ccc(-c2ccc(-c3nc(-c4cccc(-c5ccccc5)c4)nc(-c4ccc5oc6ccc7ccc8nc(-c9ccccc9)oc8c7c6c5c4)n3)cc2)cc1 ; c1ccc(-c2nc(-c3ccccc3)nc(-c3ccc(-c4ccc5oc6ccc7ccc8nc(-c9ccccc9)oc8c7c6c5c4)cc3)n2)cc1 ; c1ccc(-c2nc(-c3ccc(-c4ccc5oc6ccc7ccc8nc(-c9ccccc9)oc8c7c6c5c4)cc3)nc(-c3cccc4ccccc34)n2)cc1]

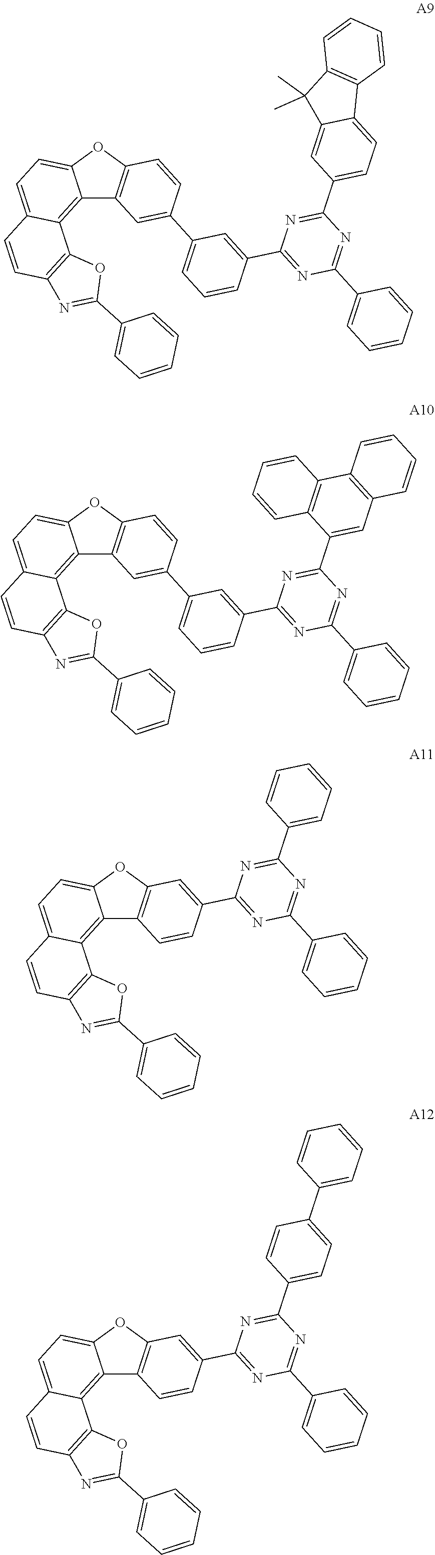 [SMILES: CC1(C)c2ccccc2-c2ccc(-c3nc(-c4ccccc4)nc(-c4cccc(-c5ccc6oc7ccc8ccc9nc(-c%10ccccc%10)oc9c8c7c6c5)c4)n3)cc21 ; c1ccc(-c2nc(-c3cccc(-c4ccc5oc6ccc7ccc8nc(-c9ccccc9)oc8c7c6c5c4)c3)nc(-c3cc4ccccc4c4ccccc34)n2)cc1 ; c1ccc(-c2nc(-c3ccccc3)nc(-c3ccc4c(c3)oc3ccc5ccc6nc(-c7ccccc7)oc6c5c34)n2)cc1 ; c1ccc(-c2ccc(-c3nc(-c4ccccc4)nc(-c4ccc5c(c4)oc4ccc6ccc7nc(-c8ccccc8)oc7c6c45)n3)cc2)cc1]

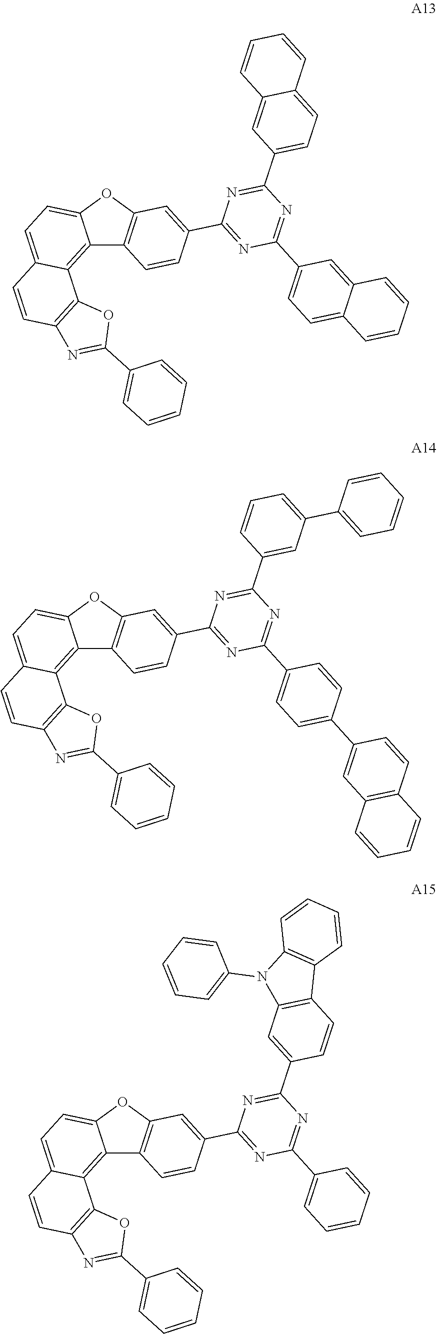 [SMILES: c1ccc(-c2nc3ccc4ccc5oc6cc(-c7nc(-c8ccc9ccccc9c8)nc(-c8ccc9ccccc9c8)n7)ccc6c5c4c3o2)cc1 ; c1ccc(-c2cccc(-c3nc(-c4ccc(-c5ccc6ccccc6c5)cc4)nc(-c4ccc5c(c4)oc4ccc6ccc7nc(-c8ccccc8)oc7c6c45)n3)c2)cc1 ; c1ccc(-c2nc(-c3ccc4c(c3)oc3ccc5ccc6nc(-c7ccccc7)oc6c5c34)nc(-c3ccc4c5ccccc5n(-c5ccccc5)c4c3)n2)cc1]

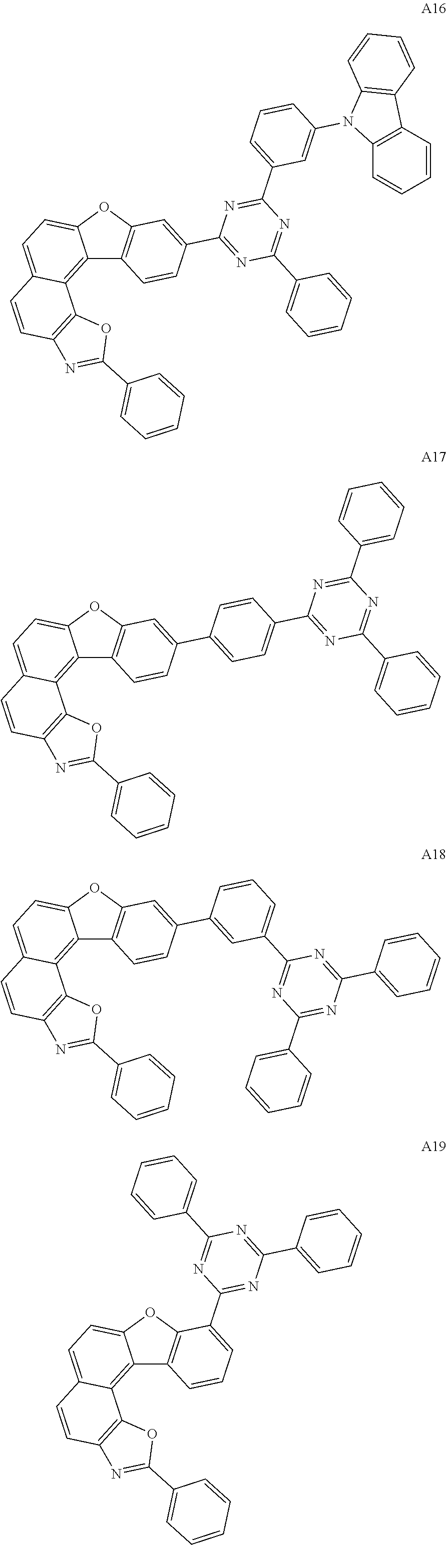

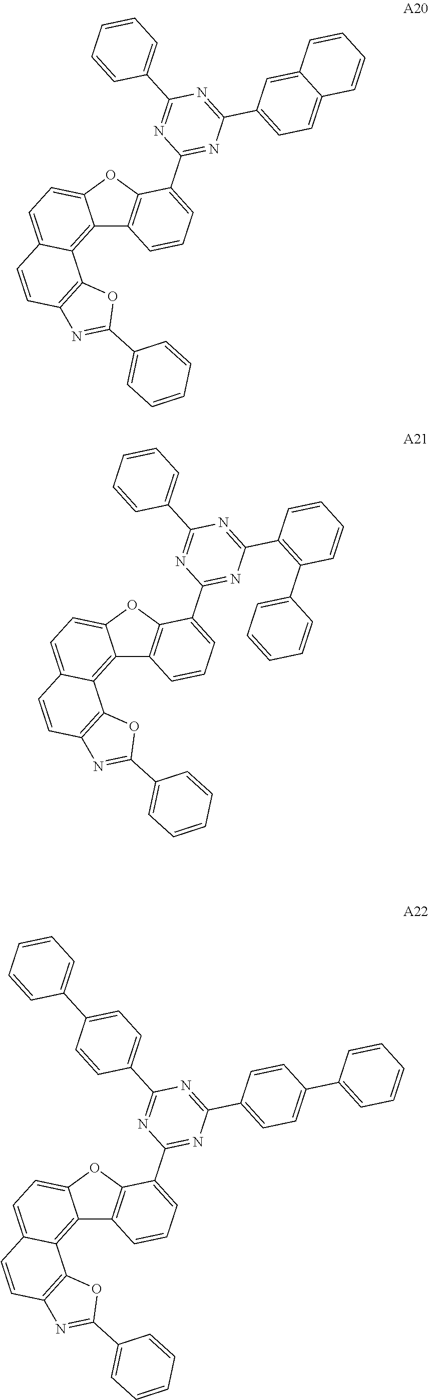

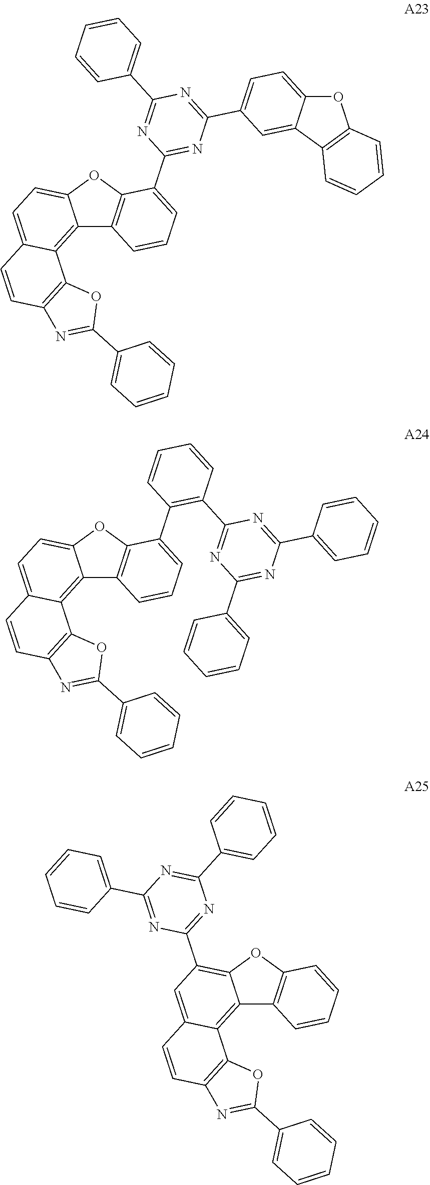 [SMILES: c1ccc(-c2nc(-c3ccc4oc5ccccc5c4c3)nc(-c3cccc4c3oc3ccc5ccc6nc(-c7ccccc7)oc6c5c34)n2)cc1 ; c1ccc(-c2nc(-c3ccccc3)nc(-c3ccccc3-c3cccc4c3oc3ccc5ccc6nc(-c7ccccc7)oc6c5c34)n2)cc1 ; c1ccc(-c2nc(-c3ccccc3)nc(-c3cc4ccc5nc(-c6ccccc6)oc5c4c4c3oc3ccccc34)n2)cc1]

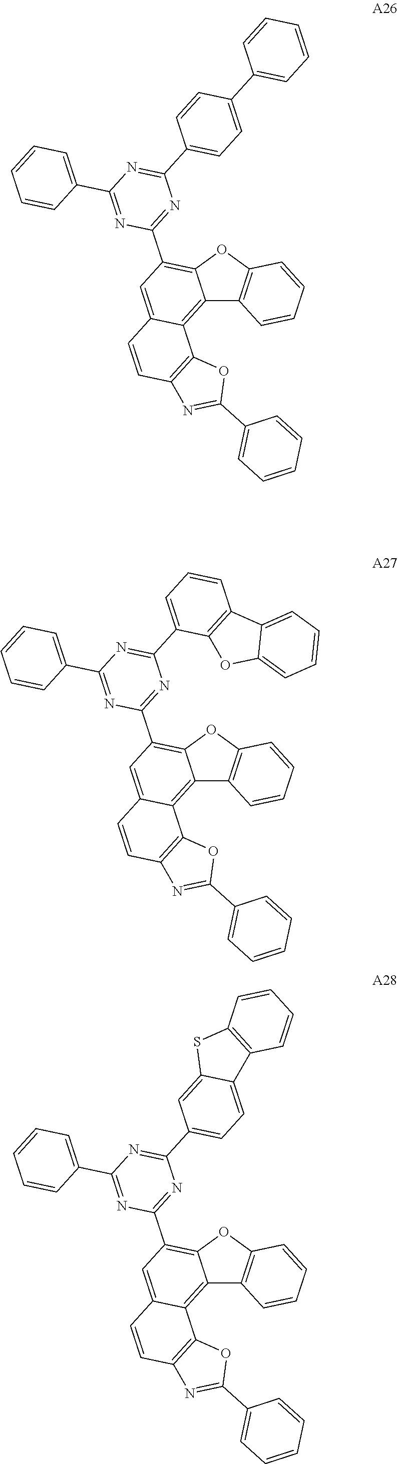

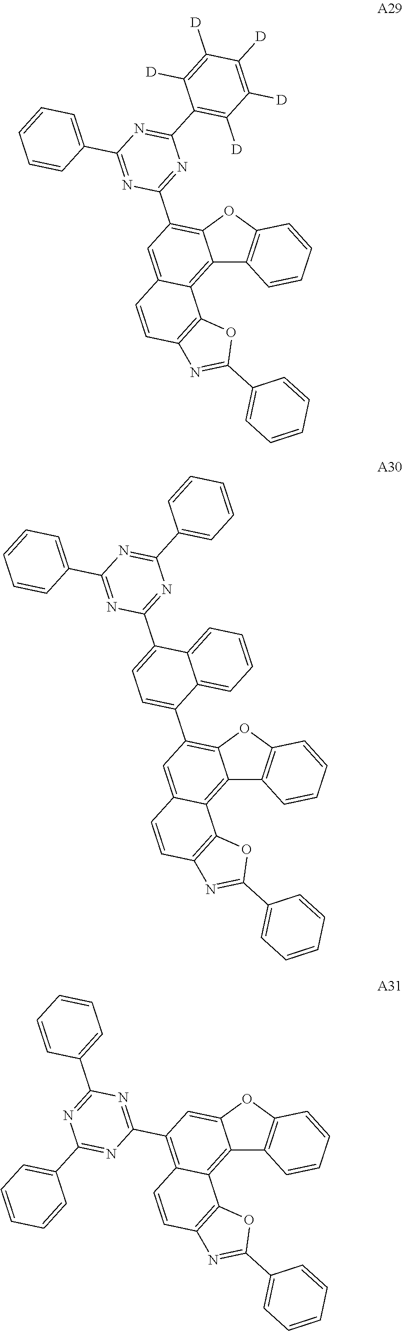 [SMILES: [2H]c1c([2H])c([2H])c(-c2nc(-c3ccccc3)nc(-c3cc4ccc5nc(-c6ccccc6)oc5c4c4c3oc3ccccc34)n2)c([2H])c1[2H] ; c1ccc(-c2nc(-c3ccccc3)nc(-c3ccc(-c4cc5ccc6nc(-c7ccccc7)oc6c5c5c4oc4ccccc45)c4ccccc34)n2)cc1 ; c1ccc(-c2nc(-c3ccccc3)nc(-c3cc4oc5ccccc5c4c4c3ccc3nc(-c5ccccc5)oc34)n2)cc1]

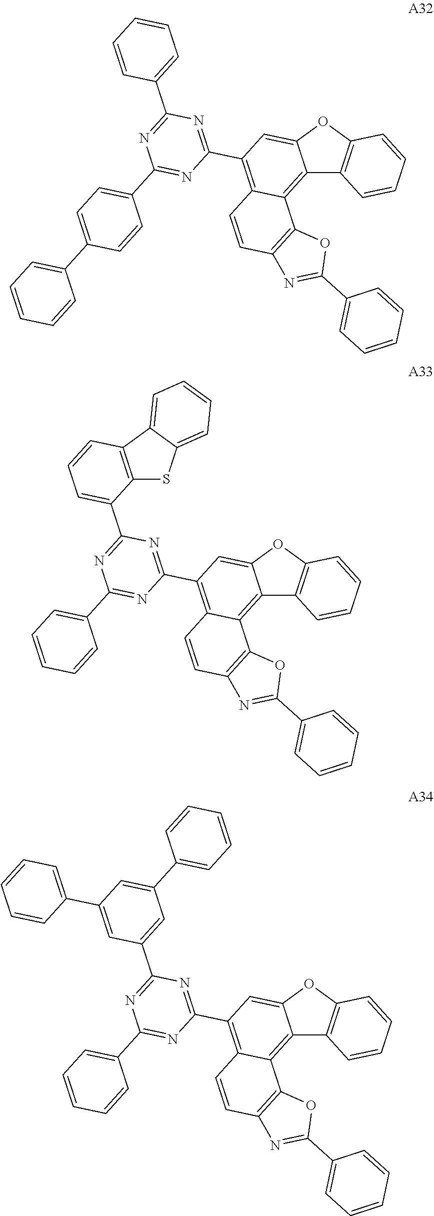 [SMILES: c1ccc(-c2ccc(-c3nc(-c4ccccc4)nc(-c4cc5oc6ccccc6c5c5c4ccc4nc(-c6ccccc6)oc45)n3)cc2)cc1 ; c1ccc(-c2nc(-c3cc4oc5ccccc5c4c4c3ccc3nc(-c5ccccc5)oc34)nc(-c3cccc4c3sc3ccccc34)n2)cc1 ; c1ccc(-c2cc(-c3ccccc3)cc(-c3nc(-c4ccccc4)nc(-c4cc5oc6ccccc6c5c5c4ccc4nc(-c6ccccc6)oc45)n3)c2)cc1]

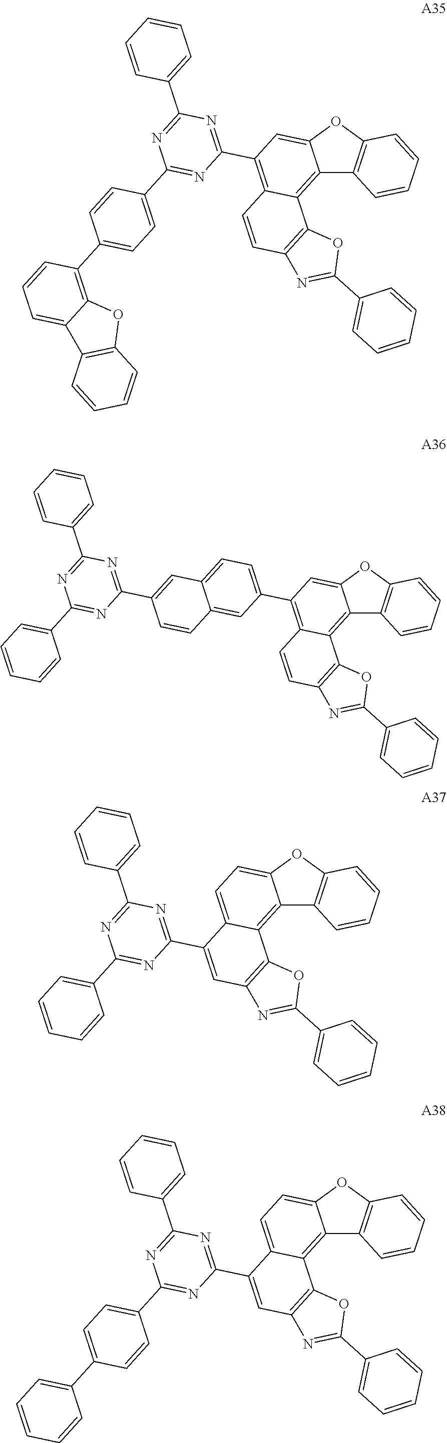 [SMILES: c1ccc(-c2nc(-c3ccc(-c4cccc5c4oc4ccccc45)cc3)nc(-c3cc4oc5ccccc5c4c4c3ccc3nc(-c5ccccc5)oc34)n2)cc1 ; c1ccc(-c2nc(-c3ccccc3)nc(-c3ccc4cc(-c5cc6oc7ccccc7c6c6c5ccc5nc(-c7ccccc7)oc56)ccc4c3)n2)cc1 ; c1ccc(-c2nc(-c3ccccc3)nc(-c3cc4nc(-c5ccccc5)oc4c4c3ccc3oc5ccccc5c34)n2)cc1 ; c1ccc(-c2ccc(-c3nc(-c4ccccc4)nc(-c4cc5nc(-c6ccccc6)oc5c5c4ccc4oc6ccccc6c45)n3)cc2)cc1]

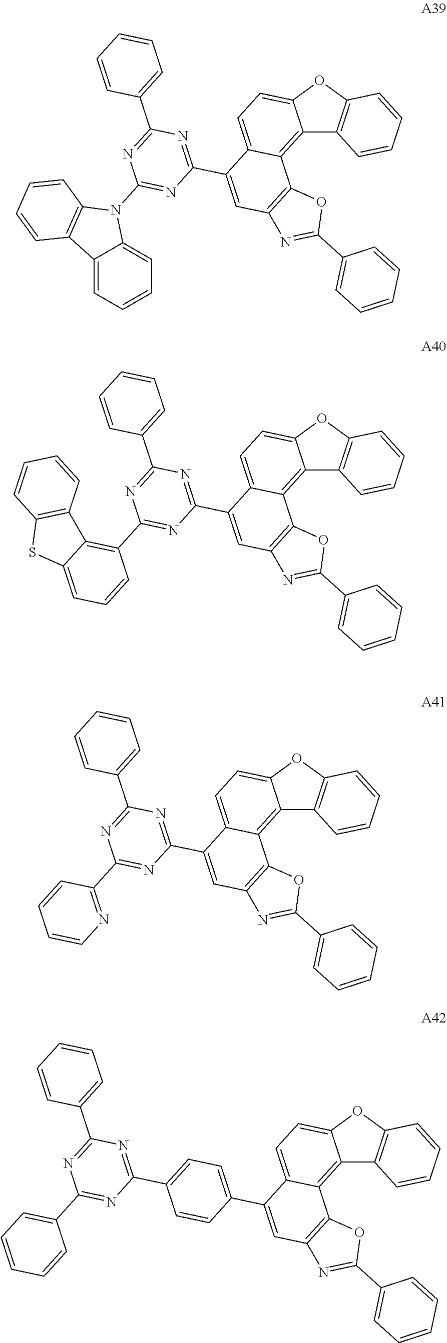 [SMILES: c1ccc(-c2nc(-c3cc4nc(-c5ccccc5)oc4c4c3ccc3oc5ccccc5c34)nc(-n3c4ccccc4c4ccccc43)n2)cc1 ; c1ccc(-c2nc(-c3cc4nc(-c5ccccc5)oc4c4c3ccc3oc5ccccc5c34)nc(-c3cccc4sc5ccccc5c34)n2)cc1 ; c1ccc(-c2nc(-c3ccccn3)nc(-c3cc4nc(-c5ccccc5)oc4c4c3ccc3oc5ccccc5c34)n2)cc1 ; c1ccc(-c2nc(-c3ccccc3)nc(-c3ccc(-c4cc5nc(-c6ccccc6)oc5c5c4ccc4oc6ccccc6c45)cc3)n2)cc1]

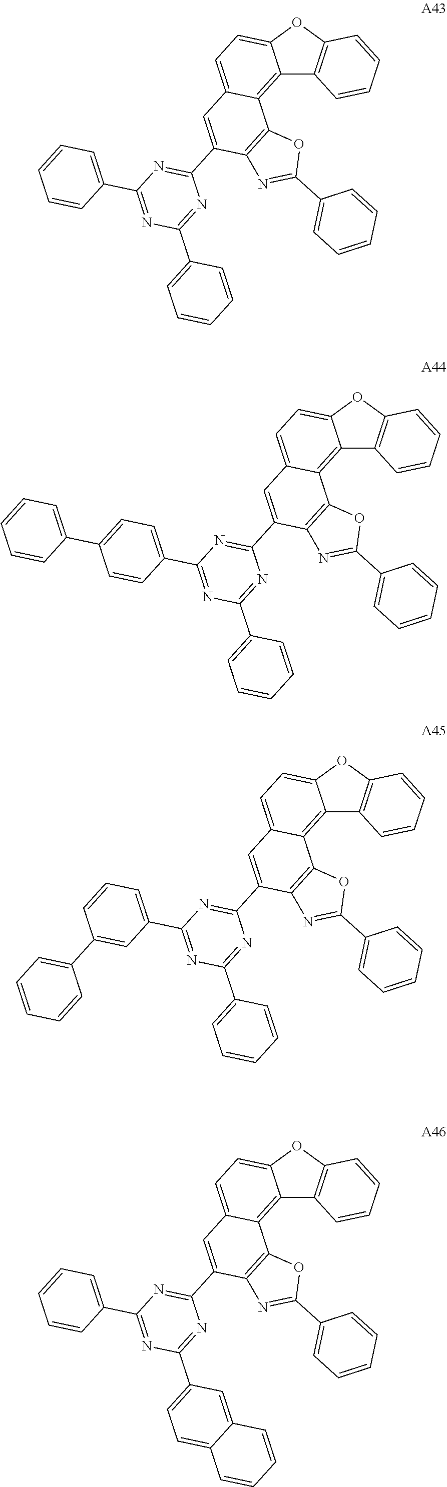 [SMILES: c1ccc(-c2nc(-c3ccccc3)nc(-c3cc4ccc5oc6ccccc6c5c4c4oc(-c5ccccc5)nc34)n2)cc1 ; c1ccc(-c2ccc(-c3nc(-c4ccccc4)nc(-c4cc5ccc6oc7ccccc7c6c5c5oc(-c6ccccc6)nc45)n3)cc2)cc1 ; c1ccc(-c2cccc(-c3nc(-c4ccccc4)nc(-c4cc5ccc6oc7ccccc7c6c5c5oc(-c6ccccc6)nc45)n3)c2)cc1 ; c1ccc(-c2nc(-c3ccc4ccccc4c3)nc(-c3cc4ccc5oc6ccccc6c5c4c4oc(-c5ccccc5)nc34)n2)cc1]

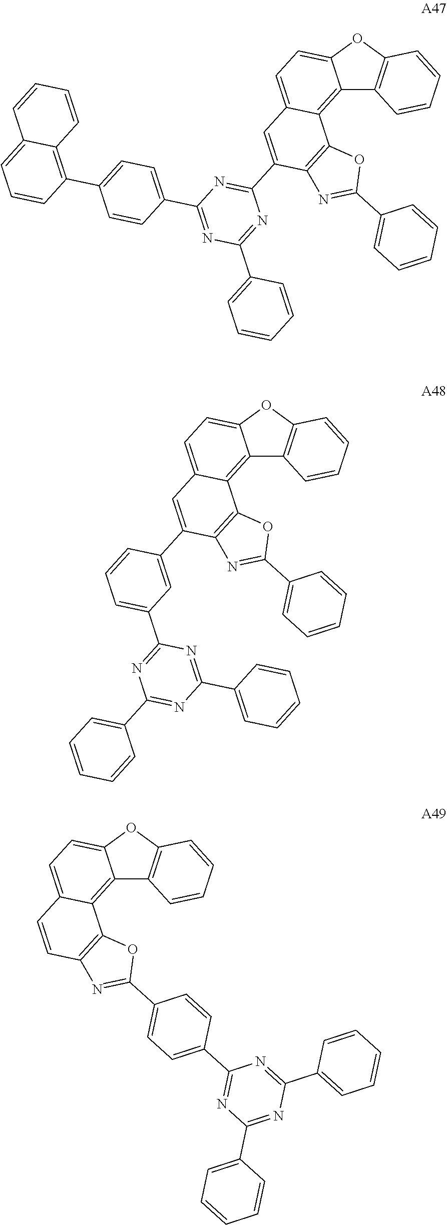 [SMILES: c1ccc(-c2nc(-c3ccc(-c4cccc5ccccc45)cc3)nc(-c3cc4ccc5oc6ccccc6c5c4c4oc(-c5ccccc5)nc34)n2)cc1 ; c1ccc(-c2nc(-c3ccccc3)nc(-c3cccc(-c4cc5ccc6oc7ccccc7c6c5c5oc(-c6ccccc6)nc45)c3)n2)cc1 ; c1ccc(-c2nc(-c3ccccc3)nc(-c3ccc(-c4nc5ccc6ccc7oc8ccccc8c7c6c5o4)cc3)n2)cc1]

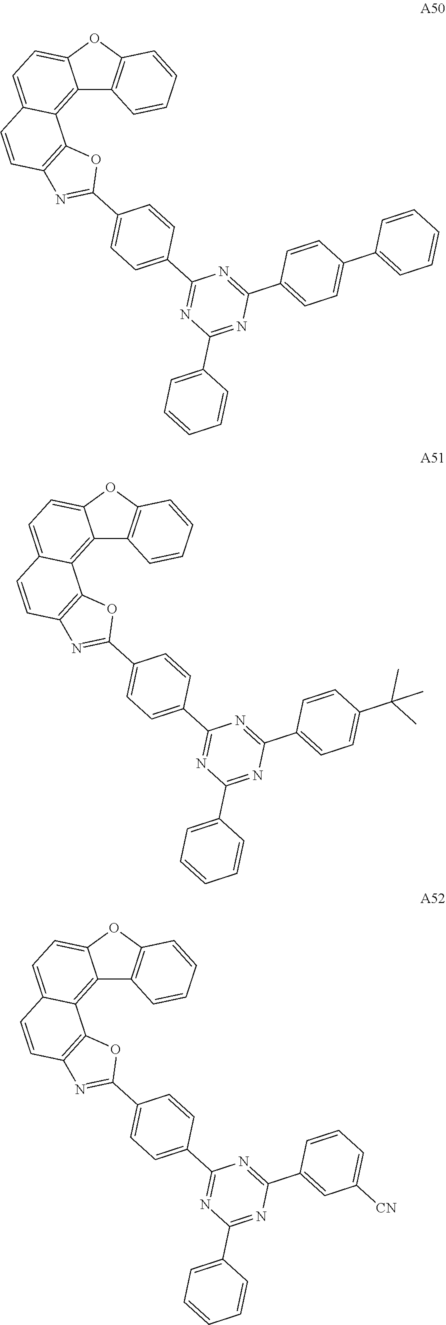 [SMILES: c1ccc(-c2ccc(-c3nc(-c4ccccc4)nc(-c4ccc(-c5nc6ccc7ccc8oc9ccccc9c8c7c6o5)cc4)n3)cc2)cc1 ; CC(C)(C)c1ccc(-c2nc(-c3ccccc3)nc(-c3ccc(-c4nc5ccc6ccc7oc8ccccc8c7c6c5o4)cc3)n2)cc1 ; N#Cc1cccc(-c2nc(-c3ccccc3)nc(-c3ccc(-c4nc5ccc6ccc7oc8ccccc8c7c6c5o4)cc3)n2)c1]

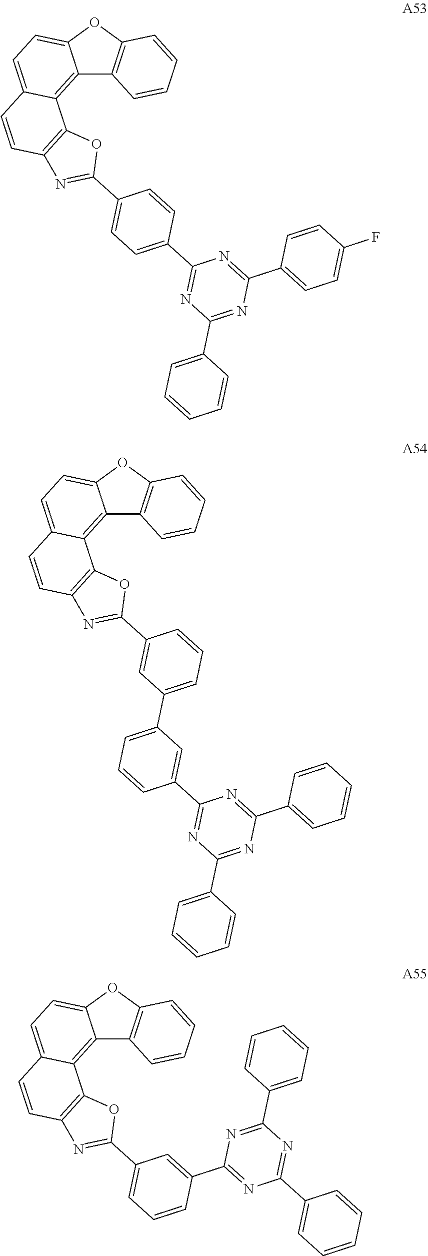 [SMILES: Fc1ccc(-c2nc(-c3ccccc3)nc(-c3ccc(-c4nc5ccc6ccc7oc8ccccc8c7c6c5o4)cc3)n2)cc1 ; c1ccc(-c2nc(-c3ccccc3)nc(-c3cccc(-c4cccc(-c5nc6ccc7ccc8oc9ccccc9c8c7c6o5)c4)c3)n2)cc1 ; c1ccc(-c2nc(-c3ccccc3)nc(-c3cccc(-c4nc5ccc6ccc7oc8ccccc8c7c6c5o4)c3)n2)cc1]

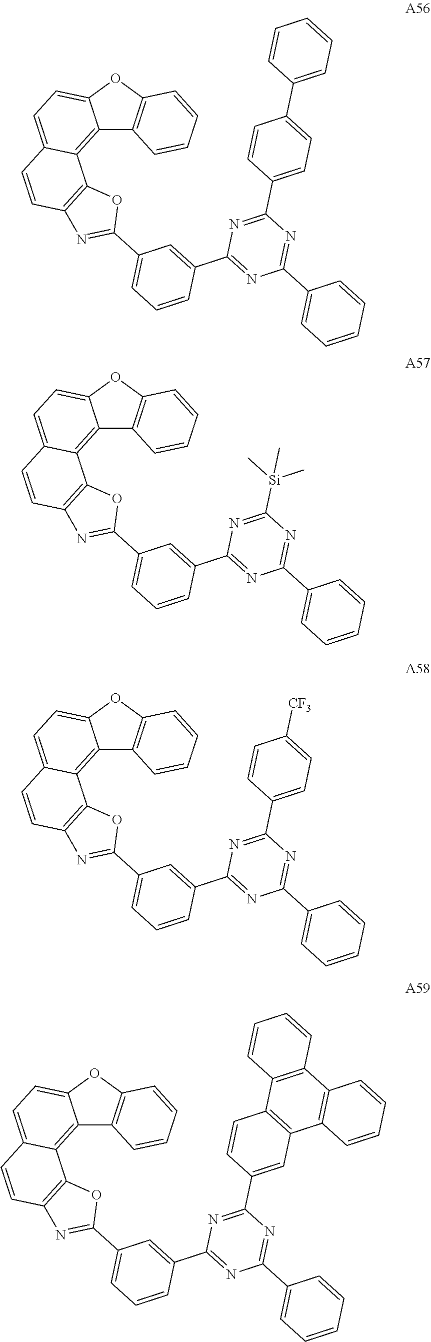 [SMILES: c1ccc(-c2ccc(-c3nc(-c4ccccc4)nc(-c4cccc(-c5nc6ccc7ccc8oc9ccccc9c8c7c6o5)c4)n3)cc2)cc1 ; C[Si](C)(C)c1nc(-c2ccccc2)nc(-c2cccc(-c3nc4ccc5ccc6oc7ccccc7c6c5c4o3)c2)n1 ; FC(F)(F)c1ccc(-c2nc(-c3ccccc3)nc(-c3cccc(-c4nc5ccc6ccc7oc8ccccc8c7c6c5o4)c3)n2)cc1 ; c1ccc(-c2nc(-c3cccc(-c4nc5ccc6ccc7oc8ccccc8c7c6c5o4)c3)nc(-c3ccc4c5ccccc5c5ccccc5c4c3)n2)cc1]

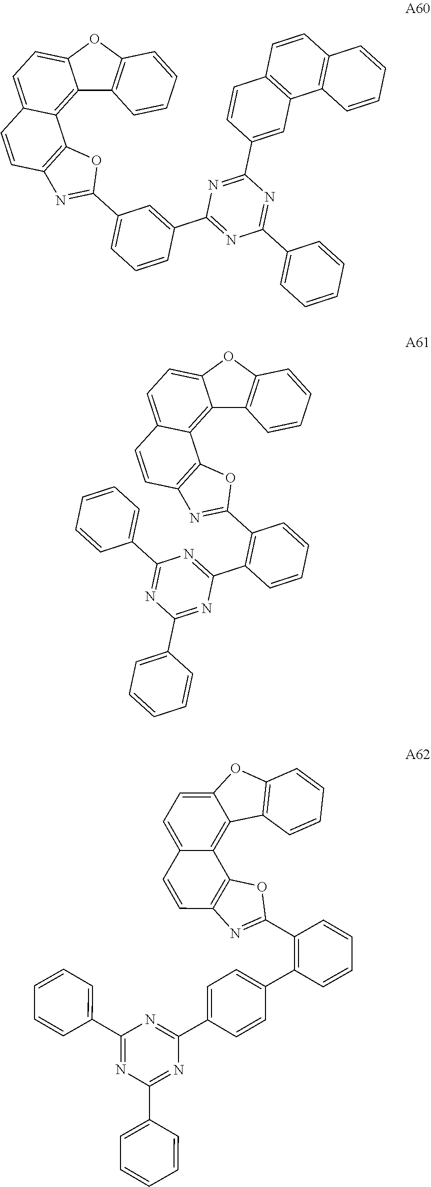 [SMILES: c1ccc(-c2nc(-c3cccc(-c4nc5ccc6ccc7oc8ccccc8c7c6c5o4)c3)nc(-c3ccc4ccc5ccccc5c4c3)n2)cc1 ; c1ccc(-c2nc(-c3ccccc3)nc(-c3ccccc3-c3nc4ccc5ccc6oc7ccccc7c6c5c4o3)n2)cc1 ; c1ccc(-c2nc(-c3ccccc3)nc(-c3ccc(-c4ccccc4-c4nc5ccc6ccc7oc8ccccc8c7c6c5o4)cc3)n2)cc1]

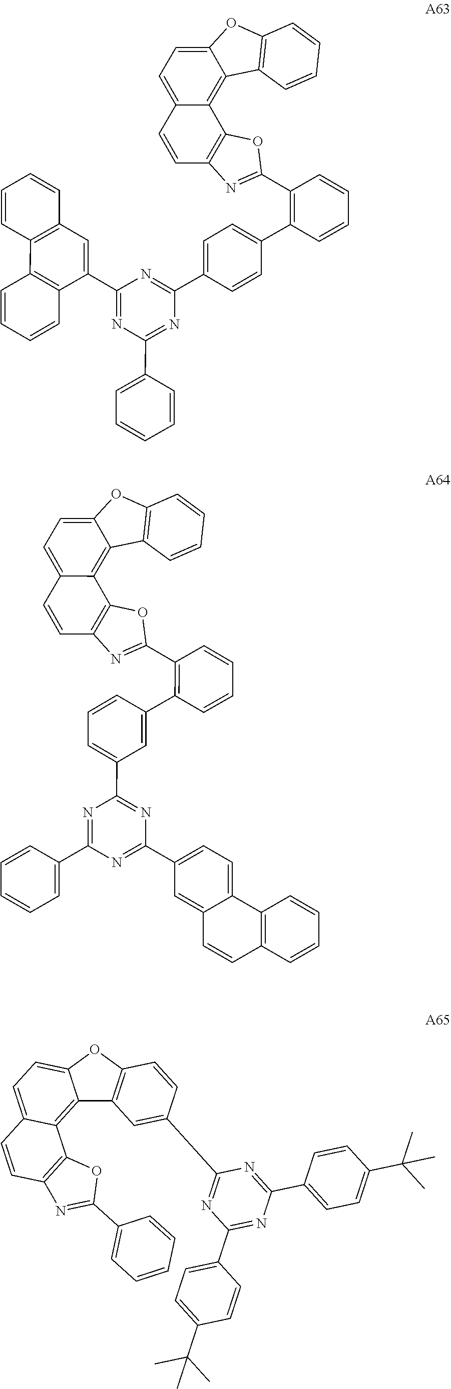 [SMILES: c1ccc(-c2nc(-c3ccc(-c4ccccc4-c4nc5ccc6ccc7oc8ccccc8c7c6c5o4)cc3)nc(-c3cc4ccccc4c4ccccc34)n2)cc1 ; c1ccc(-c2nc(-c3cccc(-c4ccccc4-c4nc5ccc6ccc7oc8ccccc8c7c6c5o4)c3)nc(-c3ccc4c(ccc5ccccc54)c3)n2)cc1 ; CC(C)(C)c1ccc(-c2nc(-c3ccc(C(C)(C)C)cc3)nc(-c3ccc4oc5ccc6ccc7nc(-c8ccccc8)oc7c6c5c4c3)n2)cc1]

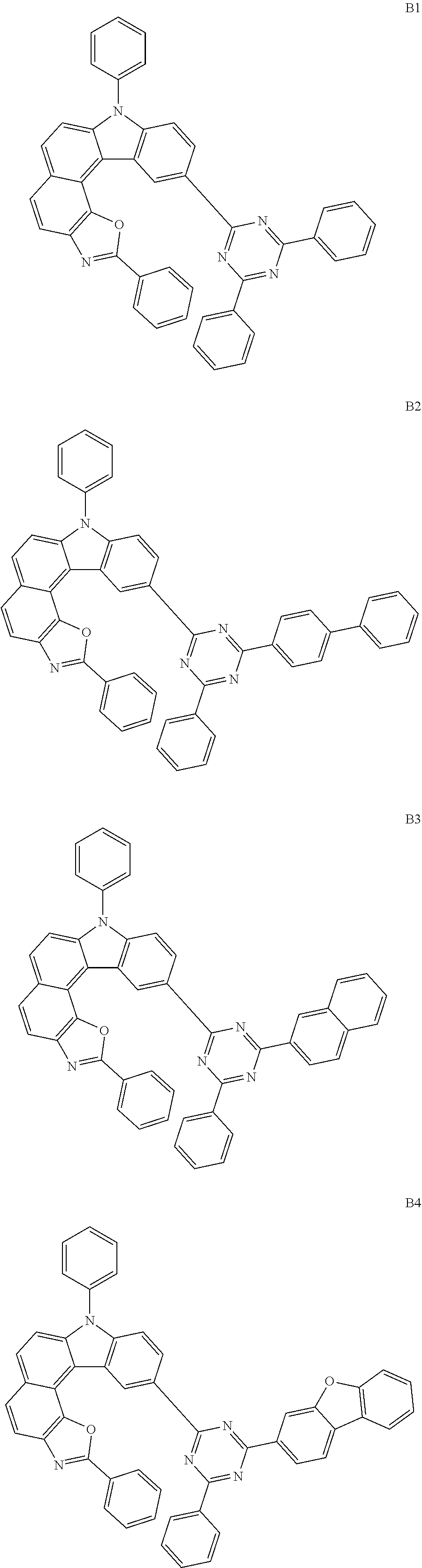 [SMILES: c1ccc(-c2nc(-c3ccccc3)nc(-c3ccc4c(c3)c3c5c(ccc6nc(-c7ccccc7)oc65)ccc3n4-c3ccccc3)n2)cc1 ; c1ccc(-c2ccc(-c3nc(-c4ccccc4)nc(-c4ccc5c(c4)c4c6c(ccc7nc(-c8ccccc8)oc76)ccc4n5-c4ccccc4)n3)cc2)cc1 ; c1ccc(-c2nc(-c3ccc4ccccc4c3)nc(-c3ccc4c(c3)c3c5c(ccc6nc(-c7ccccc7)oc65)ccc3n4-c3ccccc3)n2)cc1 ; c1ccc(-c2nc(-c3ccc4c(c3)oc3ccccc34)nc(-c3ccc4c(c3)c3c5c(ccc6nc(-c7ccccc7)oc65)ccc3n4-c3ccccc3)n2)cc1]

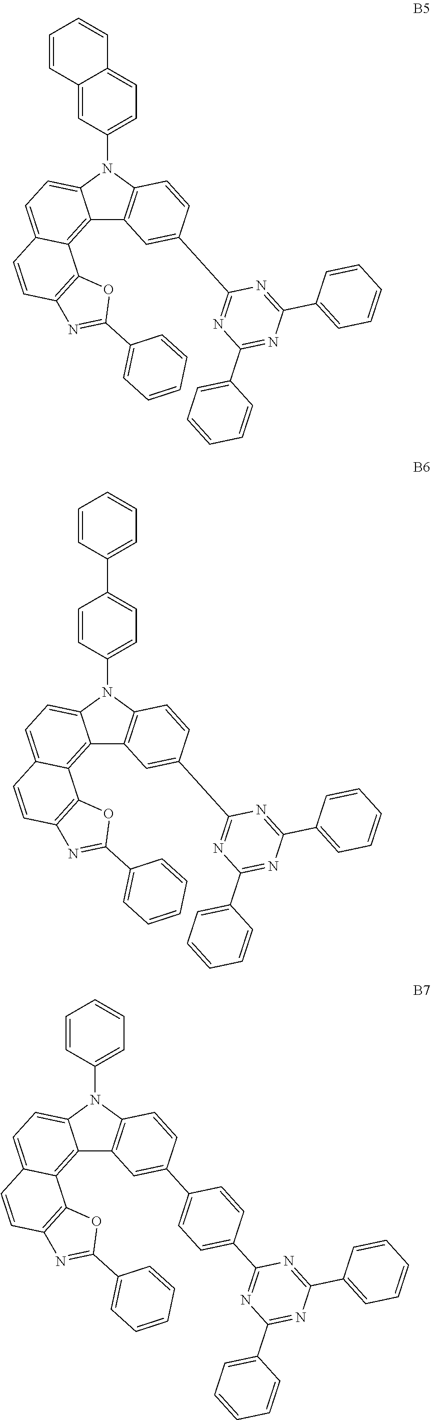 [SMILES: c1ccc(-c2nc(-c3ccccc3)nc(-c3ccc4c(c3)c3c5c(ccc6nc(-c7ccccc7)oc65)ccc3n4-c3ccc4ccccc4c3)n2)cc1 ; c1ccc(-c2ccc(-n3c4ccc(-c5nc(-c6ccccc6)nc(-c6ccccc6)n5)cc4c4c5c(ccc6nc(-c7ccccc7)oc65)ccc43)cc2)cc1 ; c1ccc(-c2nc(-c3ccccc3)nc(-c3ccc(-c4ccc5c(c4)c4c6c(ccc7nc(-c8ccccc8)oc76)ccc4n5-c4ccccc4)cc3)n2)cc1]

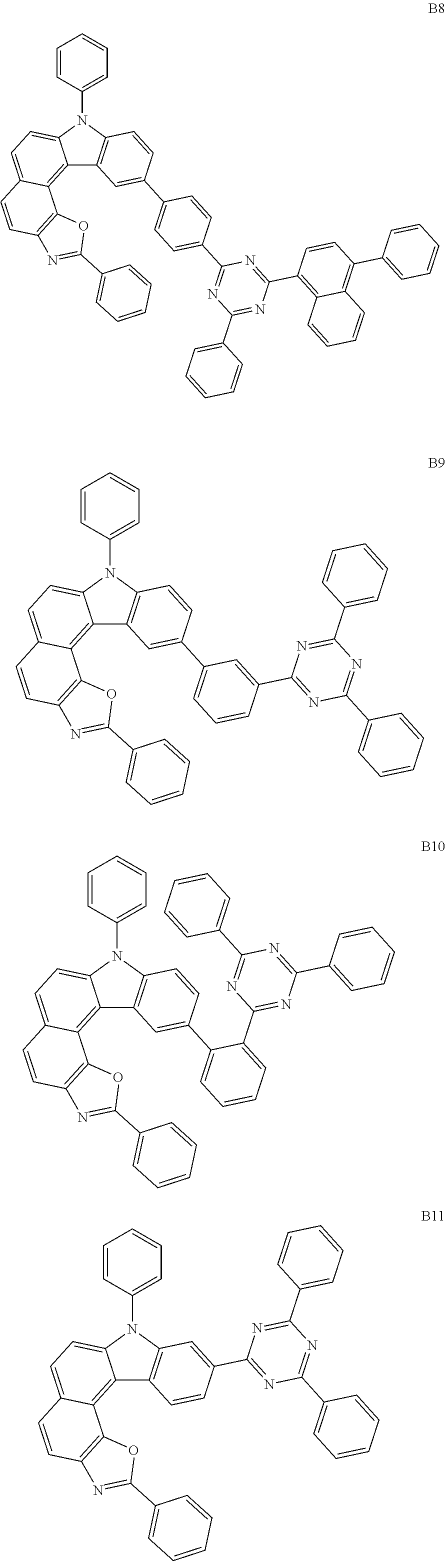 [SMILES: c1ccc(-c2nc(-c3ccc(-c4ccc5c(c4)c4c6c(ccc7nc(-c8ccccc8)oc76)ccc4n5-c4ccccc4)cc3)nc(-c3ccc(-c4ccccc4)c4ccccc34)n2)cc1 ; c1ccc(-c2nc(-c3ccccc3)nc(-c3cccc(-c4ccc5c(c4)c4c6c(ccc7nc(-c8ccccc8)oc76)ccc4n5-c4ccccc4)c3)n2)cc1 ; c1ccc(-c2nc(-c3ccccc3)nc(-c3ccccc3-c3ccc4c(c3)c3c5c(ccc6nc(-c7ccccc7)oc65)ccc3n4-c3ccccc3)n2)cc1 ; c1ccc(-c2nc(-c3ccccc3)nc(-c3ccc4c5c6c(ccc7nc(-c8ccccc8)oc76)ccc5n(-c5ccccc5)c4c3)n2)cc1]

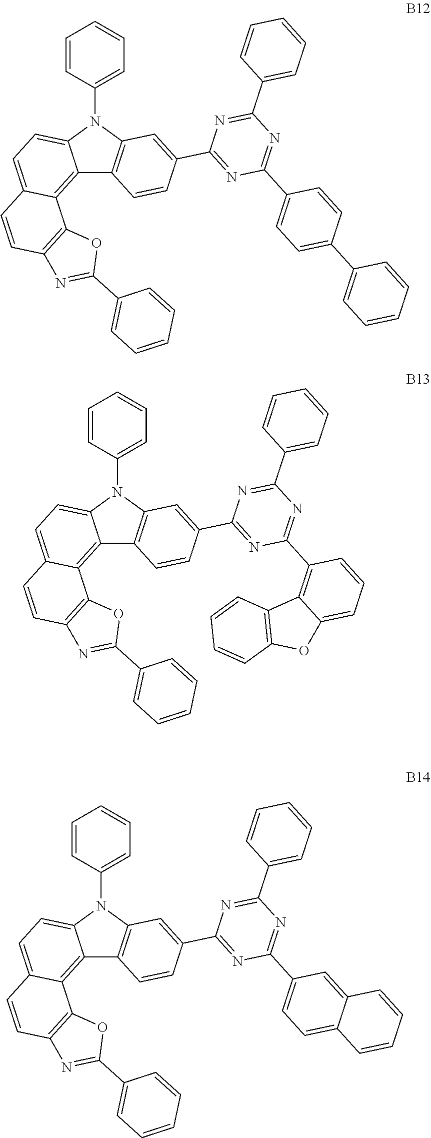

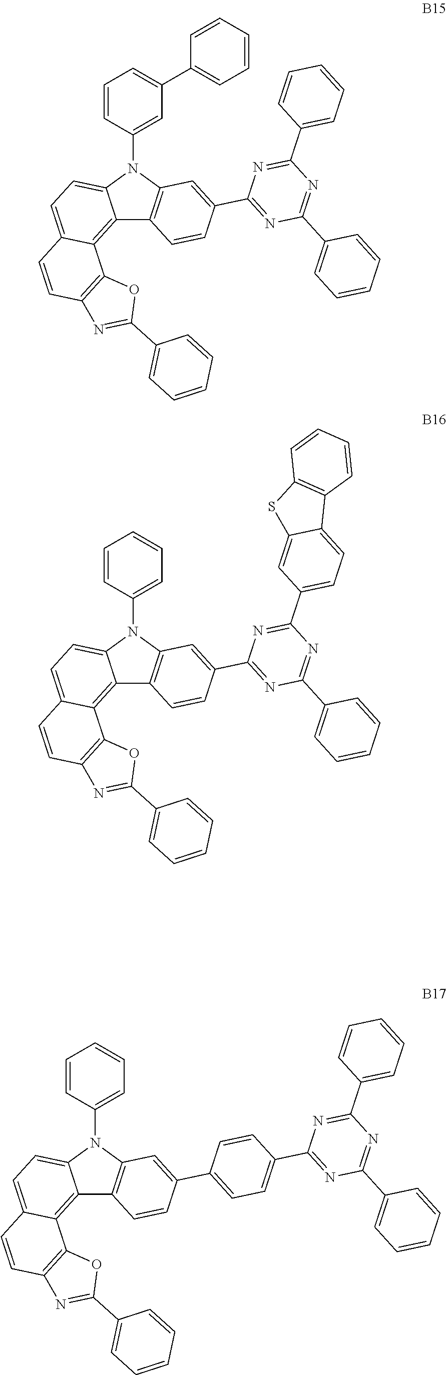 [SMILES: c1ccc(-c2cccc(-n3c4cc(-c5nc(-c6ccccc6)nc(-c6ccccc6)n5)ccc4c4c5c(ccc6nc(-c7ccccc7)oc65)ccc43)c2)cc1 ; c1ccc(-c2nc(-c3ccc4c(c3)sc3ccccc34)nc(-c3ccc4c5c6c(ccc7nc(-c8ccccc8)oc76)ccc5n(-c5ccccc5)c4c3)n2)cc1 ; c1ccc(-c2nc(-c3ccccc3)nc(-c3ccc(-c4ccc5c6c7c(ccc8nc(-c9ccccc9)oc87)ccc6n(-c6ccccc6)c5c4)cc3)n2)cc1]

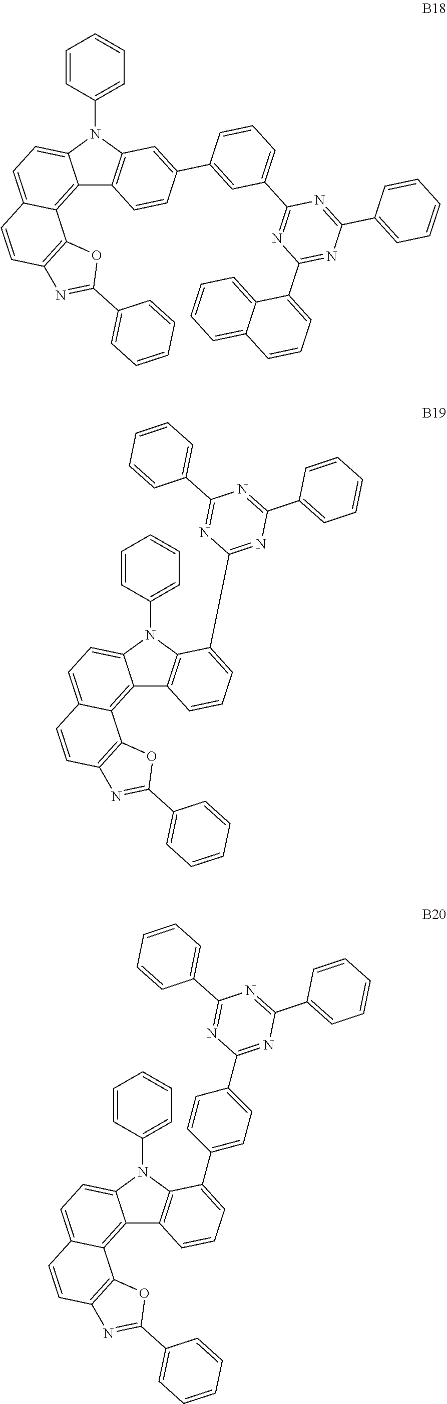 [SMILES: c1ccc(-c2nc(-c3cccc(-c4ccc5c6c7c(ccc8nc(-c9ccccc9)oc87)ccc6n(-c6ccccc6)c5c4)c3)nc(-c3cccc4ccccc34)n2)cc1 ; c1ccc(-c2nc(-c3ccccc3)nc(-c3cccc4c5c6c(ccc7nc(-c8ccccc8)oc76)ccc5n(-c5ccccc5)c34)n2)cc1 ; c1ccc(-c2nc(-c3ccccc3)nc(-c3ccc(-c4cccc5c6c7c(ccc8nc(-c9ccccc9)oc87)ccc6n(-c6ccccc6)c45)cc3)n2)cc1]

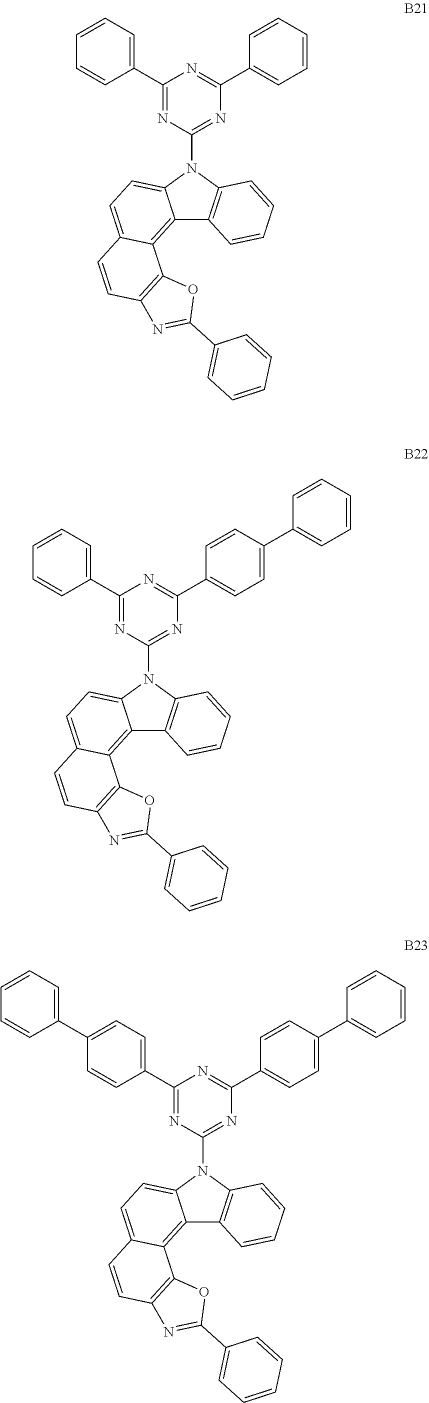 [SMILES: c1ccc(-c2nc(-c3ccccc3)nc(-n3c4ccccc4c4c5c(ccc6nc(-c7ccccc7)oc65)ccc43)n2)cc1 ; c1ccc(-c2ccc(-c3nc(-c4ccccc4)nc(-n4c5ccccc5c5c6c(ccc7nc(-c8ccccc8)oc76)ccc54)n3)cc2)cc1 ; c1ccc(-c2ccc(-c3nc(-c4ccc(-c5ccccc5)cc4)nc(-n4c5ccccc5c5c6c(ccc7nc(-c8ccccc8)oc76)ccc54)n3)cc2)cc1]

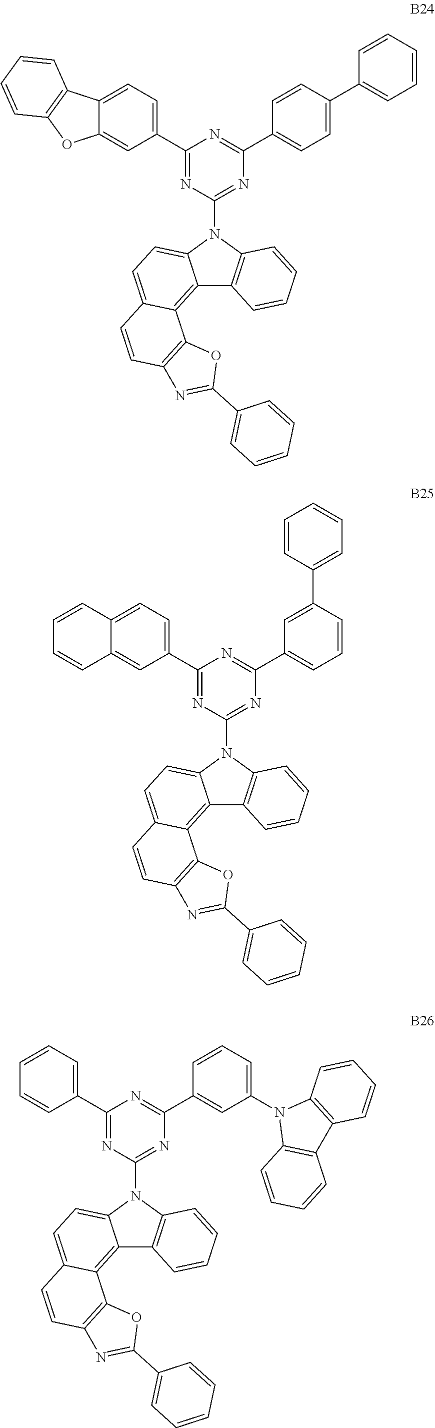 [SMILES: c1ccc(-c2ccc(-c3nc(-c4ccc5c(c4)oc4ccccc45)nc(-n4c5ccccc5c5c6c(ccc7nc(-c8ccccc8)oc76)ccc54)n3)cc2)cc1 ; c1ccc(-c2cccc(-c3nc(-c4ccc5ccccc5c4)nc(-n4c5ccccc5c5c6c(ccc7nc(-c8ccccc8)oc76)ccc54)n3)c2)cc1 ; c1ccc(-c2nc(-c3cccc(-n4c5ccccc5c5ccccc54)c3)nc(-n3c4ccccc4c4c5c(ccc6nc(-c7ccccc7)oc65)ccc43)n2)cc1]

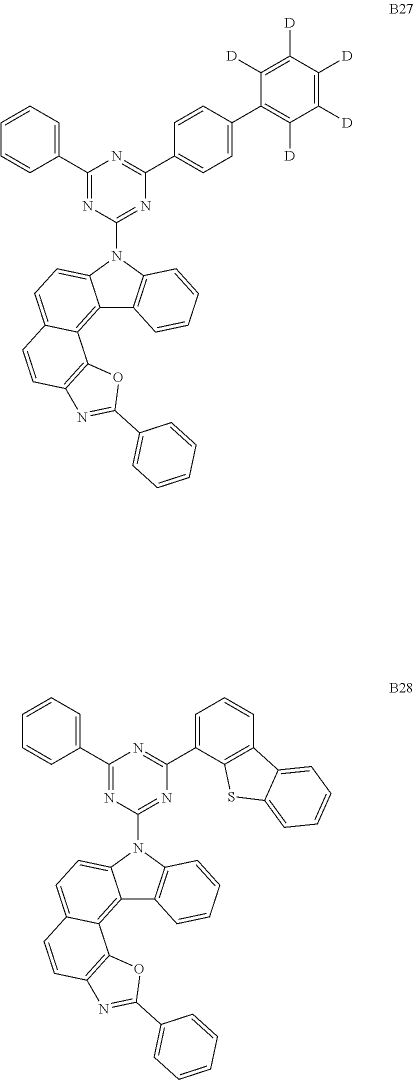 [SMILES: [2H]c1c([2H])c([2H])c(-c2ccc(-c3nc(-c4ccccc4)nc(-n4c5ccccc5c5c6c(ccc7nc(-c8ccccc8)oc76)ccc54)n3)cc2)c([2H])c1[2H] ; c1ccc(-c2nc(-c3cccc4c3sc3ccccc34)nc(-n3c4ccccc4c4c5c(ccc6nc(-c7ccccc7)oc65)ccc43)n2)cc1]

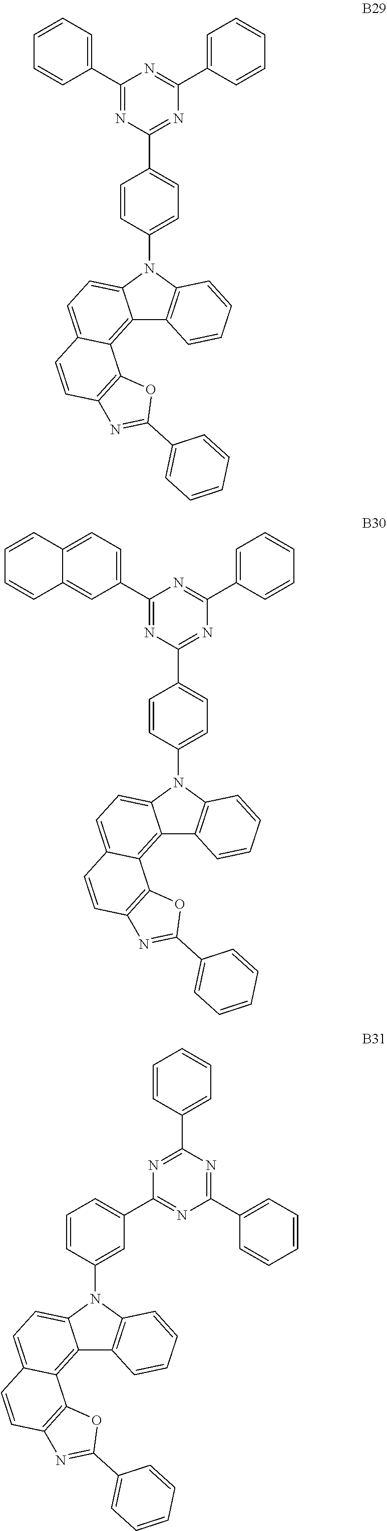 [SMILES: c1ccc(-c2nc(-c3ccccc3)nc(-c3ccc(-n4c5ccccc5c5c6c(ccc7nc(-c8ccccc8)oc76)ccc54)cc3)n2)cc1 ; c1ccc(-c2nc(-c3ccc(-n4c5ccccc5c5c6c(ccc7nc(-c8ccccc8)oc76)ccc54)cc3)nc(-c3ccc4ccccc4c3)n2)cc1 ; c1ccc(-c2nc(-c3ccccc3)nc(-c3cccc(-n4c5ccccc5c5c6c(ccc7nc(-c8ccccc8)oc76)ccc54)c3)n2)cc1]

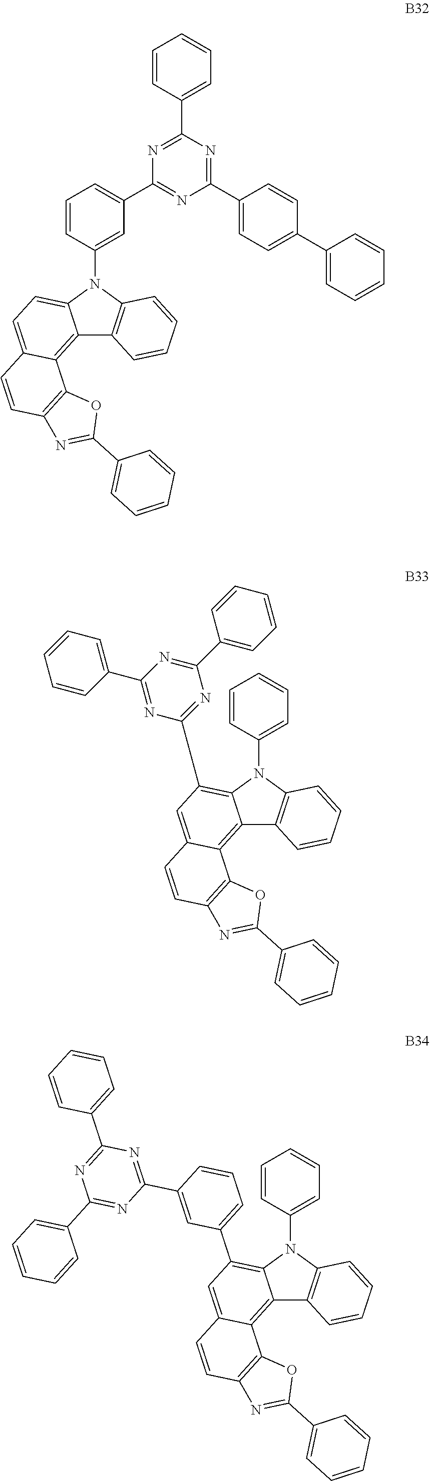 [SMILES: c1ccc(-c2ccc(-c3nc(-c4ccccc4)nc(-c4cccc(-n5c6ccccc6c6c7c(ccc8nc(-c9ccccc9)oc87)ccc65)c4)n3)cc2)cc1 ; c1ccc(-c2nc(-c3ccccc3)nc(-c3cc4ccc5nc(-c6ccccc6)oc5c4c4c5ccccc5n(-c5ccccc5)c34)n2)cc1 ; c1ccc(-c2nc(-c3ccccc3)nc(-c3cccc(-c4cc5ccc6nc(-c7ccccc7)oc6c5c5c6ccccc6n(-c6ccccc6)c45)c3)n2)cc1]

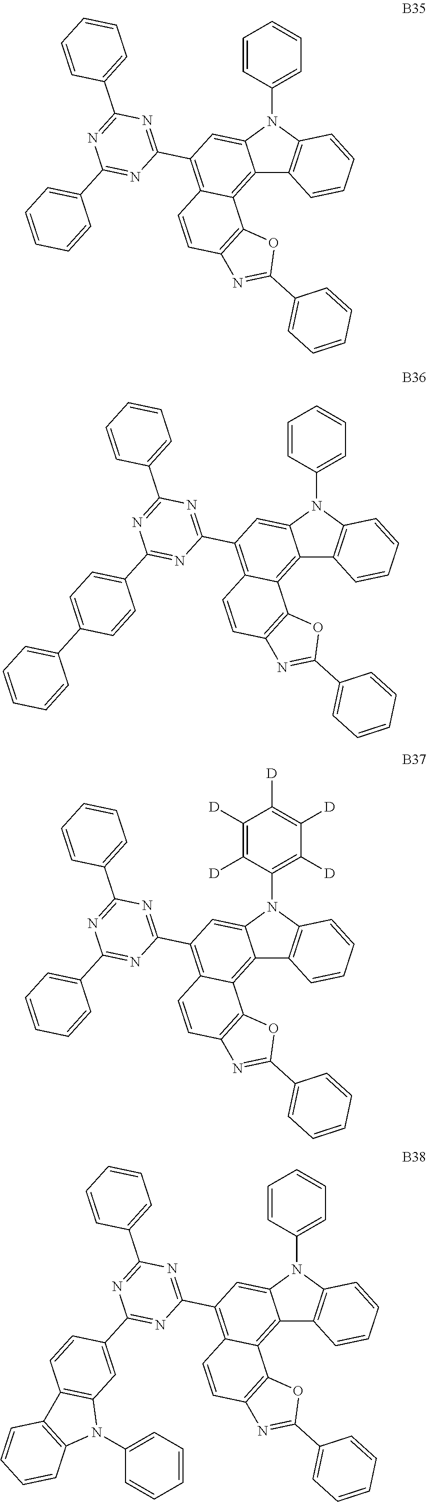

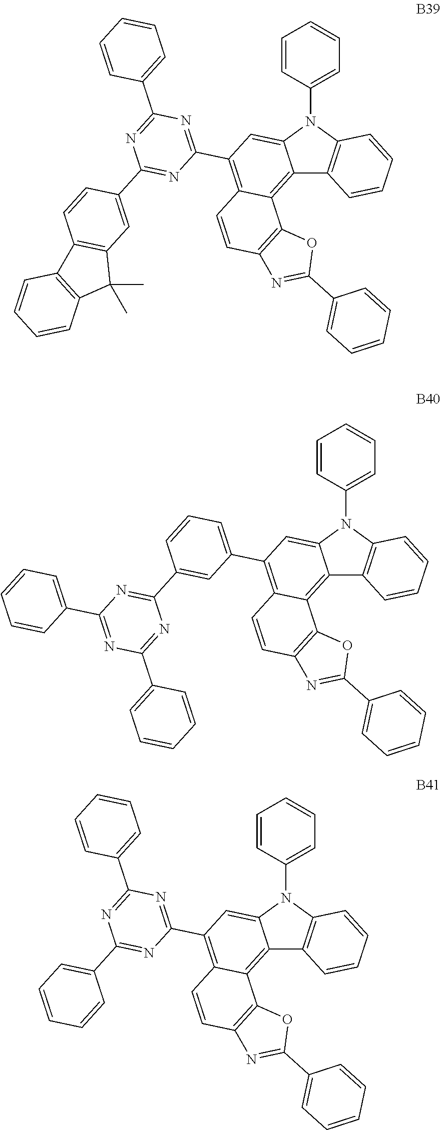 [SMILES: CC1(C)c2ccccc2-c2ccc(-c3nc(-c4ccccc4)nc(-c4cc5c(c6ccccc6n5-c5ccccc5)c5c4ccc4nc(-c6ccccc6)oc45)n3)cc21 ; c1ccc(-c2nc(-c3ccccc3)nc(-c3cccc(-c4cc5c(c6ccccc6n5-c5ccccc5)c5c4ccc4nc(-c6ccccc6)oc45)c3)n2)cc1 ; c1ccc(-c2nc(-c3ccccc3)nc(-c3cc4c(c5ccccc5n4-c4ccccc4)c4c3ccc3nc(-c5ccccc5)oc34)n2)cc1]

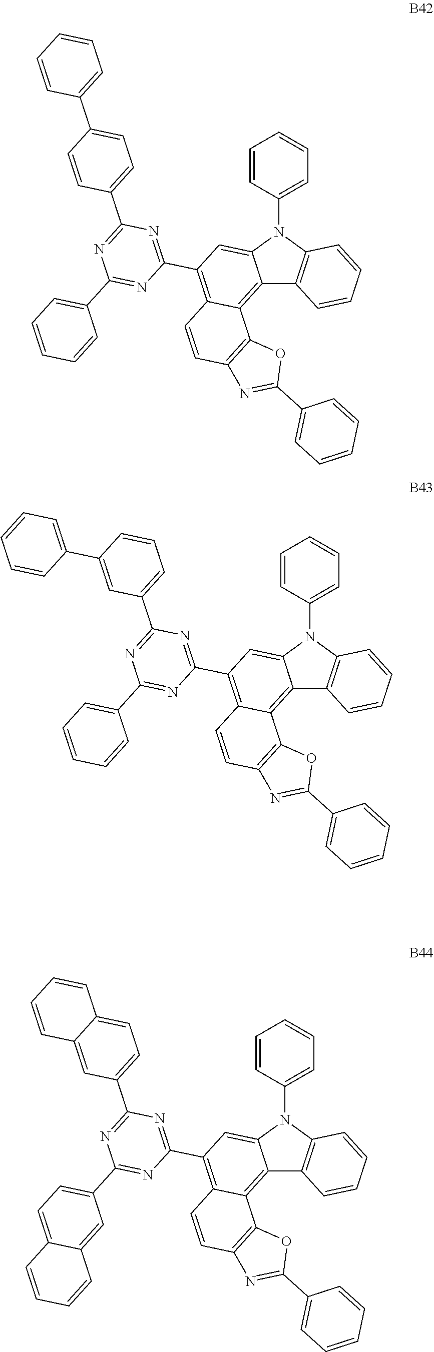 [SMILES: c1ccc(-c2ccc(-c3nc(-c4ccccc4)nc(-c4cc5c(c6ccccc6n5-c5ccccc5)c5c4ccc4nc(-c6ccccc6)oc45)n3)cc2)cc1 ; c1ccc(-c2cccc(-c3nc(-c4ccccc4)nc(-c4cc5c(c6ccccc6n5-c5ccccc5)c5c4ccc4nc(-c6ccccc6)oc45)n3)c2)cc1 ; c1ccc(-c2nc3ccc4c(-c5nc(-c6ccc7ccccc7c6)nc(-c6ccc7ccccc7c6)n5)cc5c(c6ccccc6n5-c5ccccc5)c4c3o2)cc1]

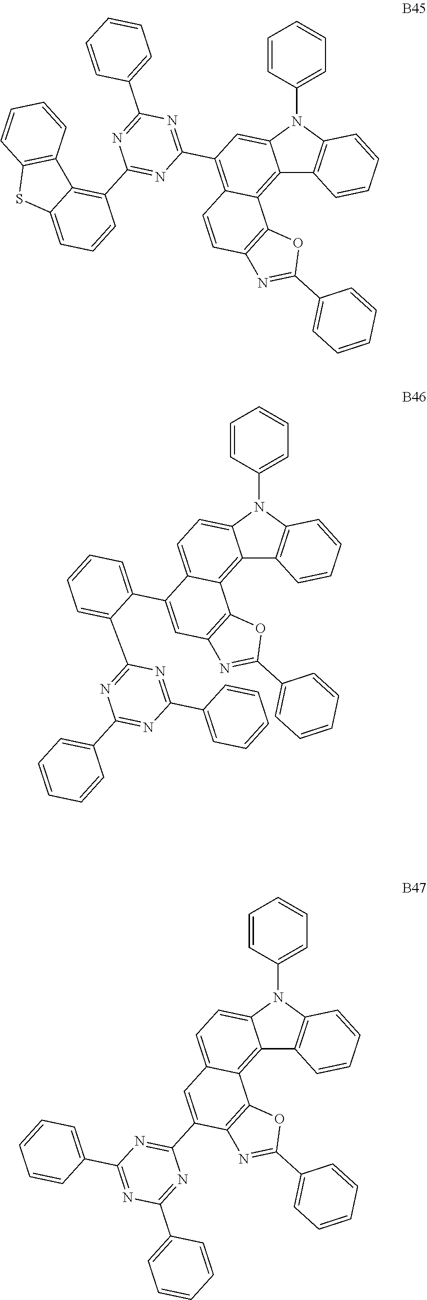 [SMILES: c1ccc(-c2nc(-c3cc4c(c5ccccc5n4-c4ccccc4)c4c3ccc3nc(-c5ccccc5)oc34)nc(-c3cccc4sc5ccccc5c34)n2)cc1 ; c1ccc(-c2nc(-c3ccccc3)nc(-c3ccccc3-c3cc4nc(-c5ccccc5)oc4c4c3ccc3c4c4ccccc4n3-c3ccccc3)n2)cc1 ; c1ccc(-c2nc(-c3ccccc3)nc(-c3cc4ccc5c(c6ccccc6n5-c5ccccc5)c4c4oc(-c5ccccc5)nc34)n2)cc1]

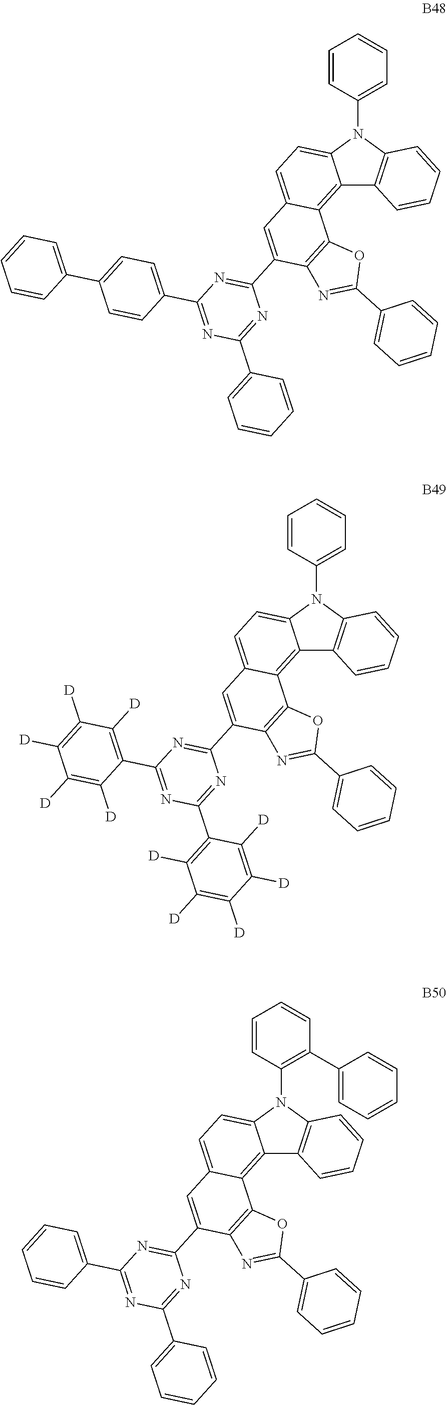 [SMILES: c1ccc(-c2ccc(-c3nc(-c4ccccc4)nc(-c4cc5ccc6c(c7ccccc7n6-c6ccccc6)c5c5oc(-c6ccccc6)nc45)n3)cc2)cc1 ; [2H]c1c([2H])c([2H])c(-c2nc(-c3c([2H])c([2H])c([2H])c([2H])c3[2H])nc(-c3cc4ccc5c(c6ccccc6n5-c5ccccc5)c4c4oc(-c5ccccc5)nc34)n2)c([2H])c1[2H] ; c1ccc(-c2nc(-c3ccccc3)nc(-c3cc4ccc5c(c6ccccc6n5-c5ccccc5-c5ccccc5)c4c4oc(-c5ccccc5)nc34)n2)cc1]

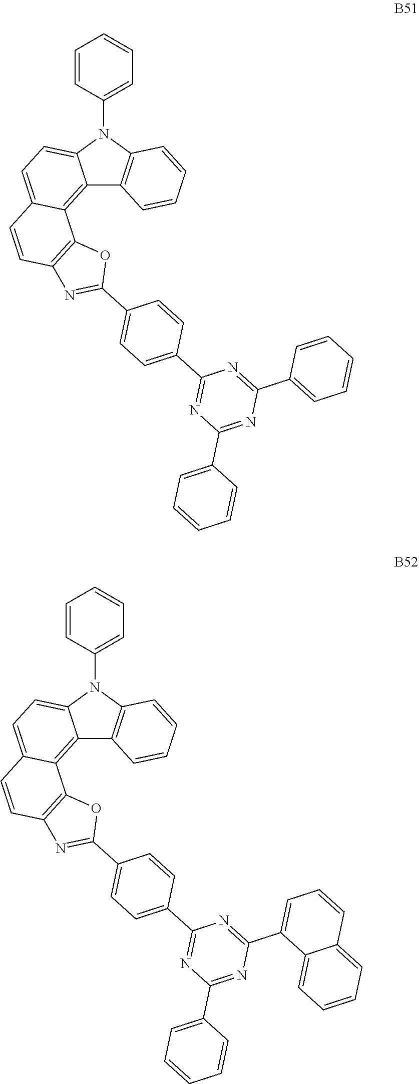 [SMILES: c1ccc(-c2nc(-c3ccccc3)nc(-c3ccc(-c4nc5ccc6ccc7c(c8ccccc8n7-c7ccccc7)c6c5o4)cc3)n2)cc1 ; c1ccc(-c2nc(-c3ccc(-c4nc5ccc6ccc7c(c8ccccc8n7-c7ccccc7)c6c5o4)cc3)nc(-c3cccc4ccccc34)n2)cc1]

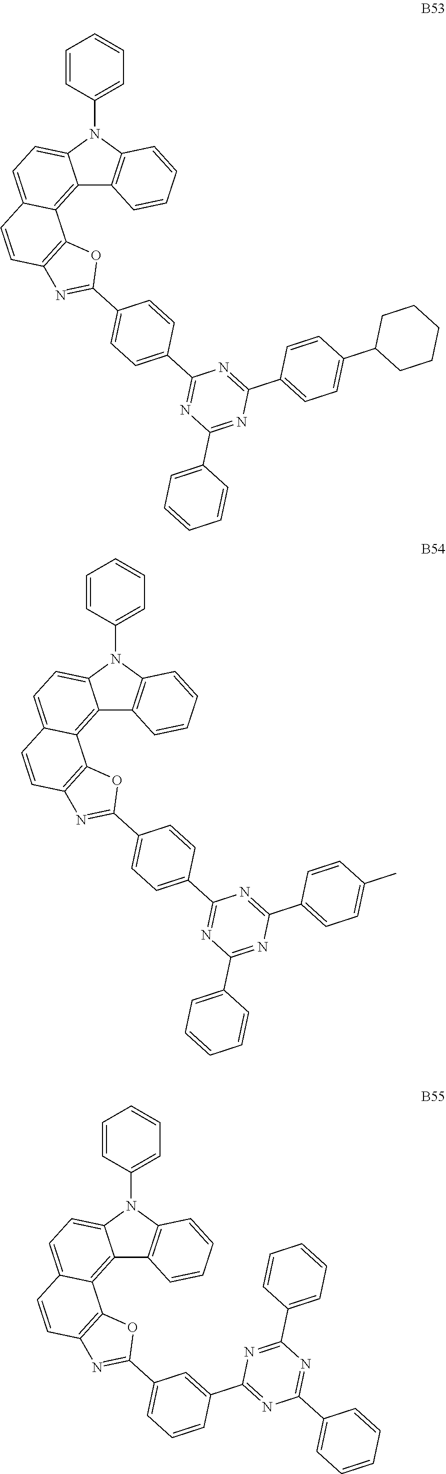 [SMILES: c1ccc(-c2nc(-c3ccc(-c4nc5ccc6ccc7c(c8ccccc8n7-c7ccccc7)c6c5o4)cc3)nc(-c3ccc(C4CCCCC4)cc3)n2)cc1 ; Cc1ccc(-c2nc(-c3ccccc3)nc(-c3ccc(-c4nc5ccc6ccc7c(c8ccccc8n7-c7ccccc7)c6c5o4)cc3)n2)cc1 ; c1ccc(-c2nc(-c3ccccc3)nc(-c3cccc(-c4nc5ccc6ccc7c(c8ccccc8n7-c7ccccc7)c6c5o4)c3)n2)cc1]

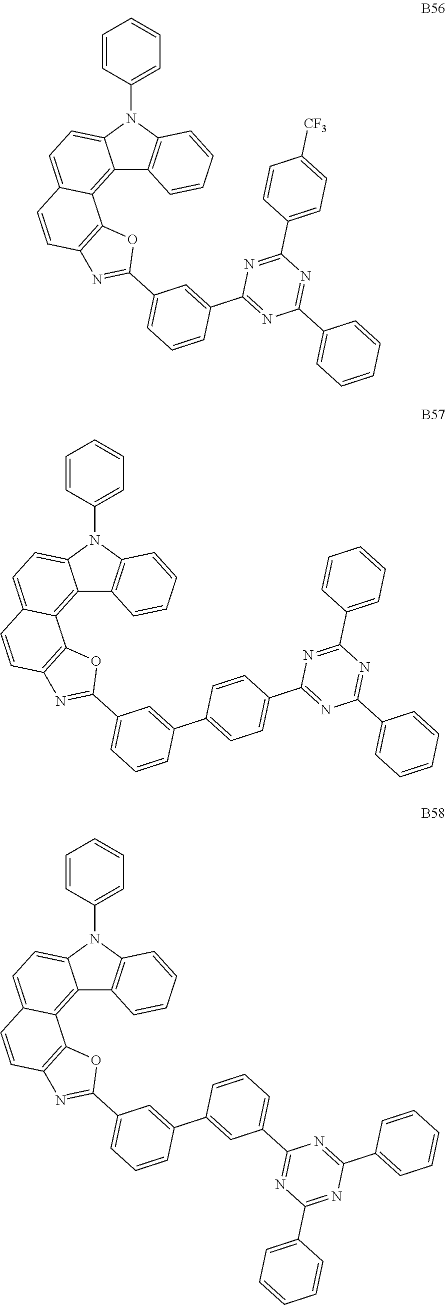 [SMILES: FC(F)(F)c1ccc(-c2nc(-c3ccccc3)nc(-c3cccc(-c4nc5ccc6ccc7c(c8ccccc8n7-c7ccccc7)c6c5o4)c3)n2)cc1 ; c1ccc(-c2nc(-c3ccccc3)nc(-c3ccc(-c4cccc(-c5nc6ccc7ccc8c(c9ccccc9n8-c8ccccc8)c7c6o5)c4)cc3)n2)cc1 ; c1ccc(-c2nc(-c3ccccc3)nc(-c3cccc(-c4cccc(-c5nc6ccc7ccc8c(c9ccccc9n8-c8ccccc8)c7c6o5)c4)c3)n2)cc1]

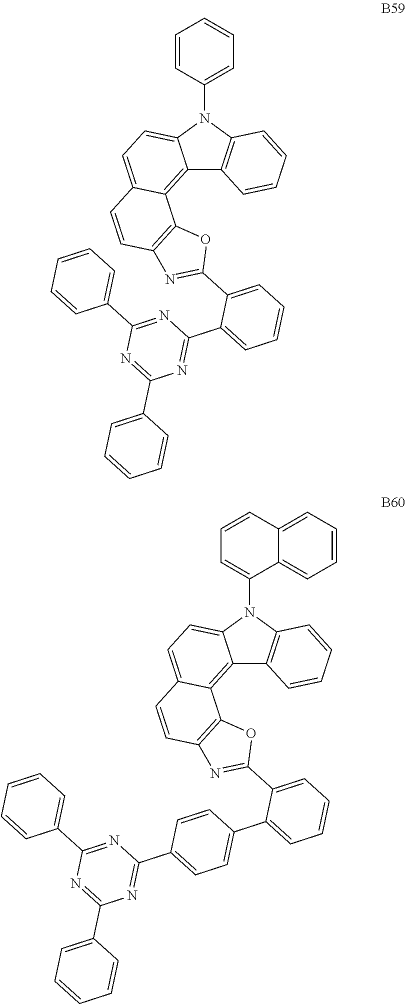 [SMILES: c1ccc(-c2nc(-c3ccccc3)nc(-c3ccccc3-c3nc4ccc5ccc6c(c7ccccc7n6-c6ccccc6)c5c4o3)n2)cc1 ; c1ccc(-c2nc(-c3ccccc3)nc(-c3ccc(-c4ccccc4-c4nc5ccc6ccc7c(c8ccccc8n7-c7cccc8ccccc78)c6c5o4)cc3)n2)cc1]

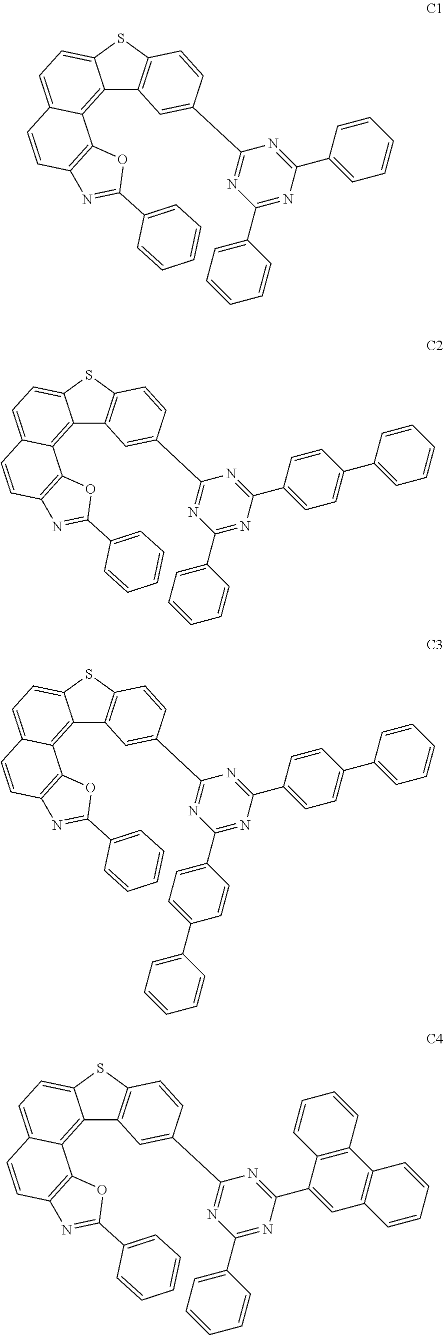 [SMILES: c1ccc(-c2nc(-c3ccccc3)nc(-c3ccc4sc5ccc6ccc7nc(-c8ccccc8)oc7c6c5c4c3)n2)cc1 ; c1ccc(-c2ccc(-c3nc(-c4ccccc4)nc(-c4ccc5sc6ccc7ccc8nc(-c9ccccc9)oc8c7c6c5c4)n3)cc2)cc1 ; c1ccc(-c2ccc(-c3nc(-c4ccc(-c5ccccc5)cc4)nc(-c4ccc5sc6ccc7ccc8nc(-c9ccccc9)oc8c7c6c5c4)n3)cc2)cc1 ; c1ccc(-c2nc(-c3ccc4sc5ccc6ccc7nc(-c8ccccc8)oc7c6c5c4c3)nc(-c3cc4ccccc4c4ccccc34)n2)cc1]

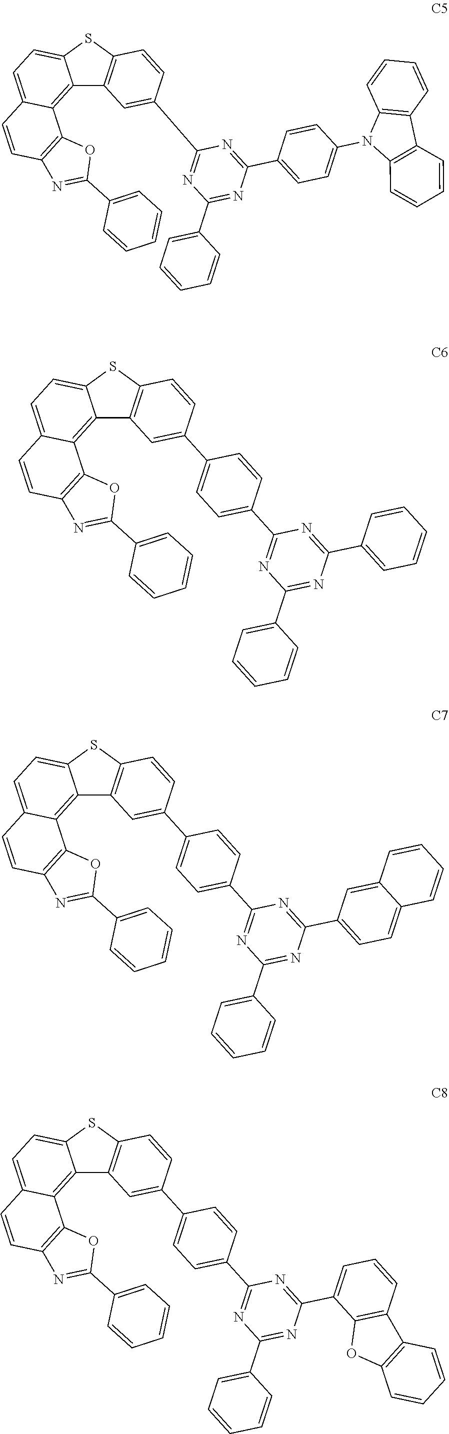 [SMILES: c1ccc(-c2nc(-c3ccc(-n4c5ccccc5c5ccccc54)cc3)nc(-c3ccc4sc5ccc6ccc7nc(-c8ccccc8)oc7c6c5c4c3)n2)cc1 ; c1ccc(-c2nc(-c3ccccc3)nc(-c3ccc(-c4ccc5sc6ccc7ccc8nc(-c9ccccc9)oc8c7c6c5c4)cc3)n2)cc1 ; c1ccc(-c2nc(-c3ccc(-c4ccc5sc6ccc7ccc8nc(-c9ccccc9)oc8c7c6c5c4)cc3)nc(-c3ccc4ccccc4c3)n2)cc1 ; c1ccc(-c2nc(-c3ccc(-c4ccc5sc6ccc7ccc8nc(-c9ccccc9)oc8c7c6c5c4)cc3)nc(-c3cccc4c3oc3ccccc34)n2)cc1]

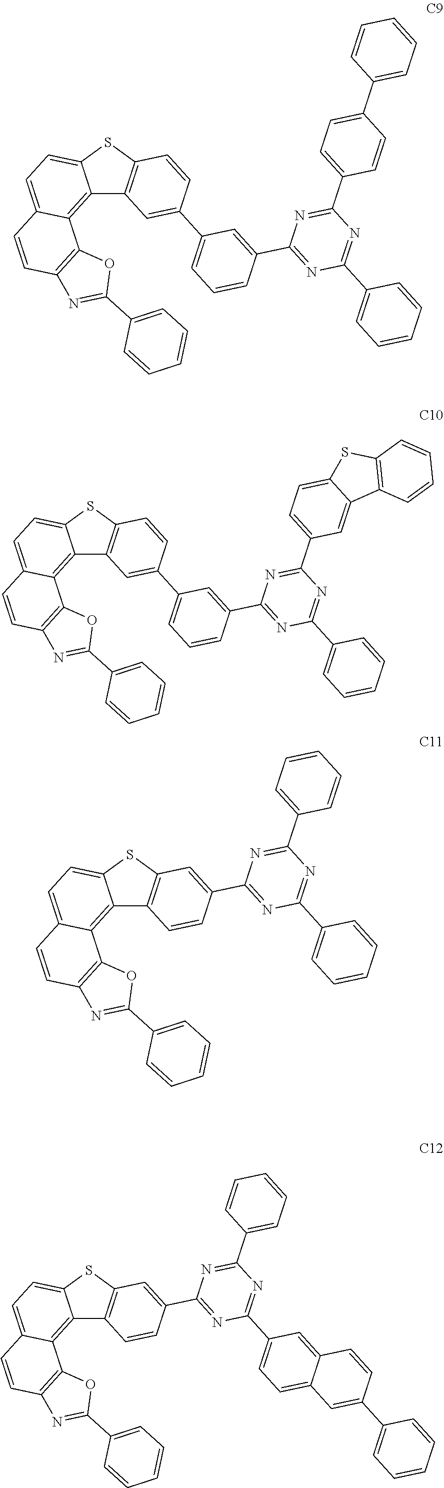 [SMILES: c1ccc(-c2ccc(-c3nc(-c4ccccc4)nc(-c4cccc(-c5ccc6sc7ccc8ccc9nc(-c%10ccccc%10)oc9c8c7c6c5)c4)n3)cc2)cc1 ; c1ccc(-c2nc(-c3cccc(-c4ccc5sc6ccc7ccc8nc(-c9ccccc9)oc8c7c6c5c4)c3)nc(-c3ccc4sc5ccccc5c4c3)n2)cc1 ; c1ccc(-c2nc(-c3ccccc3)nc(-c3ccc4c(c3)sc3ccc5ccc6nc(-c7ccccc7)oc6c5c34)n2)cc1 ; c1ccc(-c2ccc3cc(-c4nc(-c5ccccc5)nc(-c5ccc6c(c5)sc5ccc7ccc8nc(-c9ccccc9)oc8c7c56)n4)ccc3c2)cc1]

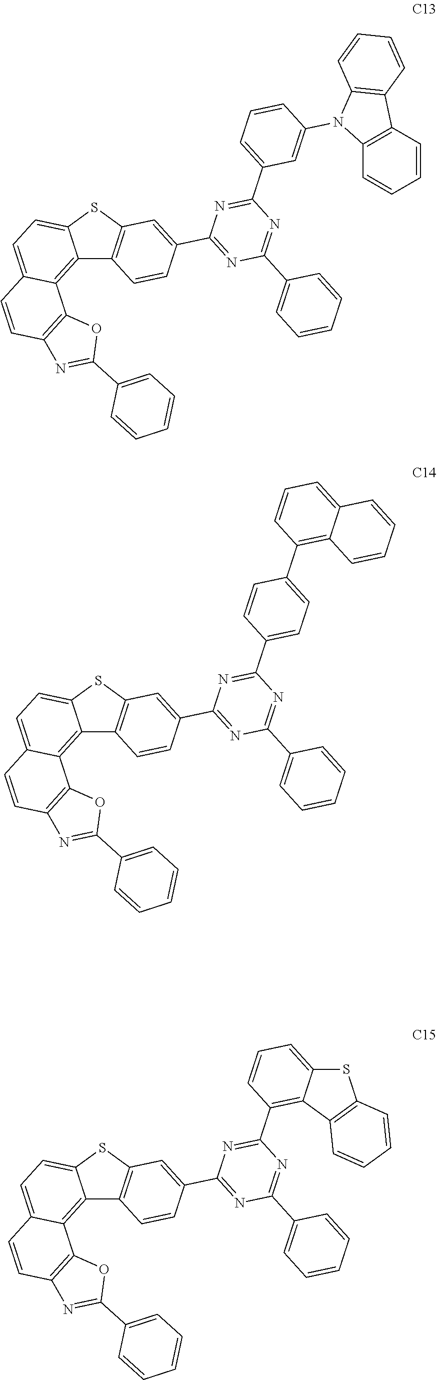 [SMILES: c1ccc(-c2nc(-c3cccc(-n4c5ccccc5c5ccccc54)c3)nc(-c3ccc4c(c3)sc3ccc5ccc6nc(-c7ccccc7)oc6c5c34)n2)cc1 ; c1ccc(-c2nc(-c3ccc(-c4cccc5ccccc45)cc3)nc(-c3ccc4c(c3)sc3ccc5ccc6nc(-c7ccccc7)oc6c5c34)n2)cc1 ; c1ccc(-c2nc(-c3ccc4c(c3)sc3ccc5ccc6nc(-c7ccccc7)oc6c5c34)nc(-c3cccc4sc5ccccc5c34)n2)cc1]

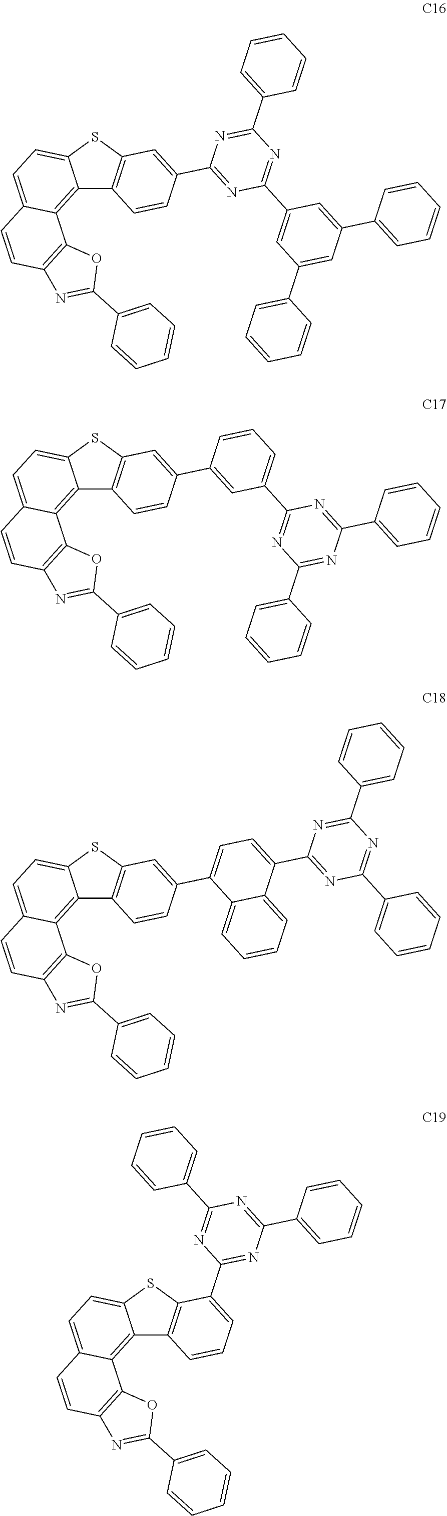 [SMILES: c1ccc(-c2cc(-c3ccccc3)cc(-c3nc(-c4ccccc4)nc(-c4ccc5c(c4)sc4ccc6ccc7nc(-c8ccccc8)oc7c6c45)n3)c2)cc1 ; c1ccc(-c2nc(-c3ccccc3)nc(-c3cccc(-c4ccc5c(c4)sc4ccc6ccc7nc(-c8ccccc8)oc7c6c45)c3)n2)cc1 ; c1ccc(-c2nc(-c3ccccc3)nc(-c3ccc(-c4ccc5c(c4)sc4ccc6ccc7nc(-c8ccccc8)oc7c6c45)c4ccccc34)n2)cc1 ; c1ccc(-c2nc(-c3ccccc3)nc(-c3cccc4c3sc3ccc5ccc6nc(-c7ccccc7)oc6c5c34)n2)cc1]

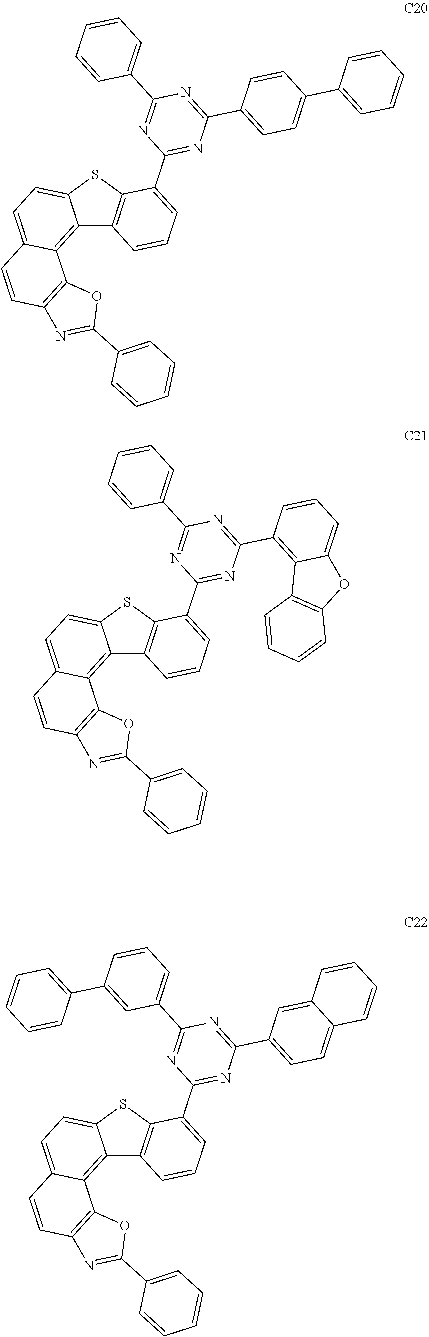

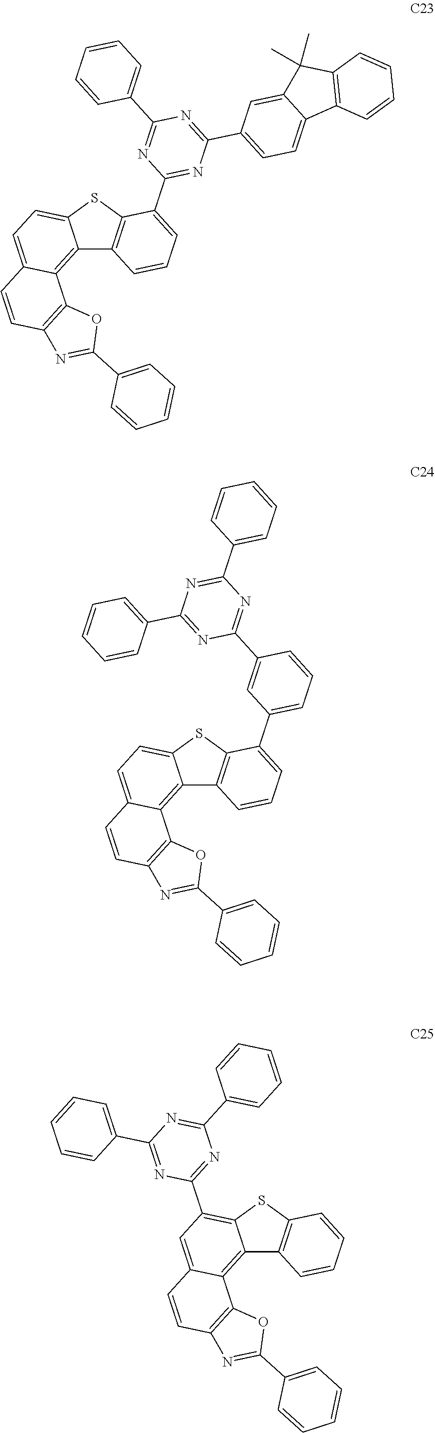 [SMILES: CC1(C)c2ccccc2-c2ccc(-c3nc(-c4ccccc4)nc(-c4cccc5c4sc4ccc6ccc7nc(-c8ccccc8)oc7c6c45)n3)cc21 ; c1ccc(-c2nc(-c3ccccc3)nc(-c3cccc(-c4cccc5c4sc4ccc6ccc7nc(-c8ccccc8)oc7c6c45)c3)n2)cc1 ; c1ccc(-c2nc(-c3ccccc3)nc(-c3cc4ccc5nc(-c6ccccc6)oc5c4c4c3sc3ccccc34)n2)cc1]

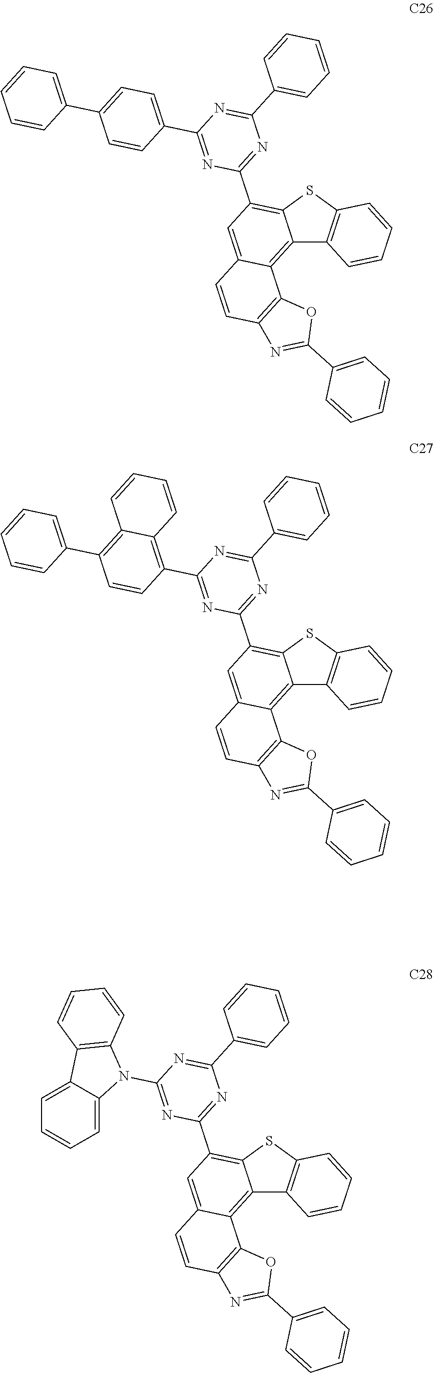 [SMILES: c1ccc(-c2ccc(-c3nc(-c4ccccc4)nc(-c4cc5ccc6nc(-c7ccccc7)oc6c5c5c4sc4ccccc45)n3)cc2)cc1 ; c1ccc(-c2nc(-c3ccc(-c4ccccc4)c4ccccc34)nc(-c3cc4ccc5nc(-c6ccccc6)oc5c4c4c3sc3ccccc34)n2)cc1 ; c1ccc(-c2nc(-c3cc4ccc5nc(-c6ccccc6)oc5c4c4c3sc3ccccc34)nc(-n3c4ccccc4c4ccccc43)n2)cc1]

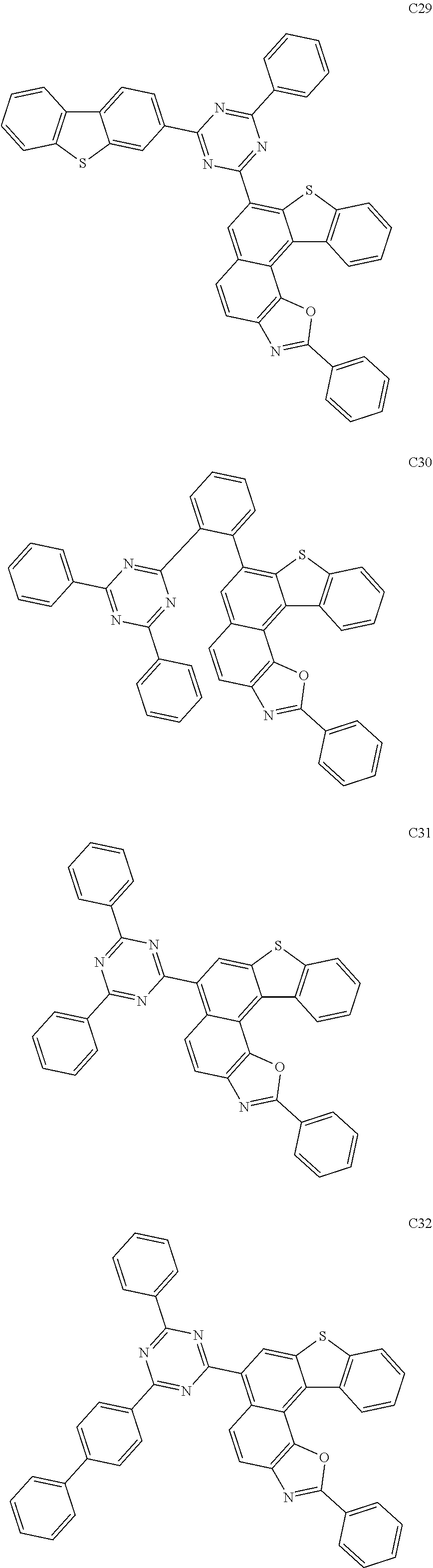 [SMILES: c1ccc(-c2nc(-c3ccc4c(c3)sc3ccccc34)nc(-c3cc4ccc5nc(-c6ccccc6)oc5c4c4c3sc3ccccc34)n2)cc1 ; c1ccc(-c2nc(-c3ccccc3)nc(-c3ccccc3-c3cc4ccc5nc(-c6ccccc6)oc5c4c4c3sc3ccccc34)n2)cc1 ; c1ccc(-c2nc(-c3ccccc3)nc(-c3cc4sc5ccccc5c4c4c3ccc3nc(-c5ccccc5)oc34)n2)cc1 ; c1ccc(-c2ccc(-c3nc(-c4ccccc4)nc(-c4cc5sc6ccccc6c5c5c4ccc4nc(-c6ccccc6)oc45)n3)cc2)cc1]

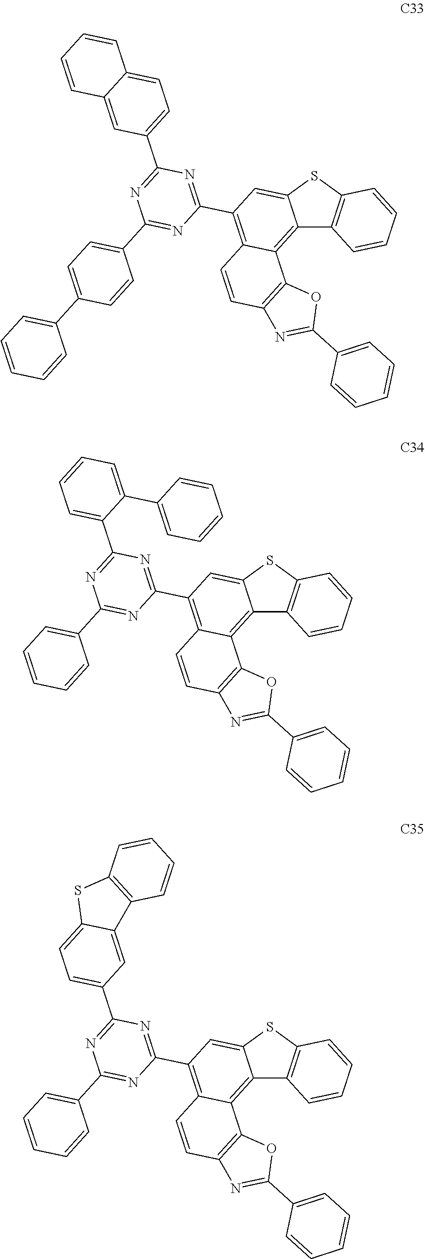 [SMILES: c1ccc(-c2ccc(-c3nc(-c4ccc5ccccc5c4)nc(-c4cc5sc6ccccc6c5c5c4ccc4nc(-c6ccccc6)oc45)n3)cc2)cc1 ; c1ccc(-c2nc(-c3ccccc3-c3ccccc3)nc(-c3cc4sc5ccccc5c4c4c3ccc3nc(-c5ccccc5)oc34)n2)cc1 ; c1ccc(-c2nc(-c3ccc4sc5ccccc5c4c3)nc(-c3cc4sc5ccccc5c4c4c3ccc3nc(-c5ccccc5)oc34)n2)cc1]

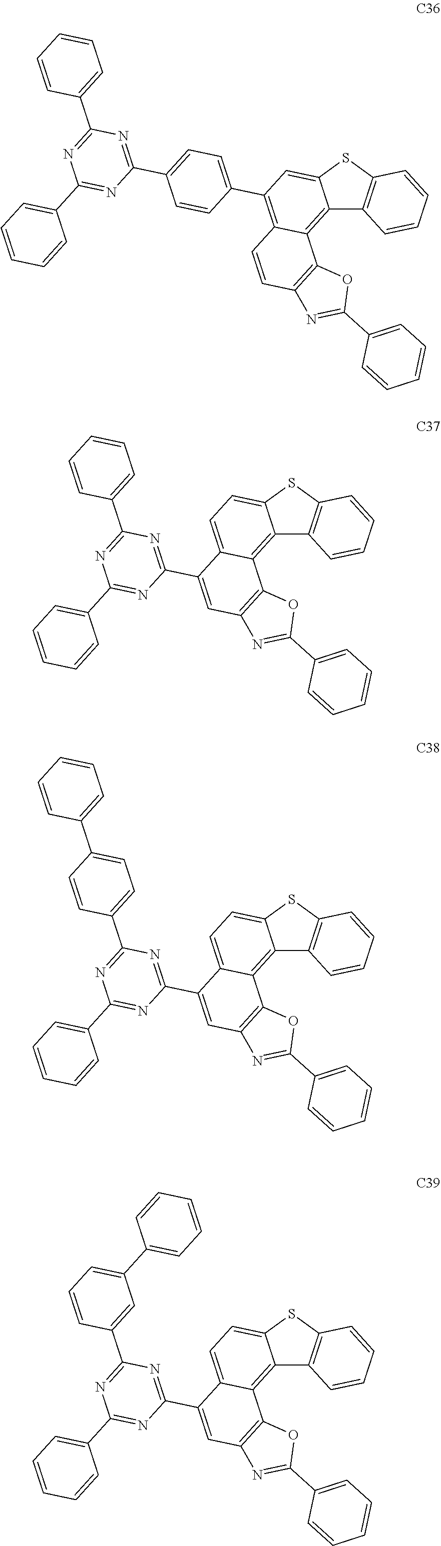 [SMILES: c1ccc(-c2nc(-c3ccccc3)nc(-c3ccc(-c4cc5sc6ccccc6c5c5c4ccc4nc(-c6ccccc6)oc45)cc3)n2)cc1 ; c1ccc(-c2nc(-c3ccccc3)nc(-c3cc4nc(-c5ccccc5)oc4c4c3ccc3sc5ccccc5c34)n2)cc1 ; c1ccc(-c2ccc(-c3nc(-c4ccccc4)nc(-c4cc5nc(-c6ccccc6)oc5c5c4ccc4sc6ccccc6c45)n3)cc2)cc1 ; c1ccc(-c2cccc(-c3nc(-c4ccccc4)nc(-c4cc5nc(-c6ccccc6)oc5c5c4ccc4sc6ccccc6c45)n3)c2)cc1]

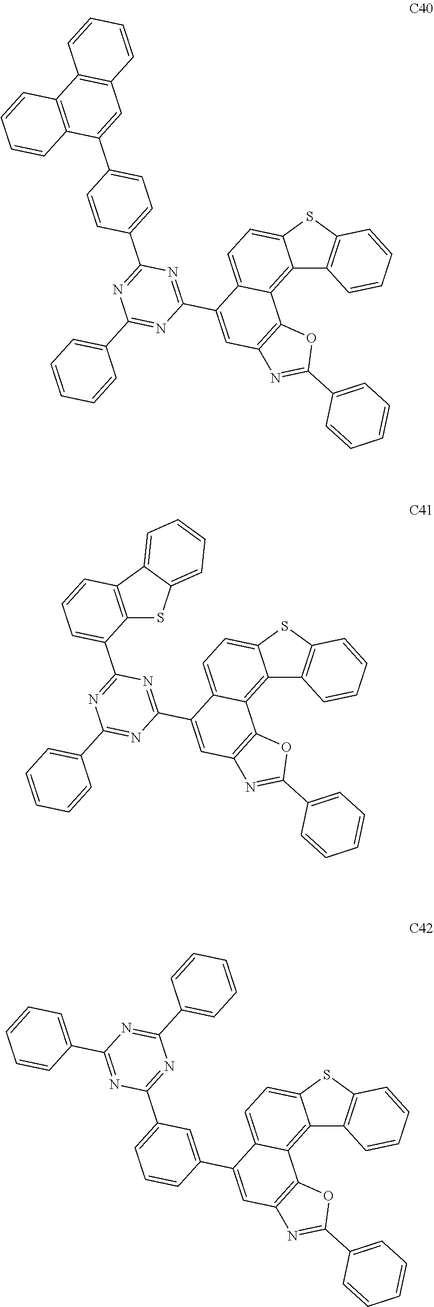 [SMILES: c1ccc(-c2nc(-c3ccc(-c4cc5ccccc5c5ccccc45)cc3)nc(-c3cc4nc(-c5ccccc5)oc4c4c3ccc3sc5ccccc5c34)n2)cc1 ; c1ccc(-c2nc(-c3cc4nc(-c5ccccc5)oc4c4c3ccc3sc5ccccc5c34)nc(-c3cccc4c3sc3ccccc34)n2)cc1 ; c1ccc(-c2nc(-c3ccccc3)nc(-c3cccc(-c4cc5nc(-c6ccccc6)oc5c5c4ccc4sc6ccccc6c45)c3)n2)cc1]

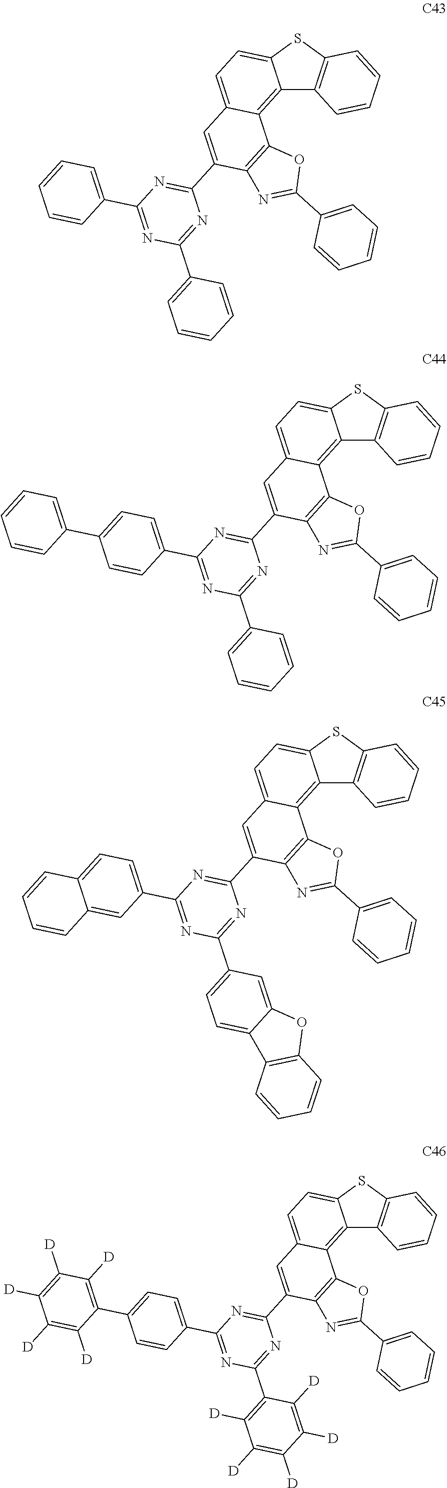 [SMILES: c1ccc(-c2nc(-c3ccccc3)nc(-c3cc4ccc5sc6ccccc6c5c4c4oc(-c5ccccc5)nc34)n2)cc1 ; c1ccc(-c2ccc(-c3nc(-c4ccccc4)nc(-c4cc5ccc6sc7ccccc7c6c5c5oc(-c6ccccc6)nc45)n3)cc2)cc1 ; c1ccc(-c2nc3c(-c4nc(-c5ccc6ccccc6c5)nc(-c5ccc6c(c5)oc5ccccc56)n4)cc4ccc5sc6ccccc6c5c4c3o2)cc1 ; [2H]c1c([2H])c([2H])c(-c2ccc(-c3nc(-c4c([2H])c([2H])c([2H])c([2H])c4[2H])nc(-c4cc5ccc6sc7ccccc7c6c5c5oc(-c6ccccc6)nc45)n3)cc2)c([2H])c1[2H]]

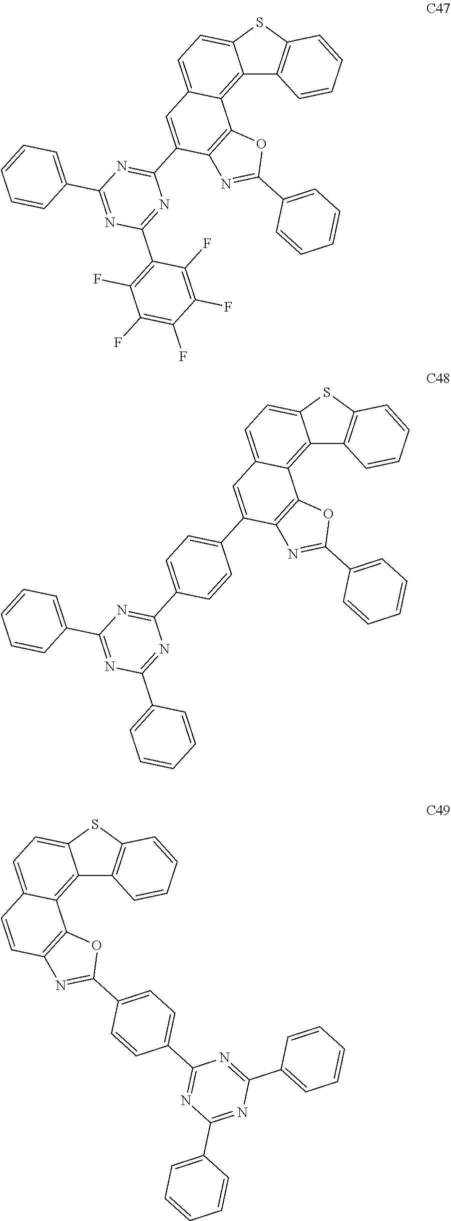 [SMILES: Fc1c(F)c(F)c(-c2nc(-c3ccccc3)nc(-c3cc4ccc5sc6ccccc6c5c4c4oc(-c5ccccc5)nc34)n2)c(F)c1F ; c1ccc(-c2nc(-c3ccccc3)nc(-c3ccc(-c4cc5ccc6sc7ccccc7c6c5c5oc(-c6ccccc6)nc45)cc3)n2)cc1 ; c1ccc(-c2nc(-c3ccccc3)nc(-c3ccc(-c4nc5ccc6ccc7sc8ccccc8c7c6c5o4)cc3)n2)cc1]

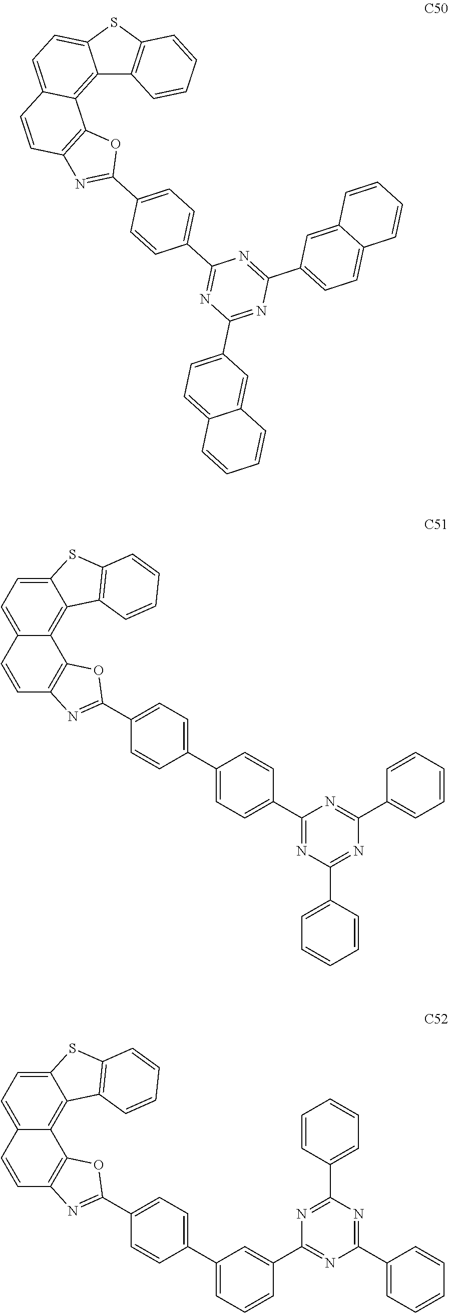 [SMILES: c1ccc2cc(-c3nc(-c4ccc(-c5nc6ccc7ccc8sc9ccccc9c8c7c6o5)cc4)nc(-c4ccc5ccccc5c4)n3)ccc2c1 ; c1ccc(-c2nc(-c3ccccc3)nc(-c3ccc(-c4ccc(-c5nc6ccc7ccc8sc9ccccc9c8c7c6o5)cc4)cc3)n2)cc1 ; c1ccc(-c2nc(-c3ccccc3)nc(-c3cccc(-c4ccc(-c5nc6ccc7ccc8sc9ccccc9c8c7c6o5)cc4)c3)n2)cc1]

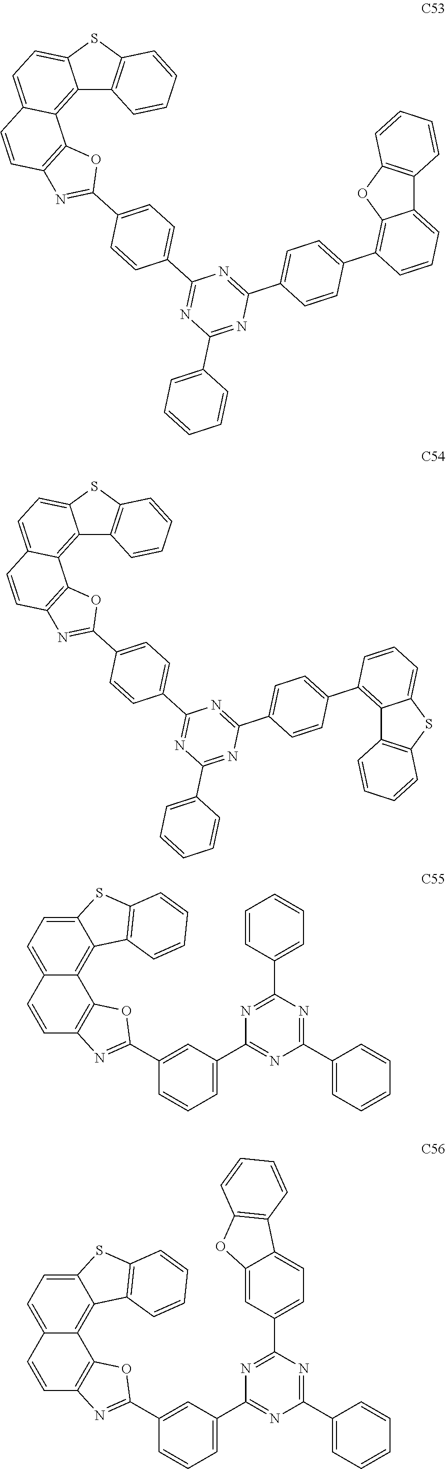

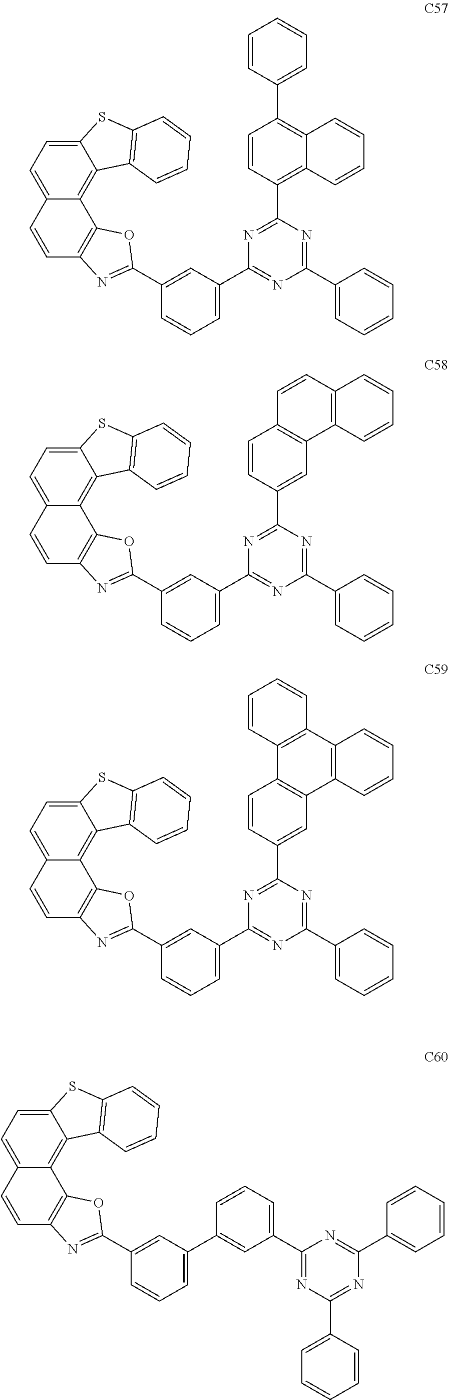

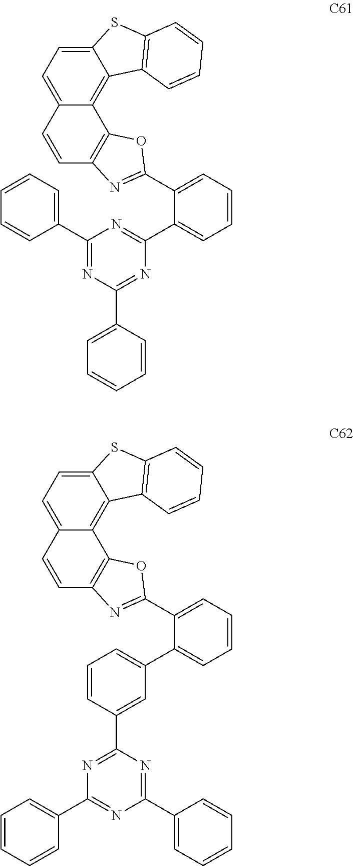 [SMILES: c1ccc(-c2nc(-c3ccccc3)nc(-c3ccccc3-c3nc4ccc5ccc6sc7ccccc7c6c5c4o3)n2)cc1 ; c1ccc(-c2nc(-c3ccccc3)nc(-c3cccc(-c4ccccc4-c4nc5ccc6ccc7sc8ccccc8c7c6c5o4)c3)n2)cc1]

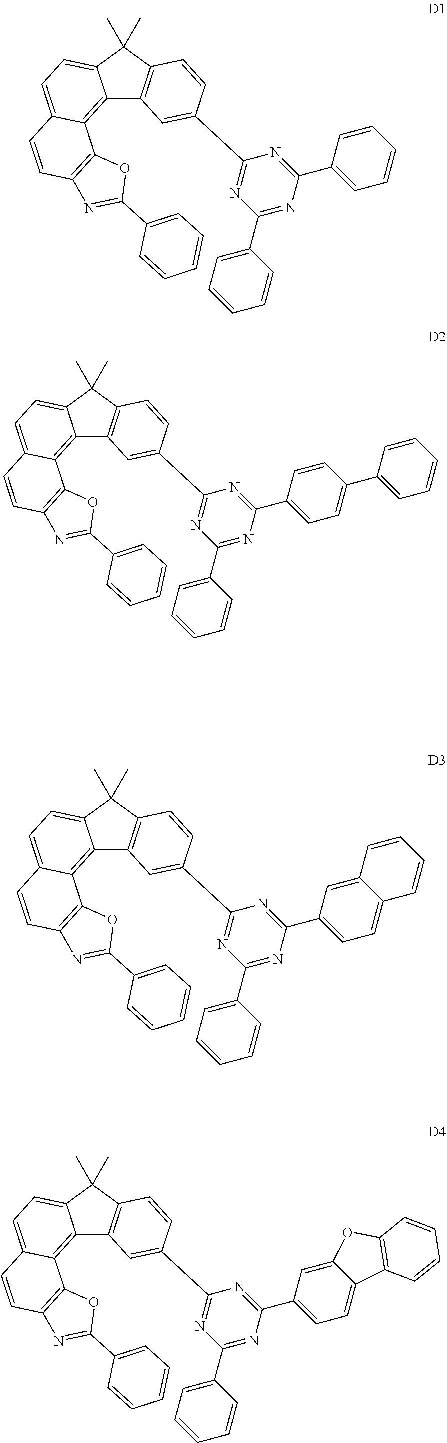 [SMILES: CC1(C)c2ccc(-c3nc(-c4ccccc4)nc(-c4ccccc4)n3)cc2-c2c1ccc1ccc3nc(-c4ccccc4)oc3c21 ; CC1(C)c2ccc(-c3nc(-c4ccccc4)nc(-c4ccc(-c5ccccc5)cc4)n3)cc2-c2c1ccc1ccc3nc(-c4ccccc4)oc3c21 ; CC1(C)c2ccc(-c3nc(-c4ccccc4)nc(-c4ccc5ccccc5c4)n3)cc2-c2c1ccc1ccc3nc(-c4ccccc4)oc3c21 ; CC1(C)c2ccc(-c3nc(-c4ccccc4)nc(-c4ccc5c(c4)oc4ccccc45)n3)cc2-c2c1ccc1ccc3nc(-c4ccccc4)oc3c21]

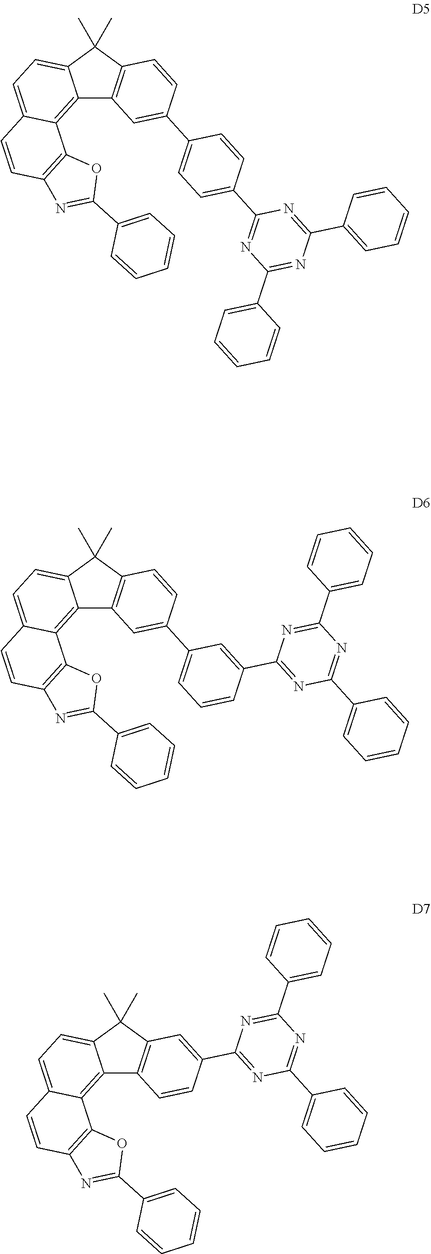 [SMILES: CC1(C)c2ccc(-c3ccc(-c4nc(-c5ccccc5)nc(-c5ccccc5)n4)cc3)cc2-c2c1ccc1ccc3nc(-c4ccccc4)oc3c21 ; CC1(C)c2ccc(-c3cccc(-c4nc(-c5ccccc5)nc(-c5ccccc5)n4)c3)cc2-c2c1ccc1ccc3nc(-c4ccccc4)oc3c21 ; CC1(C)c2cc(-c3nc(-c4ccccc4)nc(-c4ccccc4)n3)ccc2-c2c1ccc1ccc3nc(-c4ccccc4)oc3c21]

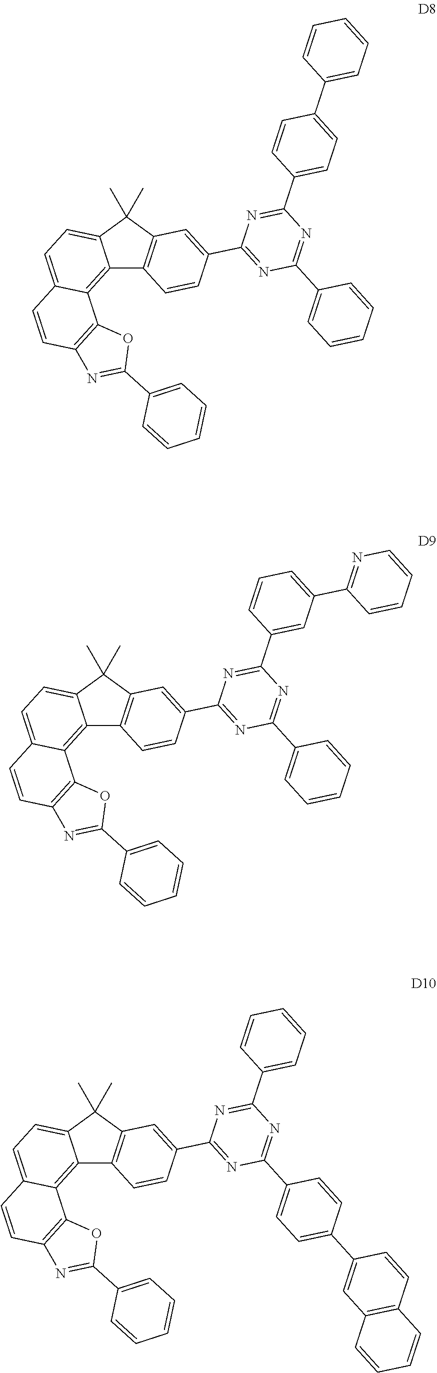 [SMILES: CC1(C)c2cc(-c3nc(-c4ccccc4)nc(-c4ccc(-c5ccccc5)cc4)n3)ccc2-c2c1ccc1ccc3nc(-c4ccccc4)oc3c21 ; CC1(C)c2cc(-c3nc(-c4ccccc4)nc(-c4cccc(-c5ccccn5)c4)n3)ccc2-c2c1ccc1ccc3nc(-c4ccccc4)oc3c21 ; CC1(C)c2cc(-c3nc(-c4ccccc4)nc(-c4ccc(-c5ccc6ccccc6c5)cc4)n3)ccc2-c2c1ccc1ccc3nc(-c4ccccc4)oc3c21]

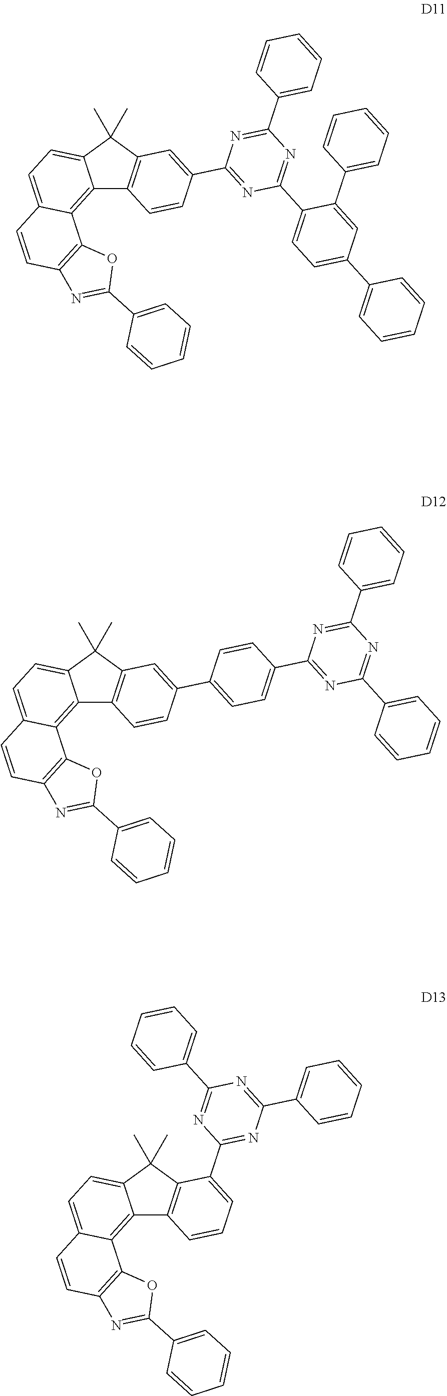 [SMILES: CC1(C)c2cc(-c3nc(-c4ccccc4)nc(-c4ccc(-c5ccccc5)cc4-c4ccccc4)n3)ccc2-c2c1ccc1ccc3nc(-c4ccccc4)oc3c21 ; CC1(C)c2cc(-c3ccc(-c4nc(-c5ccccc5)nc(-c5ccccc5)n4)cc3)ccc2-c2c1ccc1ccc3nc(-c4ccccc4)oc3c21 ; CC1(C)c2ccc3ccc4nc(-c5ccccc5)oc4c3c2-c2cccc(-c3nc(-c4ccccc4)nc(-c4ccccc4)n3)c21]

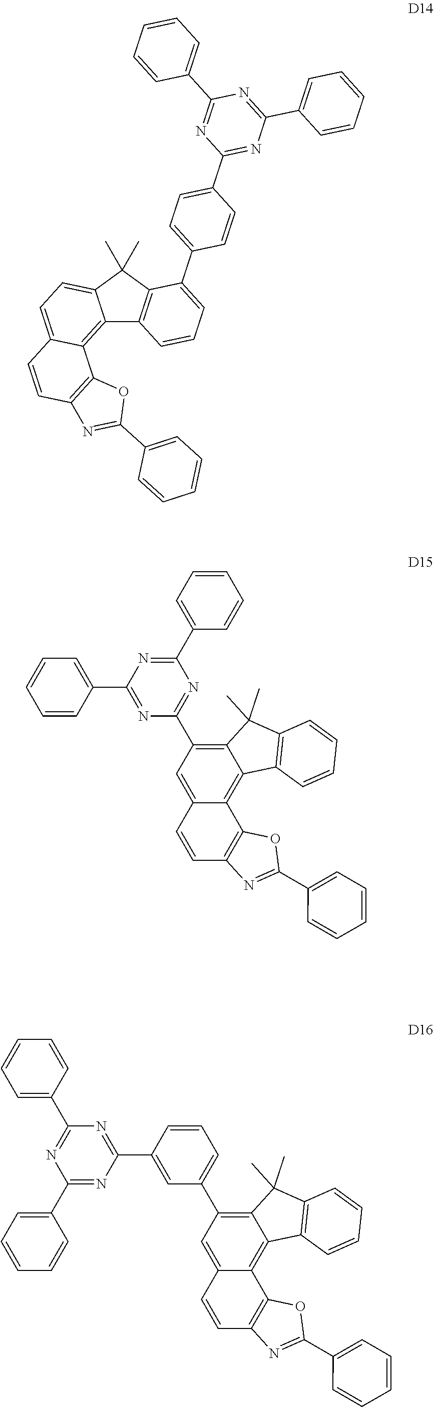 [SMILES: CC1(C)c2ccc3ccc4nc(-c5ccccc5)oc4c3c2-c2cccc(-c3ccc(-c4nc(-c5ccccc5)nc(-c5ccccc5)n4)cc3)c21 ; CC1(C)c2ccccc2-c2c1c(-c1nc(-c3ccccc3)nc(-c3ccccc3)n1)cc1ccc3nc(-c4ccccc4)oc3c21 ; CC1(C)c2ccccc2-c2c1c(-c1cccc(-c3nc(-c4ccccc4)nc(-c4ccccc4)n3)c1)cc1ccc3nc(-c4ccccc4)oc3c21]

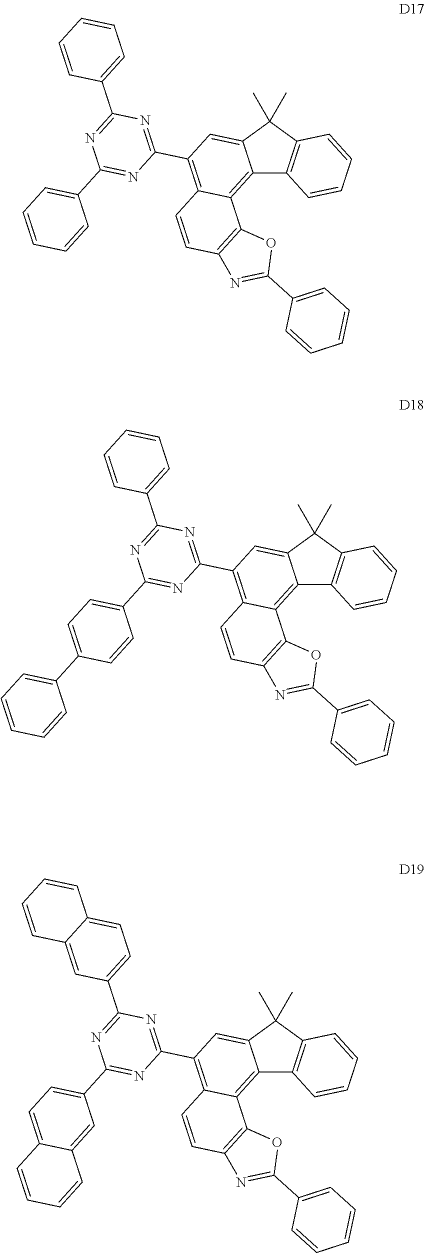 [SMILES: CC1(C)c2ccccc2-c2c1cc(-c1nc(-c3ccccc3)nc(-c3ccccc3)n1)c1ccc3nc(-c4ccccc4)oc3c21 ; CC1(C)c2ccccc2-c2c1cc(-c1nc(-c3ccccc3)nc(-c3ccc(-c4ccccc4)cc3)n1)c1ccc3nc(-c4ccccc4)oc3c21 ; CC1(C)c2ccccc2-c2c1cc(-c1nc(-c3ccc4ccccc4c3)nc(-c3ccc4ccccc4c3)n1)c1ccc3nc(-c4ccccc4)oc3c21]

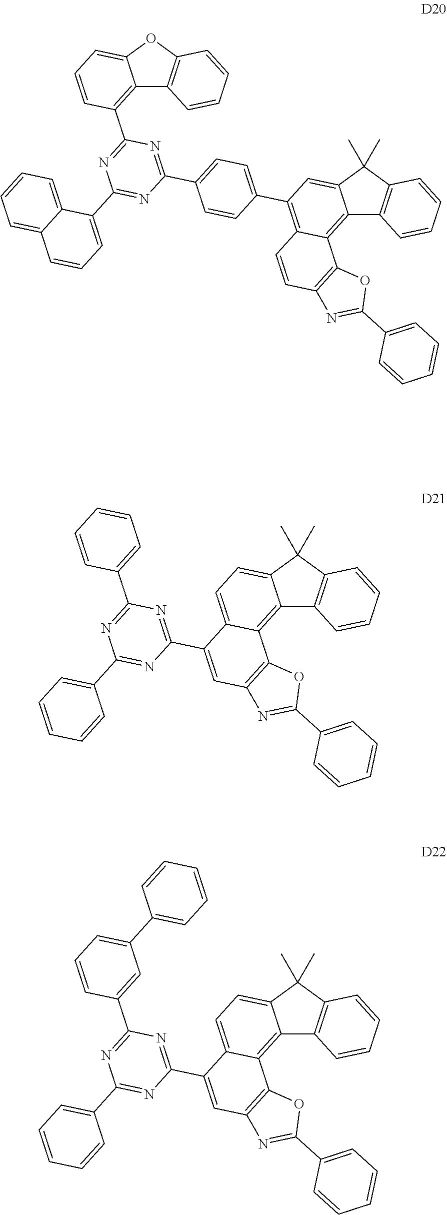 [SMILES: CC1(C)c2ccccc2-c2c1cc(-c1ccc(-c3nc(-c4cccc5ccccc45)nc(-c4cccc5oc6ccccc6c45)n3)cc1)c1ccc3nc(-c4ccccc4)oc3c21 ; CC1(C)c2ccccc2-c2c1ccc1c(-c3nc(-c4ccccc4)nc(-c4ccccc4)n3)cc3nc(-c4ccccc4)oc3c21 ; CC1(C)c2ccccc2-c2c1ccc1c(-c3nc(-c4ccccc4)nc(-c4cccc(-c5ccccc5)c4)n3)cc3nc(-c4ccccc4)oc3c21]

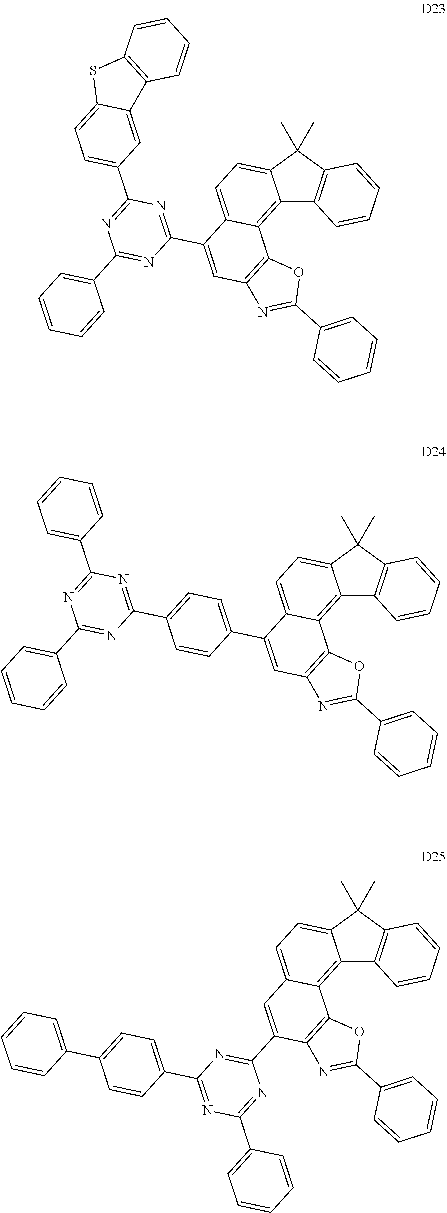 [SMILES: CC1(C)c2ccccc2-c2c1ccc1c(-c3nc(-c4ccccc4)nc(-c4ccc5sc6ccccc6c5c4)n3)cc3nc(-c4ccccc4)oc3c21 ; CC1(C)c2ccccc2-c2c1ccc1c(-c3ccc(-c4nc(-c5ccccc5)nc(-c5ccccc5)n4)cc3)cc3nc(-c4ccccc4)oc3c21 ; CC1(C)c2ccccc2-c2c1ccc1cc(-c3nc(-c4ccccc4)nc(-c4ccc(-c5ccccc5)cc4)n3)c3nc(-c4ccccc4)oc3c21]

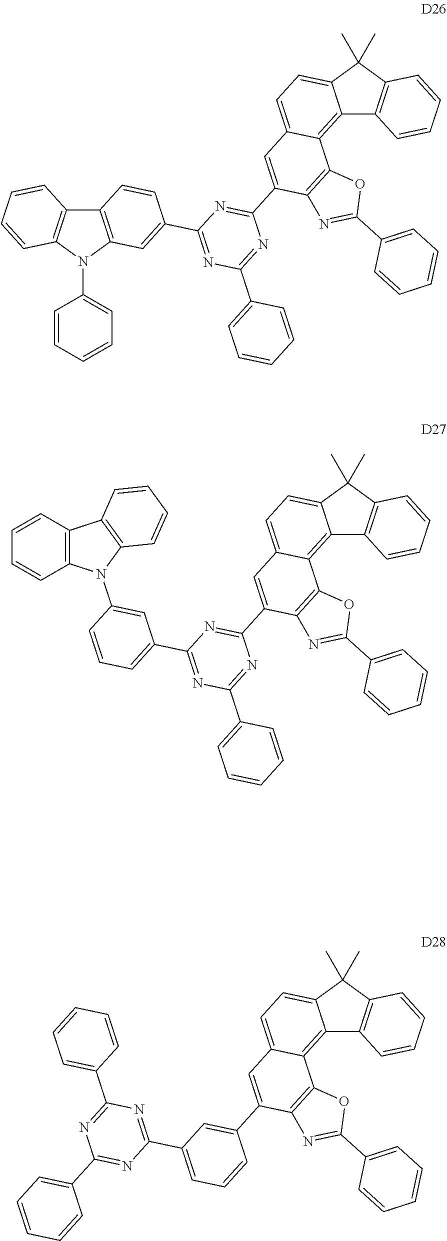 [SMILES: CC1(C)c2ccccc2-c2c1ccc1cc(-c3nc(-c4ccccc4)nc(-c4ccc5c6ccccc6n(-c6ccccc6)c5c4)n3)c3nc(-c4ccccc4)oc3c21 ; CC1(C)c2ccccc2-c2c1ccc1cc(-c3nc(-c4ccccc4)nc(-c4cccc(-n5c6ccccc6c6ccccc65)c4)n3)c3nc(-c4ccccc4)oc3c21 ; CC1(C)c2ccccc2-c2c1ccc1cc(-c3cccc(-c4nc(-c5ccccc5)nc(-c5ccccc5)n4)c3)c3nc(-c4ccccc4)oc3c21]

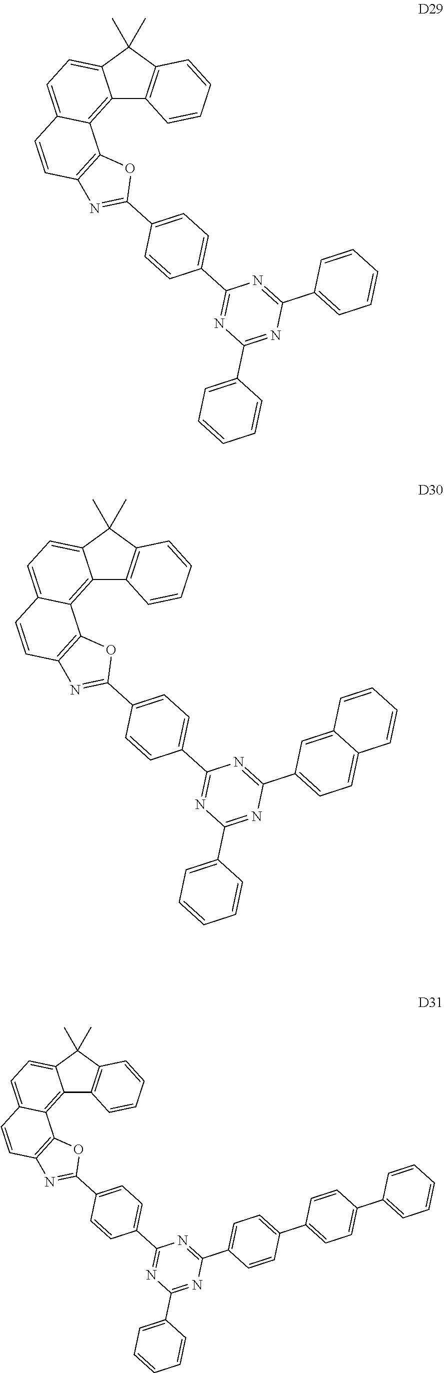 [SMILES: CC1(C)c2ccccc2-c2c1ccc1ccc3nc(-c4ccc(-c5nc(-c6ccccc6)nc(-c6ccccc6)n5)cc4)oc3c21 ; CC1(C)c2ccccc2-c2c1ccc1ccc3nc(-c4ccc(-c5nc(-c6ccccc6)nc(-c6ccc7ccccc7c6)n5)cc4)oc3c21 ; CC1(C)c2ccccc2-c2c1ccc1ccc3nc(-c4ccc(-c5nc(-c6ccccc6)nc(-c6ccc(-c7ccc(-c8ccccc8)cc7)cc6)n5)cc4)oc3c21]

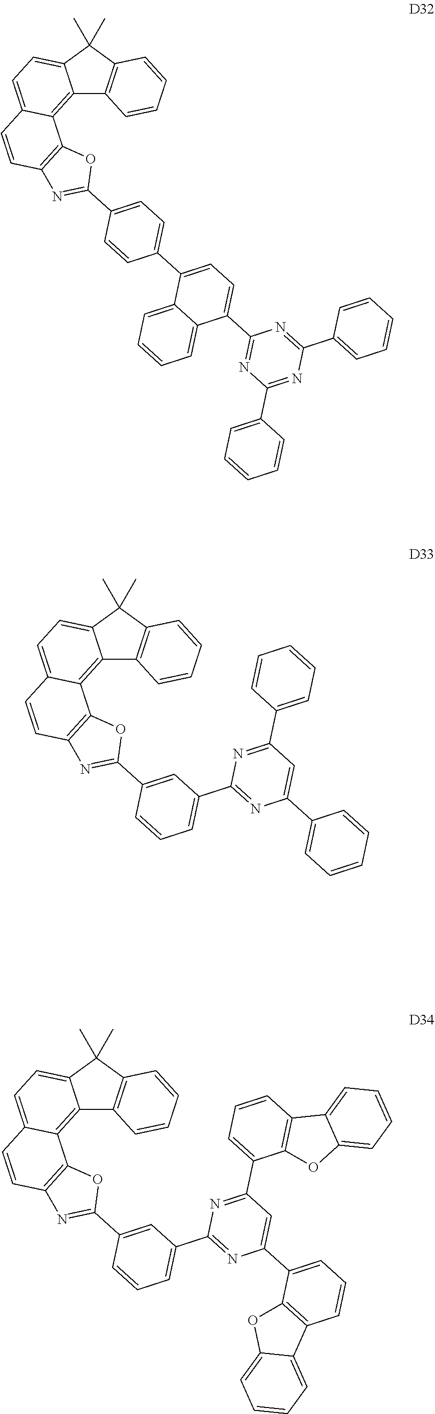 [SMILES: CC1(C)c2ccccc2-c2c1ccc1ccc3nc(-c4ccc(-c5ccc(-c6nc(-c7ccccc7)nc(-c7ccccc7)n6)c6ccccc56)cc4)oc3c21 ; CC1(C)c2ccccc2-c2c1ccc1ccc3nc(-c4cccc(-c5nc(-c6ccccc6)cc(-c6ccccc6)n5)c4)oc3c21 ; CC1(C)c2ccccc2-c2c1ccc1ccc3nc(-c4cccc(-c5nc(-c6cccc7c6oc6ccccc67)cc(-c6cccc7c6oc6ccccc67)n5)c4)oc3c21]

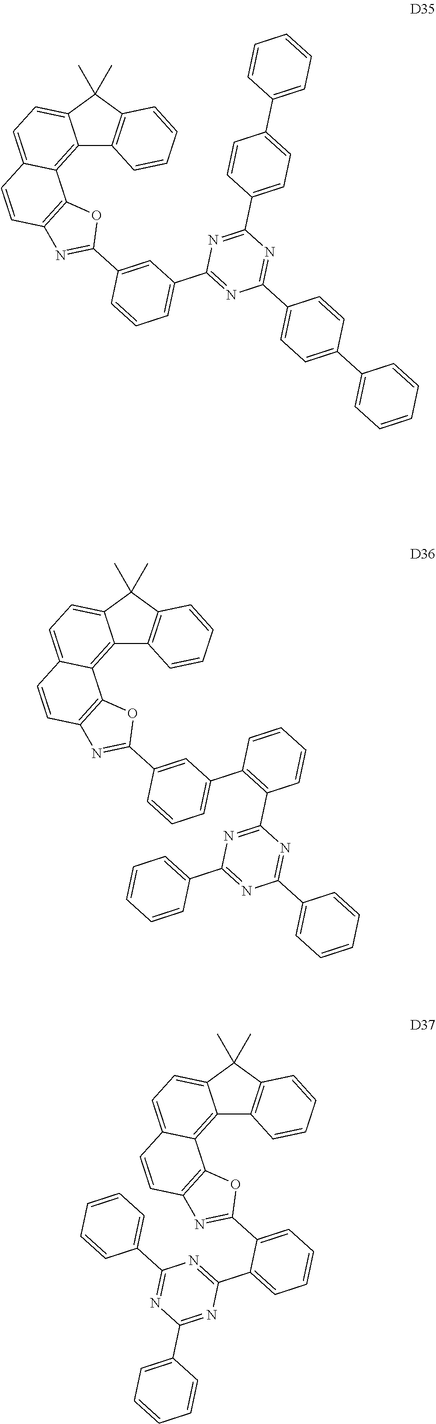 [SMILES: CC1(C)c2ccccc2-c2c1ccc1ccc3nc(-c4cccc(-c5nc(-c6ccc(-c7ccccc7)cc6)nc(-c6ccc(-c7ccccc7)cc6)n5)c4)oc3c21 ; CC1(C)c2ccccc2-c2c1ccc1ccc3nc(-c4cccc(-c5ccccc5-c5nc(-c6ccccc6)nc(-c6ccccc6)n5)c4)oc3c21 ; CC1(C)c2ccccc2-c2c1ccc1ccc3nc(-c4ccccc4-c4nc(-c5ccccc5)nc(-c5ccccc5)n4)oc3c21]

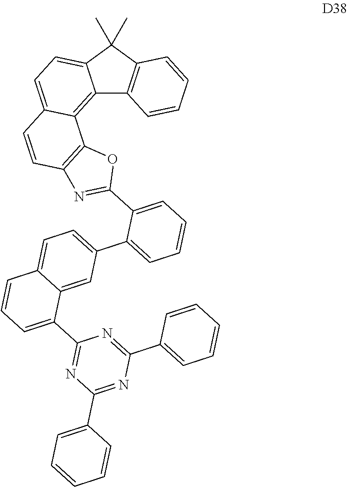 [SMILES: CC1(C)c2ccccc2-c2c1ccc1ccc3nc(-c4ccccc4-c4ccc5cccc(-c6nc(-c7ccccc7)nc(-c7ccccc7)n6)c5c4)oc3c21]

14. An organic electroluminescent device, comprising an anode and a cathode which are oppositely disposed, and a functional layer disposed between the anode and the cathode, wherein
the functional layer comprises the organic compound according to claim 1;
optionally, the functional layer comprises an organic light-emitting layer; and the organic light-emitting layer comprises the organic compound; and
optionally, the organic electroluminescent device is a red organic electroluminescent device.

15. An electronic apparatus, comprising the organic electroluminescent device according to claim 14.
